(19) United States
(12) Patent Application Publication
(10) Pub. No.: US 2026/0055083 A1
(43) Pub. Date: Feb. 26, 2026

(54) APOL1 INHIBITORS AND METHODS OF USE

(71) Applicant: Maze Therapeutics, Inc., South San Francisco, CA (US)

(72) Inventors: Patrick Sang Tae LEE, Walnut Creek, CA (US); Tood Jonathan August EWING, Walnut Creek, CA (US); Adam Neil REID, San Francisco, CA (US); Christopher Joseph SINZ, South San Francisco, CA (US); Birong ZHANG, Union City, CA (US); Sarah M. BRONNER, Oakland, CA (US); David John MORGANS, Los Altos, CA (US)

(21) Appl. No.: 19/104,696

(22) PCT Filed: Aug. 18, 2023
(86) PCT No.: PCT/US2023/072494

§ 371 Date: Feb. 18, 2025

Related U.S. Application Data
(60) Provisional application No. 63/399,638, filed on Aug. 19, 2022.

(51) Int. Cl. C07D 403/12; A61K 31/40; A61K 31/4025; A61K 31/437; A61K 31/4439; A61K 31/451; A61K 31/495; A61K 31/496; A61K 31/497; A61K 31/501; C07D 207/08; C07D 207/09; C07D 211/24; C07D 241/04; C07D 401/12; C07D 405/12; C07D 409/12; C07D 471/04

(52) U.S. Cl. CPC C07D 403/12; A61K 31/40; A61K 31/4025; A61K 31/437; A61K 31/4439; A61K 31/451; A61K 31/495; A61K 31/496; A61K 31/497; A61K 31/501; C07D 207/08; C07D 207/09; C07D 211/24; C07D 241/04; C07D 401/12; C07D 405/12; C07D 409/12; C07D 471/04

(57) ABSTRACT

Provided herein are compounds of formula (A): or a stereoisomer or tautomer thereof, or a pharmaceutically acceptable salt of any of the foregoing, wherein X¹, X², X³, X⁴, Z¹, Z², Z³, Z⁴, R^{a}, R^{b}, R^{c}, R^{d}, R^{e}, L, Y, and m are as defined herein. Also provided N are methods of inhibiting APOL1 and methods of preparing compounds of formula (A). Also provided are methods of inhibiting APOL1 and methods of treating an APOL1-mediated disease, disorder, or condition in an individual.

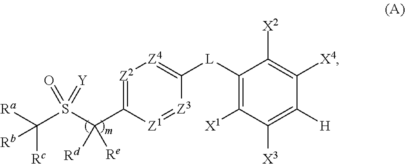

### CROSS REFERENCE TO RELATED APPLICATIONS

This application claims priority to U.S. Provisional Application No. 63/399,638, filed on Aug. 19, 2022, the entire content of which is incorporated herein by reference for all purposes.

### BACKGROUND OF THE INVENTION

Apolipoprotein L1 (APOL1) is a pore forming innate immunity factor, protecting individuals from trypanosome parasites (Vanhamme, L. et al. Nature (2003) 422, 83-87). The secreted form of APOL1 circulates in blood as part of distinct high-density lipoprotein (HDL) complexes, known as trypanosome lytic factors (TLFs) (Rifkin, M. R. Proc. Natl. Acad. Sci. USA. (1978) 75, 3450-3454; Raper, J. et al. Infect. Immun. (1999) 67, 1910-1916). TLFs are internalized by the parasites through endocytosis (Hager, K. M. et al. J. Cell Biol. (1994) 126, 155-167). Within trypanosomes, APOL1 forms cation pores, causing ion flux, swelling, and eventual lysis (Rifkin, M. R. Exp. Parasitol. (1984) 58, 81-93; Molina-Portela, M. P. et al. Mol. Biochem. Parasitol. (2005) 144, 218-226; Perez-Morga, D. et al. Science. (2005) 309, 469-472; Thomson, R. & Finkelstein, A. Proc. Natl. Acad. Sci. USA. (2015) 112, 2894-2899).

Several Trypanosoma brucei subspecies (T.b. rhodesiense and T.b. gambiense) developed resistance mechanisms to APOL1-dependent killing (Pays, E. et al. Nat. Rev. Microbiol. (2014) 12, 575-584). Positive selection resulted in APOL1 variants, G1 (S342G, I384M) and G2 (N388A, Y389A), capable of interfering with these resistance mechanisms (Genovese, G. et al. Science. (2010) 329, 841-845). However, individuals with any binary combination of these variants (G1/G1, G2/G2, or G1/G2), have a greater risk of developing a variety of chronic kidney diseases, including focal segmental glomerulosclerosis (FSGS), hypertension-attributed kidney disease, human immunodeficiency virus-associated nephropathy (HIVAN) (Genovese, G. et al. Science. (2010) 329, 841-845; Tzur, S. et al. Hum. Genet. (2010) 128, 345-350; Kopp, J. B. et al. J. Am. Soc. Nephrol. (2011) 22, 2129-2137), sickle cell nephropathy (Ashley-Koch, A. E. et al. Br. J. Haematol. (2011) 155, 386-394), lupus nephritis (Freedman, B. I. et al. Arthritis Rheumatol. (2014) 66, 390-396), and an increased rate of Glomerular Filtration Rate (GFR) decline in diabetic kidney disease (Parsa, A. et al. N. Engl. J. Med. (2013) 369, 2183-2196). The APOL1 high-risk genotype has also been associated with COVID-19 associated nephropathy and other viral nephropathies (Shetty, A. et al. J. Am. Soc. Nephrol. (2021) 32, 33-40; Chang, J. H. et al. Am. J. Kidney Dis. (2019) 73, 134-139). Moreover, decreased renal allograft survival has been observed after deceased-donor kidney transplantations from APOL1 high-risk genotype donors (Freedman, B. I. et al. Transplantation. (2016) 100, 194-202). In addition, having two APOL1 risk alleles increases risk for preeclampsia (Reidy, K. J. et al. Am. J. Hum. Genet. (2018) 103, 367-376) and sepsis (Chaudhary, N. S. et al. Clin. J. Am. Soc. Nephrol. (2019) 14, 1733-1740). There are no approved therapies for APOL1-associated nephropathy, and patients are treated based on the standard of care for their underlying form of chronic kidney disease. This presents a clear unmet need for therapies targeted to people with the APOL1 high-risk genotype.

Numerous studies have shown that APOL1 risk variants are toxic when overexpressed in human cells (Wan, G. et al. J. Biol. Chem. (2008) 283, 21540-21549; Lan, X. et al. Am. J. Physiol. Renal Physiol. (2014) 307, F326-F336; Olabisi, O. A. et al. Proc. Natd. Acad. Sci. USA. (2016) 113, 830-837; Ma, L. et al. J. Am. Soc. Nephrol. (2017) 28, 1093-1105; Lannon, H. et al. Kidney Int. (2019) 96, 1303-1307). Recent findings suggest that this toxicity is associated with APOL1 pore function (Giovinazzo, J. A. et al. eLife. (2020) 9, e51185). Thus, there is a need to develop compounds suitable for inhibiting APOL1 activity and methods for inhibiting the activity of APOL1 using such compounds.

### BRIEF SUMMARY OF THE INVENTION

This disclosure describes compounds and compositions that may be useful for the treatment of APOL1-mediated diseases, including a variety of chronic kidney diseases such as FSGS, hypertension-attributed kidney disease, HIVAN, sickle cell nephropathy, lupus nephritis, diabetic kidney disease, viral nephropathy, COVID-19 associated nephropathy, and APOL1-associated nephropathy. The compounds and compositions may also find use in treating other APOL1-mediated disorders such as preeclampsia and sepsis. Additionally, for individuals with the APOL1 high-risk genotype, the disclosed compounds and compositions could have utility in preventing the onset of non-diabetic renal disease and/or delaying the progression of any form of chronic kidney disease. The disclosed chemical matter could also have utility in preventing and/or delaying progressive renal allograft loss in patients who have received a kidney transplant from a high-risk APOL1 genotype donor.

In one aspect, provided herein is a compound of formula (A):

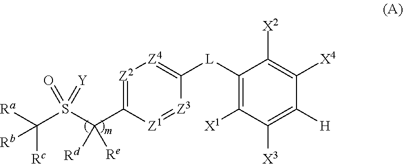

or a stereoisomer or a tautomer thereof, or a pharmaceutically acceptable salt of any of the foregoing, wherein:
m is 0 or 1;
Y is O or —N(C₁₋₆alkyl), wherein the C₁₋₆alkyl of the —N(C₁₋₆alkyl) is optionally substituted with one or more R^{g} substituents;
Z¹, Z², Z³, and Z⁴ are, independently of each other, —N—, —CH— or —C(R^{f})—; R^{a}, R^{b}, and R^{c} are each independently H, C₁₋₆alkyl, C₃₋₆cycloalkyl, or 3-8 membered heterocycle wherein,
   the C₁₋₆alkyl of R^{a}, R^{b}, or R^{c} is optionally substituted with one or more R^{h} substituents, and
   the 3-8 membered heterocycle of R^{a}, R^{b}, or R^{c} is optionally substituted with one or more Rⁱ substituents,
or any two of R^{a}, R^{b}, and R^{c} are taken together with the atoms to which they are attached to form a C₃₋₆cycloalkyl or a 3-8 membered heterocycle, wherein the 3-8 membered heterocycle is optionally substituted with one or more Rⁱ substituents, and the other of R^{a}, R^{b}, and R^{c} is H or C₁₋₆alkyl, C₃₋₆cycloalkyl, or 3-8 membered heterocycle wherein,
   the C₁₋₆alkyl of R^{a}, R^{b}, or R^{c} is optionally substituted with one or more R^{h} substituents, and
   the 3-8 membered heterocycle of R^{a}, R^{b}, or R^{c} is optionally substituted with one or more Rⁱ substituents;
R^{d} and R^{e} are each independently H or C₁₋₆alkyl,
or R^{d} and R^{e} are taken together with the atoms to which they are attached to form a C₃₋₆cycloalkyl or a 3-6 membered heterocycle;
R^{f} is, independently at each occurrence, —CN, halo, C₁₋₆alkyl, C₁₋₆alkoxy, or —N(R^{j})₂, wherein the C₁₋₆alkyl of R^{f} is optionally substituted with one or more halo;
R^{g} is, independently at each occurrence, —S(O)₂C₁₋₆alkyl; R^{h} is, independently at each occurrence, —OH, C₁₋₆alkoxy, —N(R^{j})₂, C(O)R^{k}, or —S(O)₂C₁₋₆alkyl;
Rⁱ is, independently at each occurrence, oxo, C₁₋₆alkyl, or C(O)R^{k};
R^{j} is independently at each occurrence H, C₁₋₆alkyl or C(O)C₁₋₆alkyl;
R^{k} is, independently at each occurrence C₁₋₆alkyl or C₁₋₆alkoxy;
L is selected from the group consisting of:

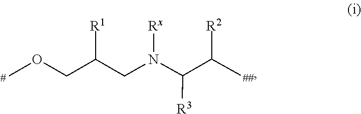

wherein
R^{x} and R¹ are taken together with the atoms to which they are attached to form a 3-8 membered heterocycle, wherein the 3-8 membered heterocycle is substituted with n independently selected R^{m} substituents, wherein n is an integer from 0-6, and R^{m} is C₁₋₆alkyl, or C(O)C₁₋₆alkyl, wherein the C₁₋₆ alkyl is optionally substituted with one or more Rⁿ substituents, and Rⁿ is, independently at each occurrence, —OH;
R² is H, —OH, or C₁₋₆alkyl, wherein the C₁₋₆alkyl of R² is optionally substituted with one or more OH; and
R³ is H or C₁₋₆alkyl, wherein the C₁₋₆alkyl of R³ is optionally substituted with one or more OH;
provided that when L is (i), either:
(1) m is 1,
(2) at least one of Z¹, Z², Z³, and Z⁴ is —N— or —C(R^{f})—,
(3) R³ is other than H,
(4) at least one of R^{a}, R^{b}, and R^{c} is heterocycle, or
(5) at least one of R^{g}, Rⁱ, R^{j}, R^{k}, and Rⁿ is present;

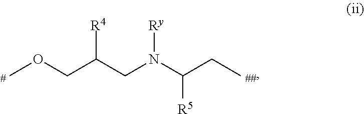

wherein
R^{y}, R⁴, and R⁵ are taken together with the atoms to which they are attached to form a 8-20 membered bicyclic heterocycle, wherein the 8-20 membered bicyclic heterocycle is substituted with n independently selected R^{m} substituents, wherein n is an integer from 0-6, and R^{m} is C₁₋₆alkyl, or C(O)C₁₋₆alkyl, wherein the C₁₋₆alkyl is optionally substituted with one or more Rⁿ substituents, and Rⁿ is, independently at each occurrence, —OH; or

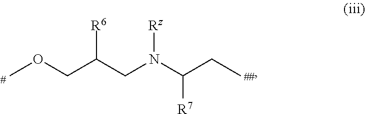

wherein
R^{z} and R⁶ are taken together with the atoms to which they are attached to form a 3-8 membered heterocycleheterocycle substituted with n independently selected R^{m} substituents, wherein n is an integer from 0-6, and R^{m} is C₁₋₆alkyl or C(O)C₁₋₆alkyl, wherein the C₁₋₆ alkyl is optionally substituted with one or more Rⁿ substituents, and Rⁿ is, independently at each occurrence, —OH; and
R⁷ is taken, together with one of X¹ and X² and the atoms to which they are attached, to form a C₄₋₈cycloalkyl;
wherein, for each of (i)-(iii), # denotes the point of attachment to the ring bearing moieties moieties Z¹-Z⁴ and ## denotes the point of attachment to the phenyl ring bearing moieties X¹-X⁴;
X¹, and X² are, independently of each other, H, halo, —CN, C₁₋₆alkyl, C₁₋₆alkoxy, or SF₅, wherein the C₁₋₆alkyl or C₁₋₆alkoxy is optionally substituted with one or more halo,
or one of X¹ and X² is taken together with R⁷ and the atoms to which it is attached to form a C₄₋₈cycloalkyl, and the other of X¹ or X² is H, halo, —CN, C₁₋₆alkyl, C₁₋₆alkoxy, or SF₅, wherein the C₁₋₆alkyl or C₁₋₆alkoxy is optionally substituted with one or more halo; and
X³, and X⁴ are, independently of each other, H, halo, —CN, C₁₋₆alkyl, C₁₋₆alkoxy, or SF₅, wherein the C₁₋₆alkyl or C₁₋₆alkoxy is optionally substituted with one or more halo.

In one aspect, provided herein is a compound of formula (I):

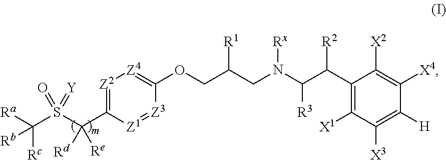

or a stereoisomer or tautomer thereof, or a pharmaceutically acceptable salt of any of the foregoing, wherein X¹, X², X³, X⁴, R¹, R², R³, R^{a}, R^{b}, R^{c}, R^{d}, R^{e}, R^{x}, Z¹, Z², Z³, Z⁴, Y, and m are as defined for a compound of formula (A), or a stereoisomer or tautomer thereof, or a pharmaceutically acceptable salt of any of the foregoing, or any variation or embodiment thereof.

In one aspect, provided herein is a compound of formula (II):

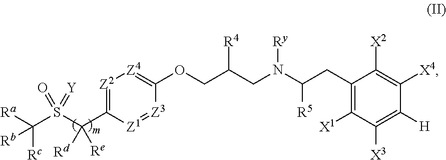

or a stereoisomer or tautomer thereof, or a pharmaceutically acceptable salt of any of the foregoing, wherein X¹, X², X³, X⁴, R⁴, R⁵, R^{a}, R^{b}, R^{c}, R^{d}, R^{e}, R^{y}, Z¹, Z², Z³, Z⁴, Y, and m are as defined for a compound of formula (A), or a stereoisomer or tautomer thereof, or a pharmaceutically acceptable salt of any of the foregoing, or any variation or embodiment thereof.

In one aspect, provided herein is a compound of formula (III):

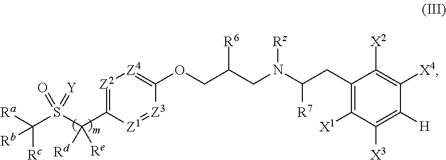

or a stereoisomer or tautomer thereof, or a pharmaceutically acceptable salt of any of the foregoing, wherein X¹, X², X³, X⁴, R⁶, R⁷, R^{a}, R^{b}, R^{c}, R^{d}, R^{e}, R^{z}, Z¹, Z², Z³, Z⁴, Y, and m are as defined for a compound of formula (A), or a stereoisomer or tautomer thereof, or a pharmaceutically acceptable salt of any of the foregoing, or any variation or embodiment thereof.

In one aspect, provided herein is a compound of formula (B):

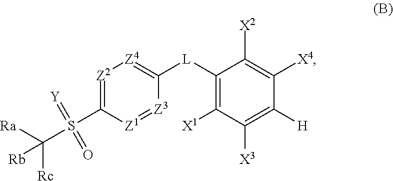

or a stereoisomer or tautomer thereof, or a pharmaceutically acceptable salt of any of the foregoing, wherein X¹, X², X³, X⁴, R^{a}, R^{b}, R^{c}, Z¹, Z², Z³, Z⁴, L, and Y are as defined for a compound of formula (A), or a stereoisomer or tautomer thereof, or a pharmaceutically acceptable salt of any of the foregoing, or any variation or embodiment thereof.

In one aspect, provided herein is a compound of formula (B-2):

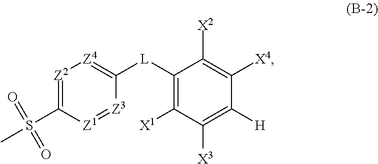

or a stereoisomer or tautomer thereof, or a pharmaceutically acceptable salt of any of the foregoing, wherein X¹, X², X³, X⁴, Z¹, Z², Z³, Z⁴, and L are as defined for a compound of formula (A), or a stereoisomer or tautomer thereof, or a pharmaceutically acceptable salt of any of the foregoing, or any variation or embodiment thereof.

In one aspect, provided herein is a compound of formula (B-4):

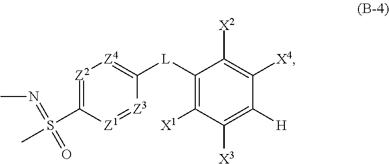

or a stereoisomer or tautomer thereof, or a pharmaceutically acceptable salt of any of the foregoing, wherein X¹, X², X³, X⁴, Z¹, Z², Z³, Z⁴, and L are as defined for a compound of formula (A), or a stereoisomer or tautomer thereof, or a pharmaceutically acceptable salt of any of the foregoing, or any variation or embodiment thereof.

In one aspect, provided herein is a compound of formula (C):

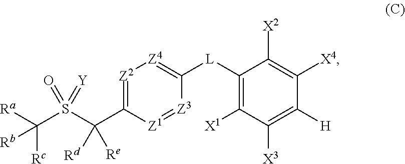

or a stereoisomer or tautomer thereof, or a pharmaceutically acceptable salt of any of the foregoing, wherein X¹, X², X³, X⁴, R^{a}, R^{b}, R^{c}, R^{d}, R^{e}, Z¹, Z², Z³, Z⁴, L and Y are as defined for a compound of formula (A), or a stereoisomer or tautomer thereof, or a pharmaceutically acceptable salt of any of the foregoing, or any variation or embodiment thereof.

In one aspect, provided herein is a compound of formula (C-1):

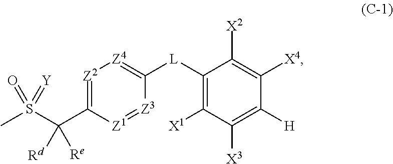

or a stereoisomer or tautomer thereof, or a pharmaceutically acceptable salt of any of the foregoing, wherein X¹, X², X³, X⁴, R^{d}, R^{e}, Z¹, Z², Z³, Z⁴, L and Y are as defined for a compound of formula (A), or a stereoisomer or tautomer thereof, or a pharmaceutically acceptable salt of any of the foregoing, or any variation or embodiment thereof.

In one aspect, provided herein is a compound of formula (D):

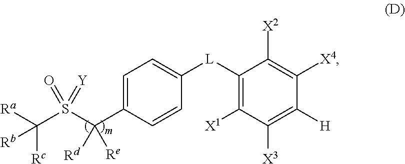

or a stereoisomer or tautomer thereof, or a pharmaceutically acceptable salt of any of the foregoing, wherein X¹, X², X³, X⁴, R^{a}, R^{b}, R^{c}, R^{d}, R^{e}, L, Y, and m are as defined for a compound of formula (A), or a stereoisomer or tautomer thereof, or a pharmaceutically acceptable salt of any of the foregoing, or any variation or embodiment thereof.

In one aspect, provided herein is a pharmaceutical composition, comprising (i) a compound of formula (A), or any embodiment or variation thereof, such as a compound of formula (I), (II), (III), (B), (B-2), (B-4), (C), (C-1), or (D), or a stereoisomer or tautomer thereof, or a pharmaceutically acceptable salt of any of the foregoing, and (ii) one or more pharmaceutically acceptable excipients.

In one aspect, provided herein is a method of modulating APOL1 in a cell, comprising exposing the cell to a composition comprising an effective amount of a compound of formula (A), or any embodiment or variation thereof, such as a compound of formula (I), (II), (III), (B), (B-2), (B-4), (C), (C-1), or (D), or a stereoisomer or tautomer thereof, or a pharmaceutically acceptable salt of any of the foregoing, or a pharmaceutical composition comprising (i) a compound of formula (A), or any embodiment or variation thereof, such as a compound of formula (I), (II), (III), (B), (B-2), (B-4), (C), (C-1), or (D), or a stereoisomer or tautomer thereof, or a pharmaceutically acceptable salt of any of the foregoing, and (ii) one or more pharmaceutically acceptable excipients.

In one aspect, provided herein is a method of inhibiting APOL1 in a cell, comprising exposing the cell to a composition comprising an effective amount of a compound of formula (A), or any embodiment or variation thereof, such as a compound of formula (I), (II), (III), (B), (B-2), (B-4), (C), (C-1), or (D), or a stereoisomer or tautomer thereof, or a pharmaceutically acceptable salt of any of the foregoing, or a pharmaceutical composition comprising (i) a compound of formula (A), or any embodiment or variation thereof, such as a compound of formula (I), (II), (III), (B), (B-2), (B-4), (C), (C-1), or (D), or a stereoisomer or tautomer thereof, or a pharmaceutically acceptable salt of any of the foregoing, and (ii) one or more pharmaceutically acceptable excipients.

In one aspect, provided herein is a method of treating an APOL1-mediated disease, disorder, or condition in an individual in need thereof, comprising administering to the individual an effective amount of a compound of formula (A), or any embodiment or variation thereof, such as a compound of formula (I), (II), (III), (B), (B-2), (B-4), (C), (C-1), or (D), or a stereoisomer or tautomer thereof, or a pharmaceutically acceptable salt of any of the foregoing, or a pharmaceutical composition comprising (i) a compound of formula (A), or any embodiment or variation thereof, such as a compound of formula (I), (II), (III), (B), (B-2), (B-4), (C), (C-1), or (D), or a stereoisomer or tautomer thereof, or a pharmaceutically acceptable salt of any of the foregoing, and (ii) one or more pharmaceutically acceptable excipients.

In one aspect, provided herein is a kit, comprising (i) a compound of formula (A), or any embodiment or variation thereof, such as a compound of formula (I), (II), (III), (B), (B-2), (B-4), (C), (C-1), or (D), or a stereoisomer or tautomer thereof, or a pharmaceutically acceptable salt of any of the foregoing, and (ii) instructions for use in treating an APOL1-mediated disease, disorder, or condition in an individual in need thereof.

In some aspect, provided herein are methods of preparing a compound of formula (A), or any embodiment or variation thereof, such as a compound of formula (I), (II), (III), (B), (B-2), (B-4), (C), (C-1), or (D), or a stereoisomer or tautomer thereof, or a pharmaceutically acceptable salt of any of the foregoing.

### DETAILED DESCRIPTION OF THE INVENTION

Unless clearly indicated otherwise, the terms “a,” “an,” and the like, refer to one or more.

As used herein, “about” a parameter or value includes and describes that parameter or value per se. For example, “about X” includes and describes X per se.

“Individual” refers to mammals and includes humans and non-human mammals. Examples of individuals include, but are not limited to, some primates and humans. In some embodiments, individual refers to a human.

As used herein, an “at risk” individual is an individual who is at risk of developing a disease or condition. An individual “at risk” may or may not have a detectable disease or condition, and may or may not have displayed detectable disease prior to the treatment methods described herein. “At risk” denotes that an individual has one or more so-called risk factors, which are measurable parameters that correlate with development of a disease or condition and are known in the art. An individual having one or more of these risk factors has a higher probability of developing the disease or condition than an individual without these risk factor(s).

“Treatment” or “treating” is an approach for obtaining beneficial or desired results including clinical results. Beneficial or desired results may include one or more of the following: decreasing one or more symptom resulting from the disease or condition; diminishing the extent of the disease or condition; slowing or arresting the development of one or more symptom associated with the disease or condition (e.g., stabilizing the disease or condition, preventing or delaying the worsening or progression of the disease or condition); and relieving the disease, such as by causing the regression of clinical symptoms (e.g., ameliorating the disease state, enhancing the effect of another medication, delaying the progression of the disease, increasing the quality of life, and/or prolonging survival).

As used herein, “delaying” development of a disease or condition means to defer, hinder, slow, retard, stabilize and/or postpone development of the disease or condition. This delay can be of varying lengths of time, depending on the history of the disease and/or individual being treated. As is evident to one skilled in the art, a sufficient or significant delay can, in effect, encompass prevention, in that the individual does not develop the disease or condition.

As used herein, the term “therapeutically effective amount” or “effective amount” intends such amount of a compound of the disclosure or a pharmaceutically salt thereof sufficient to effect treatment when administered to an individual. As is understood in the art, an effective amount may be in one or more doses, e.g., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved.

As used herein, “unit dosage form” refers to physically discrete units, suitable as unit dosages, each unit containing a predetermined quantity of active ingredient, or compound, which may be in a pharmaceutically acceptable carrier.

As used herein, by “pharmaceutically acceptable” is meant a material that is not biologically or otherwise undesirable, e.g., the material may be incorporated into a pharmaceutical composition administered to an individual without causing significant undesirable biological effects.

The term “alkyl”, as used herein, refers to an unbranched or branched saturated hydrocarbon chain. As used herein, alkyl has 1-20 carbons (i.e., C₁₋₂₀alkyl), 1-16 carbons (i.e., C₁₋₁₆alkyl), 1-12 carbons (i.e., C₁₋₁₂alkyl), 1-10 carbons (i.e., C₁₋₁₀alkyl), 1-8 carbons (i.e., C₁. 8alkyl), 1-6 carbons (i.e., C₁₋₆alkyl), 1-4 carbons (i.e., C₁₋₄alkyl), or 1-3 carbons (i.e., C₁₋₃alkyl). Examples of alkyl groups include, but are not limited to, methyl, ethyl, propyl, iso-propyl, n-butyl, sec-butyl, iso-butyl, tert-butyl, pentyl, 2-pentyl, iso-pentyl, neo-pentyl, hexyl, 2-hexyl, 3-hexyl, and 3-methylpentyl. When an alkyl residue having a specific number of carbons is named by chemical name or molecular formula, all positional isomers having that number of carbon atoms may be encompassed—for example, “butyl” includes n-butyl, sec-butyl, iso-butyl, and tert-butyl; and “propyl” includes n-propyl and iso-propyl. Certain commonly used alternative names may be used and will be understood by those of ordinary skill in the art. For instance, a divalent group, such as a divalent “alkyl” group, may be referred to as an “alkylene”.

The term “alkoxy”, as used herein, refers to an —O-alkyl moiety. Examples of alkoxy groups include, but are not limited to, methoxy, ethoxy, n-propoxy, iso-propoxy, n-butoxy, tert-butoxy, sec-butoxy, n-pentoxy, n-hexoxy, and 1,2-dimethylbutoxy.

The term “aryl”, as used herein, refers to a fully unsaturated carbocyclic ring moiety. The term “aryl” encompasses monocyclic and polycyclic fused-ring moieties. As used herein, aryl encompasses ring moieties comprising, for example, 6 to 20 annular carbon atoms (i.e., C₆₋₂₀aryl), 6 to 16 annular carbon atoms (i.e., C₆₋₁₆aryl), 6 to 12 annular carbon atoms (i.e., C₆₋₁₂aryl), or 6 to 10 annular carbon atoms (i.e., C₆₋₁₀aryl). Examples of aryl moieties include, but are not limited to, phenyl, naphthyl, fluorenyl, and anthryl.

The term “cycloalkyl”, as used herein, refers to a saturated or partially unsaturated carbocyclic ring moiety. The term “cycloalkyl” encompasses monocyclic and polycyclic ring moieties, wherein the polycyclic moieties may be fused, branched, or spiro. Cycloalkyl includes cycloalkenyl groups, wherein the ring moiety comprises at least one annular double bond. Cycloalkyl includes any polycyclic carbocyclic ring moiety comprising at least one non-aromatic ring, regardless of the point of attachment to the remainder of the molecule. As used herein, cycloalkyl includes rings comprising, for example, 3 to 20 annular carbon atoms (i.e., a C₃₋₂₀cycloalkyl), 3 to 16 annular carbon atoms (i.e., a C₃₋₁₆cycloalkyl), 3 to 12 annular carbon atoms (i.e., a C₃₋₁₂cycloalkyl), 3 to 10 annular carbon atoms (i.e., a C₃₋₁₀cycloalkyl), 3 to 8 annular carbon atoms (i.e., a C₃₋₈cycloalkyl), 3 to 6 annular carbon atoms (i.e., a C₃₋₆cycloalkyl), or 3 to 5 annular carbon atoms (i.e., a C₃₋₅cycloalkyl). Monocyclic cycloalkyl ring moieties include, for example, cyclopropyl, cyclobutyl, cyclopentyl, cyclohexyl, cycloheptyl, and cyclooctyl. Polycyclic groups include, for example, bicyclo[2.2.1]heptanyl, bicyclo[2.2.2]octanyl, adamantyl, norbomyl, decalinyl, 7,7-dimethyl-bicyclo [2.2.1]heptanyl, and the like. Still further, cycloalkyl also includes spiro cycloalkyl ring moieties, for example, spiro[2.5]octanyl, spiro[4.5]decanyl, or spiro [5.5]undecanyl.

The term “halo”, as used herein, refers to atoms occupying groups VIIA of The Periodic Table and includes fluorine (fluoro), chlorine (chloro), bromine (bromo), and iodine (iodo).

The term “heteroaryl”, as used herein, refers to an aromatic (fully unsaturated) ring moiety that comprises one or more annular heteroatoms independently selected from the group consisting of nitrogen, oxygen, and sulfur. The term “heteroaryl” includes both monocyclic and polycyclic fused-ring moieties. As used herein, a heteroaryl comprises, for example, 5 to 20 annular atoms (i.e., a 5-20 membered heteroaryl), 5 to 16 annular atoms (i.e., a 5-16 membered heteroaryl), 5 to 12 annular atoms (i.e., a 5-12 membered heteroaryl), 5 to 10 annular atoms (i.e., a 5-10 membered heteroaryl), 5 to 8 annular atoms (i.e., a 5-8 membered heteroaryl), or 5 to 6 annular atoms (i.e., a 5-6 membered heteroaryl). Any monocyclic or polycyclic aromatic ring moiety comprising one or more annular heteroatoms is considered a heteroaryl, regardless of the point of attachment to the remainder of the molecule (i.e., the heteroaryl moiety may be attached to the remainder of the molecule through any annular carbon or any annular heteroatom of the heteroaryl moiety). Examples of heteroaryl groups include, but are not limited to, acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, triazolyl, tetrazolyl, and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl, and imidazo[1,5-a]pyridinyl, wherein the heteroaryl can be bound via either ring of the fused system.

The term “heterocyclyl”, as used herein, refers to a saturated or partially unsaturated cyclic moiety that encompasses one or more annular heteroatoms independently selected from the group consisting of nitrogen, oxygen, and sulfur. The term “heterocyclyl” includes both monocyclic and polycyclic ring moieties, wherein the polycyclic ring moieties may be fused, bridged, or spiro. Any non-aromatic monocyclic or polycyclic ring moiety comprising at least one annular heteroatom is considered a heterocyclyl, regardless of the point of attachment to the remainder of the molecule (i.e., the heterocyclyl moiety may be attached to the remainder of the molecule through any annular carbon or any annular heteroatom of the heterocyclyl moiety). Further, the term heterocyclyl is intended to encompass any polycyclic ring moiety comprising at least one annular heteroatom wherein the polycyclic ring moiety comprises at least one non-aromatic ring, regardless of the point of attachment to the remainder of the molecule. As used herein, a heterocyclyl comprises, for example, 3 to 20 annular atoms (i.e., a 3-20 membered heterocyclyl), 3 to 16 annular atoms (i.e., a 3-16 membered heterocyclyl), 3 to 12 annular atoms (i.e., a 3-12 membered heterocyclyl), 3 to 10 annular atoms (i.e., a 3-10 membered heterocyclyl), 3 to 8 annular atoms (i.e., a 3-8 membered heterocyclyl), 3 to 6 annular atoms (i.e., a 3-6 membered heterocyclyl), 3 to 5 annular atoms (i.e., a 3-5 membered heterocyclyl), 5 to 8 annular atoms (i.e., a 5-8 membered heterocyclyl), or 5 to 6 annular atoms (i.e., a 5-6 membered heterocyclyl). Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiophenyl (i.e., thienyl), thiomorpholinyl, thiamorpholinyl, 1-oxo-thiomorpholinyl, and 1,1-dioxo-thiomorpholinyl. Examples of spiro heterocyclyl rings include, but are not limited to, bicyclic and tricyclic ring systems, such as oxabicyclo[2.2.2]octanyl, 2-oxa-7-azaspiro[3.5]nonanyl, 2-oxa-6-azaspiro[3.4]octanyl, and 6-oxa-1-azaspiro[3.3]heptanyl. Examples of fused heterocyclyl rings include, but are not limited to, 1,2,3,4-tetrahydroisoquinolinyl, 4,5,6,7-tetrahydrothieno[2,3-c]pyridinyl, indolinyl, and isoindolinyl, where the heterocyclyl can be bound via either ring of the fused system.

The terms “optional” and “optionally”, as used herein, mean that the subsequently described event or circumstance may or may not occur and that the description includes instances where the event or circumstance occurs and instances where it does not. Accordingly, the term “optionally substituted” infers that any one or more (e.g., 1, 2, 1 to 5, 1 to 3, 1 to 2, etc.) hydrogen atoms on the designated atom or moiety or group may be replaced or not replaced by an atom or moiety or group other than hydrogen. By way of illustration and not limitation, the phrase “methyl optionally substituted with one or more chloro” encompasses —CH₃, —CH₂Cl, —CHCl₂, and —CCl₃ moieties.

It is understood that aspects and embodiments described herein as “comprising” include “consisting of” and “consisting essentially of” embodiments.

The term “pharmaceutically acceptable salt”, as used herein, of a given compound refers to salts that retain the biological effectiveness and properties of the given compound and which are not biologically or otherwise undesirable. “Pharmaceutically acceptable salts” include, for example, salts with inorganic acids, and salts with an organic acid. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. See, e.g., Handbook of Pharmaceutical Salts Properties, Selection, and Use, International Union of Pure and Applied Chemistry, John Wiley & Sons (2008), which is incorporated herein by reference. Those skilled in the art will recognize various synthetic methodologies that may be used to prepare nontoxic pharmaceutically acceptable addition salts. Pharmaceutically acceptable acid addition salts may be prepared from inorganic or organic acids. Salts derived from inorganic acids include, e.g., hydrochloric acid, hydrobromic acid, sulfuric acid, nitric acid, phosphoric acid, and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, trifluoroacetic acid, and the like. Likewise, pharmaceutically acceptable base addition salts can be prepared from inorganic or organic bases. Salts derived from inorganic bases include, by way of example only, sodium, potassium, lithium, aluminum, ammonium, calcium, and magnesium salts. Salts derived from organic bases include, but are not limited to, salts of primary, secondary, and tertiary amines. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

Isotopically labeled forms of the compounds depicted herein may be prepared. Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I and ¹²⁵I, respectively. In some embodiments, a compound of formula (A) is provided wherein one or more hydrogen is replaced by deuterium or tritium.

In some embodiments, compounds described herein contain one or more hydrogen atoms that are replaced with deuterium, wherein deuterium is present in an amount that is greater than its natural abundance. Thus, as used herein, designation of an atom as deuterium at a position indicates that the abundance of deuterium is significantly greater than the natural abundance of deuterium. Unless otherwise stated, when a position is designated specifically as “H” or “hydrogen,” the position is understood to have hydrogen at its naturally abundant isotopic composition. Also unless otherwise stated, when a position is designated specifically as “D” or “deuterium,” the position is understood to have deuterium at an abundance that is significantly greater than the natural abundance of deuterium, e.g., at least 3000 times greater than the natural abundance of deuterium, which is about 0.015% (i.e., the term “D” or “deuterium” indicates at least about 45% incorporation of deuterium).

Some of the compounds provided herein may exist as tautomers. Tautomers are in equilibrium with one another. By way of illustration, amide containing compounds may exist in equilibrium with imidic acid tautomers. Regardless of which tautomer is shown and regardless of the nature of the equilibrium among tautomers, the compounds of this disclosure are understood by one of ordinary skill in the art to comprise both amide and imidic acid tautomers. Thus, for example, amide-containing compounds are understood to include their imidic acid tautomers. Likewise, imidic-acid containing compounds are understood to include their amide tautomers.

Also provided herein are prodrugs of the compounds depicted herein, or a pharmaceutically acceptable salt thereof. Prodrugs are compounds that may be administered to an individual and release, in vivo, a compound depicted herein as the parent drug compound. It is understood that prodrugs may be prepared by modifying a functional group on a parent drug compound in such a way that the modification is cleaved in vivo to release the parent drug compound. See, e.g., Rautio, J., Kumpulainen, H., Heimbach, T. et al. Prodrugs: design and clinical applications. Nat Rev Drug Discov 7, 255-270 (2008), which is incorporated herein by reference. In certain variations prodrugs are compounds that may release, in vivo or in vitro, a compound depicted herein as the parent drug compound.

The compounds of the present disclosure, or their pharmaceutically acceptable salts, may include an asymmetric center and may thus give rise to enantiomers, diastereomers, and other stereoisomeric forms that may be defined, in terms of absolute stereochemistry, as (R)- or (S)- (or as (D)- or (L)- for amino acids). The present disclosure is meant to include all such possible isomers, as well as their racemic and optically pure forms and mixtures thereof in any ratio. Optically active (+) and (−), (R)- and (S)-, or (D)- and (L)-isomers may be prepared using chiral synthons or chiral reagents, or may be resolved using conventional techniques, for example, chromatography and/or fractional crystallization. Conventional techniques for the preparation/isolation of individual enantiomers include chiral synthesis from a suitable optically pure precursor or the resolution of the racemate (or the racemate of a salt or derivative) using, for example, chiral high pressure liquid chromatography or high performance liquid chromatography (HPLC), and chiral SFC (supercritical fluid chromatography). When the compounds described herein contain olefinic double bonds or other centers of geometric asymmetry, unless specified otherwise, it is intended that the present disclosure includes both E and Z geometric isomers. Likewise, cis- and trans- are used in their conventional sense to describe relative spatial relationships.

A “stereoisomer” refers to a compound made up of the same atoms bonded by the same bonds, but having different three-dimensional structures, which are not interchangeable. The present disclosure contemplates various stereoisomers, or mixtures thereof, and includes “enantiomers,” which refers to two stereoisomers whose structures are non-superimposable mirror images of one another. “Diastereomers” are stereoisomers that have at least two asymmetric atoms, but which are not mirror images of each other.

Where enantiomeric and/or diastereomeric forms exist of a given structure, flat bonds indicate that all stereoisomeric forms of the depicted structure may be present, e.g.,

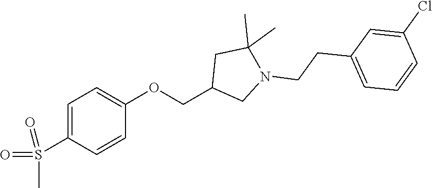 [SMILES: CC1(C)CC(COc2ccc(S(C)(=O)=O)cc2)CN1CCc1cccc(Cl)c1]

Where enantiomeric forms exist of a given structure, flat bonds and the presence of a “*” symbol indicate that the composition is made up of at least 90%, by weight, of a single isomer with unknown stereochemistry, e.g.,

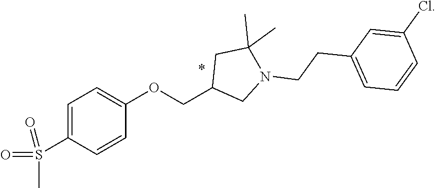 [SMILES: CC1(C)CC(COc2ccc(S(C)(=O)=O)cc2)CN1CCc1cccc(Cl)c1]

Where enantiomeric and/or diastereomeric forms exist of a given structure, wedged or hashed bonds indicate the composition is made up of at least 90%, by weight, of a single enantiomer or diastereomer with known stereochemistry, e.g.,

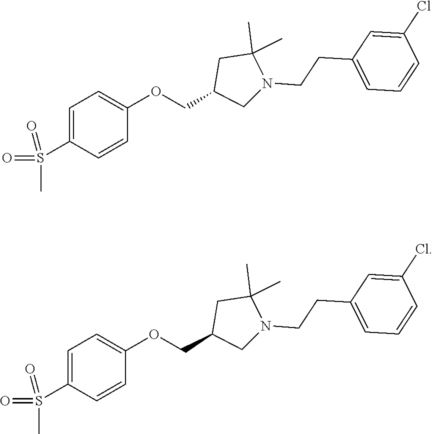 [SMILES: CC1(C)C[C@@H](COc2ccc(S(C)(=O)=O)cc2)CN1CCc1cccc(Cl)c1 ; CC1(C)C[C@H](COc2ccc(S(C)(=O)=O)cc2)CN1CCc1cccc(Cl)c1]

Where enantiomeric and/or diastereomeric forms exist of a given structure with two stereocenters, flat bonds and the presence of two “&1” symbols indicate the composition is made up of a pair of enantiomers with unknown relative stereochemistry, e.g.,

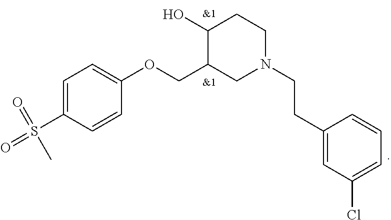 [SMILES: CS(=O)(=O)c1ccc(OCC2CN(CCc3cccc(Cl)c3)CCC2O)cc1]

Where enantiomeric and/or diastereomeric forms exist of a given structure with two stereocenters, wedged and/or dashed bonds and the presence of two “&1” symbols indicate the composition is made up of a pair of enantiomers with known relative stereochemistry, e.g.,

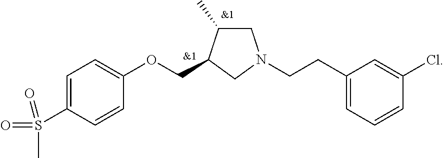 [SMILES: C[C@@H]1CN(CCc2cccc(Cl)c2)C[C@H]1COc1ccc(S(C)(=O)=O)cc1]

Where enantiomeric and/or diastereomeric forms exist of a given structure with two stereocenters, wedged and/or dashed bonds and the presence of two “or1” symbols indicate the composition is made up of at least 90%, by weight, a single stereoisomer with known relative stereochemistry but unknown absolute stereochemistry, e.g.,

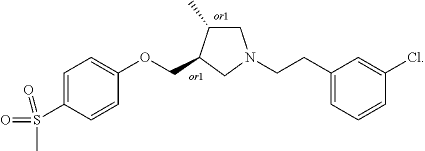 [SMILES: C[C@@H]1CN(CCc2cccc(Cl)c2)C[C@H]1COc1ccc(S(C)(=O)=O)cc1]

Where enantiomeric and/or diastereomeric forms exist of a given structure with two stereocenters, flat bonds and the presence of two “*” symbols indicate the composition is made up of at least 90%, by weight, of a single enantiomer or diastereomer with unknown stereochemistry, e.g.,

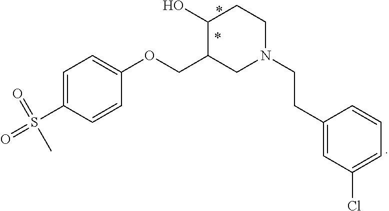 [SMILES: CS(=O)(=O)c1ccc(OCC2CN(CCc3cccc(Cl)c3)CCC2O)cc1]

Abbreviations used are those conventional in the art and are in accordance with the Periodic Table of the Elements, CAS version, Handbook of Chemistry and Physics, 75^{th} Ed. The following examples are intended to be illustrative only and not limiting in any way.

| | | | |
|---|---|---|---|
| ° C. | degrees Celsius | AIBN | azobisisobutyronitrile |
| μL | microliter | app | apparent (NMR) |
| [M + XX]⁺ | observed mass | br | broad (as in “br s” to |
| AC₅₀ | half-maximal activity | | indicate a broad singlet) |
| | concentration | BH₃ · THF | borane-tetrahydrofuran |
| AcOH | acetic acid | | complex |
| BBr₃ | boron tribromide | LiHMDS | lithium |
| Calc'd | calculated | | bis(trimethylsilyl)amide |
| Cbz-Cl or | benzyl chloroformate | mCPBA or | meta-chloroperoxybenzoic |
| CbzCl | | m-CPBA | acid |
| CO₂ | carbon dioxide | MeOH | methanol |
| Cs₂CO₃ | cesium carbonate | MeCN | acetonitrile |
| d | deuterated (NMR solvents) | m | multiplet (NMR) |
| d | doublet (NMR) | mg | milligrams |
| dd | doublet of doublets (NMR) | min | minutes |
| DCE | 1,2-dichloroethane | mL | milliliter |
| DCM | dichloromethane | mmol | millimole |
| DIAD | diisopropyl | mM | millimolar |
| | azodicarboxylate | M | molarity or molar |
| DMF | N,N-dimethylformamide | MS | mass spectrometry |
| DMP | Dess-Martin periodinane | MsCl | methanesulfonyl chloride |
| EC₅₀ | half-maximal effective | MTBE | methyl tert-butyl ether |
| | concentration | n/a | not applicable |
| EtOAc | ethyl acetate | NH₄ | ammonium |
| EtOH | ethanol | NH₄OH | ammonium hydroxide |
| g | grams | NH₄HCO₃ | ammonium bicarbonate |
| h | hours | Na₂SO₄ | sodium sulfate |
| H | hydrogen | NaBH₃CN | sodium cyanoborohydride |
| HCl | hydrochloric acid | NMR | nuclear magnetic resonance |
| HPLC | high-performance liquid | NaOH | sodium hydroxide |
| | chromatography | Pd(dppf)Cl₂ | [1,1′- |
| In vacuo | in a vacuum | | bis(diphenylphosphino)ferro |
| IUPAC | International Union of Pure | | cene]dichloropalladium(II) |
| | and Applied Chemistry | Pd/C | palladium on carbon |
| MHz | megahertz | pH | potential of hydrogen |
| J | J-coupling value (NMR) | PPh₃ | triphenyl phosphine |
| K₂CO₃ | potassium carbonate | s | singlet (NMR) |
| SFC | supercritical fluid | TFA | trifluoroacetic acid |
| | chromatography | THF | tetrahydrofuran |
| t | triplet (NMR) | TMAD | tetramethylazodicarboxamide |
| TBAB | tetrabutylammonium | | |
| | bromide | TMSCl | trimethylsilyl chloride |
| TEA | triethylamine | | |

### Compounds

Provided herein is a compound of formula (A):

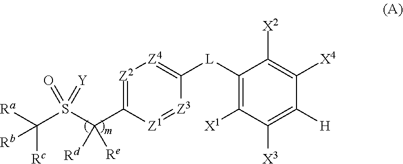

or a stereoisomer or a tautomer thereof, or a pharmaceutically acceptable salt of any of the foregoing, wherein:
m is 0 or 1;
Y is O or —N(C₁₋₆alkyl), wherein the C₁₋₆alkyl of the —N(C₁₋₆alkyl) is optionally substituted with one or more R^{g} substituents;
Z¹, Z², Z³, and Z⁴ are, independently of each other, —N—, —CH— or —C(R^{f})—;
R^{a}, R^{b}, and R^{c} are each independently H, C₁₋₆alkyl, C₃₋₆cycloalkyl, or 3-8 membered heterocycle wherein,
   the C₁₋₆alkyl of R^{a}, R^{b}, or R^{c} is optionally substituted with one or more R^{h} substituents, and
   the 3-8 membered heterocycle of R^{a}, R^{b}, or R^{c} is optionally substituted with one or more Rⁱ substituents,
or any two of R^{a}, R^{b}, and R^{c} are taken together with the atoms to which they are attached to form a C₃₋₆cycloalkyl or a 3-8 membered heterocycle, wherein the 3-8 membered heterocycle is optionally substituted with one or more Rⁱ substituents, and the other of R^{a}, R^{b}, and R^{c} is H or C₁₋₆alkyl, C₃₋₆cycloalkyl, or 3-8 membered heterocycle wherein,
   the C₁₋₆alkyl of R^{a}, R^{b}, or R^{c} is optionally substituted with one or more R^{h} substituents, and
   the 3-8 membered heterocycle of R^{a}, R^{b}, or R^{c} is optionally substituted with one or more Rⁱ substituents;
R^{d} and R^{e} are each independently H or C₁₋₆alkyl,
or R^{d} and R^{e} are taken together with the atoms to which they are attached to form a C₃₋₆cycloalkyl or a 3-6 membered heterocycle;
R^{f} is, independently at each occurrence, —CN, halo, C₁₋₆alkyl, C₁₋₆alkoxy, or —N(R^{j})₂, wherein the C₁₋₆alkyl of R^{f} is optionally substituted with one or more halo;
R^{g} is, independently at each occurrence, —S(O)₂C₁₋₆alkyl;
R^{h} is, independently at each occurrence, —OH, C₁₋₆alkoxy, —N(R^{j})₂, C(O)R^{k}, or —S(O)₂C₁₋₆alkyl;
Rⁱ is, independently at each occurrence, oxo, C₁₋₆alkyl, or C(O)R^{k};
R^{j} is independently at each occurrence H, C₁₋₆alkyl or C(O)C₁₋₆alkyl;
R^{k} is, independently at each occurrence C₁₋₆alkyl or C₁₋₆alkoxy;
L is selected from the group consisting of:

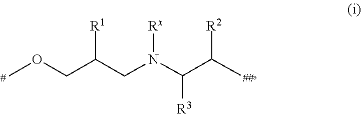

wherein
R^{x} and R¹ are taken together with the atoms to which they are attached to form a 3-8 membered heterocycle, wherein the 3-8 membered heterocycle is substituted with n independently selected R^{m} substituents, wherein n is an integer from 0-6, and R^{m} is C₁₋₆alkyl, or C(O)C₁₋₆alkyl, wherein the C₁₋₆ alkyl is optionally substituted with one or more Rⁿ substituents, and Rⁿ is, independently at each occurrence, —OH;
R² is H, —OH, or C₁₋₆alkyl, wherein the C₁₋₆alkyl of R² is optionally substituted with one or more —OH; and
R³ is H or C₁₋₆alkyl, wherein the C₁₋₆alkyl of R³ is optionally substituted with one or more —OH;
provided that when L is (i), either:
(1) m is 1,
(2) at least one of Z¹, Z², Z³, and Z⁴ is —N— or —C(R^{f})—,
(3) R³ is other than H,
(4) at least one of R^{a}, R^{b}, and R^{c} is heterocycle, or
(5) at least one of R^{g}, Rⁱ, R^{j}, R^{k}, and Rⁿ is present;

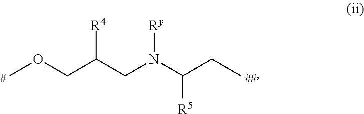

wherein
R^{y}, R⁴, and R⁵ are taken together with the atoms to which they are attached to form a 8-20 membered bicyclic heterocycle, wherein the 8-20 membered bicyclic heterocycle is substituted with n independently selected R^{m} substituents, wherein n is an integer from 0-6, and R^{m} is C₁. 6alkyl, or C(O)C₁₋₆alkyl, wherein the C₁₋₆alkyl is optionally substituted with one or more Rⁿ substituents, and Rⁿ is, independently at each occurrence, —OH; or

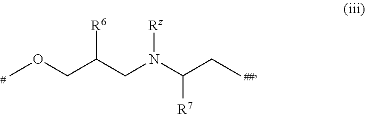

wherein
R^{z} and R⁶ are taken together with the atoms to which they are attached to form a 3-8 membered heterocycleheterocycle substituted with n independently selected R^{m} substituents, wherein n is an integer from 0-6, and R^{m} is C₁₋₆alkyl or C(O)C₁₋₆alkyl, wherein the C₁₋₆ alkyl is optionally substituted with one or more Rⁿ substituents, and Rⁿ is, independently at each occurrence, —OH; and
R⁷ is taken, together with one of X¹ and X² and the atoms to which they are attached, to form a C₄₋₈cycloalkyl;
wherein, for each of (i)-(iii), # denotes the point of attachment to the ring bearing moieties moieties Z¹-Z⁴ and ## denotes the point of attachment to the phenyl ring bearing moieties X¹-X⁴;
X¹, and X² are, independently of each other, H, halo, —CN, C₁₋₆alkyl, C₁₋₆alkoxy, or SF₅, wherein the C₁₋₆alkyl or C₁₋₆alkoxy is optionally substituted with one or more halo,
or one of X¹ and X² is taken together with R⁷ and the atoms to which it is attached to form a C₄₋₈cycloalkyl, and the other of X¹ or X² is H, halo, —CN, C₁₋₆alkyl, C₁₋₆alkoxy, or SF₅, wherein the C₁₋₆alkyl or C₁₋₆alkoxy is optionally substituted with one or more halo; and
X³, and X⁴ are, independently of each other, H, halo, —CN, C₁₋₆alkyl, C₁₋₆alkoxy, or SF₅, wherein the C₁₋₆alkyl or C₁₋₆alkoxy is optionally substituted with one or more halo.

In some embodiments, provided is a compound of formula (A), or a stereoisomer or tautomer thereof, or a pharmaceutically acceptable salt of any of the foregoing, wherein the compound of formula (A) is a compound of formula (I):

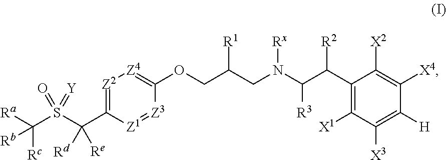

or a stereoisomer or tautomer thereof, or a pharmaceutically acceptable salt of any of the foregoing.

In some embodiments, provided herein is a compound of formula (A) or formula (I), or a pharmaceutically acceptable salt of any of the foregoing, wherein R^{x} and R¹ are taken together with the atoms to which they are attached to form a 3-8 membered heterocycle, wherein the 3-8 membered heterocycle is substituted with n independently selected R^{m} substituents, wherein n is an integer from 0-6, and R^{m} is C₁₋₆alkyl, or C(O)C₁₋₆alkyl, wherein the C₁₋₆alkyl is optionally substituted with one or more Rⁿ substituents, and Rⁿ is, independently at each occurrence, —OH. In some embodiments, R^{x} and R¹ are taken together with the atoms to which they are attached to form a 3-8 membered heterocycle, wherein the 3-8 membered heterocycle is substituted with n independently selected R^{m} substituents. In some embodiments, R^{x} and R¹ are taken together with the atoms to which they are attached to form a 5-7 membered heterocycle, wherein the 5-7 membered heterocycle is substituted with n independently selected R^{m} substituents.

In some embodiments, provided herein is a compound of formula (A) or formula (I), or a pharmaceutically acceptable salt of any of the foregoing, wherein R^{x} and R¹ are taken together with the atoms to which they are attached to form a 5-7 membered heterocycle selected from the group consisting of pyrrolidine, piperidine, azepane, imidazolidine, piperazine, 1,4-diazepane, hexahydropyrimidine, triazinane, triazepane, oxazolidine, morpholine, 1,4-oxazepane, 1,3-oxazinane, 1,4-oxazepane, thiazolidine, thiomorpholine, 1,4-thiazepane, 1,3-thiazinane, and 1,4-thiazepane. In some embodiments, the 5-7 membered heterocycle is selected from the group consisting of pyrrolidine, piperidine, azepane, imidazolidine, piperazine, 1,4-diazepane, and hexahydropyrimidine. In some embodiments, the 5-7 membered heterocycle is selected from the group consisting of pyrrolidine, piperidine, and piperazine.

In some embodiments, provided herein is a compound of formula (A) or formula (I), or a pharmaceutically acceptable salt of any of the foregoing, wherein, R^{x} and R¹ are taken together with the atoms to which they are attached to form a 5-membered heterocycle, wherein the 5-membered heterocycle is substituted with n independently selected R^{m} substituents. In some embodiments, the 5-membered heterocycle is pyrrolidine. In some embodiments, R^{x} and R¹ are taken together with the atoms to which they are attached to form a 6-membered heterocycle, wherein the 6-membered heterocycle is substituted with n independently selected R^{m} substituents. In some embodiments, the 6-membered heterocycle is piperidine, or piperazine. In some embodiments, the 6-membered heterocycle is piperidine. In some embodiments, the 6-membered heterocycle is piperazine.

In some embodiments, provided herein is a compound of formula (A) or formula (I), or a stereoisomer or tautomer thereof, or a pharmaceutically acceptable salt of any of the foregoing, wherein the compound is of formula (I-A):

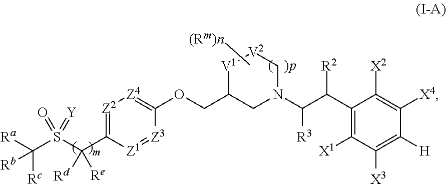

or a stereoisomer or tautomer thereof, or a pharmaceutically acceptable salt of any of the foregoing, wherein p is 0, 1, or 2, and V¹ and V² are each independently —CH₂—, —NH—, or —O—.

In some embodiments, provided herein is a compound of formula (A) or formula (I), such as a compound of formula (I-A), or a pharmaceutically acceptable salt of any of the foregoing, wherein p is 0, 1, or 2, and V¹ and V² are each independently —CH₂—, —NH—, or —O—. In some embodiments, p is 0, and V¹ and V² are each independently —CH₂—, —NH—, or —O—. In some embodiments, p is 1, and V¹ and V² are each independently —CH₂—, —NH—, or —O—. In some embodiments, p is 2, and V¹ and V² are each independently —CH₂—, —NH—, or —O—. In some embodiments, p is 0, 1, or 2, and both of V¹ and V² are —CH₂—. In some embodiments, p is 0, 1, or 2, V¹ is —CH₂—, and V² is —NH—, or —O—. In some embodiments, p is 0, 1, or 2, V¹ is —NH—, or —O—, and V² is —CH₂—. In some embodiments, p is 0, and both of V¹ and V² are —CH₂—. In some embodiments, p is 1, and both of V¹ and V² are —CH₂—. In some embodiments, p is 2, and both of V¹ and V² are —CH₂—. In some embodiments, p is 1, V¹ is —NH— and V² is —CH₂—. In some embodiments, p is 1, V¹ is —O—, and V² is —CH₂—.

In some embodiments, provided herein is a compound of formula (A) or formula (I), such as a compound of formula (I-A), or a pharmaceutically acceptable salt of any of the foregoing, wherein the compound is of formula (I-A1):

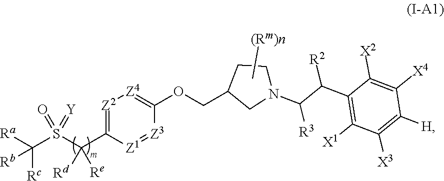

or a stereoisomer or tautomer thereof, or a pharmaceutically acceptable salt of any of the foregoing.

In some embodiments, provided herein is a compound of formula (A) or formula (I), such as a compound of formula (I-A2), or a stereoisomer or tautomer thereof, or a pharmaceutically acceptable salt of any of the foregoing, wherein n is an integer from 0-6. In some embodiments, n is an integer from 0-3. In some embodiments, n is 0 or 1. In some embodiments, n is 0. In some embodiments, n is 1.

In some embodiments, provided herein is a compound of formula (A) or formula (I), such as a compound of formula (I-A), or a pharmaceutically acceptable salt of any of the foregoing, wherein the compound is of formula (I-A2):

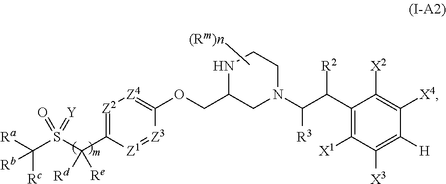

or a stereoisomer or tautomer thereof, or a pharmaceutically acceptable salt of any of the foregoing.

In some embodiments, provided herein is a compound of formula (A) or formula (I), such as a compound of formula (I-A2), or a stereoisomer or tautomer thereof, or a pharmaceutically acceptable salt of any of the foregoing, wherein n is 0.

In some embodiments, provided herein is a compound of formula (A) or formula (I), such as a compound of formula (I-A), (I-A1), or (I-A2), or a stereoisomer or tautomer thereof, or a pharmaceutically acceptable salt of any of the foregoing, wherein n is an integer from 0-6. In some embodiments, n is an integer from 0-5. In some embodiments, n is an integer from 0-4. In some embodiments, n is an integer from 0-3. In some embodiments, n is an integer from 0-2. In some embodiments, n is 0 or 1. In some embodiments, n is an integer from 1-6. In some embodiments, n is an integer from 1-5. In some embodiments, n is an integer from 1-4. In some embodiments, n is an integer from 1-3. In some embodiments, n is 1 or 2. In some embodiments, n is 0. In some embodiments, n is 1. In some embodiments, n is 2.

In some embodiments, provided herein is a compound of formula (A) or formula (I), such as a compound of formula (I-A), (I-A1), or (I-A2), or a stereoisomer or tautomer thereof, or a pharmaceutically acceptable salt of any of the foregoing, wherein R^{m} is C₁₋₆alkyl, or C(O)C₁₋₆alkyl, wherein the C₁₋₆alkyl is optionally substituted with one or more Rⁿ substituents, and Rⁿ is, independently at each occurrence, —OH. In some embodiments, R^{m} is C₁₋₃alkyl, or C(O)C₁₋₃alkyl, wherein the C₁₋₃alkyl is optionally substituted with one or more Rⁿ substituents, and Rⁿ is, independently at each occurrence, —OH. In some embodiments, R^{m} is methyl, or C(O)CH₃, wherein the methyl is optionally substituted with one or more Rⁿ substituents, and Rⁿ is, independently at each occurrence, —OH. In some embodiments, R^{m} is C₁₋₆alkyl, wherein the C₁₋₆alkyl is optionally substituted with one or more Rⁿ substituents, and Rⁿ is, independently at each occurrence, —OH. In some embodiments, R^{m} is methyl. In some embodiments, R^{m} is —CH₂OH. In some embodiments, R^{m} is C(O)C₁₋₆alkyl. In some embodiments, R^{m} is C(O)CH₃.

In some embodiments, provided herein is a compound of formula (A) or formula (I), such as a compound of formula (I-A), (I-A1), or (I-A2), or a stereoisomer or tautomer thereof, or a pharmaceutically acceptable salt of any of the foregoing, wherein n is 1 and R^{m} is C₁₋₆alkyl, wherein the C₁₋₆alkyl is optionally substituted with one or more Rⁿ substituents, and Rⁿ is, independently at each occurrence, —OH. In some embodiments, n is 1 and R^{m} is methyl. In some embodiments, n is 1 and R^{m} is CH₂OH. In some embodiments, n is 2 and each R^{m} is C₁₋₆ alkyl. In some embodiments, n is 2 and each R^{m} is methyl. In some embodiments, n is 1 and R^{m} is C(O)C₁₋₆alkyl. In some embodiments, n is 1 and R^{m} is C(O)CH₃.

In some embodiments, provided herein is a compound of formula (A) or formula (I), such as a compound of formula (I-A), (I-A1), or (I-A2), or a stereoisomer or tautomer thereof, or a pharmaceutically acceptable salt of any of the foregoing, wherein R² is H, —OH, or C₁₋₆alkyl, wherein the C₁₋₆alkyl of R² is optionally substituted with one or more OH. In some embodiments, R² is H, —OH, or C₁₋₃alkyl, wherein the C₁₋₃alkyl of R² is optionally substituted with one or more OH. In some embodiments, R² is H, —OH, or methyl, wherein the methyl of R² is optionally substituted with one or more OH. In some embodiments, R² is H. In some embodiments, R² is —OH. In some embodiments, R² is methyl. In some embodiments, R² is —CH₂OH.

In some embodiments, provided herein is a compound of formula (A) or formula (I), such as a compound of formula (I-A), (I-A1), or (I-A2), or a stereoisomer or tautomer thereof, or a pharmaceutically acceptable salt of any of the foregoing, wherein R³ is H, or C₁₋₆ alkyl, wherein the C₁₋₆alkyl of R³ is optionally substituted with one or more OH. In some embodiments, R³ is H, or C₁₋₃alkyl, wherein the C₁₋₃alkyl of R³ is optionally substituted with one or more —OH. In some embodiments, R³ is H, or methyl, wherein the methyl of R³ is optionally substituted with one or more —OH. In some embodiments, R³ is H. In some embodiments, R³ is methyl. In some embodiments, R³ is —CH₂OH.

In some embodiments, provided herein is a compound of formula (A) or formula (I), such as a compound of formula (I-A), (I-A1), or (I-A2), or a stereoisomer or tautomer thereof, or a pharmaceutically acceptable salt of any of the foregoing, wherein R² and R³ are independently H. In some embodiments, one of R² and R³ is H and the other R² and R³ is methyl. In some embodiments, R² is OH and R³ is H. In some embodiments, one of R² and R³ is H and the other R² and R³ is methyl. In some embodiments, one of R² and R³ is H and the other R² and R³ is CH₂OH. In some embodiments, one of R² and R³ is methyl and the other R² and R³ is OH.

In some embodiments, provided herein is a compound of formula (A) or formula (I), such as a compound of formula (I-A), (I-A1), or (I-A2), or a stereoisomer or tautomer thereof, or a pharmaceutically acceptable salt of any of the foregoing, wherein L is selected from the group consisting of

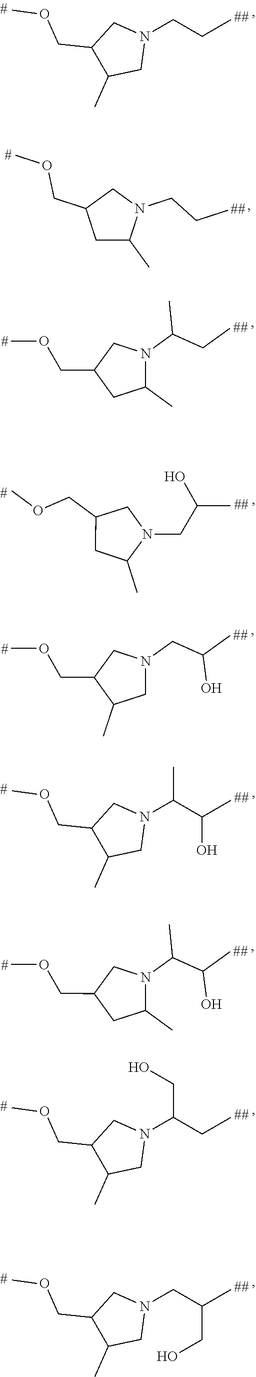

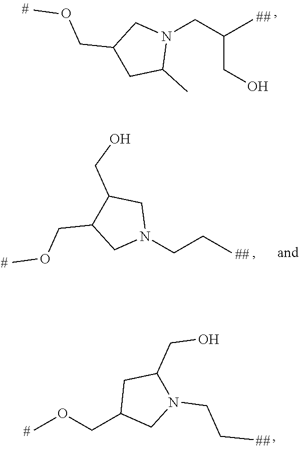

wherein # represents the point of attachment to the ring bearing Z¹, Z², Z³, and Z⁴, and ## represents the point of attachment to the remainder of the molecule. In some embodiments, L is selected from the group consisting of

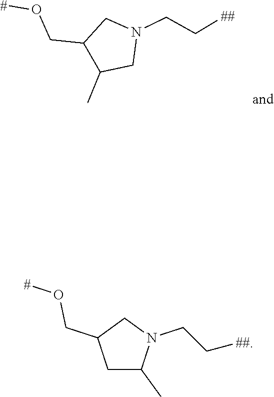

In some embodiments, provided herein is a compound of formula (A) or formula (I), such as a compound of formula (I-A), (I-A1), or (I-A2), or a stereoisomer or tautomer thereof, or a pharmaceutically acceptable salt of any of the foregoing, wherein L is selected from the group consisting of

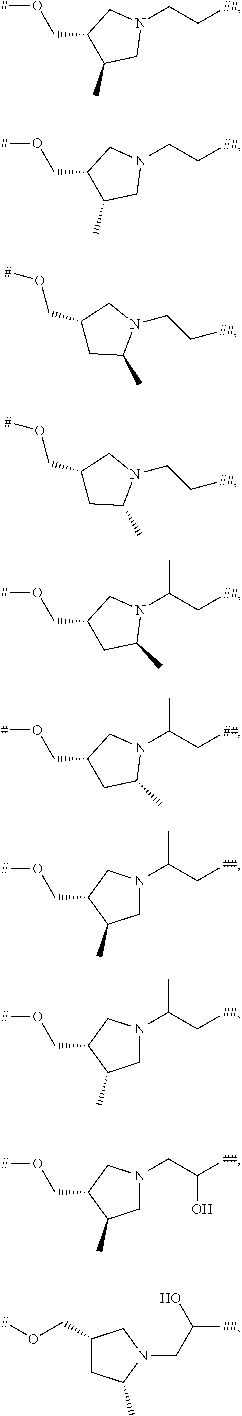

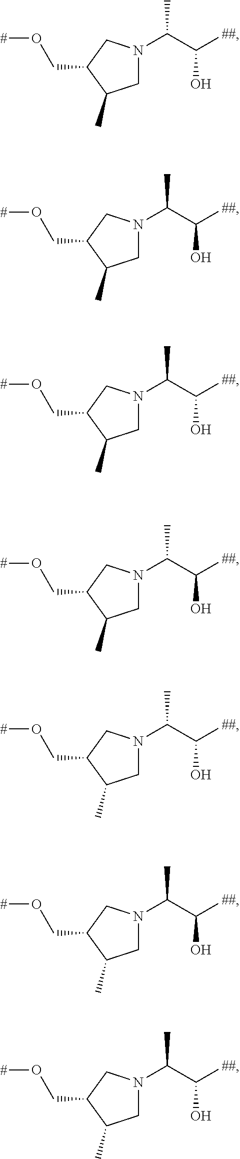

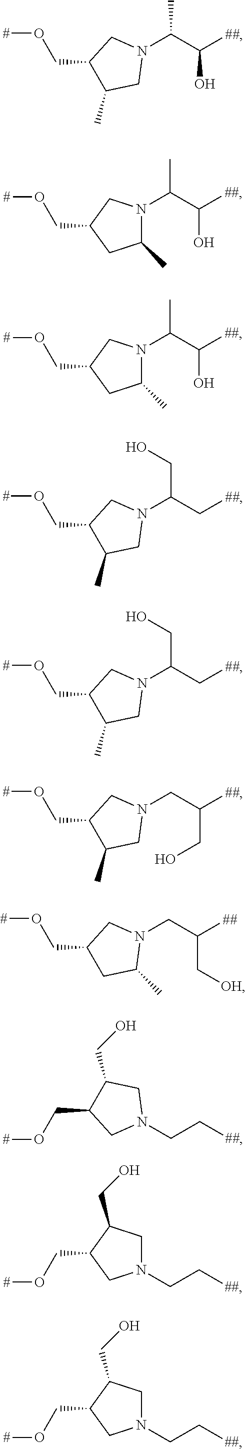

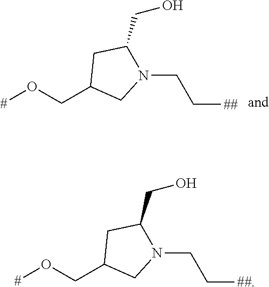

In some embodiments, L is selected from the

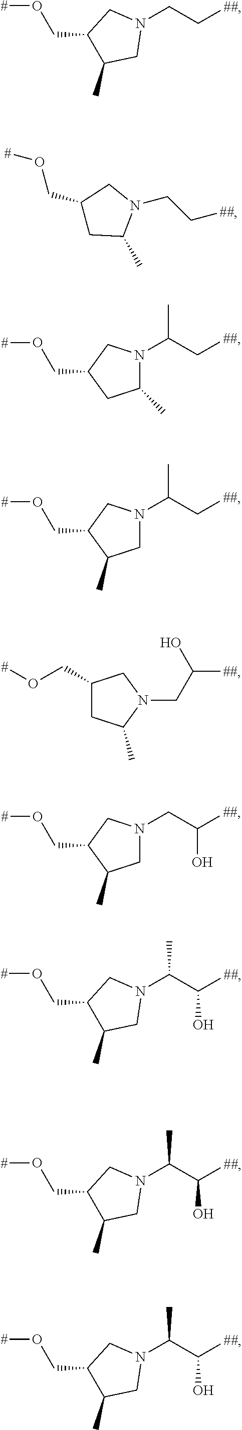

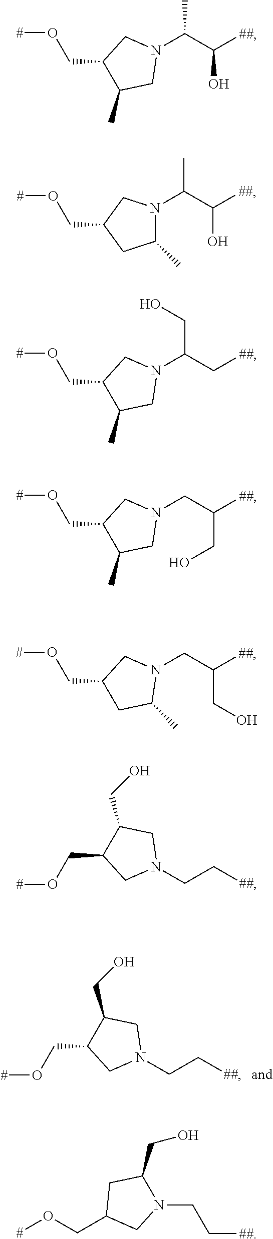

In some embodiments, L is selected from the group consisting of

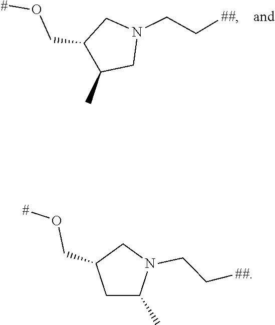

In some embodiments, provided herein is a compound of formula (A) or formula (I), such as a compound of formula (I-A), (I-A1), or (I-A2), or a stereoisomer or tautomer thereof, or a pharmaceutically acceptable salt of any of the foregoing, wherein L is

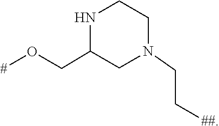

wherein # represents the point of attachment to the ring bearing Z¹, Z², Z³, and Z⁴, and ## represents the point of attachment to the remainder of the molecule. In some embodiments, L is

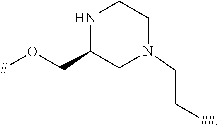

In some embodiments, provided herein is a compound of formula (A), or a stereoisomer or tautomer thereof, or a pharmaceutically acceptable salt of any of the foregoing, wherein the compound of formula A) is a compound of formula (II):

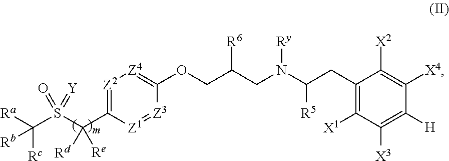

or a stereoisomer or tautomer thereof, or a pharmaceutically acceptable salt of any of the foregoing.

In some embodiments, provided herein is a compound of formula (A) or formula (II), or a stereoisomer or tautomer thereof, or a pharmaceutically acceptable salt of any of the foregoing, wherein the compound is of formula (II-A):

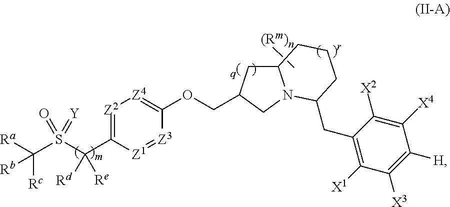

or a stereoisomer or tautomer thereof, or a pharmaceutically acceptable salt of any of the foregoing, q is 1, or 2 and r is 0 or 1.

In some embodiments, provided herein is a compound of formula (A) or formula (II), such as a compound of formula (II-A), or a pharmaceutically acceptable salt of any of the foregoing, wherein q is 1, or 2, and r is 0 or 1. In some embodiments, q is 1, and r is 0 or 1. In some embodiments, q is 2, and r is 0 or 1. In some embodiments, both of q and r are 1. In some embodiments, q is 1, and r is 0. In some embodiments, q is 1, and r is 1. In some embodiments, q is 2, and r is 0. In some embodiments, q is 2, and r is 1.

In some embodiments, provided herein is a compound of formula (A) or formula (II), such as a compound of formula (II-A), or a stereoisomer or tautomer thereof, or a pharmaceutically acceptable salt of any of the foregoing, wherein the compound is of formula (II-A1):

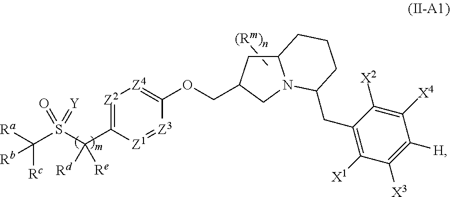

or a stereoisomer or tautomer thereof, or a pharmaceutically acceptable salt of any of the foregoing.

In some embodiments, provided herein is a compound of formula (A) or formula (iI), such as a compound of formula (II-A1), or a stereoisomer or tautomer thereof, or a pharmaceutically acceptable salt of any of the foregoing, wherein n is an integer from 0-6. In some embodiments, n is an integer from 0-5. In some embodiments, n is an integer from 0-4. In some embodiments, n is an integer from 0-3. In some embodiments, n is an integer from 0-2. In some embodiments, n is 0 or 1. In some embodiments, n is an integer from 1-6. In some embodiments, n is an integer from 1-5. In some embodiments, n is an integer from 1-4. In some embodiments, n is an integer from 1-3. In some embodiments, n is 1 or 2. In some embodiments, n is 0. In some embodiments, n is 1. In some embodiments, n is 2.

In some embodiments of the foregoing, provided herein is a compound of formula (A) or formula (II), such as a compound of formula (II-A1), or a stereoisomer or tautomer thereof, or a pharmaceutically acceptable salt of any of the foregoing, wherein L is

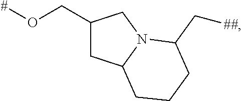

wherein # represents the point of attachment to the ring bearing Z¹, Z², Z³, and Z⁴, and ## represents the point of attachment to the remainder of the molecule. In some embodiments, L is

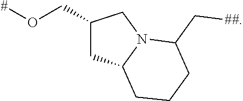

In some embodiments, provided herein is a compound of formula (A), such as a compound of formula (I), (I-A), (I-A1), (I-A2), (II), (II-A), or (II-A1), or a stereoisomer or tautomer thereof, or a pharmaceutically acceptable salt of any of the foregoing, wherein X¹, and X² are, independently of each other, H, halo, —CN, C₁₋₆alkyl, C₁₋₆alkoxy, or SF₅, wherein the C₁₋₆alkyl or C₁₋₆alkoxy is optionally substituted with one or more halo. In some embodiments, both of X¹, and X² are H. In some embodiments, one of X¹, and X² is H, and the other of X¹, and X² is halo, —CN, C₁₋₆alkyl, C₁₋₆alkoxy, or SF₅, wherein the C₁₋₆alkyl or C₁₋₆alkoxy is optionally substituted with one or more halo.

In some embodiments, provided herein is a compound of formula (A), such as a compound of formula (I), (I-A), (I-A1), (I-A2), (II), (II-A), or (II-A1), or a stereoisomer or tautomer thereof, or a pharmaceutically acceptable salt of any of the foregoing, wherein X³, and X⁴ are, independently of each other, H, halo, —CN, C₁₋₆alkyl, C₁₋₆alkoxy, or SF₅, wherein the C₁₋₆alkyl or C₁₋₆alkoxy is optionally substituted with one or more halo. In some embodiments, both of X³, and X⁴ are H. In some embodiments, one of X³, and X⁴ is H, and the other of X³, and X⁴ is halo, —CN, C₁₋₆alkyl, C₁₋₆alkoxy, or SF₅, wherein the C₁₋₆alkyl or C₁₋₆alkoxy is optionally substituted with one or more halo.

In some embodiments, provided herein is a compound of formula (A), such as a compound of formula (I), (I-A), (I-A1), (I-A2), (II), (II-A), or (II-A1), or a stereoisomer or tautomer thereof, or a pharmaceutically acceptable salt of any of the foregoing, wherein X¹, X², X³, and X⁴ are, independently of each other, H, halo, —CN, C₁₋₆alkyl, C₁₋₆alkoxy, or SF₅, wherein the C₁₋₆alkyl or C₁₋₆alkoxy is optionally substituted with one or more halo. In some embodiments, three of X¹, X², X³, and X⁴ are H and one of X¹, X², X³, and X⁴ is H, halo, —CN, C₁₋₆alkyl, C₁₋₆alkoxy, or SF₅, wherein the C₁₋₆alkyl or C₁₋₆alkoxy is optionally substituted with one or more halo. In some embodiments, two of X¹, X², X³, and X⁴ are H and two of X¹, X², X³, and X⁴ are independently H, halo, —CN, C₁₋₆alkyl, C₁₋₆alkoxy, or SF₅, wherein the C₁₋₆alkyl or C₁₋₆alkoxy is optionally substituted with one or more halo. In some embodiments, two of X¹, X², X³, and X⁴ are H and two of X¹, X², X³, and X⁴ are independently H, halo, —CN, C₁₋₆alkyl, C₁₋₆ alkoxy, or SF₅, wherein the C₁₋₃alkyl or C₁₋₃alkoxy is optionally substituted with one or more halo.

In some embodiments, provided herein is a compound of formula (A), such as a compound of formula (I), (I-A), (I-A1), (I-A2), (II), (II-A), or (II-A1), or a stereoisomer or tautomer thereof, or a pharmaceutically acceptable salt of any of the foregoing, wherein X¹ and X² are each H, one of X³ and X⁴ is H, and the other of X³ and X⁴ is halo or —CN. In some embodiments, X¹ and X² are each H, one of X³ and X⁴ is H, and the other of X³ and X⁴ is chloro. In some embodiments, X¹ and X² are each H, one of X³ and X⁴ is H, and the other of X³ and X⁴ is —CN.

In some embodiments, provided herein is a compound of formula (A), such as a compound of formula (I), (I-A), (I-A1), (I-A2), (II), (II-A), or (II-A1), or a stereoisomer or tautomer thereof, or a pharmaceutically acceptable salt of any of the foregoing, wherein X¹ and X² are each H, and X³ and X⁴ are independently, halo, —CN, C₁₋₆alkyl, C₁₋₆alkoxy, or SF₅, wherein the C₁₋₆alkyl or C₁₋₆alkoxy is optionally substituted with one or more halo. In some embodiments, X¹ and X² are each H, and X³ and X⁴ are independently, halo, —CN, C₁₋₃alkyl, C₁₋₃ alkoxy, or SF₅, wherein the C₁₋₃alkyl or C₁₋₃alkoxy is optionally substituted with one or more halo. In some embodiments, X¹ and X² are each H, and X³ and X⁴ are independently, halo, or —CN. In some embodiments, X¹ and X² are each H, and X³ and X⁴ are independently, Cl, or —CN.

In some embodiments, provided herein is a compound of formula (A), such as a compound of formula (I), (I-A), (I-A1), (I-A2), (II), (II-A), or (II-A1), or a stereoisomer or tautomer thereof, or a pharmaceutically acceptable salt of any of the foregoing, wherein one of X¹ and X² is H, the other of X¹ and X² is halo, —CN, C₁₋₆alkyl, C₁₋₆alkoxy, or SF₅, wherein the C₁₋₆alkyl or C₁₋₆alkoxy is optionally substituted with one or more halo, and X³ and X⁴ are independently, halo, —CN, C₁₋₆alkyl, C₁₋₆alkoxy, or SF₅, wherein the C₁₋₆alkyl or C₁₋₆alkoxy is optionally substituted with one or more halo. In some embodiments, one of X¹ and X² is H, the other of X¹ and X² is halo, —CN, C₁₋₃alkyl, C₁₋₃alkoxy, or SF₅, wherein the C₁₋₃alkyl or C₁. 3alkoxy is optionally substituted with one or more halo, and X³ and X⁴ are independently, halo, —CN, C₁₋₃alkyl, C₁₋₃alkoxy, or SF₅, wherein the C₁₋₃alkyl or C₁₋₃alkoxy is optionally substituted with one or more halo. In some embodiments, one of X¹ and X² is H, the other of X¹ and X² is methyl, and X³ and X⁴ are independently, halo, —CN, C₁₋₃alkyl, C₁₋₃alkoxy, or SF₅, wherein the C₁₋₃alkyl or C₁₋₃alkoxy is optionally substituted with one or more halo. In some embodiments, one of X¹ and X² is H, the other of X¹ and X² is methyl, and X³ and X⁴ are independently, Cl, or —CN.

In some embodiments, provided herein is a compound of formula (A), such as a compound of formula (I), (I-A), (I-A1), (I-A2), (II), (II-A), or (II-A1), or a stereoisomer or tautomer thereof, or a pharmaceutically acceptable salt of any of the foregoing, the phenyl ring bearing moieties X¹, X², X³, and X⁴ is selected from the group consisting of

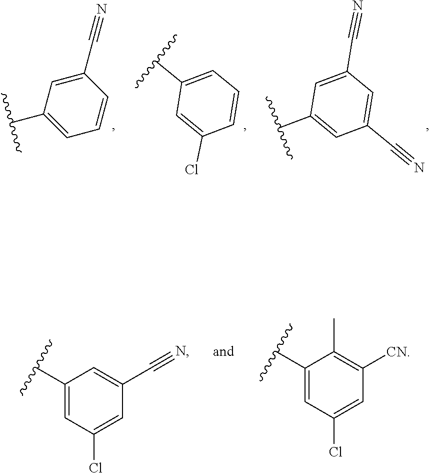

In some embodiments, the phenyl ring bearing moieties X¹, X², X³, and X⁴ is

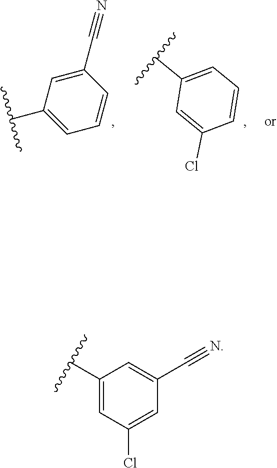 [SMILES: *c1cccc(C#N)c1 ; *c1cccc(Cl)c1 ; *c1cc(Cl)cc(C#N)c1]

In some embodiments, the phenyl ring bearing moieties X¹, X², X³, and X⁴ is

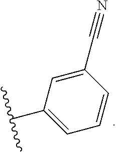 [SMILES: *c1cccc(C#N)c1]

In some embodiments, the phenyl ring bearing moieties X¹, X², X³, and X⁴ is

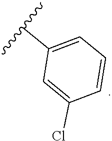 [SMILES: *c1cccc(Cl)c1]

In some embodiments, the phenyl ring bearing moieties X¹, X², X³, and X⁴ is

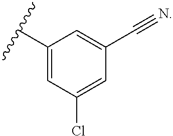 [SMILES: *c1cc(Cl)cc(C#N)c1]

In some embodiments, provided herein is a compound of formula (A), or a stereoisomer or tautomer thereof, or a pharmaceutically acceptable salt of any of the foregoing, wherein the compound is of formula (III):

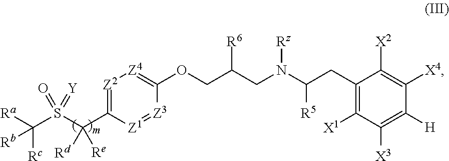

or a stereoisomer or tautomer thereof, or a pharmaceutically acceptable salt of any of the foregoing.

In some embodiments, provided herein is a compound of formula (A) or formula (III), or a stereoisomer or tautomer thereof, or a pharmaceutically acceptable salt of any of the foregoing, wherein the compound is of formula (III-A):

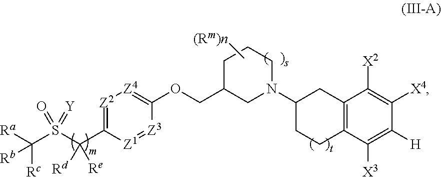

or a stereoisomer or tautomer thereof, or a pharmaceutically acceptable salt of any of the foregoing, wherein s is 0, 1, or 2 and t is 0 or 1.

In some embodiments, provided herein is a compound of formula (A) or formula (III), or a stereoisomer or tautomer thereof, or a pharmaceutically acceptable salt of any of the foregoing, wherein the compound is of formula (III-B):

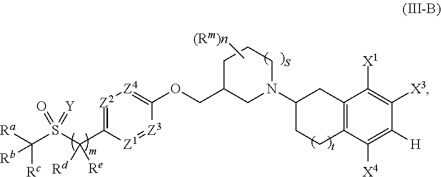

or a stereoisomer or tautomer thereof, or a pharmaceutically acceptable salt of any of the foregoing, wherein s is 0, 1, or 2 and t is 0 or 1.

In some embodiments, provided herein is a compound of formula (A) or formula (II), such as a compound of formula (II-A), or a pharmaceutically acceptable salt of any of the foregoing, wherein s is 0, 1, or 2, and t is 0 or 1. In some embodiments, s is 0, and t is 0 or 1. q is 1, and r is 0 or 1. In some embodiments, s is 1, and t is 0 or 1. In some embodiments, s is 2, and t is 0 or 1. In some embodiments, both of s and t are 0. In some embodiments, s is 0, and t is 1. In some embodiments, s is 1, and t is 0. In some embodiments, both of s and t are 1. In some embodiments, s is 2, and t is 0. In some embodiments, s is 2, and t is 1.

In some embodiments, provided herein is a compound of formula (A) or formula (III), such as a compound of formula (III-A), or a stereoisomer or tautomer thereof, or a pharmaceutically acceptable salt of any of the foregoing, wherein the compound is of formula (III-A1):

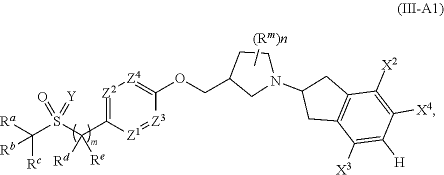

or a stereoisomer or tautomer thereof, or a pharmaceutically acceptable salt of any of the foregoing.

In some embodiments, provided herein is a compound of formula (A) or formula (III), such as a compound of formula (III-B), or a stereoisomer or tautomer thereof, or a pharmaceutically acceptable salt of any of the foregoing, wherein the compound is of formula (III-B1):

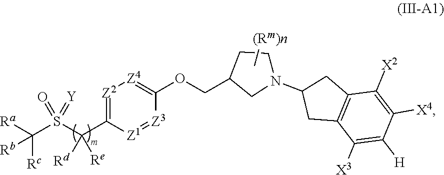

or a stereoisomer or tautomer thereof, or a pharmaceutically acceptable salt of any of the foregoing.

In some embodiments, provided herein is a compound of formula (A) or formula (III), such as a compound of formula (III-A), or a stereoisomer or tautomer thereof, or a pharmaceutically acceptable salt of any of the foregoing, wherein the compound is of formula (III-A2):

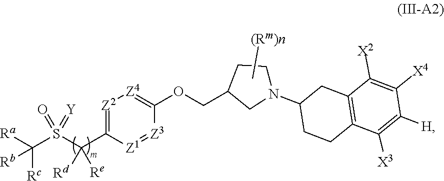

or a stereoisomer or tautomer thereof, or a pharmaceutically acceptable salt of any of the foregoing.

In some embodiments, provided herein is a compound of formula (A) or formula (III), such as a compound of formula (III-B), or a stereoisomer or tautomer thereof, or a pharmaceutically acceptable salt of any of the foregoing, wherein the compound is of formula (III-B2):

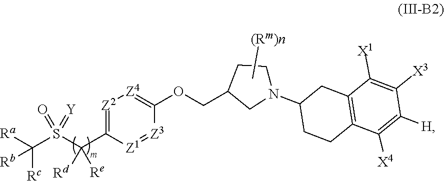

or a stereoisomer or tautomer thereof, or a pharmaceutically acceptable salt of any of the foregoing.

In some embodiments, provided herein is a compound of formula (A) or formula (III), such as a compound of formula (III-A), or a stereoisomer or tautomer thereof, or a pharmaceutically acceptable salt of any of the foregoing, wherein the compound is of formula (III-A3):

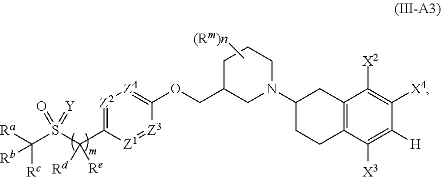

or a stereoisomer or tautomer thereof, or a pharmaceutically acceptable salt of any of the foregoing.

In some embodiments, provided herein is a compound of formula (A) or formula (III), such as a compound of formula (III-B), or a stereoisomer or tautomer thereof, or a pharmaceutically acceptable salt of any of the foregoing, wherein the compound is of formula (III-B3):

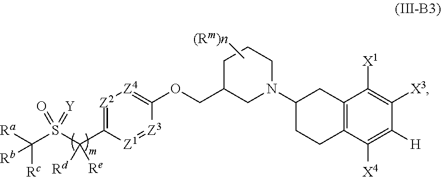

or a stereoisomer or tautomer thereof, or a pharmaceutically acceptable salt of any of the foregoing.

In some embodiments, provided herein is a compound of formula (A) or formula (III), such as a compound of formula (III-A), (III-B), (III-A1), (III-B1), (III-A2), (III-B2), (III-A3), or (III-B3), or a stereoisomer or tautomer thereof, or a pharmaceutically acceptable salt of any of the foregoing, wherein m is 0 or 1. In some embodiments, m is 0. In some embodiments, m is 1.

In some embodiments, provided herein is a compound of formula (A) or formula (III), such as a compound of formula (III-A), (III-B), (III-A1), (III-B1), (III-A2), (III-B2), (III-A3), or (III-B3), R^{d} and R^{e} are each independently H or C₁₋₆alkyl. In some embodiments, R^{d} and Rare each independently H. In some embodiments, R^{d} and R^{e} are each independently C₁₋₆alkyl. In some embodiments, one of R^{d} and R^{e} is H and the other of R^{d} and R^{e} is C₁₋₆alkyl. In some embodiments, one of R^{d} and R^{e} is H and the other of R^{d} and R^{e} is methyl.

In some embodiments, provided herein is a compound of formula (A) or formula (III), such as a compound of formula (III-A), (III-B), (III-A1), (III-B1), (III-A2), (III-B2), (III-A3), or (III-B3), wherein R^{d} and R^{e} are taken together with the atoms to which they are attached to form a C₃₋₆cycloalkyl or a 3-6 membered heterocycle. In some embodiments, R^{d} and R^{e} are taken together with the atoms to which they are attached to form a C₃₋₆cycloalkyl. In some embodiments, R^{d} and R^{e} are taken together with the atoms to which they are attached to form a C₃₋₄cycloalkyl. In some embodiments, R^{d} and R^{e} are taken together with the atoms to which they are attached to form cyclopropyl. In some embodiments, R^{d} and R^{e} are taken together with the atoms to which they are attached to form a 3-6 membered heterocycle. In some embodiments, R^{d} and R^{e} are taken together with the atoms to which they are attached to form a C₃₋₆cycloalkyl or a 3-4 membered heterocycle. In some embodiments, R^{d} and R^{e} are taken together with the atoms to which they are attached to form oxetane.

In some embodiments, provided herein is a compound of formula (A) or formula (III), such as a compound of formula (III-A), (III-B), (III-A1), (III-B1), (III-A2), (III-B2), (III-A3), or (III-B3), or a stereoisomer or tautomer thereof, or a pharmaceutically acceptable salt of any of the foregoing, wherein Y is O or —N(C₁₋₆alkyl). In some embodiments, Y is O. In some embodiments, Y is —N(C₁₋₆alkyl). In some embodiments, Y is —N(C₁₋₃alkyl). In some embodiments, Y is —N(C₃₋₆alkyl). In some embodiments, the C₁₋₆alkyl of the —N(C₁₋₆alkyl) is optionally substituted with one or more R^{g} substituents. In some embodiments, R^{g} is, independently at each occurrence, —S(O)₂C₁₋₆alkyl. In some embodiments, R^{g} is independently at each occurrence, —S(O)₂C₁₋₃alkyl. In some embodiments, R^{g} is, independently at each occurrence, —S(O)₂C₃₋₆alkyl.

In some embodiments, provided herein is a compound of formula (A) or formula (III), such as a compound of formula (III-A), (III-B), (III-A1), (III-B1), (III-A2), (III-B2), (III-A3), or (III-B3), or a stereoisomer or tautomer thereof, or a pharmaceutically acceptable salt of any of the foregoing, wherein Z¹, Z², Z³, and Z⁴ are, independently of each other, —N—, —CH— or —C(R^{f})—. In some embodiments, three or more of Z¹, Z², Z³, and Z⁴ are —N—. In some embodiments, three or more of Z¹, Z², Z³, and Z⁴ are —C(R^{f})—. In some embodiments, at least two of Z¹, Z², Z³, and Z⁴ are independently selected from —N— or —C(R^{E})—. In some embodiments, at least two of Z¹, Z², Z³, and Z⁴ are —CH—. In some embodiments, at least one of Z¹, Z², Z³, and Z⁴ is —CH—.

In some embodiments, provided herein is a compound of formula (A) or formula (III), such as a compound of formula (III-A), (III-B), (III-A1), (III-B1), (III-A2), (III-B2), (III-A3), or (III-B3), or a stereoisomer or tautomer thereof, or a pharmaceutically acceptable salt of any of the foregoing, wherein Z¹, Z², Z³, and Z⁴ are, independently of each other, —N—, —CH— or —C(R^{f})—. In some embodiments, each of Z¹, Z², Z³, and Z⁴ is —CH—. In some embodiments, three of Z¹, Z², Z³, and Z⁴ are —CH— and the other of Z¹, Z², Z³, and Z⁴ is —N—, or —C(R^{f})—. In some embodiments, two of Z¹, Z², Z³, and Z⁴ are —CH— and the other two of Z¹, Z², Z³, and Z⁴ are independently —N—, or —C(R^{f})—.

In some embodiments, provided herein is a compound of formula (A) or formula (III), such as a compound of formula (III-A), (III-B), (III-A1), (III-B1), (III-A2), (III-B2), (III-A3), or (III-B3), wherein R^{f} is independently at each occurrence, —CN, halo, C₁₋₆alkyl, C₁₋₆ alkoxy, or —N(R^{j})₂, wherein the C₁₋₆alkyl of R^{f} is optionally substituted with one or more halo. In some embodiments, R^{f} is independently at each occurrence, —CN, halo, C₁₋₃alkyl, C₁₋₃alkoxy, or —N(R^{j})₂, wherein the C₁₋₃alkyl of R^{f} is optionally substituted with one or more halo. In some embodiments, R^{f} is independently at each occurrence, —CN, Cl, F, I, methyl, —OCH₃, NH₂, or N(CH₃)₂, wherein the methyl of R^{f} is optionally substituted with one or more Cl, F, or I. In some embodiments, R^{f} is F. In some embodiments, R^{f} is methyl. In some embodiments, R^{f} is CF₃.

In some embodiments, provided herein is a compound of formula (A) or formula (III), such as a compound of formula (III-A), (III-B), (III-A1), (III-B1), (III-A2), (III-B2), (III-A3), or (III-B3), wherein the ring bearing Z¹, Z², Z³, and Z⁴ is benzene, pyridine, pyridazine, pyrimidine, pyrazine, or triazine, wherein the benzene, pyridine, pyridazine, pyrimidine, pyrazine, or triazine are optionally substituted. In some embodiments, the ring bearing Z¹, Z², Z³, and Z⁴ is benzene, pyridine, pyridazine, pyrimidine, pyrazine, or triazine. In some embodiments, the ring bearing Z¹, Z², Z³, and Z⁴ is benzene, or pyridine. In some embodiments, the ring bearing Z¹, Z², Z³, and Z⁴ is benzene. In some embodiments, the ring bearing Z¹, Z², Z³, and Z⁴ is pyridine.

In some embodiments, provided herein is a compound of formula (A) or formula (III), such as a compound of formula (III-A), (III-B), (III-A1), (III-B1), (III-A2), (III-B2), (III-A3), or (III-B3), wherein the ring bearing Z¹, Z², Z³, and Z⁴ is selected from the group consisting of

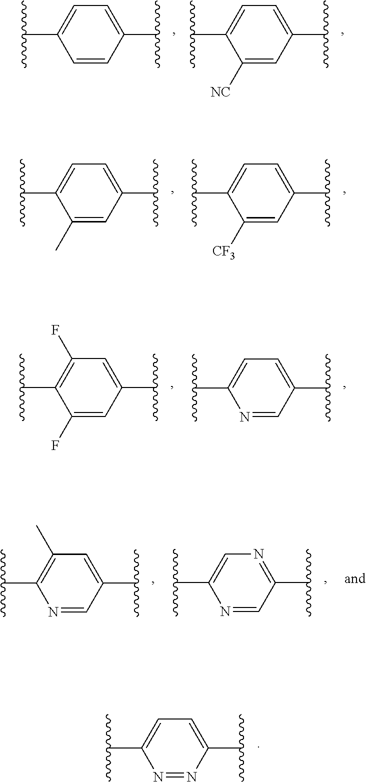

In some embodiments, the ring bearing Z¹, Z², Z³, and Z⁴ is

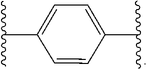 [SMILES: *c1ccc(*)cc1]

In some embodiments, provided herein is a compound of formula (A) or formula (III), such as a compound of formula (III-A), (III-B), (III-A1), (III-B1), (III-A2), (III-B2), (III-A3), or (III-B3), or a stereoisomer or tautomer thereof, or a pharmaceutically acceptable salt of any of the foregoing, wherein n is an integer from 0-6. In some embodiments, n is an integer from 0-5. In some embodiments, n is an integer from 0-4. In some embodiments, n is an integer from 0-3. In some embodiments, n is an integer from 0-2. In some embodiments, n is 0 or 1. In some embodiments, n is an integer from 1-6. In some embodiments, n is an integer from 1-5. In some embodiments, n is an integer from 1-4. In some embodiments, n is an integer from 1-3. In some embodiments, n is 1 or 2. In some embodiments, n is 0. In some embodiments, n is 1. In some embodiments, n is 2.

In some embodiments, provided herein is a compound of formula (A) or formula (III), such as a compound of formula (III-A), (III-B), (III-A1), (III-B1), (III-A2), (III-B2), (III-A3), or (III-B3), or a stereoisomer or tautomer thereof, or a pharmaceutically acceptable salt of any of the foregoing, wherein R^{m} is C₁₋₆alkyl, or C(O)C₁₋₆alkyl, wherein the C₁₋₆alkyl is optionally substituted with one or more Rⁿ substituents, and Rⁿ is, independently at each occurrence, —OH. In some embodiments, R^{m} is C₁₋₃alkyl, or C(O)C₁₋₃alkyl, wherein the C₁₋₃alkyl is optionally substituted with one or more Rⁿ substituents, and Rⁿ is, independently at each occurrence, —OH. In some embodiments, R^{m} is methyl, or C(O)CH₃, wherein the methyl is optionally substituted with one or more Rⁿ substituents, and Rⁿ is, independently at each occurrence, —OH. In some embodiments, R^{m} is C₁₋₆alkyl, wherein the C₁₋₆alkyl is optionally substituted with one or more Rⁿ substituents, and Rⁿ is, independently at each occurrence, —OH. In some embodiments, R^{m} is methyl. In some embodiments, R^{m} is —CH₂OH. In some embodiments, R^{m} is C(O)C₁₋₆alkyl. In some embodiments, R^{m} is C(O)CH₃.

In some embodiments, provided herein is a compound of formula (A) or formula (III), such as a compound of formula (III-A), (III-B), (III-A1), (III-B1), (III-A2), (III-B2), (III-A3), or (III-B3), or a stereoisomer or tautomer thereof, or a pharmaceutically acceptable salt of any of the foregoing, wherein n is 1 and R^{m} is C₁₋₆alkyl. In some embodiments, n is 1 and R^{m} is methyl. In some embodiments, n is 2 and each R^{m} is C₁₋₆alkyl. In some embodiments, n is 2 and each R^{m} is methyl. In some embodiments, n is 1 and R^{m} is C(O)C₁₋₆alkyl. In some embodiments, n is 1 and R^{m} is C(O)CH₃.

In some embodiments, provided herein is a compound of formula (A) or formula (III), such as a compound of formula (III-A), (III-B), (III-A1), (III-B1), (III-A2), (III-B2), (III-A3), or (III-B3), or a stereoisomer or tautomer thereof, or a pharmaceutically acceptable salt of any of the foregoing, wherein n is 1 and R^{m} is C₁₋₆alkyl. In some embodiments, n is 1 and R^{m} is methyl.

In some embodiments, provided herein is a compound of formula (A) or formula (I), such as a compound of formula (III-A), (III-B), (III-A1), (III-B1), (III-A2), (III-B2), (III-A3), or (III-B3), or a stereoisomer or tautomer thereof, or a pharmaceutically acceptable salt of any of the foregoing, wherein L is selected from the group consisting of

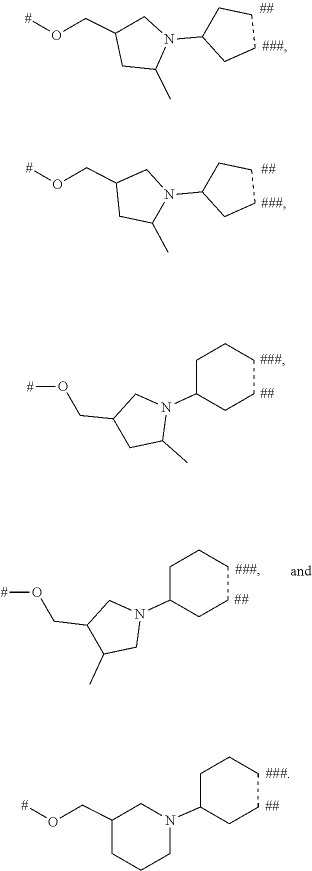

wherein # represents the point of attachment to the ring bearing Z¹, Z², Z³, and Z⁴, ### represents the point of attachment to X¹ or X², ## represents the point of attachment to the remainder of the molecule, and the dashed line represents a single or double bond. In some embodiments, L is selected from the group consisting of

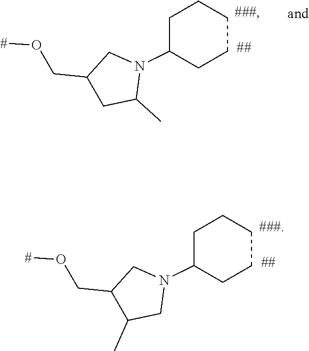

In some embodiments, L is

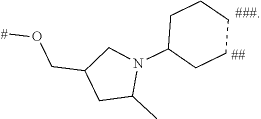

In some embodiments, provided herein is a compound of formula (A) or formula (I), such as a compound of formula (III-A), (III-B), (III-A1), (III-B1), (III-A2), (III-B2), (III-A3), or (III-B3), or a stereoisomer or tautomer thereof, or a pharmaceutically acceptable salt of any of the foregoing, wherein L is selected from the group consisting of

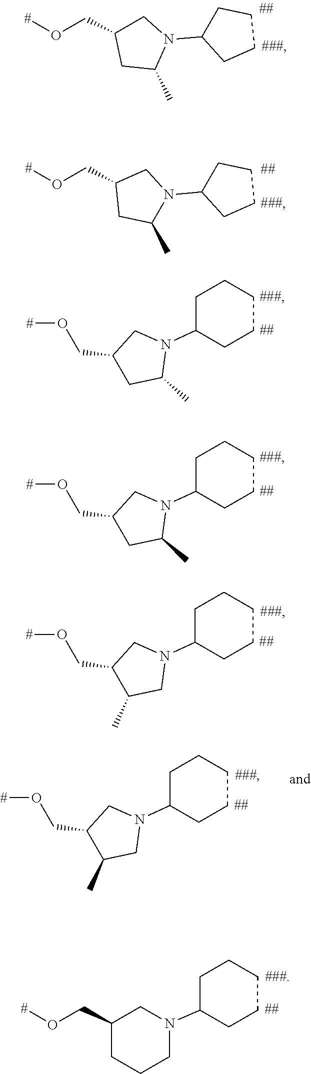

In some embodiments, L is selected from the group consisting of

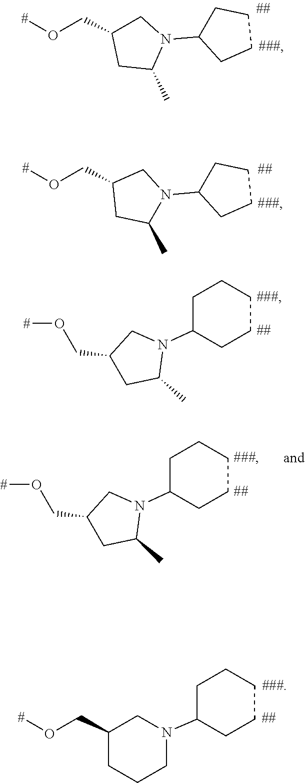

In some embodiments, L is selected from the group consisting of

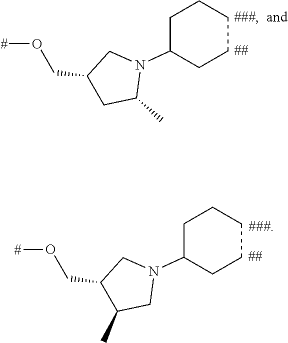

In some embodiments, L is

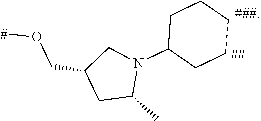

In some embodiments, L is

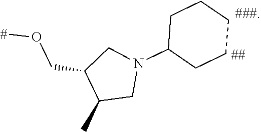

In some embodiments, provided herein is a compound of formula (A), such as a compound of formula (III), (III-A), (III-B), (III-A1), (III-B1), (III-A2), (III-B2), (III-A3), or (III-B3), or a stereoisomer or tautomer thereof, or a pharmaceutically acceptable salt of any of the foregoing, wherein one of X¹ and X² is taken together with R⁷ and the atoms to which it is attached to form a C₄₋₈cycloalkyl, and the other of X¹ or X² is H, halo, —CN, C₁₋₆alkyl, C₁₋₆alkoxy, or SF₅, wherein the C₁₋₆alkyl or C₁₋₆alkoxy is optionally substituted with one or more halo. In some embodiments, one of X¹ and X² is taken together with R⁷ and the atoms to which it is attached to form a C₄₋₈cycloalkyl, and the other of X¹ or X² is H.

In some embodiments, provided herein is a compound of formula (A), such as a compound of formula (III), (III-A), (III-B), (III-A1), (III-B1), (III-A2), (III-B2), (III-A3), or (III-B3), or a stereoisomer or tautomer thereof, or a pharmaceutically acceptable salt of any of the foregoing, wherein X³, and X⁴ are, independently of each other, H, halo, —CN, C₁₋₆alkyl, C₁₋₆ alkoxy, or SF₅, wherein the C₁₋₆alkyl or C₁₋₆alkoxy is optionally substituted with one or more halo. In some embodiments, both of X³, and X⁴ are H. In some embodiments, one of X³, and X⁴ is H, and the other of X³, and X⁴ is halo, —CN, C₁₋₆alkyl, C₁₋₆alkoxy, or SF₅, wherein the C₁₋₆alkyl or C₁₋₆alkoxy is optionally substituted with one or more halo.

In some embodiments, provided herein is a compound of formula (A), such as a compound of formula (III), (III-A), (III-B), (III-A1), (III-B1), (III-A2), (III-B2), (III-A3), or (III-B3), or a stereoisomer or tautomer thereof, or a pharmaceutically acceptable salt of any of the foregoing, wherein one of X¹ and X² is taken together with R⁷ and the atoms to which it is attached to form a C₄₋₈cycloalkyl, the other of X¹ or X² is H, halo, —CN, C₁₋₆alkyl, C₁₋₆alkoxy, or SF₅, wherein the C₁₋₆alkyl or C₁₋₆alkoxy is optionally substituted with one or more halo, and X³, and X⁴ are, independently of each other, H, halo, —CN, C₁₋₆alkyl, C₁₋₆alkoxy, or SF₅, wherein the C₁₋₆alkyl or C₁₋₆alkoxy is optionally substituted with one or more halo. In some embodiments, one of X¹ and X² is taken together with R⁷ and the atoms to which it is attached to form a C₄₋₈cycloalkyl, the other of X¹ or X² is H, one of X³, and X⁴ is H and the other of X³, and X⁴ is H, halo, —CN, C₁₋₆alkyl, C₁₋₆alkoxy, or SF₅, wherein the C₁₋₆alkyl or C₁₋₆alkoxy is optionally substituted with one or more halo.

In some embodiments, provided herein is a compound of formula (A), such as a compound of formula (III), (III-A), (III-B), (III-A1), (III-B1), (III-A2), (III-B2), (III-A3), or (III-B3), or a stereoisomer or tautomer thereof, or a pharmaceutically acceptable salt of any of the foregoing, one of X¹ and X² is taken together with R⁷ and the atoms to which it is attached to form a C₄₋₈cycloalkyl, the other of X¹ or X² is H, one of X³ and X⁴ is H, and the other of X³ and X⁴ is halo or —CN. In some embodiments, one of X¹ and X² is taken together with R⁷ and the atoms to which it is attached to form a C₄₋₈cycloalkyl, the other of X¹ or X² is H, one of X³ and X⁴ is H, and the other of X³ and X⁴ is chloro. In some embodiments, one of X¹ and X² is taken together with R⁷ and the atoms to which it is attached to form a C₄₋₈cycloalkyl, the other of X¹ or X² is H, one of X³ and X⁴ is H, and the other of X³ and X⁴ is —CN.

In some embodiments, provided herein is a compound of formula (A), such as a compound of formula (III), (III-A), (III-B), (III-A1), (III-B1), (III-A2), (III-B2), (III-A3), or (III-B3), or a stereoisomer or tautomer thereof, or a pharmaceutically acceptable salt of any of the foregoing, wherein one of X¹ and X² is taken together with R⁷ and the atoms to which it is attached to form a C₄₋₈cycloalkyl, the other of X¹ or X² is halo, —CN, C₁₋₆alkyl, C₁₋₆alkoxy, or SF₅, wherein the C₁₋₆alkyl or C₁₋₆alkoxy is optionally substituted with one or more halo, one of X³ and X⁴ is H, and the other of X³ and X⁴ is halo, —CN, C₁₋₆alkyl, C₁₋₆alkoxy, or SF₅, wherein the C₁₋₆alkyl or C₁₋₆alkoxy is optionally substituted with one or more halo. In some embodiments, one of X¹ and X² is taken together with R⁷ and the atoms to which it is attached to form a C₄₋₈cycloalkyl, the other of X¹ or X² is halo, —CN, C₁₋₃alkyl, C₁₋₃alkoxy, or SF₅, wherein the C₁₋₃alkyl or C₁₋₃alkoxy is optionally substituted with one or more halo, one of X³ and X⁴ is H, and the other of X³ and X⁴ is halo, —CN, C₁₋₃alkyl, C₁₋₃alkoxy, or SF₅, wherein the C₁₋₃alkyl or C₁₋₃alkoxy is optionally substituted with one or more halo. In some embodiments, one of X¹ and X² is taken together with R⁷ and the atoms to which it is attached to form a C₄₋₈cycloalkyl, the other of X¹ or X² is halo, or —CN, one of X³ and X⁴ is H, and the other of X³ and X⁴ is halo, or —CN. In some embodiments, one of X¹ and X² is taken together with R⁷ and the atoms to which it is attached to form a C₄₋₈cycloalkyl, the other of X¹ or X² is Cl, or —CN, one of X³ and X⁴ is H, and the other of X³ and X⁴ is Cl, or —CN.

In some embodiments, provided herein is a compound of formula (A), such as a compound of formula (III), (III-A), (III-B), (III-A1), (III-B1), (III-A2), (III-B2), (III-A3), or (III-B3), or a stereoisomer or tautomer thereof, or a pharmaceutically acceptable salt of any of the foregoing, the phenyl ring bearing moieties X¹, X², X³, and X⁴ is selected from the group consisting of

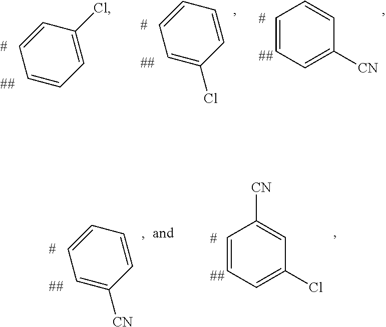 [SMILES: Clc1ccccc1 ; Clc1ccccc1 ; N#Cc1ccccc1 ; N#Cc1ccccc1 ; N#Cc1cccc(Cl)c1]

wherein # and ## represent the points of attachment to L.

In some embodiments, provided herein is a compound of formula (A), such as a compound of formula (I), (I-A), (I-A1), (I-A2), (II), (II-A), (II-A1), (III), (III-A), (III-B), (III-A1), (III-B1), (III-A2), (III-B2), (III-A3), or (III-B3), or a stereoisomer or tautomer thereof, or a pharmaceutically acceptable salt of any of the foregoing, wherein m is 0. In some embodiments, provided herein is a compound of formula (A), or any variation or embodiment thereof, or a stereoisomer or tautomer thereof, or a pharmaceutically acceptable salt of any of the foregoing, wherein the compound is of formula (B):

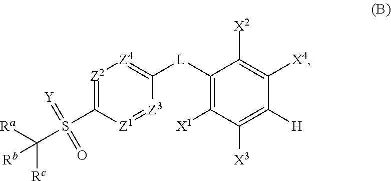

or a stereoisomer or tautomer thereof, or a pharmaceutically acceptable salt of any of the foregoing.

In some embodiments, provided herein is a compound of formula (A) or formula (B), or a stereoisomer or tautomer thereof, or a pharmaceutically acceptable salt of any of the foregoing, wherein Y is 0, wherein the compound is of formula (B-1):

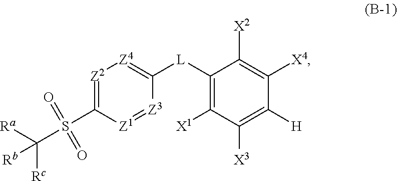

or a stereoisomer or tautomer thereof, or a pharmaceutically acceptable salt of any of the foregoing.

In some embodiments, provided herein is a compound of formula (A), formula (B), or formula (B-1), or a stereoisomer or tautomer thereof, or a pharmaceutically acceptable salt of any of the foregoing, wherein R^{a}, R^{b}, and R^{c} are each independently H, C₁₋₆alkyl, C₃. 6cycloalkyl, or 3-8 membered heterocycle wherein, the C₁₋₆alkyl of R^{a}, R^{b}, or R^{c} is optionally substituted with one or more R^{h} substituents, and the 3-8 membered heterocycle of R^{a}, R^{b}, or R^{c} is optionally substituted with one or more Rⁱ substituents. In some embodiments, two of R^{a}, R^{b}, and R^{c} are independently H, and one of R^{a}, R^{b}, and R^{c} is C₁₋₆alkyl, C₃₋₆cycloalkyl, or 3-8 membered heterocycle wherein, the C₁₋₆alkyl of R^{a}, R^{b}, or R^{c} is optionally substituted with one or more R^{h} substituents, and the 3-8 membered heterocycle of R^{a}, R^{b}, or R^{c} is optionally substituted with one or more Rⁱ substituents.

In some embodiments, provided herein is a compound of formula (A), formula (B), or formula (B-1), or a stereoisomer or tautomer thereof, or a pharmaceutically acceptable salt of any of the foregoing, R^{h} is independently at each occurrence, —OH, C₁₋₆alkoxy, —N(R^{j})₂, C(O)R^{k}, or —S(O)₂C₁₋₆alkyl. In some embodiments, R^{h} is independently at each occurrence, —OH, C₁₋₆alkoxy, —N(R^{j})₂, C(O)R^{k}, or —S(O)₂C₁₋₆alkyl; R^{j} is independently at each occurrence H, C₁₋₆ alkyl or C(O)C₁₋₆alkyl; and R^{k} is, independently at each occurrence C₁₋₆alkyl or C₁₋₆alkoxy. In some embodiments, R^{h} is independently at each occurrence, —OH, C₁₋₃alkoxy, —N(R^{j})₂, C(O)R^{k}, or —S(O)₂C₁₋₆alkyl; R^{j} is independently at each occurrence H, C₁₋₃alkyl or C(O)C₁₋₃alkyl; and R^{k} is, independently at each occurrence C₁₋₃alkyl or C₁₋₃alkoxy. In some embodiments, R^{h} is independently at each occurrence, —OH, —OCH₃, —N(R^{j})₂, C(O)R^{k}, or —S(O)₂CH₃; R^{j} is independently at each occurrence H, —CH₃ or C(O)CH₃; and R^{k} is, independently at each occurrence CH₃, or —OCH₃.

In some embodiments, provided herein is a compound of formula (A), formula (B), or formula (B-1), or a stereoisomer or tautomer thereof, or a pharmaceutically acceptable salt of any of the foregoing, Rⁱ is independently at each occurrence, oxo, C₁₋₆alkyl, or C(O)R^{k}. In some embodiments, Rⁱ is independently at each occurrence, oxo, C₁₋₆alkyl, or C(O)R^{k}; and R^{k} is, independently at each occurrence C₁₋₆alkyl or C₁₋₆alkoxy. In some embodiments, Rⁱ is independently at each occurrence, oxo, C₁₋₃alkyl, or C(O)R^{k}; and R^{k} is, independently at each occurrence C₁₋₃alkyl or C₁₋₃alkoxy. In some embodiments, Rⁱ is independently at each occurrence, oxo, CH₃, or C(O)R^{k}; and R^{k} is, independently at each occurrence CH₃ or —OCH₃.

In some embodiments, provided herein is a compound of formula (A), formula (B), or formula (B-1), or a stereoisomer or tautomer thereof, two of R^{a}, R^{b}, and R^{c} are independently H, and one of R^{a}, R^{b}, and R^{C} is

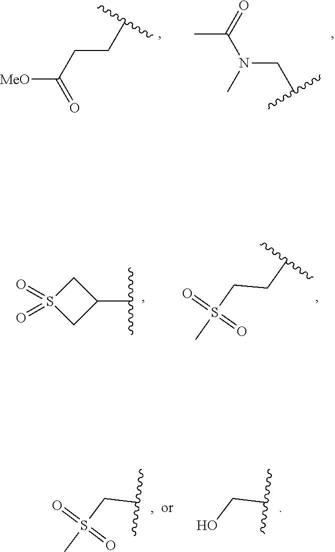

In some embodiments, provided herein is a compound of formula (A), formula (B), or formula (B-1), or a stereoisomer or tautomer thereof, or a pharmaceutically acceptable salt of any of the foregoing, wherein any two of R^{a}, R^{b}, and R^{c} are taken, together with the atoms to which they are attached, to form a C₃₋₆cycloalkyl or a 3-8 membered heterocycle, wherein the 3-8 membered heterocycle is optionally substituted with one or more Rⁱ substituents, and the other of R^{a}, R^{b}, and R^{c} is H or C₁₋₆alkyl, C₃₋₆cycloalkyl, or 3-8 membered heterocycle wherein, the C₁₋₆alkyl of R^{a}, R^{b}, or R^{c} is optionally substituted with one or more R^{h} substituents, and the 3-8 membered heterocycle of R^{a}, R^{b}, or R^{c} is optionally substituted with one or more Rⁱ substituents.

In some embodiments, provided herein is a compound of formula (A), formula (B), or formula (B-1), or a stereoisomer or tautomer thereof, or a pharmaceutically acceptable salt of any of the foregoing, wherein any two of R^{a}, R^{b}, and R^{c} are taken, together with the atoms to which they are attached, to form a 3-8 membered heterocycle, wherein the 3-8 membered heterocycle is optionally substituted with one or more Rⁱ substituents, and the other of R^{a}, R^{b}, and R^{c} is H or C₁₋₆alkyl, C₃₋₆cycloalkyl, or 3-8 membered heterocycle wherein, the C₁₋₆alkyl of R^{a}, R^{b}, or R^{c} is optionally substituted with one or more R^{h} substituents, and the 3-8 membered heterocycle of R^{a}, R^{b}, or R^{c} is optionally substituted with one or more Rⁱ substituents. In some embodiments, any two of R^{a}, R^{b}, and R^{c} are taken, together with the atoms to which they are attached, to form a oxetanyl, or azetidinyl, wherein the oxetanyl, or azetidinyl is optionally substituted with one or more Rⁱ substituents, and the other of R^{a}, R^{b}, and R^{c} is H or C₁₋₆alkyl, C₃₋₆ cycloalkyl, or 3-8 membered heterocycle wherein, the C₁₋₆alkyl of R^{a}, R^{b}, or R^{c} is optionally substituted with one or more R^{h} substituents, and the 3-8 membered heterocycle of R^{a}, R^{b}, or R^{c} is optionally substituted with one or more Rⁱ substituents. In some embodiments, any two of R^{a}, R^{b}, and R^{c} are taken, together with the atoms to which they are attached, to form

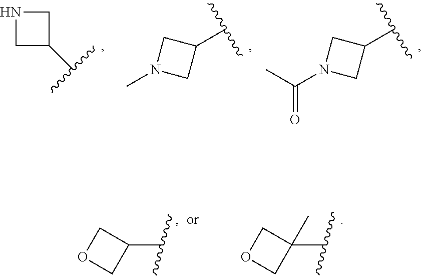

In some embodiments, provided herein is a compound of formula (A), formula (B), or formula (B-1), or a stereoisomer or tautomer thereof, or a pharmaceutically acceptable salt of any of the foregoing, wherein R^{a}, R^{b}, and R^{c} are each H, wherein the compound is of formula (B-2):

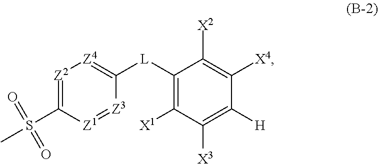

or a stereoisomer or tautomer thereof, or a pharmaceutically acceptable salt of any of the foregoing.

In some embodiments, provided herein is a compound of formula (A) or formula (B), or a stereoisomer or tautomer thereof, or a pharmaceutically acceptable salt of any of the foregoing, wherein Y is —N(C₁₋₆alkyl), wherein the C₁₋₆alkyl of the —N(C₁₋₆alkyl) is optionally substituted with one or more R^{g} substituents. In some embodiments, Y is —N(C₁₋₆alkyl), wherein the C₁₋₆alkyl of the —N(C₁₋₆alkyl) is optionally substituted with one or more R^{g} substituents, wherein the compound is of formula (B-3):

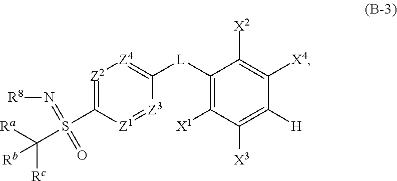

or a stereoisomer or tautomer thereof, or a pharmaceutically acceptable salt of any of the foregoing, wherein R⁸ is C₁₋₆alkyl, wherein the C₁₋₆alkyl of R^{g} is optionally substituted with one or more R^{g} substituents.

In some embodiments, provided herein is a compound of formula (A), formula (B), or formula (B-3), or a stereoisomer or tautomer thereof, or a pharmaceutically acceptable salt of any of the foregoing, wherein R⁸ is C₁₋₆alkyl, wherein the C₁₋₆alkyl of R⁸ is optionally substituted with one or more R^{g} substituents. In some embodiments, R^{g} is C₁₋₃alkyl, wherein the C₁₋₃alkyl of R⁸ is optionally substituted with one or more R^{g} substituents. In some embodiments, R⁸ is methyl, wherein the methyl of R⁸ is optionally substituted with one or more R^{g} substituents.

In some embodiments, provided herein is a compound of formula (A), formula (B), or formula (B-3), or a stereoisomer or tautomer thereof, or a pharmaceutically acceptable salt of any of the foregoing, wherein R^{g} is, independently at each occurrence, —S(O)₂C₁₋₆alkyl. In some embodiments, R^{g} is, independently at each occurrence, —S(O)₂C₁₋₃alkyl. In some embodiments, R^{g} is, independently at each occurrence, —S(O)₂CH₃.

In some embodiments, provided herein is a compound of formula (A), formula (B), or formula (B-3), or a stereoisomer or tautomer thereof, or a pharmaceutically acceptable salt of any of the foregoing, wherein R⁸ is methyl.

In some embodiments, provided herein is a compound of formula (A), formula (B), or formula (B-3), or a stereoisomer or tautomer thereof, or a pharmaceutically acceptable salt of any of the foregoing, wherein R⁸ is

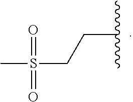 [SMILES: *CCS(C)(=O)=O]

In some embodiments, provided herein is a compound of formula (A), formula (B), or formula (B-3), or a stereoisomer or tautomer thereof, or a pharmaceutically acceptable salt of any of the foregoing, wherein R^{a}, R^{b}, and R^{c} are each H, wherein the compound is of formula (B-4):

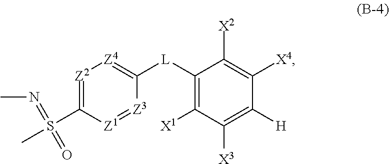

or a stereoisomer or tautomer thereof, or a pharmaceutically acceptable salt of any of the foregoing.

In some embodiments, provided herein is a compound of formula (A), such as a compound of formula (I), (I-A), (I-A1), (I-A2), (II), (II-A), (II-A1), (III), (III-A), (III-B), (III-A1), (III-B1), (III-A2), (III-B2), (III-A3), or (III-B3), or a stereoisomer or tautomer thereof, or a pharmaceutically acceptable salt of any of the foregoing, wherein m is 1. In some embodiments, provided herein is a compound of formula (A), or any variation or embodiment thereof, or a stereoisomer or tautomer thereof, or a pharmaceutically acceptable salt of any of the foregoing, wherein the compound is of formula (C):

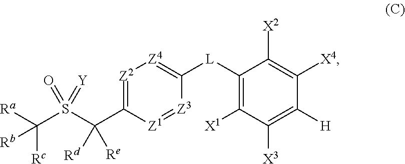

or a stereoisomer or tautomer thereof, or a pharmaceutically acceptable salt of any of the foregoing.

In some embodiments, provided herein is a compound of formula (A) or formula (C), or a stereoisomer or tautomer thereof, or a pharmaceutically acceptable salt of any of the foregoing, wherein R^{a}, R^{b}, and R^{c} are each H, wherein the compound is of formula (C-1):

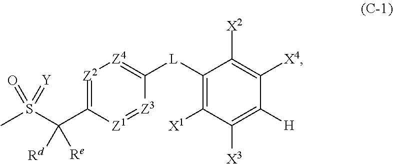

or a stereoisomer or tautomer thereof, or a pharmaceutically acceptable salt of any of the foregoing.

In some embodiments, provided herein is a compound of formula (A), formula (C), or formula (C-1), or a stereoisomer or tautomer thereof, or a pharmaceutically acceptable salt of any of the foregoing, wherein Y is 0, wherein the compound is of formula (C-2):

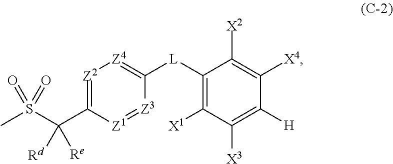

or a stereoisomer or tautomer thereof, or a pharmaceutically acceptable salt of any of the foregoing.

In some embodiments, provided herein is a compound of formula (A) or formula (I), or a stereoisomer or tautomer thereof, or a pharmaceutically acceptable salt of any of the foregoing, wherein the compound is of formula (D-1):

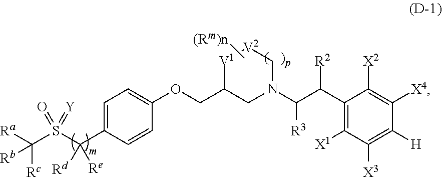

or a stereoisomer or tautomer thereof, or a pharmaceutically acceptable salt of any of the foregoing, wherein p is 0, 1, or 2, and V¹ and V² are each independently —CH₂—, —NH—, or —O—.

In some embodiments, provided herein is a compound of formula (A) or formula (II), or a stereoisomer or tautomer thereof, or a pharmaceutically acceptable salt of any of the foregoing, wherein the compound is of formula (D-2):

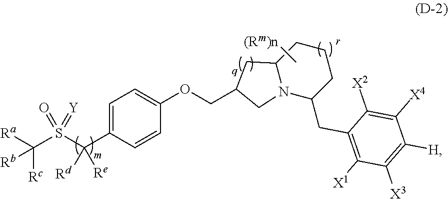

or a stereoisomer or tautomer thereof, or a pharmaceutically acceptable salt of any of the foregoing, q is 1, or 2 and r is 0 or 1.

In some embodiments, provided herein is a compound of formula (A) or formula (III), or a stereoisomer or tautomer thereof, or a pharmaceutically acceptable salt of any of the foregoing, wherein the compound is of formula (D-3A):

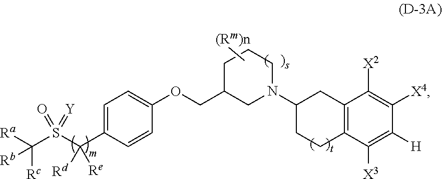

or a stereoisomer or tautomer thereof, or a pharmaceutically acceptable salt of any of the foregoing, wherein s is 0, 1, or 2 and t is 0 or 1.

In some embodiments, provided herein is a compound of formula (A) or formula (III), or a stereoisomer or tautomer thereof, or a pharmaceutically acceptable salt of any of the foregoing, wherein the compound is of formula (D-3B):

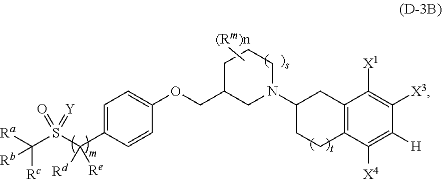

or a stereoisomer or tautomer thereof, or a pharmaceutically acceptable salt of any of the foregoing, wherein s is 0, 1, or 2 and t is 0 or 1.

In some embodiments, provided is a compound of formula (A), or a stereoisomer or tautomer thereof, or a pharmaceutically acceptable salt of any of the foregoing, wherein the compound of formula (A) is a compound of formula (E-Ia), (E-Ib), (E-IIa), (E-IIb), (E-IIIa), (E-IIIb), (E-IVa), (E-IVb), (E-Va), (E-Vb), (E-VIa), (E-VIb), (E-VIIa), or (E-VIIb):

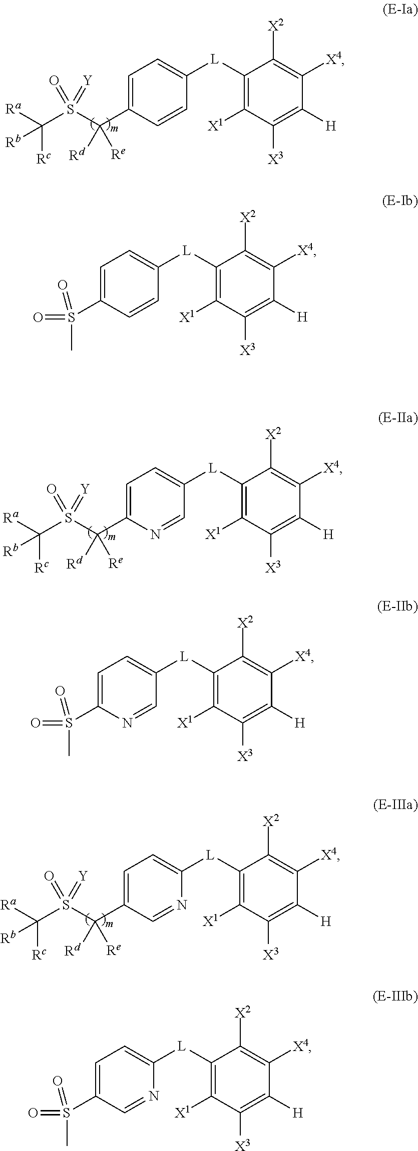

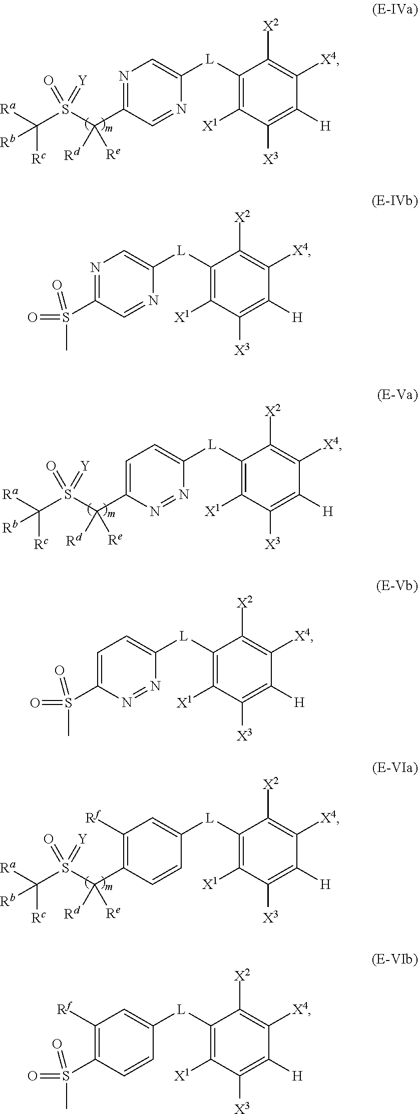

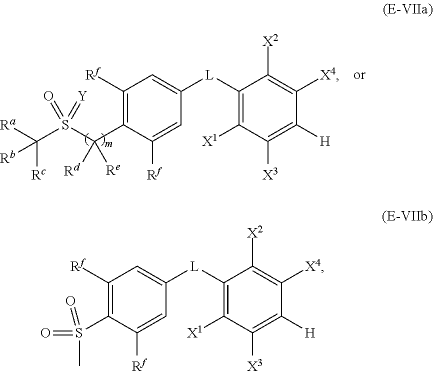

or a stereoisomer or tautomer thereof, or a pharmaceutically acceptable salt of any of the foregoing.

In some embodiments, provided herein is a compound of formula (A), such as a compound of formula (B), (B-1), (B-2), (B-3), (B-4), (C), (C-1), (C-2), (E-Ia), (E-Ib), (E-IIa), (E-IIb), (E-IIIa), (E-IIIb), (E-IVa), (E-IVb), (E-Va), (E-Vb), (E-VIa), (E-VIb), (E-VIIa), or (E-VIIb), or a stereoisomer or tautomer thereof, or a pharmaceutically acceptable salt of any of the foregoing, wherein L is

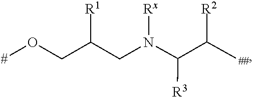

or any embodiment or variation thereof, as described elsewhere herein, including, for example, as in a compound of formula (I), (I-A1), (I-A2), (D-1), or a stereoisomer or tautomer thereof, or a pharmaceutically acceptable salt of any of the foregoing.

In some embodiments, provided herein is a compound of formula (A), such as a compound of formula (B), (B-1), (B-2), (B-3), (B-4), (C), (C-1), (C-2), (E-Ia), (E-Ib), (E-IIa), (E-IIb), (E-IIIa), (E-IIIb), (E-IVa), (E-IVb), (E-Va), (E-Vb), (E-VIa), (E-VIb), (E-VIIa), or (E-VIIb), or a stereoisomer or tatomer thereof, or a pharmaceutically acceptable salt of any of the foregoing, wherein L is

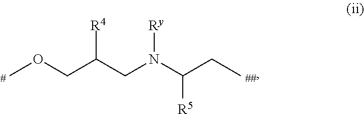

or any embodiment or variation thereof, as described elsewhere herein, including, for example, as in a compound of formula (II), (II-A), (II-A1), or (D-2), or a stereoisomer or tautomer thereof, or a pharmaceutically acceptable salt of any of the foregoing.

In some embodiments, provided herein is a compound of formula (A), such as a compound of formula (B), (B-1), (B-2), (B-3), (B-4), (C), (C-1), (C-2), (E-Ia), (E-Ib), (E-IIa), (E-IIb), (E-IIIa), (E-IIIb), (E-IVa), (E-IVb), (E-Va), (E-Vb), (E-VIa), (E-VIb), (E-VIIa), or (E-VIIb), or a stereoisomer or tatomer thereof, or a pharmaceutically acceptable salt of any of the foregoing, wherein L is

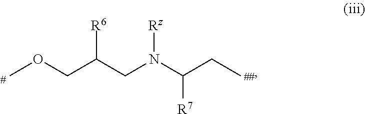

or any embodiment or variation thereof, as described elsewhere herein, including, for example, as in a compound of formula (III), (III-A), (III-B), (III-A1), (III-B1), (III-A2), (III-B32), (III-A3), (III-B33), (D-3A), or (D-3B3), or a stereoisomer or tautomer thereof, or a pharmaceutically acceptable salt of any of the foregoing.

In some embodiments, provided herein is a compound of formula (A), or a pharmaceutically acceptable salt of any of the foregoing, wherein L is selected from the group consisting of,

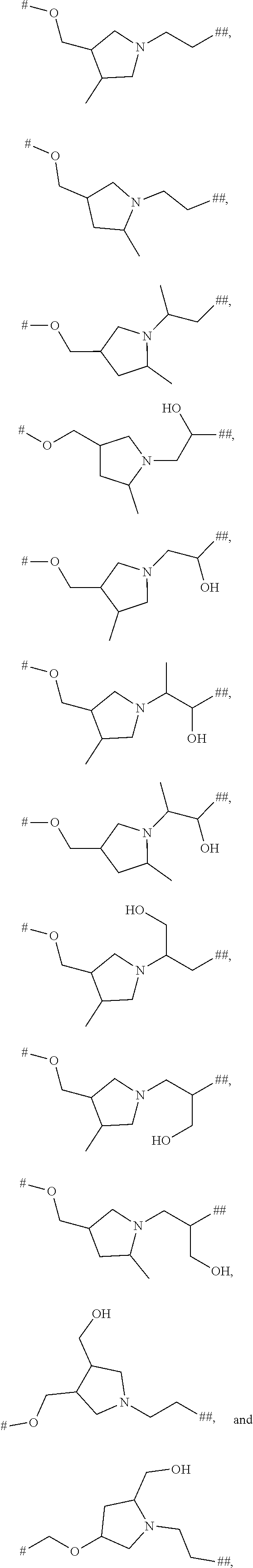

wherein # represents the point of attachment to the ring bearing Z¹, Z², Z³, and Z⁴, and ## represents the point of attachment to the remainder of the molecule. In some embodiments, L is selected from the group consisting of

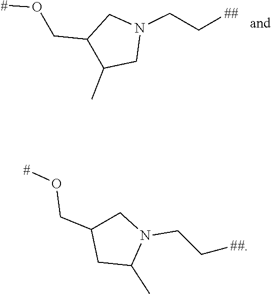

In some embodiments, L is selected from the group consisting of

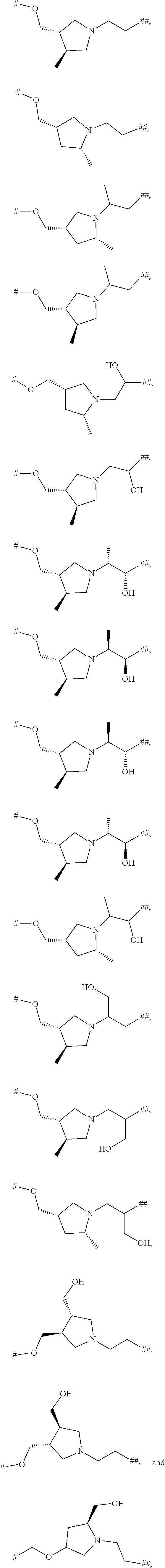

In some embodiments, L is selected from the group consisting of

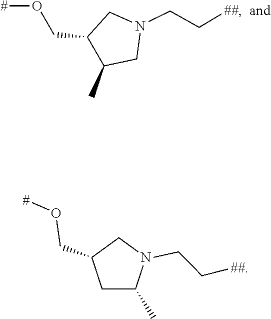

In some embodiments, provided herein is a compound of formula (A), or a pharmaceutically acceptable salt of any of the foregoing, wherein L is

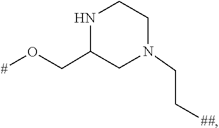

wherein # represents the point of attachment to the ring bearing Z¹, Z², Z³, and Z⁴, and ## represents the point of attachment to the remainder of the molecule. In some embodiments, L is

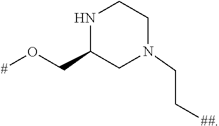

In some embodiments, provided herein is a compound of formula (A), or a pharmaceutically acceptable salt of any of the foregoing, wherein L is

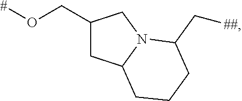

wherein # represents the point of attachment to the ring bearing Z¹, Z², Z³, and Z⁴, and ## represents the point of attachment to the remainder of the molecule. In some embodiments, L is

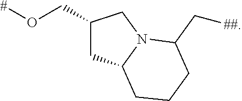

In some embodiments, provided herein is a compound of formula (A), or a pharmaceutically acceptable salt of any of the foregoing, wherein L is selected from the group consisting of

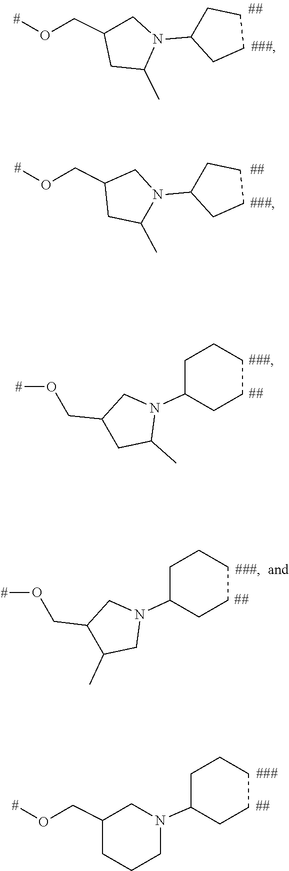

and wherein # represents the point of attachment to the ring bearing Z¹, Z², Z³, and Z⁴, ### represents the point of attachment to X¹ or X², ## represents the point of attachment to the remainder of the molecule, and the dashed line represents a single or double bond. In some embodiments, L is selected from the group consisting of

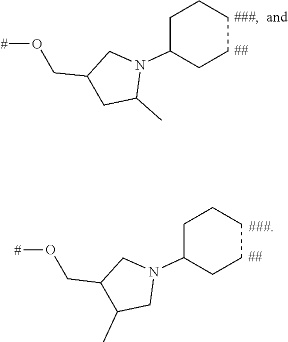

In some embodiments, L is

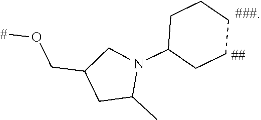

In some embodiments, L is selected from the group consisting of

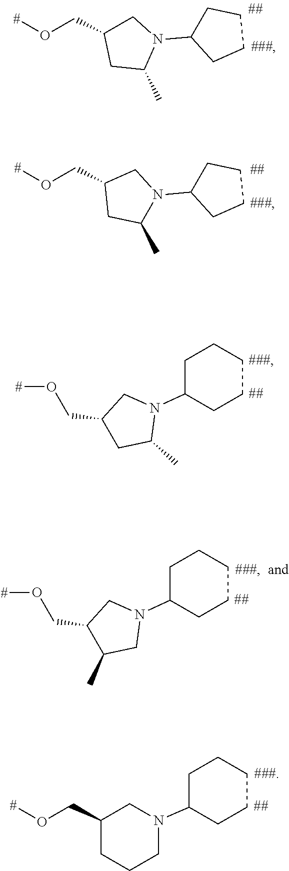

In some embodiments, L is selected from the group consisting of

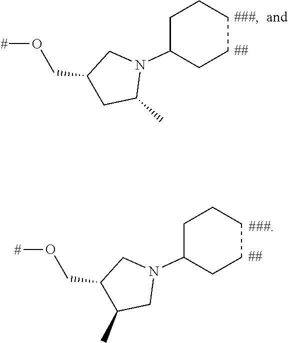

In some embodiments, L is

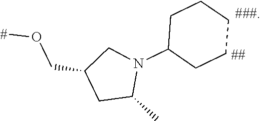

In some embodiments, provided herein is a compound of formula (A), or a pharmaceutically acceptable salt of any of the foregoing, wherein L and the phenyl ring bearing moieties X¹-X⁴ together form a structure selected from the group consisting of

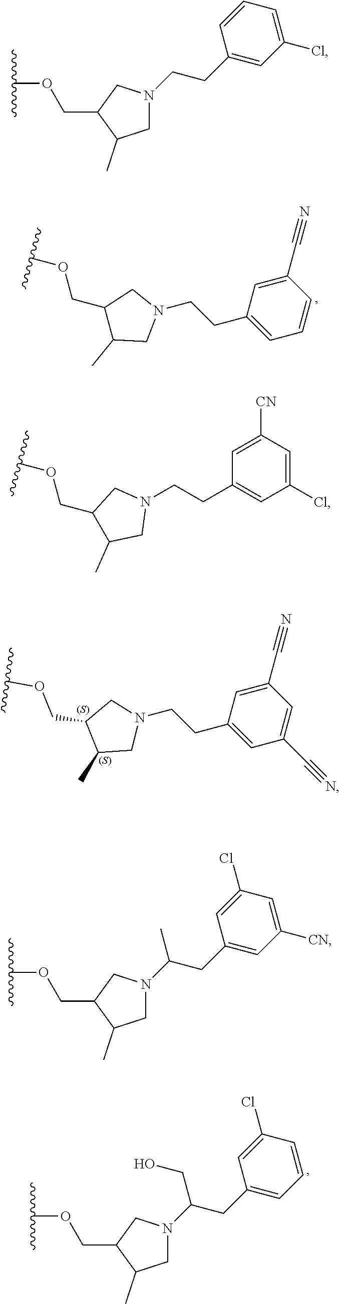

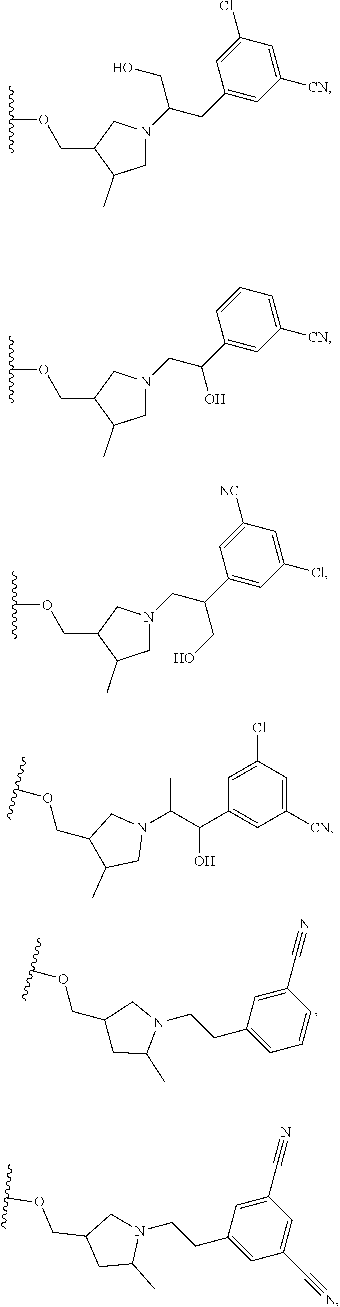 [SMILES: *OCC1CN(C(CO)Cc2cc(Cl)cc(C#N)c2)CC1C ; *OCC1CN(CC(O)c2cccc(C#N)c2)CC1C ; *OCC1CN(CC(CO)c2cc(Cl)cc(C#N)c2)CC1C ; *OCC1CN(C(C)C(O)c2cc(Cl)cc(C#N)c2)CC1C ; *OCC1CC(C)N(CCc2cccc(C#N)c2)C1 ; *OCC1CC(C)N(CCc2cc(C#N)cc(C#N)c2)C1]

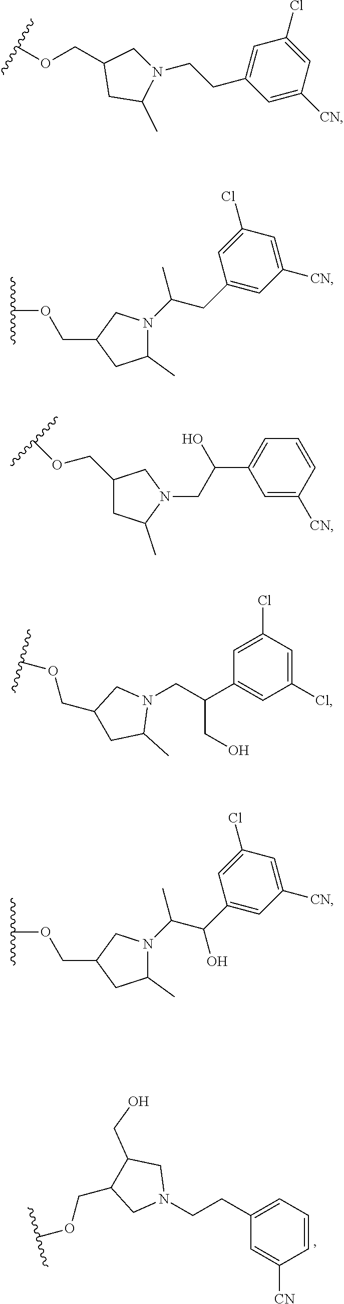 [SMILES: *OCC1CC(C)N(CCc2cc(Cl)cc(C#N)c2)C1 ; *OCC1CC(C)N(C(C)Cc2cc(Cl)cc(C#N)c2)C1 ; *OCC1CC(C)N(CC(O)c2cccc(C#N)c2)C1 ; *OCC1CC(C)N(CC(CO)c2cc(Cl)cc(Cl)c2)C1 ; *OCC1CC(C)N(C(C)C(O)c2cc(Cl)cc(C#N)c2)C1 ; *OCC1CN(CCc2cccc(C#N)c2)CC1CO]

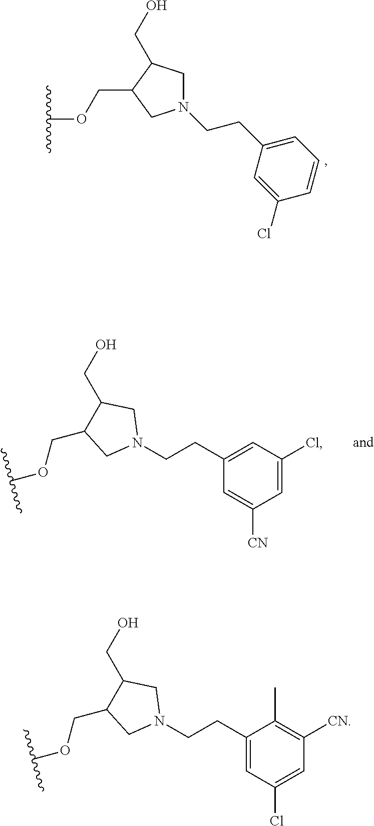 [SMILES: *OCC1CN(CCc2cccc(Cl)c2)CC1CO ; *OCC1CN(CCc2cc(Cl)cc(C#N)c2)CC1CO ; *OCC1CN(CCc2cc(Cl)cc(C#N)c2C)CC1CO]

In some embodiments, provided herein is a compound of formula (A), or a pharmaceutically acceptable salt of any of the foregoing, wherein L and the phenyl ring bearing moieties X¹-X⁴ together form a structure selected from the group consisting of

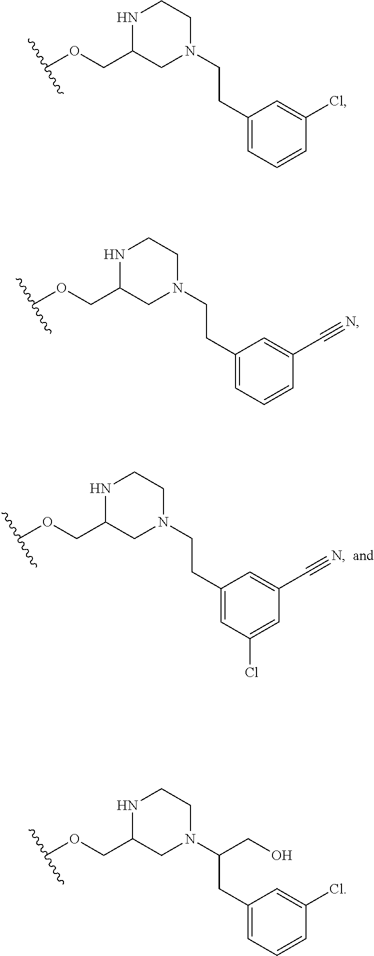

In some embodiments, provided herein is a compound of formula (A), or a pharmaceutically acceptable salt of any of the foregoing, wherein L and the phenyl ring bearing moieties X¹-X⁴ together form

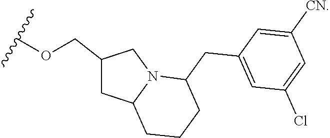 [SMILES: *OCC1CC2CCCC(Cc3cc(Cl)cc(C#N)c3)N2C1]

In some embodiments, provided herein is a compound of formula (A), or a pharmaceutically acceptable salt of any of the foregoing, wherein L and the phenyl ring bearing moieties X¹-X⁴ together form a structure selected from the group consisting of

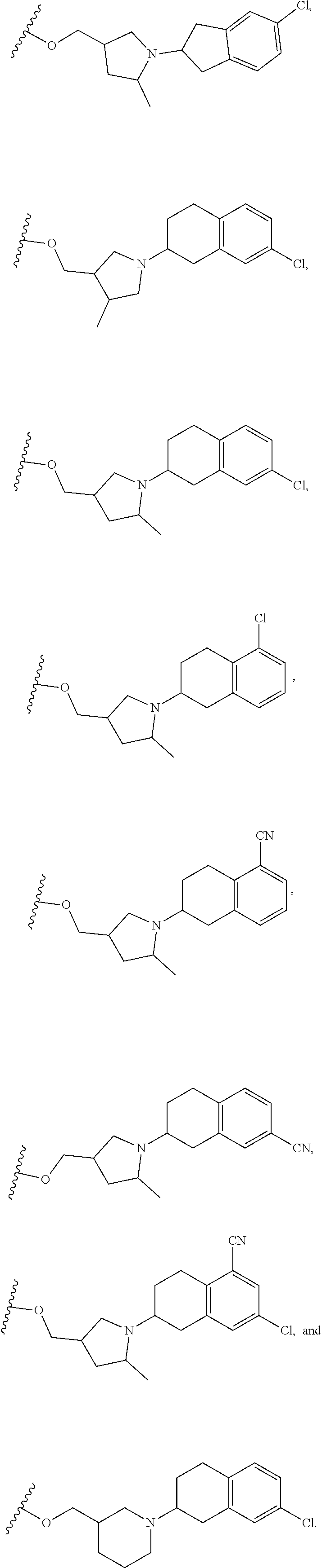

In some embodiments, provided herein is a compound of formula (A), or a pharmaceutically acceptable salt of any of the foregoing, wherein the ring bearing Z¹-Z⁴ is selected from the group consisting of

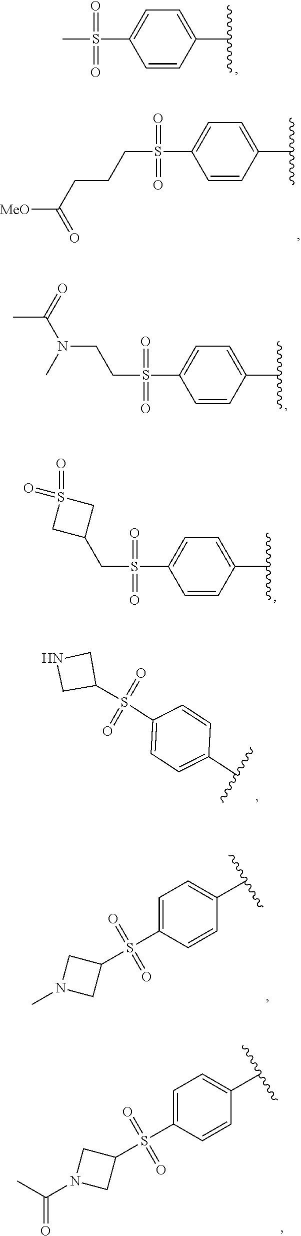

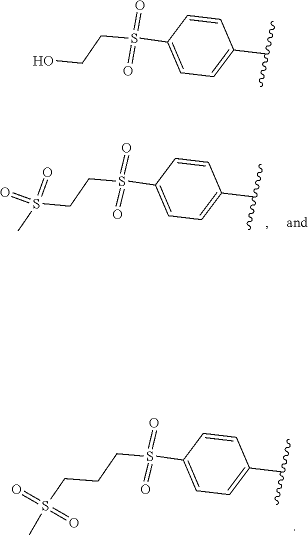 [SMILES: *c1ccc(S(=O)(=O)CCO)cc1 ; *c1ccc(S(=O)(=O)CCS(C)(=O)=O)cc1 ; *c1ccc(S(=O)(=O)CCCS(C)(=O)=O)cc1]

In some embodiments, provided herein is a compound of formula (A), or a pharmaceutically acceptable salt of any of the foregoing, wherein the ring bearing Z¹-Z⁴ is selected from the group consisting of

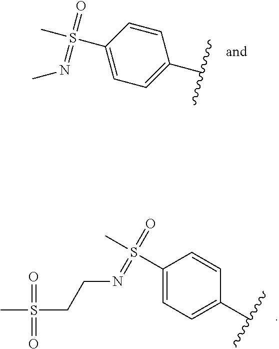 [SMILES: *c1ccc(S(C)(=O)=NC)cc1 ; *c1ccc(S(C)(=O)=NCCS(C)(=O)=O)cc1]

In some embodiments, provided herein is a compound of formula (A), or a pharmaceutically acceptable salt of any of the foregoing, wherein the ring bearing Z¹-Z⁴ is selected from the group consisting of

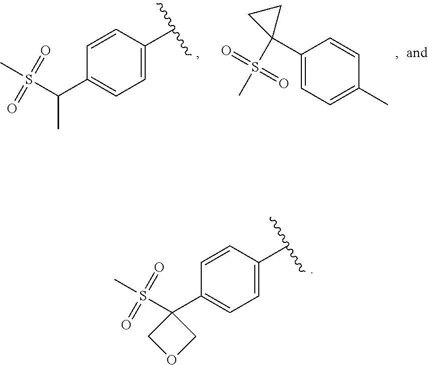

In some embodiments, provided herein is a compound of formula (A), or a pharmaceutically acceptable salt of any of the foregoing, wherein the ring bearing Z¹-Z⁴ is selected from the group consisting of

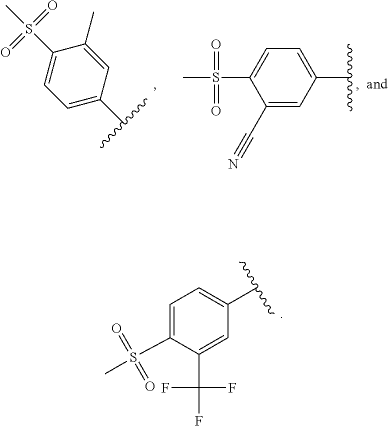 [SMILES: *c1ccc(S(C)(=O)=O)c(C)c1 ; *c1ccc(S(C)(=O)=O)c(C#N)c1 ; *c1ccc(S(C)(=O)=O)c(C(F)(F)F)c1]

In some embodiments, provided herein is a compound of formula (A), or a pharmaceutically acceptable salt of any of the foregoing, wherein the ring bearing Z¹-Z⁴ is

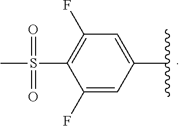 [SMILES: *c1cc(F)c(S(C)(=O)=O)c(F)c1]

In some embodiments, provided herein is a compound of formula (A), or a pharmaceutically acceptable salt of any of the foregoing, wherein the ring bearing Z¹-Z⁴ is

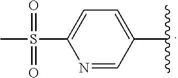 [SMILES: *c1ccc(S(C)(=O)=O)nc1]

In some embodiments, provided herein is a compound of formula (A), or a pharmaceutically acceptable salt of any of the foregoing, wherein the ring bearing Z¹-Z⁴ is

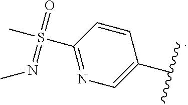 [SMILES: *c1ccc(S(C)(=O)=NC)nc1]

In some embodiments, provided herein is a compound of formula (A), or a pharmaceutically acceptable salt of any of the foregoing, wherein the ring bearing Z¹-Z⁴ is

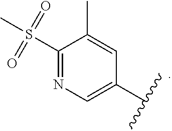 [SMILES: *c1cnc(S(C)(=O)=O)c(C)c1]

In some embodiments, provided herein is a compound of formula (A), or a pharmaceutically acceptable salt of any of the foregoing, wherein the ring bearing Z¹-Z⁴ is selected from the group consisting of

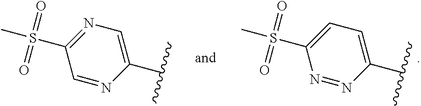 [SMILES: *c1cnc(S(C)(=O)=O)cn1 ; *c1ccc(S(C)(=O)=O)nn1]

In some embodiments, provided herein is a compound of formula (A), or a pharmaceutically acceptable salt of any of the foregoing, wherein L and the ring bearing Z¹-Z⁴ together form a structure selected from the group consisting of

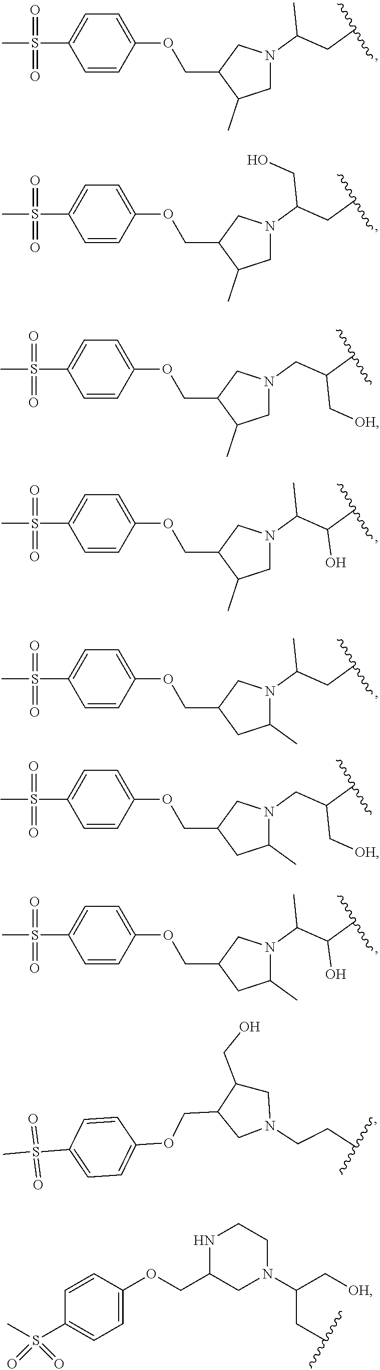

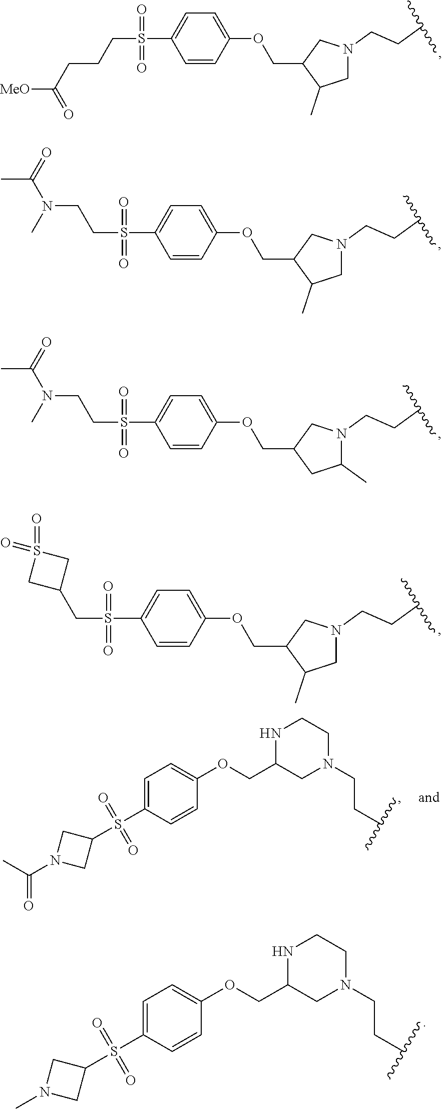

In some embodiments, provided herein is a compound of formula (A), or a pharmaceutically acceptable salt of any of the foregoing, wherein L and the ring bearing Z¹-Z⁴ together form

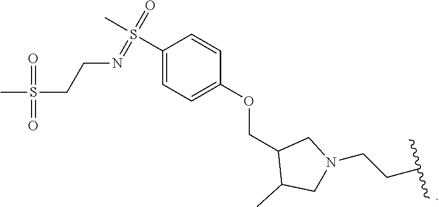 [SMILES: *CCN1CC(C)C(COc2ccc(S(C)(=O)=NCCS(C)(=O)=O)cc2)C1]

In some embodiments, provided herein is a compound of formula (A), or a pharmaceutically acceptable salt of any of the foregoing, wherein L and the ring bearing Z¹-Z⁴ together form a structure selected from the group consisting of

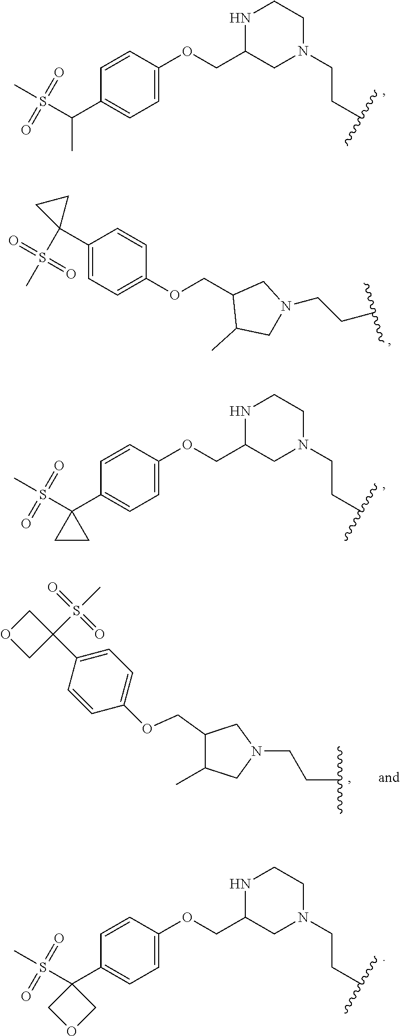

In some embodiments, provided herein is a compound of formula (A), or a pharmaceutically acceptable salt of any of the foregoing, wherein L and the ring bearing Z¹-Z⁴ together form

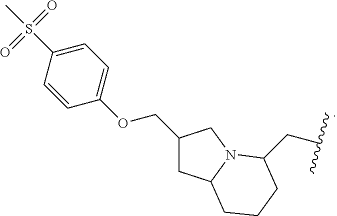 [SMILES: *CC1CCCC2CC(COc3ccc(S(C)(=O)=O)cc3)CN12]

In some embodiments, provided herein is a compound of formula (A), or a pharmaceutically acceptable salt of any of the foregoing, wherein L and the ring bearing Z¹-Z⁴ together form a structure selected from the group consisting of

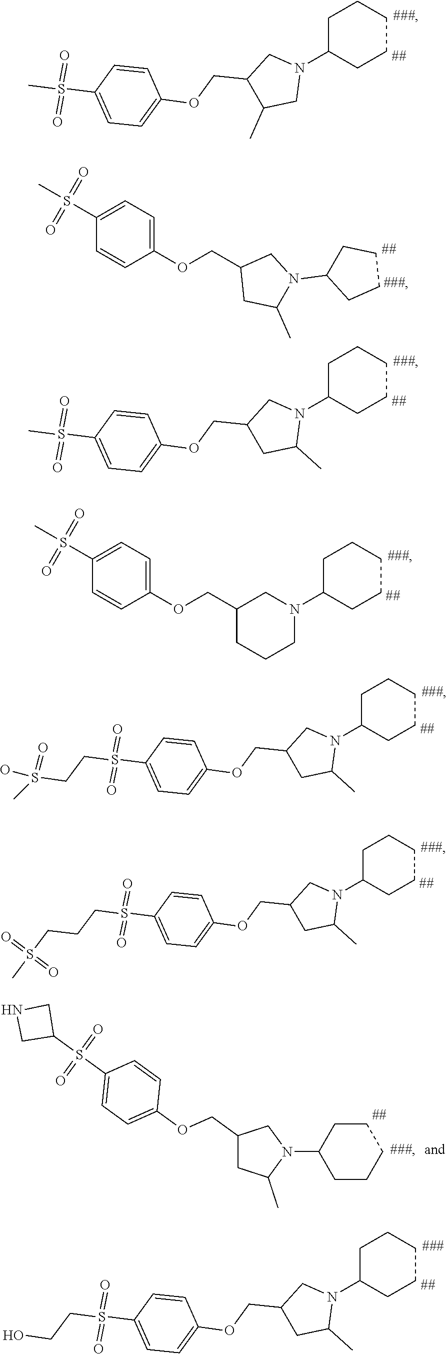

wherein # represents the point of attachment to the ring bearing Z¹, Z², Z³, and Z⁴, ### represents the point of attachment to X¹ or X², ## represents the point of attachment to the remainder of the molecule, and the dashed line represents a single or double bond.

In some embodiments, provided herein is a compound of formula (A), or a pharmaceutically acceptable salt of any of the foregoing, wherein L and the ring bearing Z¹-Z⁴ together form a

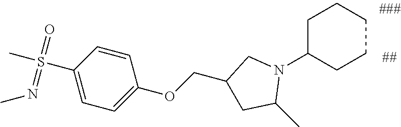

wherein # represents the point of attachment to the ring bearing Z¹, Z², Z³, and Z⁴, ### represents the point of attachment to X¹ or X², ## represents the point of attachment to the remainder of the molecule, and the dashed line represents a single or double bond.

In some embodiments, provided herein is a compound of formula (A), or a pharmaceutically acceptable salt of any of the foregoing, wherein L and the ring bearing Z¹-Z⁴ together form a structure selected from the group consisting of

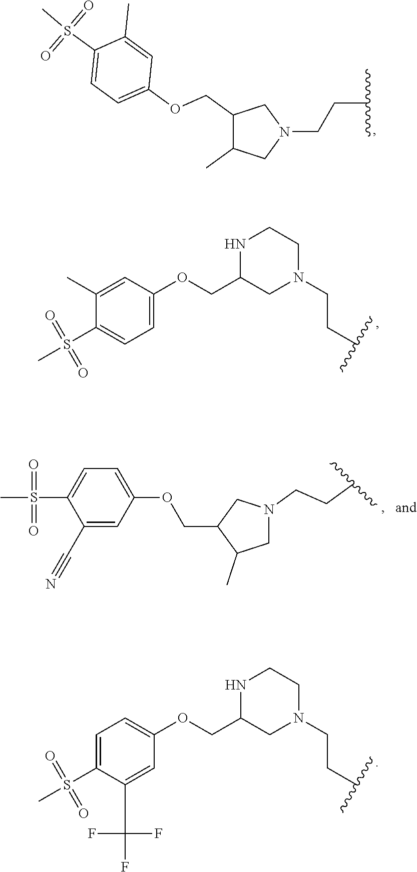 [SMILES: *CCN1CC(C)C(COc2ccc(S(C)(=O)=O)c(C)c2)C1 ; *CCN1CCNC(COc2ccc(S(C)(=O)=O)c(C)c2)C1 ; *CCN1CC(C)C(COc2ccc(S(C)(=O)=O)c(C#N)c2)C1 ; *CCN1CCNC(COc2ccc(S(C)(=O)=O)c(C(F)(F)F)c2)C1]

In some embodiments, provided herein is a compound of formula (A), or a pharmaceutically acceptable salt of any of the foregoing, wherein L and the ring bearing Z¹-Z⁴ together form

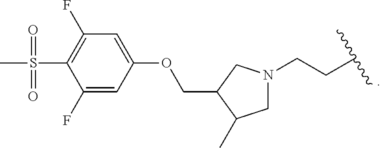 [SMILES: *CCN1CC(C)C(COc2cc(F)c(S(C)(=O)=O)c(F)c2)C1]

In some embodiments, provided herein is a compound of formula (A), or a pharmaceutically acceptable salt of any of the foregoing, wherein L and the ring bearing Z¹-Z⁴ together form a structure selected from the group consisting of

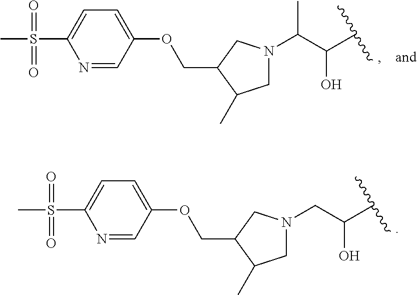 [SMILES: *C(O)C(C)N1CC(C)C(COc2ccc(S(C)(=O)=O)nc2)C1 ; *C(O)CN1CC(C)C(COc2ccc(S(C)(=O)=O)nc2)C1]

In some embodiments, provided herein is a compound of formula (A), or a pharmaceutically acceptable salt of any of the foregoing, wherein L and the ring bearing Z¹-Z⁴ together form

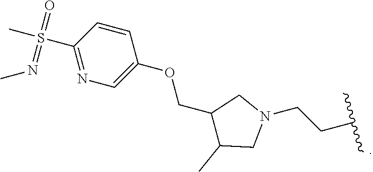 [SMILES: *CCN1CC(C)C(COc2ccc(S(C)(=O)=NC)nc2)C1]

In some embodiments, provided herein is a compound of formula (A), or a pharmaceutically acceptable salt of any of the foregoing, wherein L and the ring bearing Z¹-Z⁴ together form a structure selected from the group consisting of

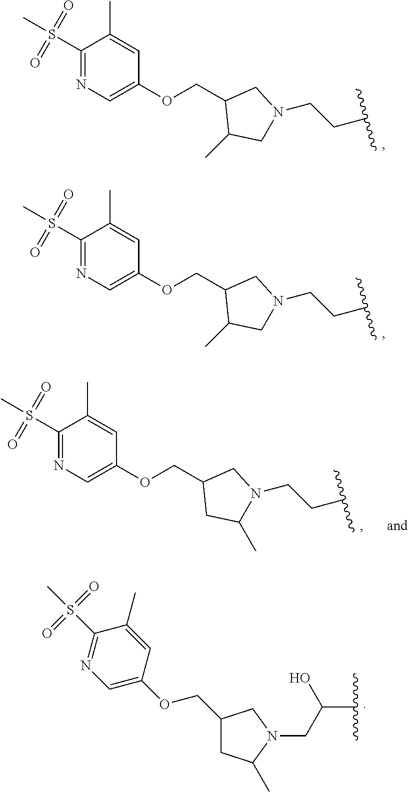 [SMILES: *CCN1CC(C)C(COc2cnc(S(C)(=O)=O)c(C)c2)C1 ; *CCN1CC(C)C(COc2cnc(S(C)(=O)=O)c(C)c2)C1 ; *CCN1CC(COc2cnc(S(C)(=O)=O)c(C)c2)CC1C ; *C(O)CN1CC(COc2cnc(S(C)(=O)=O)c(C)c2)CC1C]

In some embodiments, provided herein is a compound of formula (A), or a pharmaceutically acceptable salt of any of the foregoing, wherein L and the ring bearing Z¹-Z⁴ together form a structure selected from the groups consisting of

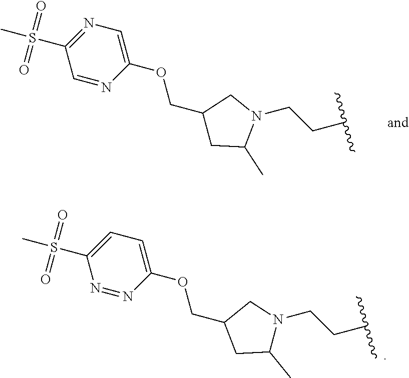 [SMILES: *CCN1CC(COc2cnc(S(C)(=O)=O)cn2)CC1C ; *CCN1CC(COc2ccc(S(C)(=O)=O)nn2)CC1C]

In other variations, provided herein is a compound of formula (X):

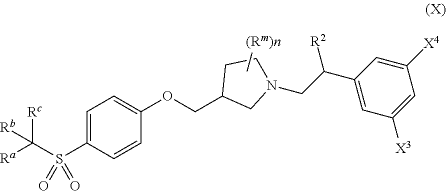

or a stereoisomer or a tautomer thereof, or a pharmaceutically acceptable salt of any of the foregoing, wherein:
R^{a}, R^{b}, and R^{c} are each independently H;
or any two of R^{a}, R^{b}, and R^{c} are taken, together with the atoms to which they are attached, to form a 3-6 membered heterocyclyl, and the other of R^{a}, R^{b}, and R^{c} is C₁₋₆alkyl;
n is 1;
R^{m} is C₁₋₆alkyl;
R² is H, or —OH; and
X³, and X⁴ are, independently of each other, H, halo, or —CN;
provided that at least one of X³, and X⁴ is halo, or —CN.

It is to be understood that any variation or embodiment of R¹, R², R³, R⁴, R⁵, R, R⁷, R⁸, R^{a}, R^{b}, R^{c}, R^{d}, R^{e}, R^{f}, R^{g}, R^{h}, Rⁱ, R^{j}, R^{k}, R^{m}, Rⁿ, R^{x}, R^{y}, R^{z}, X¹, X², X³, X⁴, Z¹, Z², Z³, Z⁴, L, Y, m, n, p, q, r, s, and t provided herein can be combined with every other variation or embodiment of R¹, R², R³, R⁴, R⁵, R⁶, R⁷, R⁸, R^{a}, R^{b}, R^{c}, R^{d}, R^{e}, R^{f}, R^{g}, R^{h}, Rⁱ, R^{j}, R^{k}, R^{m}, Rⁿ, R^{x}, R^{y}, R^{z}, X¹, X², X³, X⁴, Z¹, Z², Z³, Z⁴, L, Y, m, n, p, q, r, s, and t provided, the same as if each and every combination had been individually and specifically described.

In some embodiments, provided herein is a compound of formula (A), or any variation of embodiment thereof, or a stereoisomer or tautomer thereof, or a pharmaceutically acceptable salt of any of the foregoing, wherein the compound is a compound of Table 1, or a stereoisomer or tautomer thereof, or a pharmaceutically acceptable salt of any of the foregoing.

TABLE 1

| Compound | | |
|---|---|---|
| No. | Structure | Name |
| 1 | 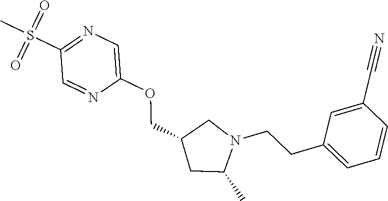 [SMILES: C[C@@H]1C[C@H](COc2cnc(S(C)(=O)=O)cn2)CN1CCc1cccc(C#N)c1] | 3-(2-((2R,4S)-2-methyl-4- (((5-(methylsulfonyl)pyr- azin-2-yl)oxy)methyl)pyr- rolidin-1-yl)-ethyl)benzo- nitrile |
| 2 | 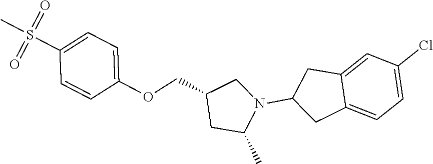 [SMILES: C[C@@H]1C[C@H](COc2ccc(S(C)(=O)=O)cc2)CN1C1Cc2ccc(Cl)cc2C1] | (2R,4S)-1-[5-chloro-2,3- dihydro-1H-inden-2-yl]-4- [(4-methanesulfonylphen- oxy)-methyl]-2-methylpyr- rolidine |
| 3 | 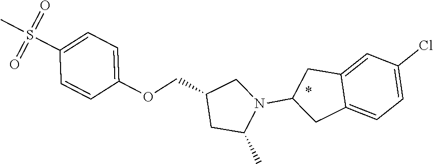 [SMILES: C[C@@H]1C[C@H](COc2ccc(S(C)(=O)=O)cc2)CN1C1Cc2ccc(Cl)cc2C1] | (2R,4S)-1-[(2R or 2S)-5- chloro-2,3-dihydro-1H- inden-2-yl]-4-[(4-methane- sulfonylphenoxy)methyl]- 2-methylpyrrolidine |
| 4 | 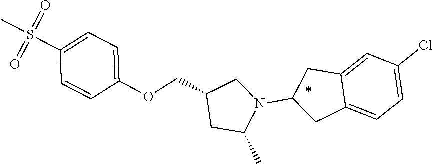 [SMILES: C[C@@H]1C[C@H](COc2ccc(S(C)(=O)=O)cc2)CN1C1Cc2ccc(Cl)cc2C1] | (2R,4S)-1-[(2S or 2R)-5- chloro-2,3-dihydro-1H- inden-2-yl]-4-[(4-methane- sulfonylphenoxy)methyl]- 2-methylpyrrolidine |
| 5 | 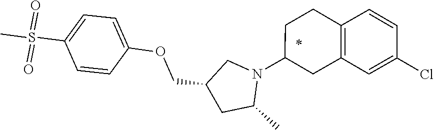 [SMILES: C[C@@H]1C[C@H](COc2ccc(S(C)(=O)=O)cc2)CN1C1CCc2ccc(Cl)cc2C1] | (2R,4S)-1-[(2S or 2R)-7- chloro-1,2,3,4-tetrahydro- naphthalen-2-yl]-4-[(4- methanesulfonylphenoxy)- methyl]-2-methylpyrrol- idine |
| 6 | 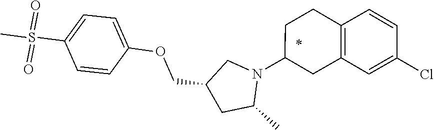 [SMILES: C[C@@H]1C[C@H](COc2ccc(S(C)(=O)=O)cc2)CN1C1CCc2ccc(Cl)cc2C1] | (2R,4S)-1-[(2R or 2S)-7- chloro-1,2,3,4-tetrahydro- naphthalen-2-yl]-4-[(4- methanesulfonylphenoxy)- methyl]-2-methylpyrrol- idine |
| 7 | 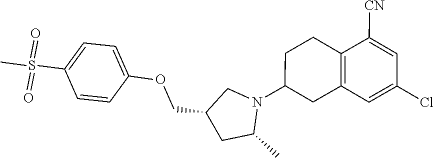 [SMILES: C[C@@H]1C[C@H](COc2ccc(S(C)(=O)=O)cc2)CN1C1CCc2c(C#N)cc(Cl)cc2C1] | (6R or 6S)-3-chloro-6- [(2R,4S)-4-[(4-methane- sulfonylphenoxy)methyl]- 2-methylpyrrolidin-1-yl]- 5,6,7,8-tetrahydronaph- thalene-1-carbonitrile |
| 8 | 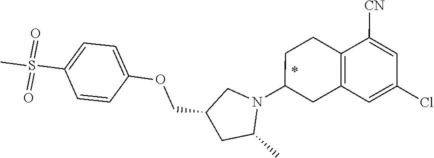 [SMILES: C[C@@H]1C[C@H](COc2ccc(S(C)(=O)=O)cc2)CN1C1CCc2c(C#N)cc(Cl)cc2C1] | (6R or 6S)-3-chloro-6- [(2R,4S)-4-[(4-methane- sulfonylphenoxy)methyl]- 2-methylpyrrolidin-1-yl]- 5,6,7,8-tetrahydronaph- thalene-1-carbonitrile |
| 9 | 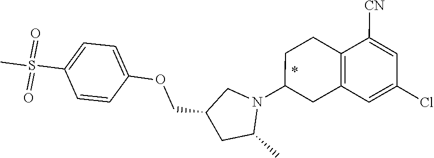 [SMILES: C[C@@H]1C[C@H](COc2ccc(S(C)(=O)=O)cc2)CN1C1CCc2c(C#N)cc(Cl)cc2C1] | (6S or 6R)-3-chloro-6- [(2R,4S)-4-[(4-methane- sulfonylphenoxy)methyl]- 2-methylpyrrolidin-1-yl]- 5,6,7,8-tetrahydronaph- thalene-1-carbonitrile |
| 10 | 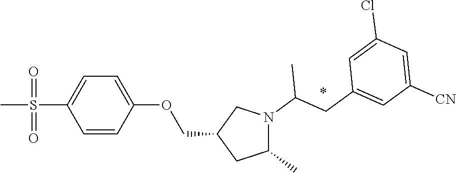 [SMILES: CC(Cc1cc(Cl)cc(C#N)c1)N1C[C@@H](COc2ccc(S(C)(=O)=O)cc2)C[C@H]1C] | 3-chloro-5-[(2S or 2R)-2- [(2R,4S)-4-[(4-methane- sulfonylphenoxy)methyl]- 2-methylpyrrolidin-1-yl] propyl]benzonitrile |
| 11 | 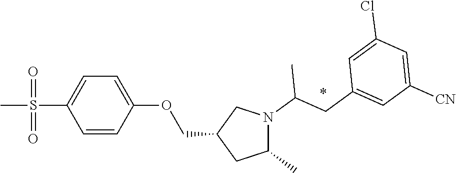 [SMILES: CC(Cc1cc(Cl)cc(C#N)c1)N1C[C@@H](COc2ccc(S(C)(=O)=O)cc2)C[C@H]1C] | 3-chloro-5-[(2R or 2S)-2- [(2R,4S)-4-[(4-methane- sulfonylphenoxy)methyl]- 2-methylpyrrolidin-1-yl] propyl]benzonitrile |
| 12 | 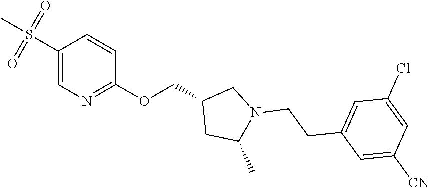 [SMILES: C[C@@H]1C[C@H](COc2ccc(S(C)(=O)=O)cn2)CN1CCc1cc(Cl)cc(C#N)c1] | 3-chloro-5-{2-[(2R,4S)-4- {[(5-methanesulfonylpyr- idin-2-yl)oxy]methyl}-2- methylpyrrolidin-1-yl] ethyl}benzonitrile |
| 13 | 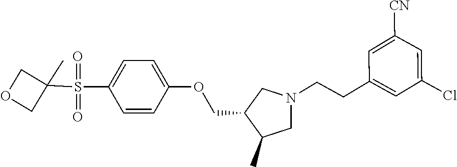 [SMILES: C[C@@H]1CN(CCc2cc(Cl)cc(C#N)c2)C[C@H]1COc1ccc(S(=O)(=O)C2(C)COC2)cc1] | 3-chloro-5-{2-[(3S,4S)-3- methyl-4-({4-[(3-methyl- oxetan-3-yl)sulfonyl]phen- oxy}methyl)pyrrolidin-1- yl]ethyl}benzonitrile |
| 14 | 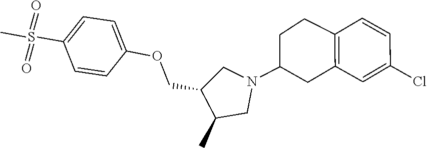 [SMILES: C[C@@H]1CN(C2CCc3ccc(Cl)cc3C2)C[C@H]1COc1ccc(S(C)(=O)=O)cc1] | (3S,4S)-1-[7-chloro-1,2,3,4- tetrahydronaphthalen-2- yl]-3-[(4-methanesulfonyl- phenoxy)methyl]-4-methyl- pyrrolidine |
| 15 | 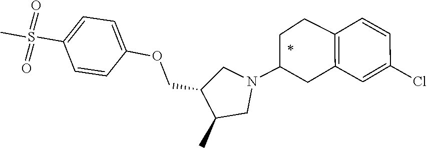 [SMILES: C[C@@H]1CN(C2CCc3ccc(Cl)cc3C2)C[C@H]1COc1ccc(S(C)(=O)=O)cc1] | (3S,4S)-1-[(2S or 2R)-7- chloro-1,2,3,4-tetrahydro- naphthalen-2-yl]-3-[(4- methanesulfonylphenoxy)- methyl]-4-methylpyrrol- idine |
| 16 | 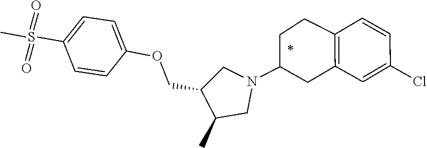 [SMILES: C[C@@H]1CN(C2CCc3ccc(Cl)cc3C2)C[C@H]1COc1ccc(S(C)(=O)=O)cc1] | (3S,4S)-1-[(2R or 2S)-7- chloro-1,2,3,4-tetrahydro- naphthalen-2-yl]-3-[(4- methanesulfonylphenoxy)- methyl]-4-methylpyrrol- idine |
| 17 | 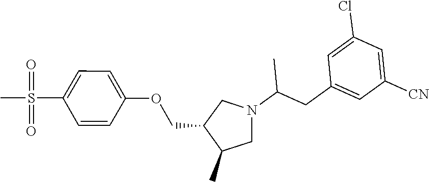 [SMILES: CC(Cc1cc(Cl)cc(C#N)c1)N1C[C@@H](COc2ccc(S(C)(=O)=O)cc2)[C@H](C)C1] | 3-chloro-5-[2-[(3S,4S)-3- [(4-methanesulfonylphen- oxy)methyl]-4-methylpyr- rolidin-1-yl]propyl]benzo- nitrile |
| 18 | 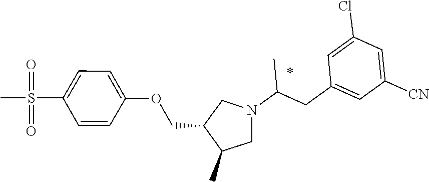 [SMILES: CC(Cc1cc(Cl)cc(C#N)c1)N1C[C@@H](COc2ccc(S(C)(=O)=O)cc2)[C@H](C)C1] | 3-chloro-5-[(2S or 2R)-2- [(3S,4S)-3-[(4-methane- sulfonylphenoxy)methyl]- 4-methylpyrrolidin-1-yl] propyl]benzonitrile |
| 19 | 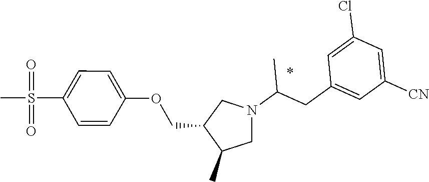 [SMILES: CC(Cc1cc(Cl)cc(C#N)c1)N1C[C@@H](COc2ccc(S(C)(=O)=O)cc2)[C@H](C)C1] | 3-chloro-5-[(2R or 2S)-2- [(3S,4S)-3-[(4-methane- sulfonylphenoxy)methyl]- 4-methylpyrrolidin-1-yl] propyl]benzonitrile |
| 20 | 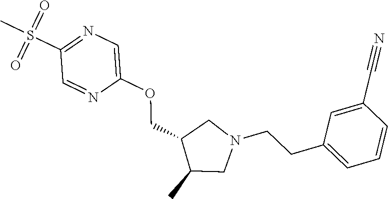 [SMILES: C[C@@H]1CN(CCc2cccc(C#N)c2)C[C@H]1COc1cnc(S(C)(=O)=O)cn1] | 3-{2-[(3S,4S)-3-{[(5- methanesulfonylpyrazin-2- yl)oxy]methyl}-4-methyl- pyrrolidin-1-yl]ethyl} benzonitrile |
| 21 | 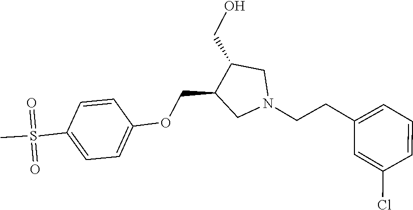 [SMILES: CS(=O)(=O)c1ccc(OC[C@@H]2CN(CCc3cccc(Cl)c3)C[C@H]2CO)cc1] | [(3S,4S)-1-[2-(3-chloro- phenyl)ethyl]-4-[(4- methanesulfonylphenoxy)- methyl]pyrrolidin-3-yl] methanol |
| 22 | 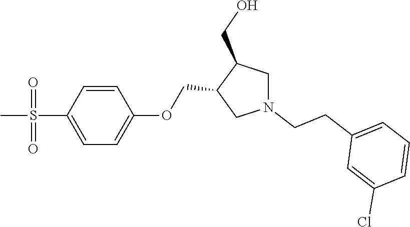 [SMILES: CS(=O)(=O)c1ccc(OC[C@H]2CN(CCc3cccc(Cl)c3)C[C@@H]2CO)cc1] | [(3R,4R)-1-[2-(3-chloro- phenyl)ethyl]-4-[(4- methanesulfonylphenoxy)- methyl]pyrrolidin-3-yl] methanol |
| 23 | 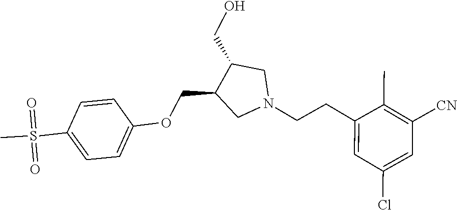 [SMILES: Cc1c(C#N)cc(Cl)cc1CCN1C[C@@H](CO)[C@H](COc2ccc(S(C)(=O)=O)cc2)C1] | 5-chloro-3-{2-[(3S,4S)-3- (hydroxymethyl)-4-[(4- methanesulfonylphenoxy)- methyl]pyrrolidin-1-yl] ethyl}-2-methylbenzonitrile |
| 24 | 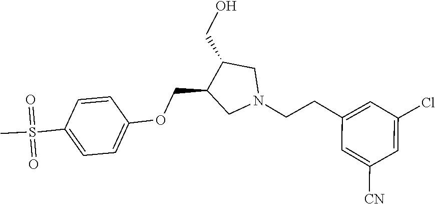 [SMILES: CS(=O)(=O)c1ccc(OC[C@@H]2CN(CCc3cc(Cl)cc(C#N)c3)C[C@H]2CO)cc1] | 3-chloro-5-{2-[(3S,4S)-3- (hydroxymethyl)-4-[(4- methanesulfonylphenoxy)- methyl]pyrrolidin-1-yl] ethyl}benzonitrile |
| 25 | 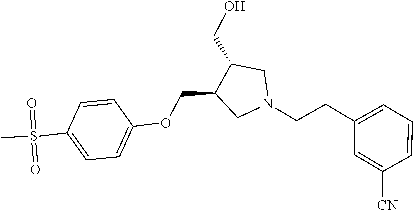 [SMILES: CS(=O)(=O)c1ccc(OC[C@@H]2CN(CCc3cccc(C#N)c3)C[C@H]2CO)cc1] | 3-{2-[(3S,4S)-3-(hydroxy- methyl)-4-[(4-methane- sulfonylphenoxy)methyl] pyrrolidin-1-yl]ethyl} benzonitrile |
| 26 | 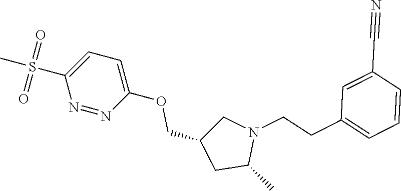 [SMILES: C[C@@H]1C[C@H](COc2ccc(S(C)(=O)=O)nn2)CN1CCc1cccc(C#N)c1] | 3-{2-[(2R,4S)-4-{[(6- methanesulfonylpyridazin- 3-yl)oxy]methyl}-2-meth- ylpyrrolidin-1-ylethyl} benzonitrile |
| 27 | 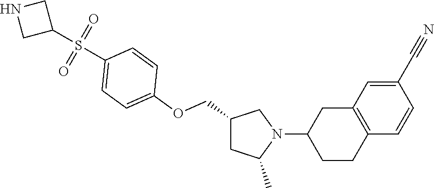 [SMILES: C[C@@H]1C[C@H](COc2ccc(S(=O)(=O)C3CNC3)cc2)CN1C1CCc2ccc(C#N)cc2C1] | 7-[(2R,4S)-4-{[4-(azeti- dine-3-sulfonyl)phenoxy] methyl}-2-methylpyrrol- idin-1-yl]-5,6,7,8-tetra- hydronaphthalene-2- carbonitrile |
| 28 | 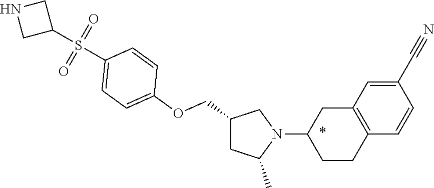 [SMILES: C[C@@H]1C[C@H](COc2ccc(S(=O)(=O)C3CNC3)cc2)CN1C1CCc2ccc(C#N)cc2C1] | 7-[(2R,4S)-4-{[4-(azeti- dine-3-sulfonyl)phenoxy] methyl}-2-methylpyrrol- idin-1-yl]-5,6,7,8-tetra- hydronaphthalene-2- carbonitrile |
| 29 | 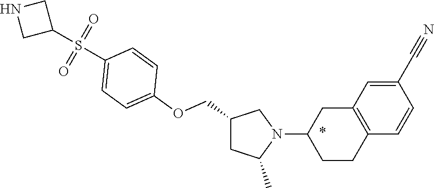 [SMILES: C[C@@H]1C[C@H](COc2ccc(S(=O)(=O)C3CNC3)cc2)CN1C1CCc2ccc(C#N)cc2C1] | 7-[(2R,4S)-4-{[4-(azeti- dine-3-sulfonyl)phenoxy] methyl}-2-methylpyrrol- idin-1-yl]-5,6,7,8-tetra- hydronaphthalene-2- carbonitrile |
| 30 | 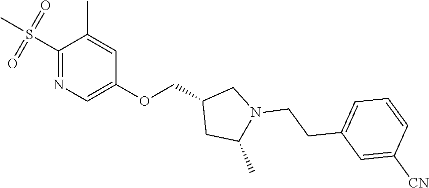 [SMILES: Cc1cc(OC[C@H]2C[C@@H](C)N(CCc3cccc(C#N)c3)C2)cnc1S(C)(=O)=O] | 3-{2-[(2R,4S)-4-{[(6- methanesulfonyl-5-methyl- pyridin-3-yl)oxy]methyl}- 2-methylpyrrolidin-1-yl] ethyl}benzonitrile |
| 31 | 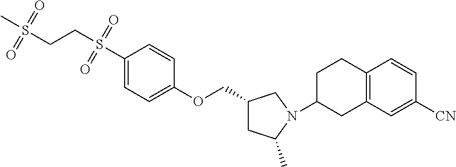 [SMILES: C[C@@H]1C[C@H](COc2ccc(S(=O)(=O)CCS(C)(=O)=O)cc2)CN1C1CCc2ccc(C#N)cc2C1] | 7-[(2R,4S)-4-{[4-(2- methanesulfonylethane- sulfonyl)phenoxy]methyl}- 2-methylpyrrolidin-1-yl]- 5,6,7,8-tetrahydronaph- thalene-2-carbonitrile |
| 32 | 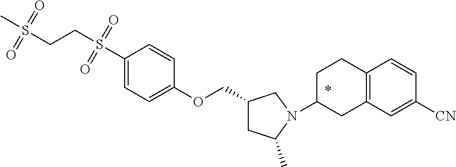 [SMILES: C[C@@H]1C[C@H](COc2ccc(S(=O)(=O)CCS(C)(=O)=O)cc2)CN1C1CCc2ccc(C#N)cc2C1] | 7-[(2R,4S)-4-{[4-(2- methanesulfonylethane- sulfonyl)phenoxy]methyl}- 2-methylpyrrolidin-1-yl]- 5,6,7,8-tetrahydronaph- thalene-2-carbonitrile |
| 33 | 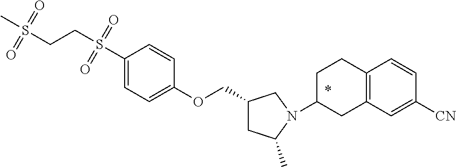 [SMILES: C[C@@H]1C[C@H](COc2ccc(S(=O)(=O)CCS(C)(=O)=O)cc2)CN1C1CCc2ccc(C#N)cc2C1] | 7S or 7R)-7-[(2R,4S)-4- {[4-(2-methanesulfonyl- ethanesulfonyl)phenoxy] methyl}-2-methylpyrrol- idin-1-yl]-5,6,7,8-tetra- hydronaphthalene-2- carbonitrile |
| 34 | 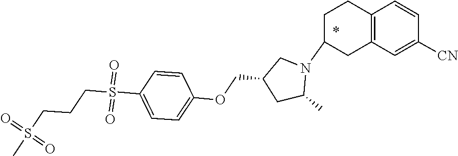 [SMILES: C[C@@H]1C[C@H](COc2ccc(S(=O)(=O)CCCS(C)(=O)=O)cc2)CN1C1CCc2ccc(C#N)cc2C1] | (7R or 7S)-7-[(2R,4S)-4- {[4-(3-methanesulfonyl- propanesulfonyl)phenoxy] methyl}-2-methylpyrrol- idin-1-yl]-5,6,7,8-tetra- hydronaphthalene-2- carbonitrile |
| 35 | 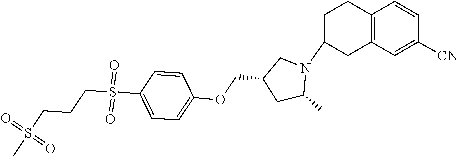 [SMILES: C[C@@H]1C[C@H](COc2ccc(S(=O)(=O)CCCS(C)(=O)=O)cc2)CN1C1CCc2ccc(C#N)cc2C1] | 7-[(2R,4S)-4-{[4-(3- methanesulfonylpropane- sulfonyl)phenoxy]methyl}- 2-methylpyrrolidin-1-yl]- 5,6,7,8-tetrahydronaph- thalene-2-carbonitrile |
| 36 | 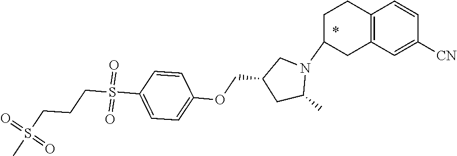 [SMILES: C[C@@H]1C[C@H](COc2ccc(S(=O)(=O)CCCS(C)(=O)=O)cc2)CN1C1CCc2ccc(C#N)cc2C1] | (7S or 7R)-7-[(2R,4S)-4- {[4-(3-methanesulfonyl- propanesulfonyl)phenoxy] methyl}-2-methylpyrrol- idin-1-yl]-5,6,7,8-tetra- hydronaphthalene-2- carbonitrile |
| 37 | 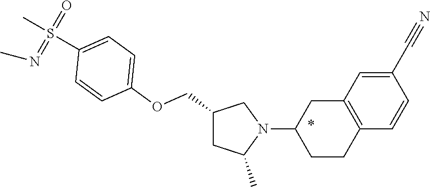 [SMILES: CN=S(C)(=O)c1ccc(OC[C@H]2C[C@@H](C)N(C3CCc4ccc(C#N)cc4C3)C2)cc1] | (7R or 7S)-7-[(2R,4S)-2- methyl-4-({4-[methyl- (methylimino)oxo-λ⁶- sulfanyl]phenoxy}methyl)- pyrrolidin-1-yl]-5,6,7,8- tetrahydronaphthalene-2- carbonitrile |
| 38 | 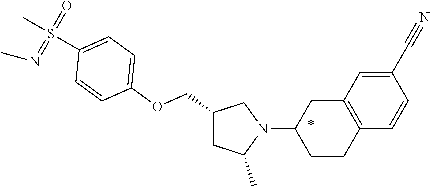 [SMILES: CN=S(C)(=O)c1ccc(OC[C@H]2C[C@@H](C)N(C3CCc4ccc(C#N)cc4C3)C2)cc1] | (7S or 7R)-7-[(2R,4S)-2- methyl-4-({4-[methyl- (methylimino)oxo-λ⁶- sulfanyl]phenoxy}methyl)- pyrrolidin-1-yl]-5,6,7,8- tetrahydronaphthalene-2- carbonitrile |
| 39 | 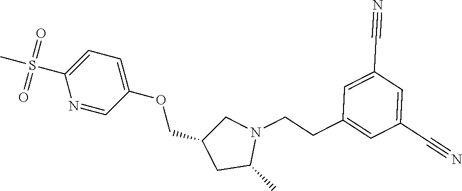 [SMILES: C[C@@H]1C[C@H](COc2ccc(S(C)(=O)=O)nc2)CN1CCc1cc(C#N)cc(C#N)c1] | 5-{2-[(2R,4S)-4-{[(6- methanesulfonylpyridin-3- yl)oxy]methyl}-2-methyl- pyrrolidin-1-yl]ethyl} benzene-1,3-dicarbonitrile |
| 40 | 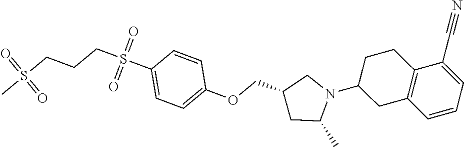 [SMILES: C[C@@H]1C[C@H](COc2ccc(S(=O)(=O)CCCS(C)(=O)=O)cc2)CN1C1CCc2c(C#N)cccc2C1] | 6-[(2R,4S)-4-{[4-(3- methanesulfonylpropane- sulfonyl)phenoxy]methyl}- 2-methylpyrrolidin-1-yl]- 5,6,7,8-tetrahydronaph- thalene-1-carbonitrile |
| 41 | 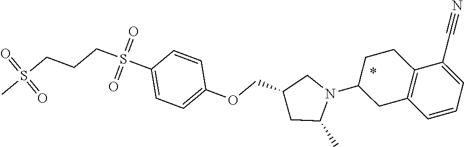 [SMILES: C[C@@H]1C[C@H](COc2ccc(S(=O)(=O)CCCS(C)(=O)=O)cc2)CN1C1CCc2c(C#N)cccc2C1] | (6S or 6R)-6-[(2R,4S)-4- {[4-(3-methanesulfonyl- propanesulfonyl)phenoxy] methyl}-2-methylpyrroli- din-1-yl]-5,6,7,8-tetra- hydronaphthalene-1- carbonitrile |
| 42 | 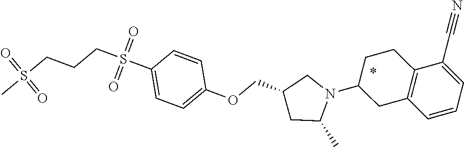 [SMILES: C[C@@H]1C[C@H](COc2ccc(S(=O)(=O)CCCS(C)(=O)=O)cc2)CN1C1CCc2c(C#N)cccc2C1] | (6R or 6S)-6-[(2R,4S)-4- {[4-(3-methanesulfonyl- propanesulfonyl)phenoxy] methyl}-2-methylpyrrol- idin-1-yl]-5,6,7,8-tetra- hydronaphthalene-1- carbonitrile |
| 43 | 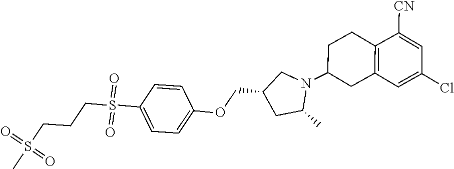 [SMILES: C[C@@H]1C[C@H](COc2ccc(S(=O)(=O)CCCS(C)(=O)=O)cc2)CN1C1CCc2c(C#N)cc(Cl)cc2C1] | 3-chloro-6-[(2R,4S)-4- {[4-(3-methanesulfonyl- propanesulfonyl)phenoxy] methyl}-2-methylpyrrol- idin-1-yl]-5,6,7,8-tetra- hydronaphthalene-1- carbonitrile |
| 44 | 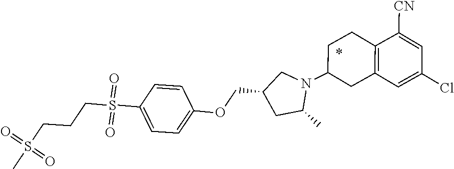 [SMILES: C[C@@H]1C[C@H](COc2ccc(S(=O)(=O)CCCS(C)(=O)=O)cc2)CN1C1CCc2c(C#N)cc(Cl)cc2C1] | (6R or 6S)-3-chloro-6- [(2R,4S)-4-{[4-(3-methane- sulfonylpropanesulfonyl)- phenoxy]methyl}-2- methylpyrrolidin-1-yl]- 5,6,7,8-tetrahydronaph- thalene-1-carbonitrile |
| 45 | 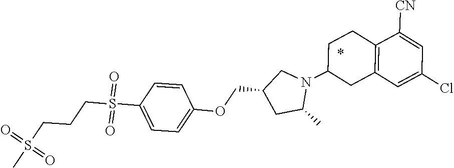 [SMILES: C[C@@H]1C[C@H](COc2ccc(S(=O)(=O)CCCS(C)(=O)=O)cc2)CN1C1CCc2c(C#N)cc(Cl)cc2C1] | (6S or 6R)-3-chloro-6- [(2R,4S)-4-{[4-(3-methane- sulfonylpropanesulfonyl)- phenoxy]methyl}-2- methylpyrrolidin-1-yl]- 5,6,7,8-tetrahydronaph- thalene-1-carbonitrile |
| 46 | 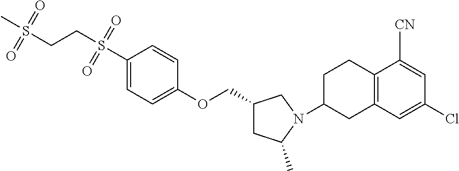 [SMILES: C[C@@H]1C[C@H](COc2ccc(S(=O)(=O)CCS(C)(=O)=O)cc2)CN1C1CCc2c(C#N)cc(Cl)cc2C1] | 3-chloro-6-[(2R,4S)-4-{[4- (2-methanesulfonylethane- sulfonyl)phenoxy]methyl}- 2-methylpyrrolidin-1-yl]- 5,6,7,8-tetrahydronaph- thalene-1-carbonitrile |
| 47 | 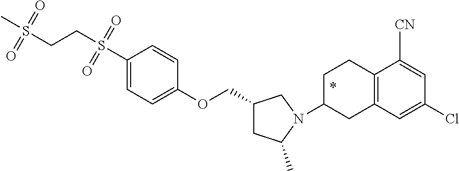 [SMILES: C[C@@H]1C[C@H](COc2ccc(S(=O)(=O)CCS(C)(=O)=O)cc2)CN1C1CCc2c(C#N)cc(Cl)cc2C1] | (6S or 6R)-3-chloro-6- [(2R,4S)-4-{[4-(2-methane- sulfonylethanesulfonyl)- phenoxy]methyl}-2-meth- ylpyrrolidin-1-yl]-5,6,7,8- tetrahydronaphthalene-1- carbonitrile |
| 48 | 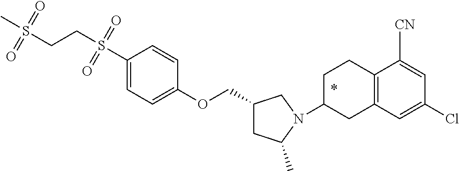 [SMILES: C[C@@H]1C[C@H](COc2ccc(S(=O)(=O)CCS(C)(=O)=O)cc2)CN1C1CCc2c(C#N)cc(Cl)cc2C1] | (6R or 6S)-3-chloro-6- [(2R,4S)-4-{[4-(2-methane- sulfonylethanesulfonyl)- phenoxy]methyl}-2-meth- ylpyrrolidin-1-yl]-5,6,7,8- tetrahydronaphthalene-1- carbonitrile |
| 49 | 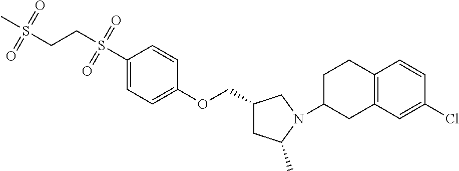 [SMILES: C[C@@H]1C[C@H](COc2ccc(S(=O)(=O)CCS(C)(=O)=O)cc2)CN1C1CCc2ccc(Cl)cc2C1] | (2R,4S)-1-[7-chloro- 1,2,3,4-tetrahydronaph- thalen-2-yl]-4-{[4-(2- methanesulfonylethane- sulfonyl)phenoxy]methyl}- 2-methylpyrrolidine |
| 50 | 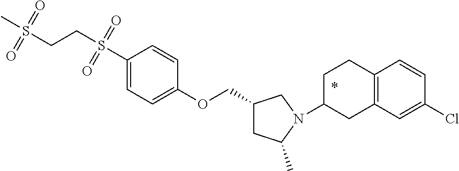 [SMILES: C[C@@H]1C[C@H](COc2ccc(S(=O)(=O)CCS(C)(=O)=O)cc2)CN1C1CCc2ccc(Cl)cc2C1] | (2R,4S)-1-[(2S or 2R)-7- chloro-1,2,3,4-tetrahydro- naphthalen-2-yl]-4-{[4-(2- methanesulfonylethane- sulfonyl)phenoxy]methyl}- 2-methylpyrrolidine |
| 51 | 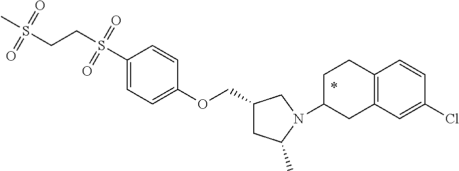 [SMILES: C[C@@H]1C[C@H](COc2ccc(S(=O)(=O)CCS(C)(=O)=O)cc2)CN1C1CCc2ccc(Cl)cc2C1] | (2R,4S)-1-[(2R or 2S)-7- chloro-1,2,3,4-tetrahydro- naphthalen-2-yl]-4-{[4-(2- methanesulfonylethane- sulfonyl)phenoxy]methyl}- 2-methylpyrrolidine |
| 52 | 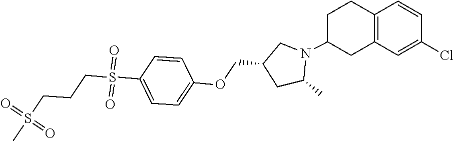 [SMILES: C[C@@H]1C[C@H](COc2ccc(S(=O)(=O)CCCS(C)(=O)=O)cc2)CN1C1CCc2ccc(Cl)cc2C1] | (2R,4S)-1-[7-chloro- 1,2,3,4-tetrahydronaph- thalen-2-yl]-4-{[4-(3- methanesulfonylpropane- sulfonyl)phenoxy]methyl}- 2-methylpyrrolidine |
| 53 | 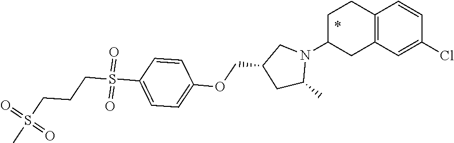 [SMILES: C[C@@H]1C[C@H](COc2ccc(S(=O)(=O)CCCS(C)(=O)=O)cc2)CN1C1CCc2ccc(Cl)cc2C1] | (2R,4S)-1-[(2R or 2S)-7- chloro-1,2,3,4-tetrahydro- naphthalen-2-yl]-4-{[4-(3- methanesulfonylpropane- sulfonyl)phenoxy]methyl}- 2-methylpyrrolidine |
| 54 | 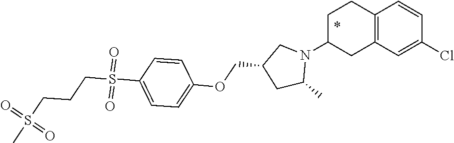 [SMILES: C[C@@H]1C[C@H](COc2ccc(S(=O)(=O)CCCS(C)(=O)=O)cc2)CN1C1CCc2ccc(Cl)cc2C1] | (2R,4S)-1-[(2S or 2R)-7- chloro-1,2,3,4-tetrahydro- naphthalen-2-yl]-4-{[4-(3- methanesulfonylpropane- sulfonyl)phenoxy]methyl}- 2-methylpyrrolidine |
| 55 | 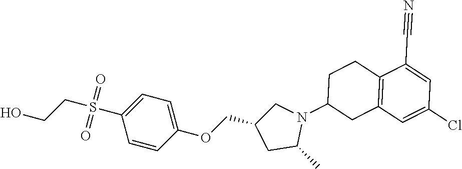 [SMILES: C[C@@H]1C[C@H](COc2ccc(S(=O)(=O)CCO)cc2)CN1C1CCc2c(C#N)cc(Cl)cc2C1] | 3-chloro-6-[(2R,4S)-4- {[4-(2-hydroxyethanesul- fonyl)phenoxy]methyl}-2- methylpyrrolidin-1-yl]- 5,6,7,8-tetrahydronaph- thalene-1-carbonitrile |
| 56 | 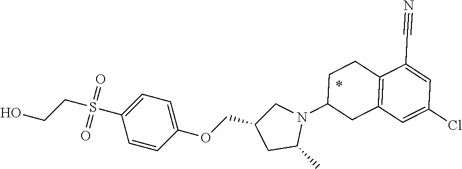 [SMILES: C[C@@H]1C[C@H](COc2ccc(S(=O)(=O)CCO)cc2)CN1C1CCc2c(C#N)cc(Cl)cc2C1] | (6R or 6S)-3-chloro-6- [(2R,4S)-4-{[4-(2-hydroxy- ethanesulfonyl)phenoxy] methyl}-2-methylpyrrol- idin-1-yl]-5,6,7,8-tetra- hydronaphthalene-1- carbonitrile |
| 57 | 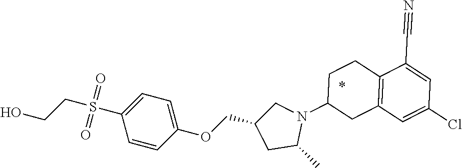 [SMILES: C[C@@H]1C[C@H](COc2ccc(S(=O)(=O)CCO)cc2)CN1C1CCc2c(C#N)cc(Cl)cc2C1] | (6S or 6R)-3-chloro-6- [(2R,4S)-4-{[4-(2-hydroxy- ethanesulfonyl)phenoxy] methyl}-2-methylpyrrol- idin-1-yl]-5,6,7,8-tetra- hydronaphthalene-1- carbonitrile |
| 58 | 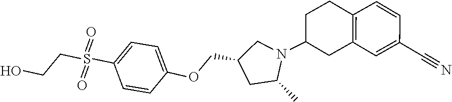 [SMILES: C[C@@H]1C[C@H](COc2ccc(S(=O)(=O)CCO)cc2)CN1C1CCc2ccc(C#N)cc2C1] | 7-[(2R,4S)-4-{[4-(2- hydroxyethanesulfonyl)- phenoxy]methyl}-2-meth- ylpyrrolidin-1-yl]-5,6,7,8- tetrahydronaphthalene-2- carbonitrile |
| 59 | 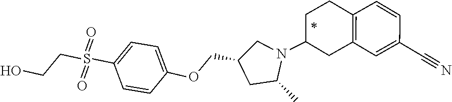 [SMILES: C[C@@H]1C[C@H](COc2ccc(S(=O)(=O)CCO)cc2)CN1C1CCc2ccc(C#N)cc2C1] | (7R or 7S)-7-[(2R,4S)-4- {[4-(2-hydroxyethanesul- fonyl)phenoxy]methyl}-2- methylpyrrolidin-1-yl]- 5,6,7,8-tetrahydronaph- thalene-2-carbonitrile |
| 60 | 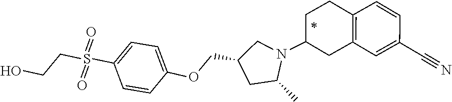 [SMILES: C[C@@H]1C[C@H](COc2ccc(S(=O)(=O)CCO)cc2)CN1C1CCc2ccc(C#N)cc2C1] | (7S or 7R)-7-[(2R,4S)-4- {[4-(2-hydroxyethane- sulfonyl)phenoxy]methyl}- 2-methylpyrrolidin-1-yl]- 5,6,7,8-tetrahydronaph- thalene-2-carbonitrile |
| 61 | 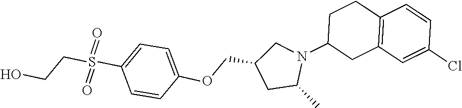 [SMILES: C[C@@H]1C[C@H](COc2ccc(S(=O)(=O)CCO)cc2)CN1C1CCc2ccc(Cl)cc2C1] | 2-(4-{[(3S,5R)-1-[7- chloro-1,2,3,4-tetrahydro- naphthalen-2-yl]-5-meth- ylpyrrolidin-3-yl]methoxy} benzenesulfonyl)ethan-1-ol |
| 62 | 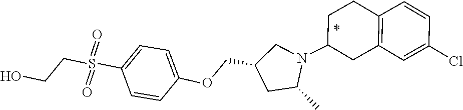 [SMILES: C[C@@H]1C[C@H](COc2ccc(S(=O)(=O)CCO)cc2)CN1C1CCc2ccc(Cl)cc2C1] | 2-(4-{[(3S,5R)-1-[(2R or 2S)-7-chloro-1,2,3,4-tetra- hydronaphthalen-2-yl]-5- methylpyrrolidin-3-yl] methoxy}benzenesulfon- yl)ethan-1-ol |
| 63 | 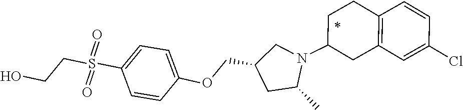 [SMILES: C[C@@H]1C[C@H](COc2ccc(S(=O)(=O)CCO)cc2)CN1C1CCc2ccc(Cl)cc2C1] | 2-(4-{[(3S,5R)-1-[(2S or 2R)-7-chloro-1,2,3,4-tetra- hydronaphthalen-2-yl]-5- methylpyrrolidin-3-yl] methoxy}benzenesulfon- yl)ethan-1-ol |
| 64 | 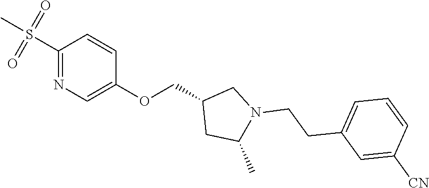 [SMILES: C[C@@H]1C[C@H](COc2ccc(S(C)(=O)=O)nc2)CN1CCc1cccc(C#N)c1] | 3-{2-[(2R,4S)-4-{[(6- methanesulfonylpyridin-3- yl)oxy]methyl}-2-methyl- pyrrolidin-1-yl]ethyl} benzonitrile |
| 65 | 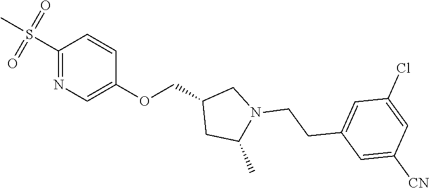 [SMILES: C[C@@H]1C[C@H](COc2ccc(S(C)(=O)=O)nc2)CN1CCc1cc(Cl)cc(C#N)c1] | 3-chloro-5-{2-[(2R,4S)-4- {[(6-methanesulfonylpyr- idin-3-yl)oxy]methyl}-2- methylpyrrolidin-1-ylethyl} benzonitrile |
| 66 | 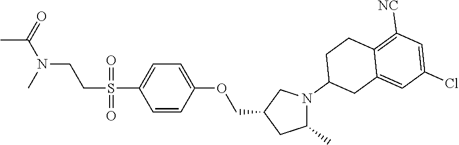 [SMILES: CC(=O)N(C)CCS(=O)(=O)c1ccc(OC[C@H]2C[C@@H](C)N(C3CCc4c(C#N)cc(Cl)cc4C3)C2)cc1] | N-[2-(4-{[(3S,5R)-1-[2-(3- chloro-5-cyanophenyl)- ethyl]-5-methylpyrrolidin- 3-yl]methoxy}benzenesul- fonyl)ethyl]-N-methyl- acetamide |
| 67 | 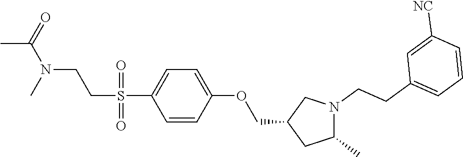 [SMILES: CC(=O)N(C)CCS(=O)(=O)c1ccc(OC[C@H]2C[C@@H](C)N(CCc3cccc(C#N)c3)C2)cc1] | N-[2-(4-{[(3S,5R)-1-[2- (3-cyanophenyl)ethyl]-5- methylpyrrolidin-3-yl] methoxy}benzenesulfon- yl)ethyl]-N-methyl- acetamide |
| 68 | 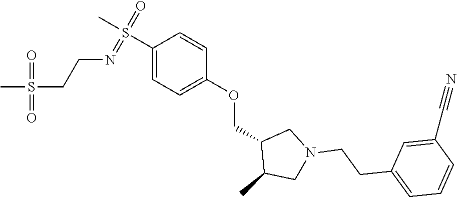 [SMILES: C[C@@H]1CN(CCc2cccc(C#N)c2)C[C@H]1COc1ccc(S(C)(=O)=NCCS(C)(=O)=O)cc1] | 3-{2-[(3S,4S)-3-[(4-{[(2- methanesulfonylethyl)- imino](methyl)oxo-λ⁶- sulfanyl}phenoxy)methyl]- 4-methylpyrrolidin-1-yl] ethyl}benzonitrile |
| 69 | 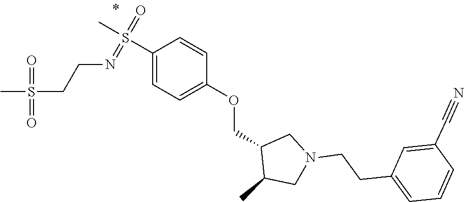 [SMILES: C[C@@H]1CN(CCc2cccc(C#N)c2)C[C@H]1COc1ccc(S(C)(=O)=NCCS(C)(=O)=O)cc1] | 3-{2-[(3S,4S)-3-[(4-{[(2- methanesulfonylethyl)- imino](methyl)oxo-λ⁶- sulfanyl}phenoxy)methyl]- 4-methylpyrrolidin-1-yl] ethyl}benzonitrile |
| 70 | 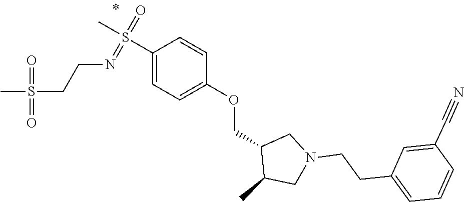 [SMILES: C[C@@H]1CN(CCc2cccc(C#N)c2)C[C@H]1COc1ccc(S(C)(=O)=NCCS(C)(=O)=O)cc1] | 3-{2-[(3S,4S)-3-[(4-{[(2- methanesulfonylethyl)- imino](methyl)oxo-λ⁶- sulfanyl}phenoxy)methyl]- 4-methylpyrrolidin-1-yl] ethyl}benzonitrile |
| 71 | 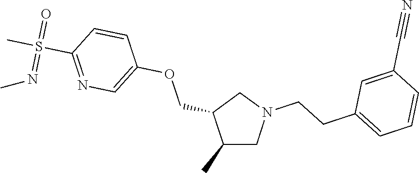 [SMILES: CN=S(C)(=O)c1ccc(OC[C@@H]2CN(CCc3cccc(C#N)c3)C[C@H]2C)cn1] | 3-{2-[(3S,4S)-3-methyl-4- [{{6-[methyl(methylimino)- oxo-λ⁶-sulfanyl]pyridin-3- yl}oxy)methyl]pyrrolidin- 1-yl]ethyl}benzonitrile |
| 72 | 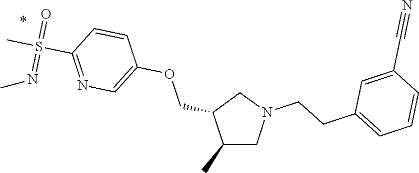 [SMILES: CN=S(C)(=O)c1ccc(OC[C@@H]2CN(CCc3cccc(C#N)c3)C[C@H]2C)cn1] | 3-{2-[(3S,4S)-3-methyl-4- [{{6-[methyl(methylimino)- oxo-λ⁶-sulfanyl]pyridin-3- yl}oxy)methyl]pyrrolidin- 1-yl]ethyl}benzonitrile |
| 73 | 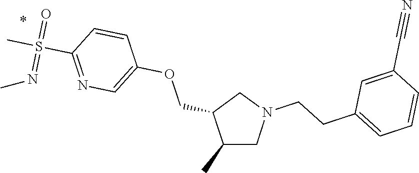 [SMILES: CN=S(C)(=O)c1ccc(OC[C@@H]2CN(CCc3cccc(C#N)c3)C[C@H]2C)cn1] | 3-{2-[(3S,4S)-3-methyl-4- [({6-[methyl(methylimino)- oxo-λ⁶-sulfanyl]pyridin-3- yl}oxy)methyl]pyrrolidin- 1-yl]ethyl}benzonitrile |
| 74 | 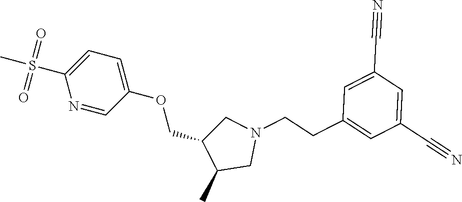 [SMILES: C[C@@H]1CN(CCc2cc(C#N)cc(C#N)c2)C[C@H]1COc1ccc(S(C)(=O)=O)nc1] | 5-{2-[(3S,4S)-3-{[(6- methanesulfonylpyridin-3- yl)oxy]methyl}-4-methyl- pyrrolidin-1-yl]ethyl} benzene-1,3-dicarbonitrile |
| 75 | 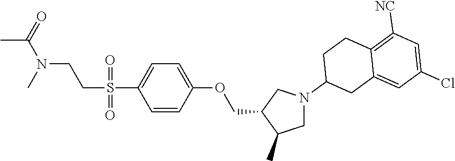 [SMILES: CC(=O)N(C)CCS(=O)(=O)c1ccc(OC[C@@H]2CN(C3CCc4c(C#N)cc(Cl)cc4C3)C[C@H]2C)cc1] | N-[2-(4-{[(3S,4S)-1-[2-(3- chloro-5-cyanophenyl)- ethyl]-4-methylpyrrolidin- 3-yl]methoxy}benzenesul- fonyl)ethyl]-N-methyl- acetamide |
| 76 | 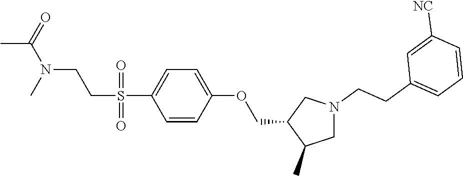 [SMILES: CC(=O)N(C)CCS(=O)(=O)c1ccc(OC[C@@H]2CN(CCc3cccc(C#N)c3)C[C@H]2C)cc1] | N-[2-(4-{[(3S,4S)-1-[2-(3- cyanophenyl)ethyl]-4- methylpyrrolidin-3-yl] methoxy}benzenesulfon- yl)ethyl]-N-methyl- acetamide |
| 77 | 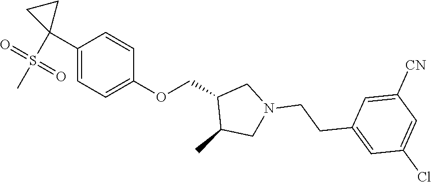 [SMILES: C[C@@H]1CN(CCc2cc(Cl)cc(C#N)c2)C[C@H]1COc1ccc(C2(S(C)(=O)=O)CC2)cc1] | 3-chloro-5-{2-[(3S,4S)-3- {[4-(1-methanesulfonyl- cyclopropyl)phenoxy] methyl}-4-methylpyrrol- idin-1-yl]ethyl}benzonitrile |
| 78 | 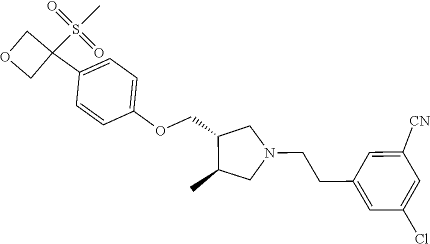 [SMILES: C[C@@H]1CN(CCc2cc(Cl)cc(C#N)c2)C[C@H]1COc1ccc(C2(S(C)(=O)=O)COC2)cc1] | 3-chloro-5-{2-[(3S,4S)-3- {[4-(3-methanesulfonyl- oxetan-3-yl)phenoxy] methyl}-4-methylpyrrol- idin-1-yl]ethyl}benzonitrile |
| 79 | 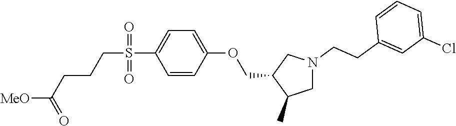 [SMILES: COC(=O)CCCS(=O)(=O)c1ccc(OC[C@@H]2CN(CCc3cccc(Cl)c3)C[C@H]2C)cc1] | methyl 4-(4-{[(3S,4S)-1- [2-(3-chlorophenyl)ethyl]- 4-methylpyrrolidin-3-yl] methoxy}benzenesulfon- yl)butanoate |
| 80 | 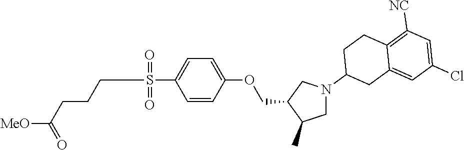 [SMILES: COC(=O)CCCCS(=O)(=O)c1ccc(OC[C@@H]2CN(C3CCc4c(C#N)cc(Cl)cc4C3)C[C@H]2C)cc1] | methyl 4-(4-{[(3S,4S)-1- [2-(3-chloro-5-cyanophen- yl)ethyl]-4-methylpyrrol- idin-3-yl]methoxy}ben- zenesulfonyl)butanoate |
| 81 | 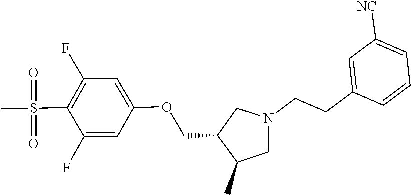 [SMILES: C[C@@H]1CN(CCc2cccc(C#N)c2)C[C@H]1COc1cc(F)c(S(C)(=O)=O)c(F)c1] | 3-{2-[(3S,4S)-3-[(3,5- difluoro-4-methanesulfon- ylphenoxy)methyl]-4- methylpyrrolidin-1-yl] ethyl}benzonitrile |
| 82 | 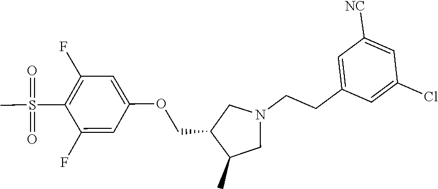 [SMILES: C[C@@H]1CN(CCc2cc(Cl)cc(C#N)c2)C[C@H]1COc1cc(F)c(S(C)(=O)=O)c(F)c1] | 3-chloro-5-{2-[(3S,4S)-3- [(3,5-difluoro-4-methane- sulfonylphenoxy)methyl]- 4-methylpyrrolidin-1-yl] ethyl}benzonitrile |
| 83 | 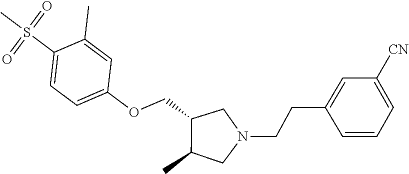 [SMILES: Cc1cc(OC[C@@H]2CN(CCc3cccc(C#N)c3)C[C@H]2C)ccc1S(C)(=O)=O] | 3-{2-[(3S,4S)-3-[(4- methanesulfonyl-3-methyl- phenoxy)methyl]-4-meth- ylpyrrolidin-1-yl]ethyl} benzonitrile |
| 84 | 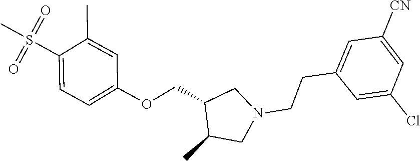 [SMILES: Cc1cc(OC[C@@H]2CN(CCc3cc(Cl)cc(C#N)c3)C[C@H]2C)ccc1S(C)(=O)=O] | 3-chloro-5-{2-[(3S,4S)-3- [(4-methanesulfonyl-3- methylphenoxy)methyl]- 4-methylpyrrolidin-1-yl- ethyl}benzonitrile |
| 85 | 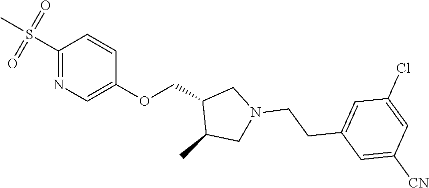 [SMILES: C[C@@H]1CN(CCc2cc(Cl)cc(C#N)c2)C[C@H]1COc1ccc(S(C)(=O)=O)nc1] | 3-chloro-5-{2-[(3S,4S)-3- {[(6-methanesulfonylpyr- idin-3-yl)oxy]methyl}-4- methylpyrrolidin-1-yl] ethyl}benzonitrile |
| 86 | 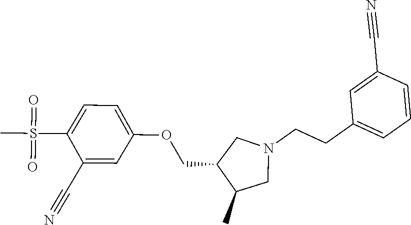 [SMILES: C[C@@H]1CN(CCc2cccc(C#N)c2)C[C@H]1COc1ccc(S(C)(=O)=O)c(C#N)c1] | 5-{[(3S,4S)-1-[2-(3-cyano- phenyl)ethyl]-4-methyl- pyrrolidin-3-yl]methoxy}- 2-methanesulfonylbenzo- nitrile |
| 87 | 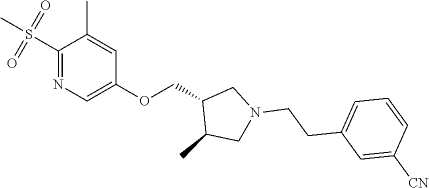 [SMILES: Cc1cc(OC[C@@H]2CN(CCc3cccc(C#N)c3)C[C@H]2C)cnc1S(C)(=O)=O] | 3-{2-[(3S,4S)-3-{[(6- methanesulfonyl-5-methyl- pyridin-3-yl)oxy]methyl}- 4-methylpyrrolidin-1-yl] ethyl}benzonitrile |
| 88 | 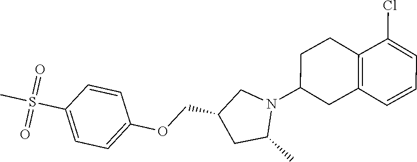 [SMILES: C[C@@H]1C[C@H](COc2ccc(S(C)(=O)=O)cc2)CN1C1CCc2c(Cl)cccc2C1] | (2R,4S)-1-(5-chloro- 1,2,3,4-tetrahydronaph- thalen-2-yl)-4-[(4-methane- sulfonylphenoxy)methyl]- 2-methylpyrrolidine |
| 89 | 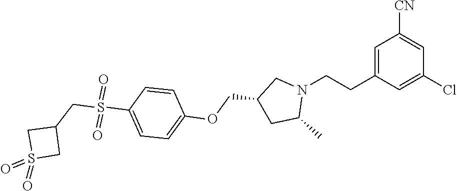 [SMILES: C[C@@H]1C[C@H](COc2ccc(S(=O)(=O)CC3CS(=O)(=O)C3)cc2)CN1CCc1cc(Cl)cc(C#N)c1] | 3-chloro-5-{2-[(3S,4S)- 3-({4-[(1,1-dioxo-1λ⁶- thietan-3-yl)methanesul- fonyl]phenoxy}methyl)-4- methylpyrrolidin-1-yl] ethyl}benzonitrile |
| 90 | 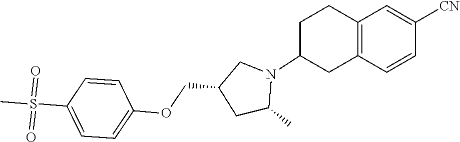 [SMILES: C[C@@H]1C[C@H](COc2ccc(S(C)(=O)=O)cc2)CN1C1CCc2cc(C#N)ccc2C1] | 7-[(2R,4S)-4-[(4-methane- sulfonylphenoxy)methyl]- 2-methylpyrrolidin-1-yl]- 5,6,7,8-tetrahydronaph- thalene-2-carbonitrile |
| 91 | 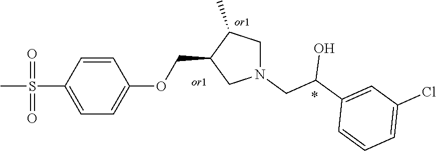 [SMILES: C[C@@H]1CN(CC(O)c2cccc(Cl)c2)C[C@H]1COc1ccc(S(C)(=O)=O)cc1] | (1R) or (1S)-1-(3-chloro- phenyl)-2-[trans-3-[(4- methanesulfonylphenoxy)- methyl]-4-methylpyrrol- idin-1-yl]ethan-1-ol |
| 92 | 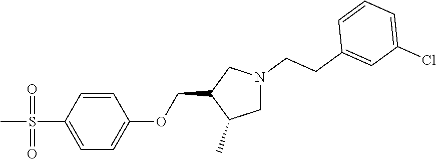 | (3R,4R)-1-[2-(3-chloro- phenyl)ethyl]-3-[(4- methanesulfonylphen- oxy)methyl]-4-methyl- pyrrolidine |
| 93 | 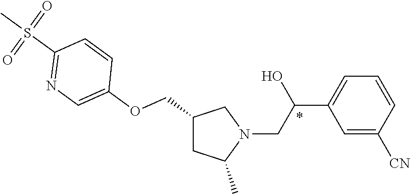 [SMILES: C[C@@H]1C[C@H](COc2ccc(S(C)(=O)=O)nc2)CN1CC(O)c1cccc(C#N)c1] | 3-[(1R or 1S)-1-hydroxy- 2-[(2R,4S)-4-{[(6-methane- sulfonylpyridin-3-yl)oxy] methyl}-2-methylpyrrol- idin-1-yl]ethyl]benzonitrile |
| 94 | 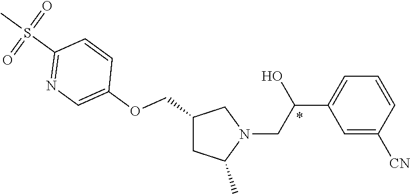 [SMILES: C[C@@H]1C[C@H](COc2ccc(S(C)(=O)=O)nc2)CN1CC(O)c1cccc(C#N)c1] | 3-[(1S or 1R)-1-hydroxy- 2-[(2R,4S)-4-{[(6-methane- sulfonylpyridin-3-yl)oxy] methyl}-2-methylpyrrol- idin-1-yl]ethyl]benzonitrile |
| 95 | 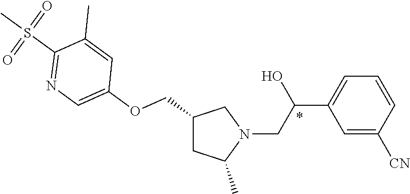 [SMILES: Cc1cc(OC[C@H]2C[C@@H](C)N(CC(O)c3cccc(C#N)c3)C2)cnc1S(C)(=O)=O] | 3-[(1R or 1S)-1-hydroxy- 2-[(2R,4S)-4-{[(6-methane- sulfonyl-5-methylpyridin- 3-yl)oxy]methyl}-2-meth- ylpyrrolidin-1-yl]ethyl] benzonitrile |
| 96 | 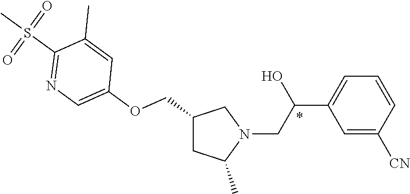 [SMILES: Cc1cc(OC[C@H]2C[C@@H](C)N(CC(O)c3cccc(C#N)c3)C2)cnc1S(C)(=O)=O] | 3-[(1S or 1R)-1-hydroxy- 2-[(2R,4S)-4-{[(6-methane- sulfonyl-5-methylpyridin- 3-yl)oxy]methyl}-2-meth- ylpyrrolidin-1-yl]ethyl] benzonitrile |
| 97 | 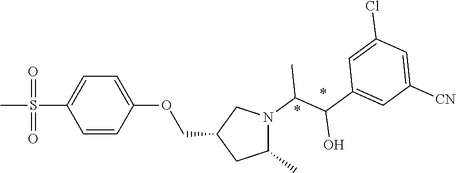 [SMILES: CC(C(O)c1cc(Cl)cc(C#N)c1)N1C[C@@H](COc2ccc(S(C)(=O)=O)cc2)C[C@H]1C] | 3-chloro-5-[(1S,2S or 1R,2R or 1R,2S or 1S,2R)- 1-hydroxy-2-[(2R,4S)-4- [(4-methanesulfonylphen- oxy)methyl]-2-methylpyr- rolidin-1-yl]propyl]benzo- nitrile |
| 98 | 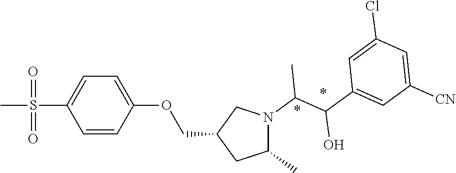 [SMILES: CC(C(O)c1cc(Cl)cc(C#N)c1)N1C[C@@H](COc2ccc(S(C)(=O)=O)cc2)C[C@H]1C] | 3-chloro-5-[(1R,2S or 1S,2R or 1S,2S or 1R,2R)- 1-hydroxy-2-[(2R,4S)-4- [(4-methanesulfonylphen- oxy)methyl]-2-methylpyr- rolidin-1-yl]propyl]benzo- nitrile |
| 99 | 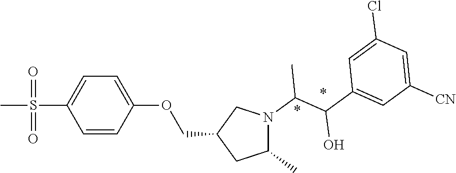 [SMILES: CC(C(O)c1cc(Cl)cc(C#N)c1)N1C[C@@H](COc2ccc(S(C)(=O)=O)cc2)C[C@H]1C] | 3-chloro-5-[(1S,2R or 1R,2S or 1S,2S or 1R,2R)- 1-hydroxy-2-[(2R,4S)-4- [(4-methanesulfonylphen- oxy)methyl]-2-methyl- pyrrolidin-1-yl]propyl] benzonitrile |
| 100 | 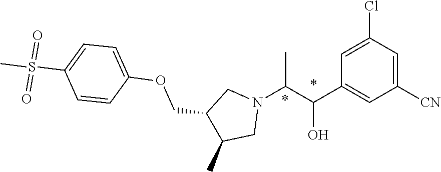 [SMILES: CC(C(O)c1cc(Cl)cc(C#N)c1)N1C[C@@H](COc2ccc(S(C)(=O)=O)cc2)[C@H](C)C1] | 3-chloro-5-((1R,2S or 1S,2R)-1-hydroxy-2- ((3S,4S)-3-methyl-4-((4- (methylsulfonyl)phenoxy)- methyl)pyrrolidin-1-yl)- propyl)benzonitrile |
| 101 | 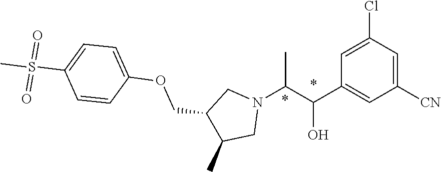 [SMILES: CC(C(O)c1cc(Cl)cc(C#N)c1)N1C[C@@H](COc2ccc(S(C)(=O)=O)cc2)[C@H](C)C1] | 3-chloro-5-((1S,2R or 1R,2S)-1-hydroxy-2- ((3S,4S)-3-methyl-4-((4- (methylsulfonyl)phenoxy)- methyl)pyrrolidin-1-yl)- propyl)benzonitrile |
| 102 | 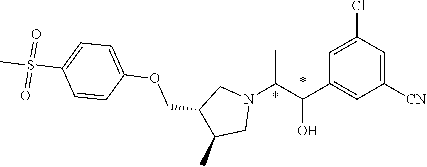 [SMILES: CC(C(O)c1cc(Cl)cc(C#N)c1)N1C[C@@H](COc2ccc(S(C)(=O)=O)cc2)[C@H](C)C1] | 3-chloro-5-((1R,2R or 1S,2S)-1-hydroxy-2- ((3S,4S)-3-methyl-4-((4- (methylsulfonyl)phenoxy)- methyl)pyrrolidin-1-yl)- propyl)benzonitrile |
| 103 | 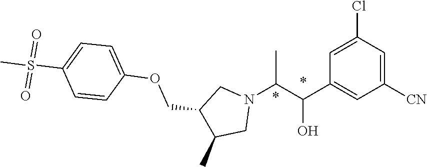 [SMILES: CC(C(O)c1cc(Cl)cc(C#N)c1)N1C[C@@H](COc2ccc(S(C)(=O)=O)cc2)[C@H](C)C1] | 3-chloro-5-((1S,2S or 1R,2R)-1-hydroxy-2- ((3S,4S)-3-methyl-4-((4- (methylsulfonyl)phenoxy)- methyl)pyrrolidin-1-yl)- propyl)benzonitrile |
| 104 | 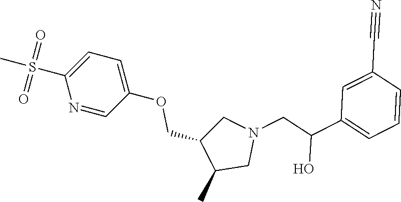 [SMILES: C[C@@H]1CN(CC(O)c2cccc(C#N)c2)C[C@H]1COc1ccc(S(C)(=O)=O)nc1] | 3-{1-hydroxy-2-[(3S,4S)- 3-{[(6-methanesulfonyl- pyridin-3-yl)oxy]methyl}- 4-methylpyrrolidin-1-yl] ethyl}benzonitrile |
| 105 | 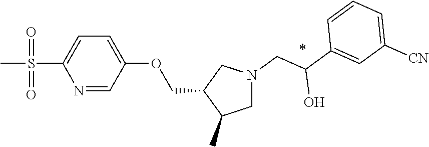 [SMILES: C[C@@H]1CN(CC(O)c2cccc(C#N)c2)C[C@H]1COc1ccc(S(C)(=O)=O)nc1] | 3-[(1S or 1R)-1-hydroxy- 2-[(3S,4S)-3-{[(6-methane- sulfonylpyridin-3-yl)oxy] methyl}-4-methylpyrrol- idin-1-yl]ethyl]benzonitrile |
| 106 | 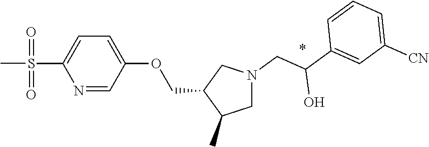 [SMILES: C[C@@H]1CN(CC(O)c2cccc(C#N)c2)C[C@H]1COc1ccc(S(C)(=O)=O)nc1] | 3-[(1R or 1S)-1-hydroxy- 2-[(3S,4S)-3-{[(6-methane- sulfonylpyridin-3-yl)oxy] methyl}-4-methylpyrrol- idin-1-yl]ethyl]benzonitrile |
| 107 | 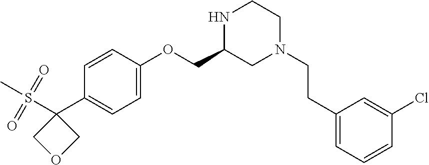 [SMILES: CS(=O)(=O)C1(c2ccc(OC[C@@H]3CN(CCc4cccc(Cl)c4)CCN3)cc2)COC1] | (3S)-1-[2-(3-chlorophen- yl)ethyl]-3-{[4-(3-methane- sulfonyloxetan-3-yl)phen- oxy]methyl}piperazine |
| 108 | 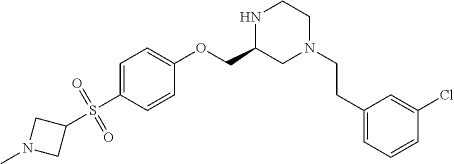 [SMILES: CN1CC(S(=O)(=O)c2ccc(OC[C@@H]3CN(CCc4cccc(Cl)c4)CCN3)cc2)C1] | (3S)-1-[2-(3-chlorophen- yl)ethyl]-3-({4-[(1-methyl- azetidin-3-yl)sulfonyl] phenoxy}methyl)piperazine |
| 109 | 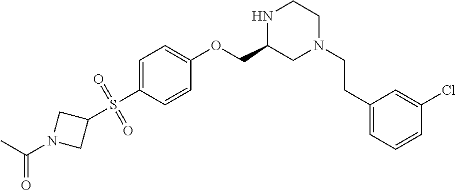 [SMILES: CC(=O)N1CC(S(=O)(=O)c2ccc(OC[C@@H]3CN(CCc4cccc(Cl)c4)CCN3)cc2)C1] | 1-[3-(4-{[(2S)-4-[2-(3- chlorophenyl)ethyl]pipera- zin-2-yl]methoxy}benzene- sulfonyl)azetidin-1-yl] ethan-1-one |
| 110 | 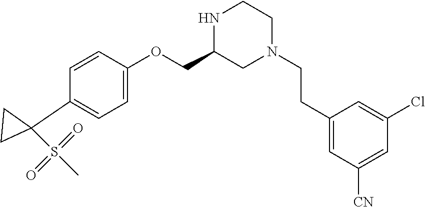 [SMILES: CS(=O)(=O)C1(c2ccc(OC[C@@H]3CN(CCc4cc(Cl)cc(C#N)c4)CCN3)cc2)CC1] | 3-chloro-5-{2-[(3S)-3- {[4-(1-methanesulfonyl- cyclopropyl)phenoxy] methyl}piperazin-1- yl]ethyl}benzonitrile |
| 111 | 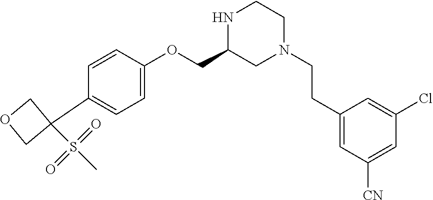 [SMILES: CS(=O)(=O)C1(c2ccc(OC[C@@H]3CN(CCc4cc(Cl)cc(C#N)c4)CCN3)cc2)COC1] | 3-chloro-5-{2-[(3S)-3- {[4-(3-methanesulfonyl- oxetan-3-yl)phenoxy] methyl}piperazin-1-yl] ethyl}benzonitrile |
| 112 | 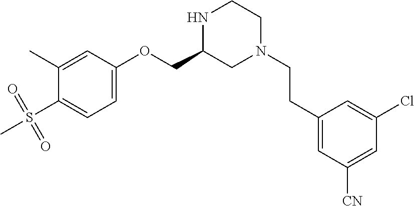 [SMILES: Cc1cc(OC[C@@H]2CN(CCc3cc(Cl)cc(C#N)c3)CCN2)ccc1S(C)(=O)=O] | 3-chloro-5-{2-[(3S)-3-[(4- methanesulfonyl-3-meth- ylphenoxy)methyl]pipera- zin-1-yl]ethyl}benzonitrile |
| 113 | 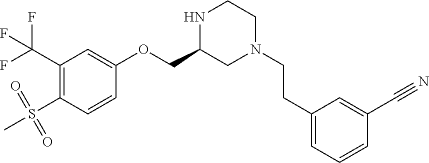 [SMILES: CS(=O)(=O)c1ccc(OC[C@@H]2CN(CCc3cccc(C#N)c3)CCN2)cc1C(F)(F)F] | 3-{2-[(3S)-3-{[4-methane- sulfonyl-3-(trifluorometh- yl)phenoxy]methyl}pipera- zin-1-yl]ethyl}benzonitrile |
| 114 | 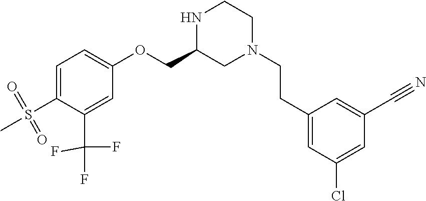 [SMILES: CS(=O)(=O)c1ccc(OC[C@@H]2CN(CCc3cc(Cl)cc(C#N)c3)CCN2)cc1C(F)(F)F] | 3-chloro-5-{2-[(3S)-3-{[4- methanesulfonyl-3-(tri- fluoromethyl)phenoxy] methyl}piperazin-1-yl- ethyl}benzonitrile |
| 115 | 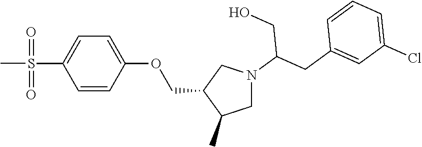 [SMILES: C[C@@H]1CN(C(CO)Cc2cccc(Cl)c2)C[C@H]1COc1ccc(S(C)(=O)=O)cc1] | 3-(3-chlorophenyl)-2- [(3S,4S)-3-[(4-methanesul- fonylphenoxy)methyl]-4- methylpyrrolidin-1-yl] propan-1-ol |
| 116 | 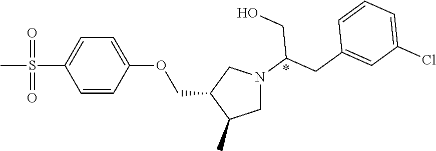 [SMILES: C[C@@H]1CN(C(CO)Cc2cccc(Cl)c2)C[C@H]1COc1ccc(S(C)(=O)=O)cc1] | (2R or 2S)-3-(3-chloro- phenyl)-2-[(3S,4S)-3-[(4- methanesulfonylphenoxy)- methyl]-4-methylpyrrol- idin-1-yl]propan-1-ol |
| 117 | 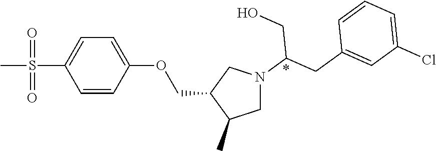 [SMILES: C[C@@H]1CN(C(CO)Cc2cccc(Cl)c2)C[C@H]1COc1ccc(S(C)(=O)=O)cc1] | (2S or 2R)-3-(3-chloro- phenyl)-2-[(3S,4S)-3-[(4- methanesulfonylphenoxy)- methyl]-4-methylpyrrol- idin-1-yl]propan-1-ol |
| 118 | 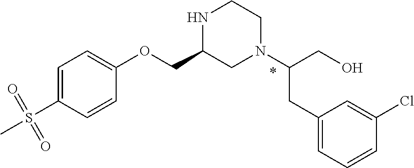 [SMILES: CS(=O)(=O)c1ccc(OC[C@@H]2CN(C(CO)Cc3cccc(Cl)c3)CCN2)cc1] | (2R or 2S)-3-(3-chloro- phenyl)-2-[(3S)-3-[(4- methanesulfonylphenoxy)- methyl]piperazin-1-yl] propan-1-ol |
| 119 | 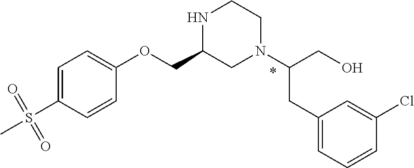 [SMILES: CS(=O)(=O)c1ccc(OC[C@@H]2CN(C(CO)Cc3cccc(Cl)c3)CCN2)cc1] | (2S or 2R)-3-(3-chloro- phenyl)-2-[(3S)-3-[(4- methanesulfonylphenoxy)- methyl]piperazin-1-yl] propan-1-ol |
| 120 | 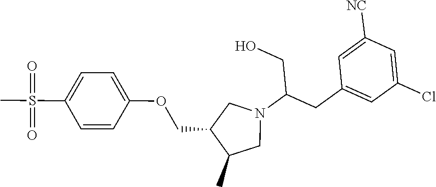 [SMILES: C[C@@H]1CN(C(CO)Cc2cc(Cl)cc(C#N)c2)C[C@H]1COc1ccc(S(C)(=O)=O)cc1] | 3-chloro-5-[3-hydroxy-2- [(3S,4S)-3-[(4-methanesul- fonylphenoxy)methyl]-4- methylpyrrolidin-1-yl] propyl]benzonitrile |
| 121 | 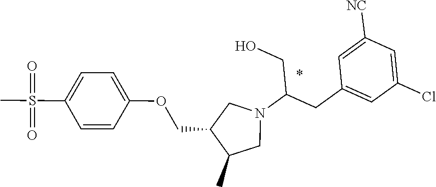 [SMILES: C[C@@H]1CN(C(CO)Cc2cc(Cl)cc(C#N)c2)C[C@H]1COc1ccc(S(C)(=O)=O)cc1] | 3-chloro-5-[(2R or 2S)-3- hydroxy-2-[(3S,4S)-3-[(4- methanesulfonylphenoxy)- methyl]-4-methylpyrrolidin- 1-yl]propyl]benzonitrile |
| 122 | 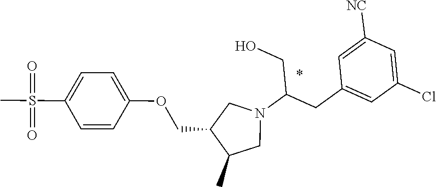 [SMILES: C[C@@H]1CN(C(CO)Cc2cc(Cl)cc(C#N)c2)C[C@H]1COc1ccc(S(C)(=O)=O)cc1] | 3-chloro-5-[(2S or 2R)-3- hydroxy-2-[(3S,4S)-3-[(4- methanesulfonylphenoxy)- methyl]-4-methylpyrrolidin- 1-yl]propyl]benzonitrile |
| 123 | 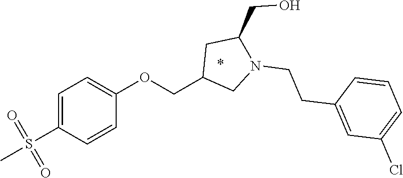 [SMILES: CS(=O)(=O)c1ccc(OCC2C[C@@H](CO)N(CCc3cccc(Cl)c3)C2)cc1] | [(2S,4S or 4R)-1-[2-(3- chlorophenyl)ethyl]-4-[(4- methanesulfonylphenoxy)- methyl]pyrrolidin-2-yl] methanol |
| 124 | 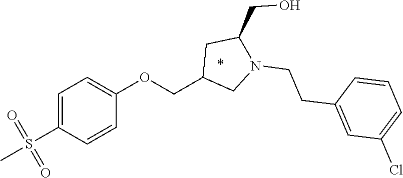 [SMILES: CS(=O)(=O)c1ccc(OCC2C[C@@H](CO)N(CCc3cccc(Cl)c3)C2)cc1] | [(2S,4R or 4S)-1-[2-(3- chlorophenyl)ethyl]-4-[(4- methanesulfonylphenoxy)- methyl]pyrrolidin-2-yl] methanol |
| 125 | 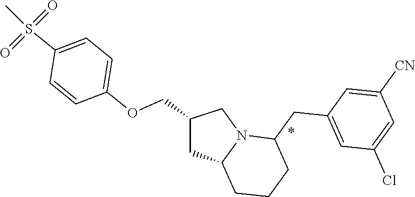 [SMILES: CS(=O)(=O)c1ccc(OC[C@H]2C[C@H]3CCCC(Cc4cc(Cl)cc(C#N)c4)N3C2)cc1] | 3-{[(2S,5R or 5S,8aR)-2- [(4-methanesulfonylphen- oxy)methyl]-octahydro- indolizin-5-yl]methyl}-5- chlorobenzonitrile |
| 126 | 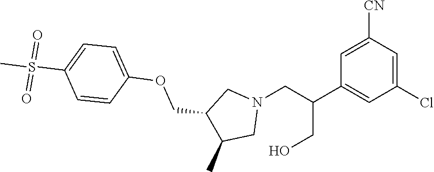 [SMILES: C[C@@H]1CN(CC(CO)c2cc(Cl)cc(C#N)c2)C[C@H]1COc1ccc(S(C)(=O)=O)cc1] | 3-chloro-5-(1-hydroxy-3- ((3S,4S)-3-methyl-4-((4- (methylsulfonyl)phenoxy)- methyl)pyrrolidin-1-yl)- propan-2-yl)benzonitrile |
| 127 | 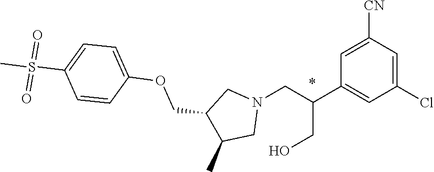 [SMILES: C[C@@H]1CN(CC(CO)c2cc(Cl)cc(C#N)c2)C[C@H]1COc1ccc(S(C)(=O)=O)cc1] | 3-chloro-5-[(2R or 2S)-1- hydroxy-3-[(3S,4S)-3-[(4- methanesulfonylphenoxy)- methyl]-4-methylpyrrol- idin-1-yl]propan-2-yl] benzonitrile |
| 128 | 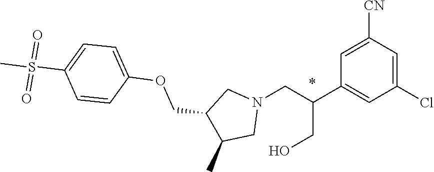 [SMILES: C[C@@H]1CN(CC(CO)c2cc(Cl)cc(C#N)c2)C[C@H]1COc1ccc(S(C)(=O)=O)cc1] | 3-chloro-5-[(2S or 2R)-1- hydroxy-3-[(3S,4S)-3-[(4- methanesulfonylphenoxy)- methyl]-4-methylpyrrolidin- 1-yl]propan-2-yl]benzo- nitrile |
| 129 | 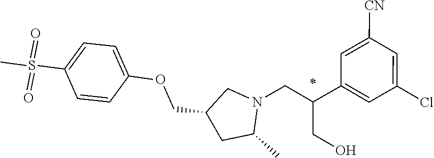 [SMILES: C[C@@H]1C[C@H](COc2ccc(S(C)(=O)=O)cc2)CN1CC(CO)c1cc(Cl)cc(C#N)c1] | 3-chloro-5-[(2R or 2S)-1- hydroxy-3-[(2R,4S)-4-[(4- methanesulfonylphenoxy)- methyl]-2-methylpyrrolidin- 1-yl]propan-2-yl]benzo- nitrile |
| 130 | 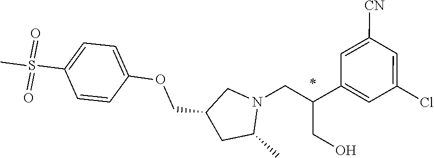 [SMILES: C[C@@H]1C[C@H](COc2ccc(S(C)(=O)=O)cc2)CN1CC(CO)c1cc(Cl)cc(C#N)c1] | 3-chloro-5-[(2S or 2R)-1- hydroxy-3-[(2R,4S)-4-[(4- methanesulfonylphenoxy)- methyl]-2-methylpyrrolidin- 1-yl]propan-2-yl]benzo- nitrile |
| 131 | 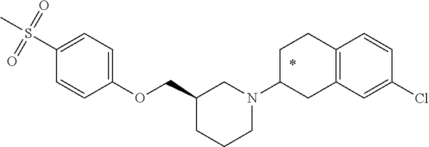 [SMILES: CS(=O)(=O)c1ccc(OC[C@@H]2CCCN(C3CCc4ccc(Cl)cc4C3)C2)cc1] | (3R)-1-(7-chloro-1,2,3,4- tetrahydronaphthalen-2- yl)-3-[(4-methanesulfonyl- phenoxy)methyl]piperidine |
| 132 | 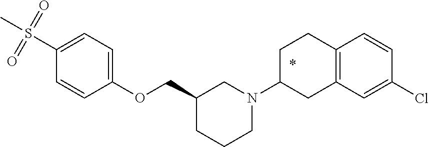 [SMILES: CS(=O)(=O)c1ccc(OC[C@@H]2CCCN(C3CCc4ccc(Cl)cc4C3)C2)cc1] | (3R)-1-[(2S or 2R)-7- chloro-1,2,3,4-tetrahydro- naphthalen-2-yl]-3-[(4- methanesulfonylphenoxy)- methyl]piperidine |
| 133 | 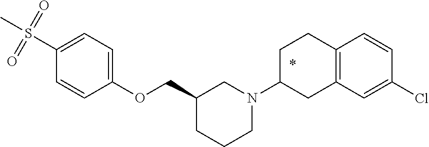 [SMILES: CS(=O)(=O)c1ccc(OC[C@@H]2CCCN(C3CCc4ccc(Cl)cc4C3)C2)cc1] | (3R)-1-[(2R or 2S)-7- chloro-1,2,3,4-tetrahydro- naphthalen-2-yl]-3-[(4- methanesulfonylphenoxy)- methyl]piperidine |
| 134 | 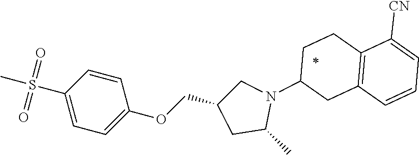 [SMILES: C[C@@H]1C[C@H](COc2ccc(S(C)(=O)=O)cc2)CN1C1CCc2c(C#N)cccc2C1] | (6R or 6S)-6-[(2R,4S)-4- [(4-methanesulfonyl- phenoxy)methyl]-2-methyl- pyrrolidin-1-yl]-5,6,7,8- tetrahydronaphthalene-1- carbonitrile |
| 135 | 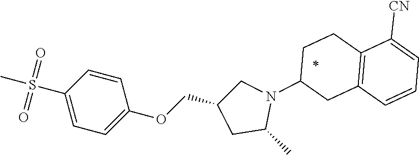 [SMILES: C[C@@H]1C[C@H](COc2ccc(S(C)(=O)=O)cc2)CN1C1CCc2c(C#N)cccc2C1] | (6S or 6R)-6-[(2R,4S)-4- [(4-methanesulfonylphen- oxy)methyl]-2-methyl- pyrrolidin-1-yl]-5,6,7,8- tetrahydronaphthalene-1- carbonitrile |
| 136 | 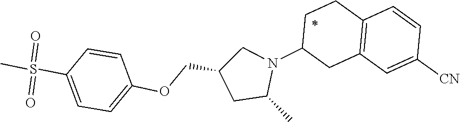 [SMILES: C[C@@H]1C[C@H](COc2ccc(S(C)(=O)=O)cc2)CN1C1CCc2ccc(C#N)cc2C1] | (7S or 7R)-7-[(2R,4S)-4- [(4-methanesulfonylphen- oxy)methyl]-2-methyl- pyrrolidin-1-yl]-5,6,7,8- tetrahydronaphthalene-2- carbonitrile |
| 137 | 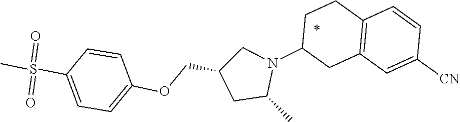 [SMILES: C[C@@H]1C[C@H](COc2ccc(S(C)(=O)=O)cc2)CN1C1CCc2ccc(C#N)cc2C1] | (7R or 7S)-7-[(2R,4S)-4- [(4-methanesulfonylphen- oxy)methyl]-2-methyl- pyrrolidin-1-yl]-5,6,7,8- tetrahydronaphthalene-2- carbonitrile |
| 138 | 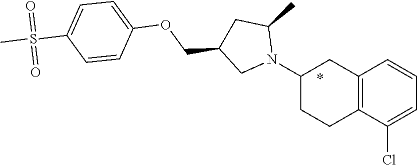 [SMILES: C[C@@H]1C[C@H](COc2ccc(S(C)(=O)=O)cc2)CN1C1CCc2c(Cl)cccc2C1] | (2R,4S)-1-[(2R or 2S)-5- chloro-1,2,3,4-tetrahydro- naphthalen-2-yl]-4-[(4- methanesulfonylphenoxy)- methyl]-2-methylpyrrolidine |
| 139 | 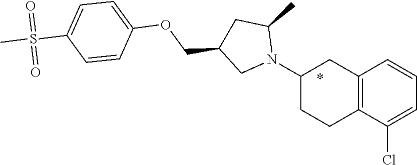 [SMILES: C[C@@H]1C[C@H](COc2ccc(S(C)(=O)=O)cc2)CN1C1CCc2c(Cl)cccc2C1] | (2R,4S)-1-[(2S or 2R)-5- chloro-1,2,3,4-tetrahydro- naphthalen-2-yl]-4-[(4- methanesulfonylphenoxy)- methyl]-2-methylpyrrolidine |
| 140 | 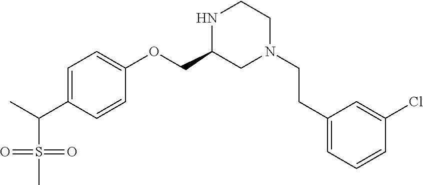 [SMILES: CC(c1ccc(OC[C@@H]2CN(CCc3cccc(Cl)c3)CCN2)cc1)S(C)(=O)=O] | (3S)-1-[2-(3-chlorophen- yl)ethyl]-3-{[4-(1-methane- sulfonylethyl)phenoxy] methyl}piperazine |
| 141 | 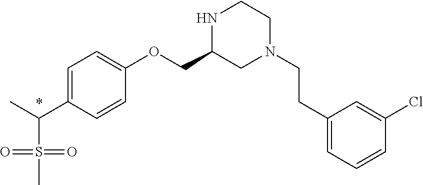 [SMILES: CC(c1ccc(OC[C@@H]2CN(CCc3cccc(Cl)c3)CCN2)cc1)S(C)(=O)=O] | (3S)-1-[2-(3-chlorophen- yl)ethyl]-3-({4-[(1R) or (1S)-1-methanesulfonyl- ethyl]phenoxy}methyl)- piperazine |
| 142 | 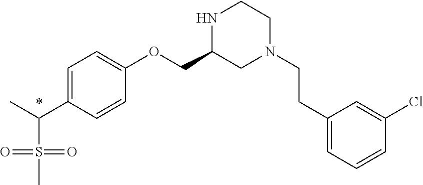 [SMILES: CC(c1ccc(OC[C@@H]2CN(CCc3cccc(Cl)c3)CCN2)cc1)S(C)(=O)=O] | (3S)-1-[2-(3-chlorophen- yl)ethyl]-3-({4-[(1S) or (1R)-1-methanesulfonyl- ethyl]phenoxy}methyl)- piperazine |
| 143 | 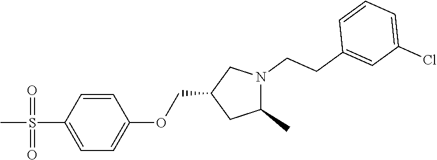 [SMILES: C[C@H]1C[C@H](COc2ccc(S(C)(=O)=O)cc2)CN1CCc1cccc(Cl)c1] | (2S,4S)-1-[2-(3-chloro- phenyl)ethyl]-4-[(4- methanesulfonylphenoxy)- methyl]-2-methylpyrrolidine |
| 144 | 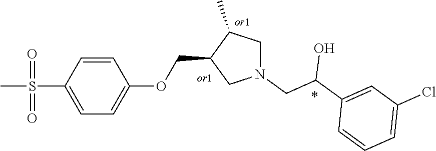 [SMILES: C[C@@H]1CN(CC(O)c2cccc(Cl)c2)C[C@H]1COc1ccc(S(C)(=O)=O)cc1] | (1S) or (1R)-1-(3-chloro- phenyl)-2-[trans-3-[(4- methanesulfonylphenoxy)- methyl]-4-methylpyrrol- idin-1-yl]ethan-1-ol |
| 145 | 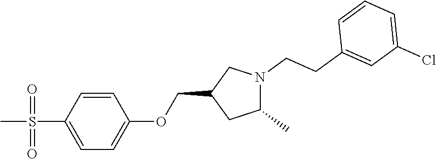 | (2R,4R)-1-[2-(3-chloro- phenyl)ethyl]-4-[(4- methanesulfonylphenoxy)- methyl]-2-methylpyrrolidine |

In some embodiments, a compound of formula (A) is selected from the group consisting of:
3-(2-(2-methyl-4-(((5-(methylsulfonyl)pyrazin-2-yl)oxy)methyl)pyrrolidin-1-yl)ethyl)benzonitrile;
1-[5-chloro-2,3-dihydro-1H-inden-2-yl]-4-[(4-methanesulfonylphenoxy)methyl]-2-methylpyrrolidine;
1-[7-chloro-1,2,3,4-tetrahydronaphthalen-2-yl]-4-[(4-methanesulfonylphenoxy)methyl]-2-methylpyrrolidine;
3-chloro-6-[4-[(4-methanesulfonylphenoxy)methyl]-2-methylpyrrolidin-1-yl]-5,6,7,8-tetrahydronaphthalene-1-carbonitrile;
3-chloro-5-[2-[4-[(4-methanesulfonylphenoxy)methyl]-2-methylpyrrolidin-1-yl]propyl]benzonitrile;
3-chloro-5-{2-[4-{[(5-methanesulfonylpyridin-2-yl)oxy]methyl}-2-methylpyrrolidin-1-yl]ethyl}benzonitrile;
1-[7-chloro-1,2,3,4-tetrahydronaphthalen-2-yl]-3-[(4-methanesulfonylphenoxy)methyl]-4-methylpyrrolidine;
3-chloro-5-[2-[3-[(4-methanesulfonylphenoxy)methyl]-4-methylpyrrolidin-1-yl]propyl]benzonitrile;
3-{2-[3-{[(5-methanesulfonylpyrazin-2-yl)oxy]methyl}-4-methylpyrrolidin-1-yl]ethyl}benzonitrile;
[1-[2-(3-chlorophenyl)ethyl]-4-[(4-methanesulfonylphenoxy)methyl]pyrrolidin-3-yl]methanol;
5-chloro-3-{2-[3-(hydroxymethyl)-4-[(4-methanesulfonylphenoxy)methyl]pyrrolidin-1-yl]ethyl}-2-methylbenzonitrile;
3-chloro-5-{2-[3-(hydroxymethyl)-4-[(4-methanesulfonylphenoxy)methyl]pyrrolidin-1-yl]ethyl}benzonitrile;
3-{2-[3-(hydroxymethyl)-4-[(4-methanesulfonylphenoxy)methyl]pyrrolidin-1-yl]ethyl}benzonitrile;
3-{2-[4-{[(6-methanesulfonylpyridazin-3-yl)oxy]methyl}-2-methylpyrrolidin-1-yl]ethyl}benzonitrile;
7-[4-{[4-(azetidine-3-sulfonyl)phenoxy]methyl}-2-methylpyrrolidin-1-yl]-5,6,7,8-tetrahydronaphthalene-2-carbonitrile;
3-{2-[4-{[(6-methanesulfonyl-5-methylpyridin-3-yl)oxy]methyl}-2-methylpyrrolidin-1-yl]ethyl}benzonitrile;
7-[4-{[4-(2-methanesulfonylethanesulfonyl)phenoxy]methyl}-2-methylpyrrolidin-1-yl]-5,6,7,8-tetrahydronaphthalene-2-carbonitrile;
7-[4-{[4-(3-methanesulfonylpropanesulfonyl)phenoxy]methyl}-2-methylpyrrolidin-1-yl]-5,6,7,8-tetrahydronaphthalene-2-carbonitrile;
7-[2-methyl-4-({4-[methyl(methylimino)oxo-λ⁶-sulfanyl]phenoxy}methyl)pyrrolidin-1-yl]-5,6,7,8-tetrahydronaphthalene-2-carbonitrile;
5-{2-[4-{[(6-methanesulfonylpyridin-3-yl)oxy]methyl}-2-methylpyrrolidin-1-yl]ethyl}benzene-1,3-dicarbonitrile;
6-[4-{[4-(3-methanesulfonylpropanesulfonyl)phenoxy]methyl}-2-methylpyrrolidin-1-yl]-5,6,7,8-tetrahydronaphthalene-1-carbonitrile;
3-chloro-6-[4-{[4-(3-methanesulfonylpropanesulfonyl)phenoxy]methyl}-2-methylpyrrolidin-1-yl]-5,6,7,8-tetrahydronaphthalene-1-carbonitrile;
3-chloro-6-[4-{[4-(2-methanesulfonylethanesulfonyl)phenoxy]methyl}-2-methylpyrrolidin-1-yl]-5,6,7,8-tetrahydronaphthalene-1-carbonitrile;
1-[7-chloro-1,2,3,4-tetrahydronaphthalen-2-yl]-4-{[4-(2-methanesulfonylethanesulfonyl)phenoxy]methyl}-2-methylpyrrolidine;
1-[7-chloro-1,2,3,4-tetrahydronaphthalen-2-yl]-4-{[4-(3-methanesulfonylpropanesulfonyl)phenoxy]methyl}-2-methylpyrrolidine;
3-chloro-6-[4-{[4-(2-hydroxyethanesulfonyl)phenoxy]methyl}-2-methylpyrrolidin-1-yl]-5,6,7,8-tetrahydronaphthalene-1-carbonitrile;
7-[4-{[4-(2-hydroxyethanesulfonyl)phenoxy]methyl}-2-methylpyrrolidin-1-yl]-5,6,7,8-tetrahydronaphthalene-2-carbonitrile;
2-(4-{[1-[7-chloro-1,2,3,4-tetrahydronaphthalen-2-yl]-5-methylpyrrolidin-3-yl]methoxy}benzenesulfonyl)ethan-1-ol;
3-{2-[4-{[(6-methanesulfonylpyridin-3-yl)oxy]methyl}-2-methylpyrrolidin-1-yl]ethyl}benzonitrile;
3-chloro-5-{2-[4-{[(6-methanesulfonylpyridin-3-yl)oxy]methyl}-2-methylpyrrolidin-1-yl]ethyl}benzonitrile;
N-[2-(4-{[1-[2-(3-chloro-5-cyanophenyl)ethyl]-5-methylpyrrolidin-3-yl]methoxy}benzenesulfonyl)ethyl]-N-methylacetamide;
N-[2-(4-{[1-[2-(3-cyanophenyl)ethyl]-5-methylpyrrolidin-3-yl]methoxy}benzenesulfonyl)ethyl]-N-methylacetamide;
3-{2-[3-[(4-{[(2-methanesulfonylethyl)imino](methyl)oxo-λ⁶-sulfanyl}phenoxy)methyl]-4-methylpyrrolidin-1-yl]ethyl}benzonitrile;
3-{2-[3-methyl-4-[({6-[methyl(methylimino)oxo-λ⁶-sulfanyl]pyridin-3-yl}oxy)methyl]pyrrolidin-1-yl]ethyl}benzonitrile;
5-{2-[3-{[(6-methanesulfonylpyridin-3-yl)oxy]methyl}-4-methylpyrrolidin-1-yl]ethyl}benzene-1,3-dicarbonitrile;
N-[2-(4-{[1-[2-(3-chloro-5-cyanophenyl)ethyl]-4-methylpyrrolidin-3-yl]methoxy}benzenesulfonyl)ethyl]-N-methylacetamide;
N-[2-(4-{[1-[2-(3-cyanophenyl)ethyl]-4-methylpyrrolidin-3-yl]methoxy}benzenesulfonyl)ethyl]-N-methylacetamide;
3-chloro-5-{2-[3-{[4-(1-methanesulfonylcyclopropyl)phenoxy]methyl}-4-methylpyrrolidin-1-yl]ethyl}benzonitrile;
3-chloro-5-{2-[3-{[4-(3-methanesulfonyloxetan-3-yl)phenoxy]methyl}-4-methylpyrrolidin-1-yl]ethyl}benzonitrile;
methyl 4-(4-{[1-[2-(3-chlorophenyl)ethyl]-4-methylpyrrolidin-3-yl]methoxy}benzenesulfonyl)butanoate;
methyl 4-(4-{[1-[2-(3-chloro-5-cyanophenyl)ethyl]-4-methylpyrrolidin-3-yl]methoxy}benzenesulfonyl)butanoate;
3-{2-[3-[(3,5-difluoro-4-methanesulfonylphenoxy)methyl]-4-methylpyrrolidin-1-yl]ethyl}benzonitrile;
3-chloro-5-{2-[3-[(3,5-difluoro-4-methanesulfonylphenoxy)methyl]-4-methylpyrrolidin-1-yl]ethyl}benzonitrile;
3-{2-[3-[(4-methanesulfonyl-3-methylphenoxy)methyl]-4-methylpyrrolidin-1-yl]ethyl}benzonitrile;
3-chloro-5-{2-[3-[(4-methanesulfonyl-3-methylphenoxy)methyl]-4-methylpyrrolidin-1-yl]ethyl}benzonitrile;
3-chloro-5-{2-[3-{[(6-methanesulfonylpyridin-3-yl)oxy]methyl}-4-methylpyrrolidin-1-yl]ethyl}benzonitrile;
5-{[1-[2-(3-cyanophenyl)ethyl]-4-methylpyrrolidin-3-yl]methoxy}-2-methanesulfonylbenzonitrile;
3-{2-[3-{[(6-methanesulfonyl-5-methylpyridin-3-yl)oxy]methyl}-4-methylpyrrolidin-1-yl]ethyl}benzonitrile;
1-(5-chloro-1,2,3,4-tetrahydronaphthalen-2-yl)-4-[(4-methanesulfonylphenoxy)methyl]-2-methylpyrrolidine;
3-chloro-5-{2-[3-({4-[(1,1-dioxo-1λ⁶-thietan-3-yl)methanesulfonyl]phenoxy}methyl)-4-methylpyrrolidin-1-yl]ethyl}benzonitrile;
7-[4-[(4-methanesulfonylphenoxy)methyl]-2-methylpyrrolidin-1-yl]-5,6,7,8-tetrahydronaphthalene-2-carbonitrile;
3-[1-hydroxy-2-[4-{[(6-methanesulfonylpyridin-3-yl)oxy]methyl}-2-methylpyrrolidin-1-yl]ethyl]benzonitrile;
3-[1-hydroxy-2-[4-{[(6-methanesulfonyl-5-methylpyridin-3-yl)oxy]methyl}-2-methylpyrrolidin-1-yl]ethyl]benzonitrile;
3-chloro-5-[1-hydroxy-2-[4-[(4-methanesulfonylphenoxy)methyl]-2-methylpyrrolidin-1-yl]propyl]benzonitrile;
3-chloro-5-(1-hydroxy-2-(3-methyl-4-((4-(methylsulfonyl)phenoxy)methyl)pyrrolidin-1-yl)propyl)benzonitrile;
3-{1-hydroxy-2-[3-{[(6-methanesulfonylpyridin-3-yl)oxy]methyl}-4-methylpyrrolidin-1-yl]ethyl}benzonitrile;
3-[1-hydroxy-2-[3-{[(6-methanesulfonylpyridin-3-yl)oxy]methyl}-4-methylpyrrolidin-1-yl]ethyl]benzonitrile;
1-[2-(3-chlorophenyl)ethyl]-3-{[4-(3-methanesulfonyloxetan-3-yl)phenoxy]methyl}piperazine;
1-[2-(3-chlorophenyl)ethyl]-3-({4-[(1-methylazetidin-3-yl)sulfonyl]phenoxy}methyl)piperazine;
1-[3-(4-{[4-[2-(3-chlorophenyl)ethyl]piperazin-2-yl]methoxy}benzenesulfonyl)azetidin-1-yl]ethan-1-one;
3-chloro-5-{2-[3-{[4-(1-methanesulfonylcyclopropyl)phenoxy]methyl}piperazin-1-yl]ethyl}benzonitrile;
3-chloro-5-{2-[3-{[4-(3-methanesulfonyloxetan-3-yl)phenoxy]methyl}piperazin-1-yl]ethyl}benzonitrile;
3-chloro-5-{2-[3-[(4-methanesulfonyl-3-methylphenoxy)methyl]piperazin-1-yl]ethyl}benzonitrile;
3-{2-[3-{[4-methanesulfonyl-3-(trifluoromethyl)phenoxy]methyl}piperazin-1-yl]ethyl}benzonitrile;
3-chloro-5-{2-[3-{[4-methanesulfonyl-3-(trifluoromethyl)phenoxy]methyl}piperazin-1-yl]ethyl}benzonitrile;
3-(3-chlorophenyl)-2-[3-[(4-methanesulfonylphenoxy)methyl]-4-methylpyrrolidin-1-yl]propan-1-ol;
3-(3-chlorophenyl)-2-[3-[(4-methanesulfonylphenoxy)methyl]piperazin-1-yl]propan-1-ol;
3-chloro-5-[3-hydroxy-2-[3-[(4-methanesulfonylphenoxy)methyl]-4-methylpyrrolidin-1-yl]propyl]benzonitrile;
[1-[2-(3-chlorophenyl)ethyl]-4-[(4-methanesulfonylphenoxy)methyl]pyrrolidin-2-yl]methanol;
3-{[2-[(4-methanesulfonylphenoxy)methyl]-octahydroindolizin-5-yl]methyl}-5-chlorobenzonitrile;
3-chloro-5-(1-hydroxy-3-(3-methyl-4-((4-(methylsulfonyl)phenoxy)methyl)pyrrolidin-1-yl)propan-2-yl)benzonitrile;
3-chloro-5-[1-hydroxy-3-[3-[(4-methanesulfonylphenoxy)methyl]-4-methylpyrrolidin-1-yl]propan-2-yl]benzonitrile;
3-chloro-5-[1-hydroxy-3-[4-[(4-methanesulfonylphenoxy)methyl]-2-methylpyrrolidin-1-yl]propan-2-yl]benzonitrile;
1-(7-chloro-1,2,3,4-tetrahydronaphthalen-2-yl)-3-[(4-methanesulfonylphenoxy)methyl]piperidine;
1-[7-chloro-1,2,3,4-tetrahydronaphthalen-2-yl]-3-[(4-methanesulfonylphenoxy)methyl]piperidine;
6-[4-[(4-methanesulfonylphenoxy)methyl]-2-methylpyrrolidin-1-yl]-5,6,7,8-tetrahydronaphthalene-1-carbonitrile;
7-[4-[(4-methanesulfonylphenoxy)methyl]-2-methylpyrrolidin-1-yl]-5,6,7,8-tetrahydronaphthalene-2-carbonitrile;
1-[5-chloro-1,2,3,4-tetrahydronaphthalen-2-yl]-4-[(4-methanesulfonylphenoxy)methyl]-2-methylpyrrolidine; and
1-[2-(3-chlorophenyl)ethyl]-3-{[4-(1-methanesulfonylethyl)phenoxy]methyl}piperazine; and
   or a tautomer thereof, or a pharmaceutically acceptable salt of any of the foregoing. Isotopically labeled forms of any of the foregoing are also embraced, such as deuterated or tritiated forms (wherein at least one hydrogen is replaced by at least one deuterium or tritium) of any of the specific compounds detailed herein. Mixtures of any of the foregoing are also embraced and described. Prodrugs of any of the foregoing are also embraced herein.

In some embodiments, a compound of formula (A) is selected from the group consisting of:
3-(2-((2R,4S)-2-methyl-4-(((5-(methylsulfonyl)pyrazin-2-yl)oxy)methyl)pyrrolidin-1-yl)ethyl)benzonitrile;
(2R,4S)-1-[5-chloro-2,3-dihydro-1H-inden-2-yl]-4-[(4-methanesulfonylphenoxy)methyl]-2-methylpyrrolidine;
(2R,4S)-1-[(2R or 2S)-5-chloro-2,3-dihydro-1H-inden-2-yl]-4-[(4-methanesulfonylphenoxy)methyl]-2-methylpyrrolidine;
(2R,4S)-1-[(2S or 2R)-5-chloro-2,3-dihydro-1H-inden-2-yl]-4-[(4-methanesulfonylphenoxy)methyl]-2-methylpyrrolidine;
(2R,4S)-1-[(2S or 2R)-7-chloro-1,2,3,4-tetrahydronaphthalen-2-yl]-4-[(4-methanesulfonylphenoxy)methyl]-2-methylpyrrolidine;
(2R,4S)-1-[(2R or 2S)-7-chloro-1,2,3,4-tetrahydronaphthalen-2-yl]-4-[(4-methanesulfonylphenoxy)methyl]-2-methylpyrrolidine;
(6R or 6S)-3-chloro-6-[(2R,4S)-4-[(4-methanesulfonylphenoxy)methyl]-2-methylpyrrolidin-1-yl]-5,6,7,8-tetrahydronaphthalene-1-carbonitrile;
(6R or 6S)-3-chloro-6-[(2R,4S)-4-[(4-methanesulfonylphenoxy)methyl]-2-methylpyrrolidin-1-yl]-5,6,7,8-tetrahydronaphthalene-1-carbonitrile;
(6S or 6R)-3-chloro-6-[(2R,4S)-4-[(4-methanesulfonylphenoxy)methyl]-2-methylpyrrolidin-1-yl]-5,6,7,8-tetrahydronaphthalene-1-carbonitrile;
3-chloro-5-[(2S or 2R)-2-[(2R,4S)-4-[(4-methanesulfonylphenoxy)methyl]-2-methylpyrrolidin-1-yl]propyl]benzonitrile;
3-chloro-5-[(2R or 2S)-2-[(2R,4S)-4-[(4-methanesulfonylphenoxy)methyl]-2-methylpyrrolidin-1-yl]propyl]benzonitrile;
3-chloro-5-{2-[(2R,4S)-4-{[(5-methanesulfonylpyridin-2-yl)oxy]methyl}-2-methylpyrrolidin-1-yl]ethyl}benzonitrile;
(3S,4S)-1-[7-chloro-1,2,3,4-tetrahydronaphthalen-2-yl]-3-[(4-methanesulfonylphenoxy)methyl]-4-methylpyrrolidine;
(3S,4S)-1-[(2S or 2R)-7-chloro-1,2,3,4-tetrahydronaphthalen-2-yl]-3-[(4-methanesulfonylphenoxy)methyl]-4-methylpyrrolidine;
(3S,4S)-1-[(2R or 2S)-7-chloro-1,2,3,4-tetrahydronaphthalen-2-yl]-3-[(4-methanesulfonylphenoxy)methyl]-4-methylpyrrolidine;
3-chloro-5-[2-[(3S,4S)-3-[(4-methanesulfonylphenoxy)methyl]-4-methylpyrrolidin-1-yl]propyl]benzonitrile;
3-chloro-5-[(2S or 2R)-2-[(3S,4S)-3-[(4-methanesulfonylphenoxy)methyl]-4-methylpyrrolidin-1-yl]propyl]benzonitrile;
3-chloro-5-[(2R or 2S)-2-[(3S,4S)-3-[(4-methanesulfonylphenoxy)methyl]-4-methylpyrrolidin-1-yl]propyl]benzonitrile;
3-{2-[(3S,4S)-3-{[(5-methanesulfonylpyrazin-2-yl)oxy]methyl}-4-methylpyrrolidin-1-yl]ethyl}benzonitrile;
[(3S,4S)-1-[2-(3-chlorophenyl)ethyl]-4-[(4-methanesulfonylphenoxy)methyl]pyrrolidin-3-yl]methanol;
[(3R,4R)-1-[2-(3-chlorophenyl)ethyl]-4-[(4-methanesulfonylphenoxy)methyl]pyrrolidin-3-yl]methanol;
5-chloro-3-{2-[(3S,4S)-3-(hydroxymethyl)-4-[(4-methanesulfonylphenoxy)methyl]pyrrolidin-1-yl]ethyl}-2-methylbenzonitrile;
3-chloro-5-{2-[(3S,4S)-3-(hydroxymethyl)-4-[(4-methanesulfonylphenoxy)methyl]pyrrolidin-1-yl]ethyl}benzonitrile;
3-{2-[(3S,4S)-3-(hydroxymethyl)-4-[(4-methanesulfonylphenoxy)methyl]pyrrolidin-1-yl]ethyl}benzonitrile;
3-{2-[(2R,4S)-4-{[(6-methanesulfonylpyridazin-3-yl)oxy]methyl}-2-methylpyrrolidin-1-yl]ethyl}benzonitrile;
7-[(2R,4S)-4-{[4-(azetidine-3-sulfonyl)phenoxy]methyl}-2-methylpyrrolidin-1-yl]-5,6,7,8-tetrahydronaphthalene-2-carbonitrile;
7-[(2R,4S)-4-{[4-(azetidine-3-sulfonyl)phenoxy]methyl}-2-methylpyrrolidin-1-yl]-5,6,7,8-tetrahydronaphthalene-2-carbonitrile;
7-[(2R,4S)-4-{[4-(azetidine-3-sulfonyl)phenoxy]methyl}-2-methylpyrrolidin-1-yl]-5,6,7,8-tetrahydronaphthalene-2-carbonitrile;
3-{2-[(2R,4S)-4-{[(6-methanesulfonyl-5-methylpyridin-3-yl)oxy]methyl}-2-methylpyrrolidin-1-yl]ethyl}benzonitrile;
7-[(2R,4S)-4-{[4-(2-methanesulfonylethanesulfonyl)phenoxy]methyl}-2-methylpyrrolidin-1-yl]-5,6,7,8-tetrahydronaphthalene-2-carbonitrile;
7-[(2R,4S)-4-{[4-(2-methanesulfonylethanesulfonyl)phenoxy]methyl}-2-methylpyrrolidin-1-yl]-5,6,7,8-tetrahydronaphthalene-2-carbonitrile;
7S or 7R)-7-[(2R,4S)-4-{[4-(2-methanesulfonylethanesulfonyl)phenoxy]methyl}-2-methylpyrrolidin-1-yl]-5,6,7,8-tetrahydronaphthalene-2-carbonitrile;
(7R or 7S)-7-[(2R,4S)-4-{[4-(3-methanesulfonylpropanesulfonyl)phenoxy]methyl}-2-methylpyrrolidin-1-yl]-5,6,7,8-tetrahydronaphthalene-2-carbonitrile;
7-[(2R,4S)-4-{[4-(3-methanesulfonylpropanesulfonyl)phenoxy]methyl}-2-methylpyrrolidin-1-yl]-5,6,7,8-tetrahydronaphthalene-2-carbonitrile;
(7S or 7R)-7-[(2R,4S)-4-{[4-(3-methanesulfonylpropanesulfonyl)phenoxy]methyl}-2-methylpyrrolidin-1-yl]-5,6,7,8-tetrahydronaphthalene-2-carbonitrile;
(7R or 7S)-7-[(2R,4S)-2-methyl-4-({4-[methyl(methylimino)oxo-λ⁶-sulfanyl]phenoxy}methyl)pyrrolidin-1-yl]-5,6,7,8-tetrahydronaphthalene-2-carbonitrile;
(7S or 7R)-7-[(2R,4S)-2-methyl-4-({4-[methyl(methylimino)oxo-λ⁶-sulfanyl]phenoxy}methyl)pyrrolidin-1-yl]-5,6,7,8-tetrahydronaphthalene-2-carbonitrile;
5-{2-[(2R,4S)-4-{[(6-methanesulfonylpyridin-3-yl)oxy]methyl}-2-methylpyrrolidin-1-yl]ethyl}benzene-1,3-dicarbonitrile;
6-[(2R,4S)-4-{[4-(3-methanesulfonylpropanesulfonyl)phenoxy]methyl}-2-methylpyrrolidin-1-yl]-5,6,7,8-tetrahydronaphthalene-1-carbonitrile;
(6S or 6R)-6-[(2R,4S)-4-{[4-(3-methanesulfonylpropanesulfonyl)phenoxy]methyl}-2-methylpyrrolidin-1-yl]-5,6,7,8-tetrahydronaphthalene-1-carbonitrile;
(6R or 6S)-6-[(2R,4S)-4-{[4-(3-methanesulfonylpropanesulfonyl)phenoxy]methyl}-2-methylpyrrolidin-1-yl]-5,6,7,8-tetrahydronaphthalene-1-carbonitrile;
3-chloro-6-[(2R,4S)-4-{[4-(3-methanesulfonylpropanesulfonyl)phenoxy]methyl}-2-methylpyrrolidin-1-yl]-5,6,7,8-tetrahydronaphthalene-1-carbonitrile;
(6R or 6S)-3-chloro-6-[(2R,4S)-4-{[4-(3-methanesulfonylpropanesulfonyl)phenoxy]methyl}-2-methylpyrrolidin-1-yl]-5,6,7,8-tetrahydronaphthalene-1-carbonitrile;
(6S or 6R)-3-chloro-6-[(2R,4S)-4-{[4-(3-methanesulfonylpropanesulfonyl)phenoxy]methyl}-2-methylpyrrolidin-1-yl]-5,6,7,8-tetrahydronaphthalene-1-carbonitrile;
3-chloro-6-[(2R,4S)-4-{[4-(2-methanesulfonylethanesulfonyl)phenoxy]methyl}-2-methylpyrrolidin-1-yl]-5,6,7,8-tetrahydronaphthalene-1-carbonitrile;
(6S or 6R)-3-chloro-6-[(2R,4S)-4-{[4-(2-methanesulfonylethanesulfonyl)phenoxy]methyl}-2-methylpyrrolidin-1-yl]-5,6,7,8-tetrahydronaphthalene-1-carbonitrile;
(6R or 6S)-3-chloro-6-[(2R,4S)-4-{[4-(2-methanesulfonylethanesulfonyl)phenoxy]methyl}-2-methylpyrrolidin-1-yl]-5,6,7,8-tetrahydronaphthalene-1-carbonitrile;
(2R,4S)-1-[7-chloro-1,2,3,4-tetrahydronaphthalen-2-yl]-4-{[4-(2-methanesulfonylethanesulfonyl)phenoxy]methyl}-2-methylpyrrolidine;
(2R,4S)-1-[(2S or 2R)-7-chloro-1,2,3,4-tetrahydronaphthalen-2-yl]-4-{[4-(2-methanesulfonylethanesulfonyl)phenoxy]methyl}-2-methylpyrrolidine;
(2R,4S)-1-[(2R or 2S)-7-chloro-1,2,3,4-tetrahydronaphthalen-2-yl]-4-{[4-(2-methanesulfonylethanesulfonyl)phenoxy]methyl}-2-methylpyrrolidine;
(2R,4S)-1-[7-chloro-1,2,3,4-tetrahydronaphthalen-2-yl]-4-{[4-(3-methanesulfonylpropanesulfonyl)phenoxy]methyl}-2-methylpyrrolidine;
(2R,4S)-1-[(2R or 2S)-7-chloro-1,2,3,4-tetrahydronaphthalen-2-yl]-4-{[4-(3-methanesulfonylpropanesulfonyl)phenoxy]methyl}-2-methylpyrrolidine;
(2R,4S)-1-[(2S or 2R)-7-chloro-1,2,3,4-tetrahydronaphthalen-2-yl]-4-{[4-(3-methanesulfonylpropanesulfonyl)phenoxy]methyl}-2-methylpyrrolidine;
3-chloro-6-[(2R,4S)-4-{[4-(2-hydroxyethanesulfonyl)phenoxy]methyl}-2-methylpyrrolidin-1-yl]-5,6,7,8-tetrahydronaphthalene-1-carbonitrile;
(6R or 6S)-3-chloro-6-[(2R,4S)-4-{[4-(2-hydroxyethanesulfonyl)phenoxy]methyl}-2-methylpyrrolidin-1-yl]-5,6,7,8-tetrahydronaphthalene-1-carbonitrile;
(6S or 6R)-3-chloro-6-[(2R,4S)-4-{[4-(2-hydroxyethanesulfonyl)phenoxy]methyl}-2-methylpyrrolidin-1-yl]-5,6,7,8-tetrahydronaphthalene-1-carbonitrile;
7-[(2R,4S)-4-{[4-(2-hydroxyethanesulfonyl)phenoxy]methyl}-2-methylpyrrolidin-1-yl]-5,6,7,8-tetrahydronaphthalene-2-carbonitrile;
(7R or 7S)-7-[(2R,4S)-4-{[4-(2-hydroxyethanesulfonyl)phenoxy]methyl}-2-methylpyrrolidin-1-yl]-5,6,7,8-tetrahydronaphthalene-2-carbonitrile;
(7S or 7R)-7-[(2R,4S)-4-{[4-(2-hydroxyethanesulfonyl)phenoxy]methyl}-2-methylpyrrolidin-1-yl]-5,6,7,8-tetrahydronaphthalene-2-carbonitrile;
2-(4-{[(3S,5R)-1-[7-chloro-1,2,3,4-tetrahydronaphthalen-2-yl]-5-methylpyrrolidin-3-yl]methoxy}benzenesulfonyl)ethan-1-ol;
2-(4-{[(3S,5R)-1-[(2R or 2S)-7-chloro-1,2,3,4-tetrahydronaphthalen-2-yl]-5-methylpyrrolidin-3-yl]methoxy}benzenesulfonyl)ethan-1-ol;
2-(4-{[(3S,5R)-1-[(2S or 2R)-7-chloro-1,2,3,4-tetrahydronaphthalen-2-yl]-5-methylpyrrolidin-3-yl]methoxy}benzenesulfonyl)ethan-1-ol;
3-{2-[(2R,4S)-4-{[(6-methanesulfonylpyridin-3-yl)oxy]methyl}-2-methylpyrrolidin-1-yl]ethyl}benzonitrile;
3-chloro-5-{2-[(2R,4S)-4-{[(6-methanesulfonylpyridin-3-yl)oxy]methyl}-2-methylpyrrolidin-1-yl]ethyl}benzonitrile;
N-[2-(4-{[(3S,5R)-1-[2-(3-chloro-5-cyanophenyl)ethyl]-5-methylpyrrolidin-3-yl]methoxy}benzenesulfonyl)ethyl]-N-methylacetamide;
N-[2-(4-{[(3S,5R)-1-[2-(3-cyanophenyl)ethyl]-5-methylpyrrolidin-3-yl]methoxy}benzenesulfonyl)ethyl]-N-methylacetamide;
3-{2-[(3S,4S)-3-[(4-{[(2-methanesulfonylethyl)imino](methyl)oxo-λ⁶-sulfanyl}phenoxy)methyl]-4-methylpyrrolidin-1-yl]ethyl}benzonitrile;
3-{2-[(3S,4S)-3-[(4-{[(2-methanesulfonylethyl)imino](methyl)oxo-λ⁶-sulfanyl}phenoxy)methyl]-4-methylpyrrolidin-1-yl]ethyl}benzonitrile;
3-{2-[(3S,4S)-3-[(4-{[(2-methanesulfonylethyl)imino](methyl)oxo-λ⁶-sulfanyl}phenoxy)methyl]-4-methylpyrrolidin-1-yl]ethyl}benzonitrile;
3-{2-[(3S,4S)-3-methyl-4-[({6-[methyl(methylimino)oxo-λ⁶-sulfanyl]pyridin-3-yl}oxy)methyl]pyrrolidin-1-yl]ethyl}benzonitrile;
3-{2-[(3S,4S)-3-methyl-4-[({6-[methyl(methylimino)oxo-λ⁶-sulfanyl]pyridin-3-yl}oxy)methyl]pyrrolidin-1-yl]ethyl}benzonitrile;
3-{2-[(3S,4S)-3-methyl-4-[({6-[methyl(methylimino)oxo-λ⁶-sulfanyl]pyridin-3-yl}oxy)methyl]pyrrolidin-1-yl]ethyl}benzonitrile;
5-{2-[(3S,4S)-3-{[(6-methanesulfonylpyridin-3-yl)oxy]methyl}-4-methylpyrrolidin-1-yl]ethyl}benzene-1,3-dicarbonitrile;
N-[2-(4-{[(3S,4S)-1-[2-(3-chloro-5-cyanophenyl)ethyl]-4-methylpyrrolidin-3-yl]methoxy}benzenesulfonyl)ethyl]-N-methylacetamide;
N-[2-(4-{[(3S,4S)-1-[2-(3-cyanophenyl)ethyl]-4-methylpyrrolidin-3-yl]methoxy}benzenesulfonyl)ethyl]-N-methylacetamide;
3-chloro-5-{2-[(3S,4S)-3-{[4-(1-methanesulfonylcyclopropyl)phenoxy]methyl}-4-methylpyrrolidin-1-yl]ethyl}benzonitrile;
3-chloro-5-{2-[(3S,4S)-3-{[4-(3-methanesulfonyloxetan-3-yl)phenoxy]methyl}-4-methylpyrrolidin-1-yl]ethyl}benzonitrile;
methyl 4-(4-{[(3S,4S)-1-[2-(3-chlorophenyl)ethyl]-4-methylpyrrolidin-3-yl]methoxy}benzenesulfonyl)butanoate;
methyl 4-(4-{[(3S,4S)-1-[2-(3-chloro-5-cyanophenyl)ethyl]-4-methylpyrrolidin-3-yl]methoxy}benzenesulfonyl)butanoate;
3-{2-[(3S,4S)-3-[(3,5-difluoro-4-methanesulfonylphenoxy)methyl]-4-methylpyrrolidin-1-yl]ethyl}benzonitrile;
3-chloro-5-{2-[(3S,4S)-3-[(3,5-difluoro-4-methanesulfonylphenoxy)methyl]-4-methylpyrrolidin-1-yl]ethyl}benzonitrile;
3-{2-[(3S,4S)-3-[(4-methanesulfonyl-3-methylphenoxy)methyl]-4-methylpyrrolidin-1-yl]ethyl}benzonitrile;
3-chloro-5-{2-[(3S,4S)-3-[(4-methanesulfonyl-3-methylphenoxy)methyl]-4-methylpyrrolidin-1-yl]ethyl}benzonitrile;
3-chloro-5-{2-[(3S,4S)-3-{[(6-methanesulfonylpyridin-3-yl)oxy]methyl}-4-methylpyrrolidin-1-yl]ethyl}benzonitrile;
5-{[(3S,4S)-1-[2-(3-cyanophenyl)ethyl]-4-methylpyrrolidin-3-yl]methoxy}-2-methanesulfonylbenzonitrile;
3-{2-[(3S,4S)-3-{[(6-methanesulfonyl-5-methylpyridin-3-yl)oxy]methyl}-4-methylpyrrolidin-1-yl]ethyl}benzonitrile;
(2R,4S)-1-(5-chloro-1,2,3,4-tetrahydronaphthalen-2-yl)-4-[(4-methanesulfonylphenoxy)methyl]-2-methylpyrrolidine;
3-chloro-5-{2-[(3S,4S)-3-({4-[(1,1-dioxo-1λ⁶-thietan-3-yl)methanesulfonyl]phenoxy}methyl)-4-methylpyrrolidin-1-yl]ethyl}benzonitrile;
7-[(2R,4S)-4-[(4-methanesulfonylphenoxy)methyl]-2-methylpyrrolidin-1-yl]-5,6,7,8-tetrahydronaphthalene-2-carbonitrile;
3-[(1R or 1S)-1-hydroxy-2-[(2R,4S)-4-{[(6-methanesulfonylpyridin-3-yl)oxy]methyl}-2-methylpyrrolidin-1-yl]ethyl]benzonitrile;
3-[(1S or 1R)-1-hydroxy-2-[(2R,4S)-4-{[(6-methanesulfonylpyridin-3-yl)oxy]methyl}-2-methylpyrrolidin-1-yl]ethyl]benzonitrile;
3-[(1R or 1S)-1-hydroxy-2-[(2R,4S)-4-{[(6-methanesulfonyl-5-methylpyridin-3-yl)oxy]methyl}-2-methylpyrrolidin-1-yl]ethyl]benzonitrile;
3-[(1S or 1R)-1-hydroxy-2-[(2R,4S)-4-{[(6-methanesulfonyl-5-methylpyridin-3-yl)oxy]methyl}-2-methylpyrrolidin-1-yl]ethyl]benzonitrile;
3-chloro-5-[(1S,2S or 1R,2R or 1R,2S or 1S,2R)-1-hydroxy-2-[(2R,4S)-4-[(4-methanesulfonylphenoxy)methyl]-2-methylpyrrolidin-1-yl]propyl]benzonitrile;
3-chloro-5-[(1R,2S or 1S,2R or 1S,2S or 1R,2R)-1-hydroxy-2-[(2R,4S)-4-[(4-methanesulfonylphenoxy)methyl]-2-methylpyrrolidin-1-yl]propyl]benzonitrile;
3-chloro-5-[(1S,2R or 1R,2S or 1S,2S or 1R,2R)-1-hydroxy-2-[(2R,4S)-4-[(4-methanesulfonylphenoxy)methyl]-2-methylpyrrolidin-1-yl]propyl]benzonitrile;
3-chloro-5-((1R,2S or 1S,2R)-1-hydroxy-2-((3S,4S)-3-methyl-4-((4-(methylsulfonyl)phenoxy)methyl)pyrrolidin-1-yl)propyl)benzonitrile;
3-chloro-5-((1S,2R or 1R,2S)-1-hydroxy-2-((3S,4S)-3-methyl-4-((4-(methylsulfonyl)phenoxy)methyl)pyrrolidin-1-yl)propyl)benzonitrile;
3-chloro-5-((1R,2R or 1S,2S)-1-hydroxy-2-((3S,4S)-3-methyl-4-((4-(methylsulfonyl)phenoxy)methyl)pyrrolidin-1-yl)propyl)benzonitrile;
3-chloro-5-((1S,2S or 1R,2R)-1-hydroxy-2-((3S,4S)-3-methyl-4-((4-(methylsulfonyl)phenoxy)methyl)pyrrolidin-1-yl)propyl)benzonitrile;
3-{1-hydroxy-2-[(3S,4S)-3-{[(6-methanesulfonylpyridin-3-yl)oxy]methyl}-4-methylpyrrolidin-1-yl]ethyl}benzonitrile;
3-[(1S or 1R)-1-hydroxy-2-[(3S,4S)-3-{[(6-methanesulfonylpyridin-3-yl)oxy]methyl}-4-methylpyrrolidin-1-yl]ethyl]benzonitrile;
3-[(1R or 1S)-1-hydroxy-2-[(3S,4S)-3-{[(6-methanesulfonylpyridin-3-yl)oxy]methyl}-4-methylpyrrolidin-1-yl]ethyl]benzonitrile;
(3S)-1-[2-(3-chlorophenyl)ethyl]-3-{[4-(3-methanesulfonyloxetan-3-yl)phenoxy]methyl}piperazine;
(3S)-1-[2-(3-chlorophenyl)ethyl]-3-({4-[(1-methylazetidin-3-yl)sulfonyl]phenoxy}methyl)piperazine;
1-[3-(4-{[(2S)-4-[2-(3-chlorophenyl)ethyl]piperazin-2-yl]methoxy}benzenesulfonyl)azetidin-1-yl]ethan-1-one;
3-chloro-5-{2-[(3S)-3-{[4-(1-methanesulfonylcyclopropyl)phenoxy]methyl}piperazin-1-yl]ethyl}benzonitrile;
3-chloro-5-{2-[(3S)-3-{[4-(3-methanesulfonyloxetan-3-yl)phenoxy]methyl}piperazin-1-yl]ethyl}benzonitrile;
3-chloro-5-{2-[(3S)-3-[(4-methanesulfonyl-3-methylphenoxy)methyl]piperazin-1-yl]ethyl}benzonitrile;
3-{2-[(3S)-3-{[4-methanesulfonyl-3-(trifluoromethyl)phenoxy]methyl}piperazin-1-yl]ethyl}benzonitrile;
3-chloro-5-{2-[(3S)-3-{[4-methanesulfonyl-3-(trifluoromethyl)phenoxy]methyl}piperazin-1-yl]ethyl}benzonitrile;
3-(3-chlorophenyl)-2-[(3S,4S)-3-[(4-methanesulfonylphenoxy)methyl]-4-methylpyrrolidin-1-yl]propan-1-ol;
(2R or 2S)-3-(3-chlorophenyl)-2-[(3S,4S)-3-[(4-methanesulfonylphenoxy)methyl]-4-methylpyrrolidin-1-yl]propan-1-ol;
(2S or 2R)-3-(3-chlorophenyl)-2-[(3S,4S)-3-[(4-methanesulfonylphenoxy)methyl]-4-methylpyrrolidin-1-yl]propan-1-ol;
(2R or 2S)-3-(3-chlorophenyl)-2-[(3S)-3-[(4-methanesulfonylphenoxy)methyl]piperazin-1-yl]propan-1-ol;
(2S or 2R)-3-(3-chlorophenyl)-2-[(3S)-3-[(4-methanesulfonylphenoxy)methyl]piperazin-1-yl]propan-1-ol;
3-chloro-5-[3-hydroxy-2-[(3S,4S)-3-[(4-methanesulfonylphenoxy)methyl]-4-methylpyrrolidin-1-yl]propyl]benzonitrile;
3-chloro-5-[(2R or 2S)-3-hydroxy-2-[(3S,4S)-3-[(4-methanesulfonylphenoxy)methyl]-4-methylpyrrolidin-1-yl]propyl]benzonitrile;
3-chloro-5-[(2S or 2R)-3-hydroxy-2-[(3S,4S)-3-[(4-methanesulfonylphenoxy)methyl]-4-methylpyrrolidin-1-yl]propyl]benzonitrile;
[(2S,4S or 4R)-1-[2-(3-chlorophenyl)ethyl]-4-[(4-methanesulfonylphenoxy)methyl]pyrrolidin-2-yl]methanol;
[(2S,4R or 4S)-1-[2-(3-chlorophenyl)ethyl]-4-[(4-methanesulfonylphenoxy)methyl]pyrrolidin-2-yl]methanol;
3-{[(2S,5R or 5S,8aR)-2-[(4-methanesulfonylphenoxy)methyl]-octahydroindolizin-5-yl]methyl}-5-chlorobenzonitrile;
3-chloro-5-(1-hydroxy-3-((3S,4S)-3-methyl-4-((4-(methylsulfonyl)phenoxy)methyl)pyrrolidin-1-yl)propan-2-yl)benzonitrile;
3-chloro-5-[(2R or 2S)-1-hydroxy-3-[(3S,4S)-3-[(4-methanesulfonylphenoxy)methyl]-4-methylpyrrolidin-1-yl]propan-2-yl]benzonitrile;
3-chloro-5-[(2S or 2R)-1-hydroxy-3-[(3S,4S)-3-[(4-methanesulfonylphenoxy)methyl]-4-methylpyrrolidin-1-yl]propan-2-yl]benzonitrile;
3-chloro-5-[(2R or 2S)-1-hydroxy-3-[(2R,4S)-4-[(4-methanesulfonylphenoxy)methyl]-2-methylpyrrolidin-1-yl]propan-2-yl]benzonitrile;
3-chloro-5-[(2S or 2R)-1-hydroxy-3-[(2R,4S)-4-[(4-methanesulfonylphenoxy)methyl]-2-methylpyrrolidin-1-yl]propan-2-yl]benzonitrile;
(3R)-1-(7-chloro-1,2,3,4-tetrahydronaphthalen-2-yl)-3-[(4-methanesulfonylphenoxy)methyl]piperidine;
(3R)-1-[(2S or 2R)-7-chloro-1,2,3,4-tetrahydronaphthalen-2-yl]-3-[(4-methanesulfonylphenoxy)methyl]piperidine;
(3R)-1-[(2R or 2S)-7-chloro-1,2,3,4-tetrahydronaphthalen-2-yl]-3-[(4-methanesulfonylphenoxy)methyl]piperidine;
(6R or 6S)-6-[(2R,4S)-4-[(4-methanesulfonylphenoxy)methyl]-2-methylpyrrolidin-1-yl]-5,6,7,8-tetrahydronaphthalene-1-carbonitrile;
6S or 6R)-6-[(2R,4S)-4-[(4-methanesulfonylphenoxy)methyl]-2-methylpyrrolidin-1-yl]-5,6,7,8-tetrahydronaphthalene-1-carbonitrile;
(7S or 7R)-7-[(2R,4S)-4-[(4-methanesulfonylphenoxy)methyl]-2-methylpyrrolidin-1-yl]-5,6,7,8-tetrahydronaphthalene-2-carbonitrile;
(7R or 7S)-7-[(2R,4S)-4-[(4-methanesulfonylphenoxy)methyl]-2-methylpyrrolidin-1-yl]-5,6,7,8-tetrahydronaphthalene-2-carbonitrile;
(2R,4S)-1-[(2R or 2S)-5-chloro-1,2,3,4-tetrahydronaphthalen-2-yl]-4-[(4-methanesulfonylphenoxy)methyl]-2-methylpyrrolidine;
(2R,4S)-1-[(2S or 2R)-5-chloro-1,2,3,4-tetrahydronaphthalen-2-yl]-4-[(4-methanesulfonylphenoxy)methyl]-2-methylpyrrolidine;
(3S)-1-[2-(3-chlorophenyl)ethyl]-3-{[4-(1-methanesulfonylethyl)phenoxy]methyl}piperazine;
(3S)-1-[2-(3-chlorophenyl)ethyl]-3-({4-[(1R) or (1S)-1-methanesulfonylethyl]phenoxy}methyl)piperazine; and
(3S)-1-[2-(3-chlorophenyl)ethyl]-3-({4-[(1S) or (1R)-1-methanesulfonylethyl]phenoxy}methyl)piperazine;
   or a stereoisomer or tautomer thereof, or a pharmaceutically acceptable salt of any of the foregoing. Isotopically labeled forms of any of the foregoing are also embraced, such as deuterated or tritiated forms (wherein at least one hydrogen is replaced by at least one deuterium or tritium) of any of the specific compounds detailed herein. Mixtures of any of the foregoing are also embraced and described. Prodrugs of any of the foregoing are also embraced herein.

In some embodiments, a compound of formula (X) is selected from the group consisting of:
3-chloro-5-{2-[3-methyl-4-({4-[(3-methyloxetan-3-yl)sulfonyl]phenoxy}methyl)pyrrolidin-1-yl]ethyl}benzonitrile;
1-(3-chlorophenyl)-2-[trans-3-[(4-methanesulfonylphenoxy)methyl]-4-methylpyrrolidin-1-yl]ethan-1-ol;
1-[2-(3-chlorophenyl)ethyl]-3-[(4-methanesulfonylphenoxy)methyl]-4-methylpyrrolidine;
1-[2-(3-chlorophenyl)ethyl]-4-[(4-methanesulfonylphenoxy)methyl]-2-methylpyrrolidine;
1-(3-chlorophenyl)-2-[trans-3-[(4-methanesulfonylphenoxy)methyl]-4-methylpyrrolidin-1-yl]ethan-1-ol; and
1-[2-(3-chlorophenyl)ethyl]-4-[(4-methanesulfonylphenoxy)methyl]-2-methylpyrrolidine
   or a tautomer thereof, or a pharmaceutically acceptable salt of any of the foregoing. Isotopically labeled forms of any of the foregoing are also embraced, such as deuterated or tritiated forms (wherein at least one hydrogen is replaced by at least one deuterium or tritium) of any of the specific compounds detailed herein. Mixtures of any of the foregoing are also embraced and described. Prodrugs of any of the foregoing are also embraced herein.

In some embodiments, a compound of formula (X) is selected from the group consisting of:
3-chloro-5-{2-[(3S,4S)-3-methyl-4-({4-[(3-methyloxetan-3-yl)sulfonyl]phenoxy}methyl)pyrrolidin-1-yl]ethyl}benzonitrile;
(1R) or (1S)-1-(3-chlorophenyl)-2-[trans-3-[(4-methanesulfonylphenoxy)methyl]-4-methylpyrrolidin-1-yl]ethan-1-ol;
(3R,4R)-1-[2-(3-chlorophenyl)ethyl]-3-[(4-methanesulfonylphenoxy)methyl]-4-methylpyrrolidine;
(2S,4S)-1-[2-(3-chlorophenyl)ethyl]-4-[(4-methanesulfonylphenoxy)methyl]-2-methylpyrrolidine;
(1S) or (1R)-1-(3-chlorophenyl)-2-[trans-3-[(4-methanesulfonylphenoxy)methyl]-4-methylpyrrolidin-1-yl]ethan-1-ol; and
(2R,4R)-1-[2-(3-chlorophenyl)ethyl]-4-[(4-methanesulfonylphenoxy)methyl]-2-methylpyrrolidine
   or a stereoisomer or a tautomer thereof, or a pharmaceutically acceptable salt of any of the foregoing. Isotopically labeled forms of any of the foregoing are also embraced, such as deuterated or tritiated forms (wherein at least one hydrogen is replaced by at least one deuterium or tritium) of any of the specific compounds detailed herein. Mixtures of any of the foregoing are also embraced and described. Prodrugs of any of the foregoing are also embraced herein.

Compound Names included in Table 1 and in the lists in the paragraphs above were generated using ChemDraw® software version 18.1.0.458 or Collaborative Drug Discovery Inc. (CDD) CDD Vault update #3.

### Compositions

Provided herein are pharmaceutical compositions comprising one or more compounds of formula (A), or any variation or embodiment thereof, as described elsewhere herein, or a stereoisomer or tautomer thereof, or a pharmaceutically acceptable salt of any of the foregoing. In some embodiments, provided herein is a pharmaceutical composition comprising (i) of formula (A), or any variation or embodiment thereof, as described elsewhere herein, or a stereoisomer or tautomer thereof, or a pharmaceutically acceptable salt of any of the foregoing, and (ii) one or more pharmaceutically acceptable excipients.

Suitable pharmaceutically acceptable excipients may include, for example, fillers, diluents, sterile aqueous solutions and various organic solvents, permeation enhancers, solubilizers, and adjuvants. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Examples of suitable excipients are well-known to those skilled in the art. Such compositions are prepared in a manner well known in the pharmaceutical art. See, e.g., Remington's Pharmaceutical Sciences, Academic Press, 23^{rd} ed. (2020), which is incorporated herein by reference.

The pharmaceutical compositions may be administered in either single or multiple doses. The pharmaceutical composition may be administered by various methods including, for example, oral, rectal, buccal, intranasal, and transdermal routes. In certain embodiments, the pharmaceutical composition may be administered by intra-arterial injection, intravenously, intraperitoneally, parenterally, intramuscularly, subcutaneously, orally, topically, or as an inhalant.

Compounds as described herein may be administered to individuals in a form of generally accepted oral compositions, such as tablets, coated tablets, gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical formulations may contain preservatives, solubilizers, stabilizers, re-wetting agents, emulgators, sweeteners, dyes, adjusters, salts for the adjustment of osmotic pressure, buffers, coating agents or antioxidants.

The specific dose level of a compound as described herein will depend upon a variety of factors such as the age, body weight and sex of the individual as well as the route of administration and other factors. In some embodiments, a dosage is expressed as a number of milligrams of a compound described herein per kilogram of the individual's body weight (mg/kg). Dosages of between about 0.1 mg/kg and 100-150 mg/kg may be appropriate.

The compound may be administered to an individual in accordance with an effective dosing regimen for a desired period of time or duration, such as at least about one month, at least about 2 months, at least about 3 months, at least about 6 months, or at least about 12 months or longer, which in some variations may be for the duration of the individual's life.

### Methods of Treatment

Provided herein is a method of modulating APOL1 in a cell, comprising exposing the cell to an effective amount of a compound of formula (A), or any variation or embodiment thereof, as described elsewhere herein, or a stereoisomer or tautomer thereof, or a pharmaceutically acceptable salt of any of the foregoing. Also provided herein is a method of modulating APOL1 in a cell, comprising exposing the cell to a composition comprising an effective amount of a compound of formula (A), or any variation or embodiment thereof, as described elsewhere herein, or a stereoisomer or tautomer thereof, or a pharmaceutically acceptable salt of any of the foregoing, and one or more pharmaceutically acceptable excipients. Isotopically labeled forms of any of the foregoing are also embraced, including, but not limited to, deuterated or tritiated forms (wherein at least one hydrogen is replaced by at least one deuterium or tritium) of any of the specific compounds detailed herein.

Provided herein is a method of inhibiting APOL1 in a cell, comprising exposing the cell to an effective amount of a compound of formula (A), or any variation or embodiment thereof, as described elsewhere herein, or a stereoisomer or tautomer thereof, or a pharmaceutically acceptable salt of any of the foregoing. Also provided herein is a method of inhibiting APOL1 in a cell, comprising exposing the cell to a pharmaceutical composition comprising an effective amount of a compound of formula (A), or any variation or embodiment thereof, as described elsewhere herein, or a stereoisomer or tautomer thereof, or a pharmaceutically acceptable salt of any of the foregoing, and one or more pharmaceutically acceptable excipients.

Provided herein is a method of inhibiting APOL1 in an individual, comprising administering to the individual an effective amount of a compound of formula (A), or any variation or embodiment thereof, as described elsewhere herein, or a stereoisomer or tautomer thereof, or a pharmaceutically acceptable salt of any of the foregoing. Also provided herein is a method of inhibiting APOL1 in an individual, comprising administering to the individual a pharmaceutical composition comprising an effective amount of a compound of formula (A), or any variation or embodiment thereof, as described elsewhere herein, or a stereoisomer or tautomer thereof, or a pharmaceutically acceptable salt of any of the foregoing, and one or more pharmaceutically acceptable excipients.

In some embodiments, the compounds provided herein inhibit APOL1 at a concentration of less than 10 μM, less than 1 μM, less than 0.5 μM, or less than 0.1 μM. In some embodiments, the compounds provided herein inhibit APOL1 at a concentration of 1 to 10 μM, 0.01 to 1 μM, or 0.01 to 10 μM.

In some embodiments, the compounds provided herein reduce cell death caused by overexpression of APOL1. In some embodiments, the compounds provided herein reduce cell death caused by overexpression APOL1 at a concentration of less than 10 μM, less than 1 μM, less than 0.5 μM, or less than 0.1 μM. In some embodiments, the compounds provided herein reduce cell death caused by APOL1 overexpression at a concentration of 1 to 10 μM, 0.01 to 1 μM, or 0.01 to 10 μM.

In some embodiments, compounds provided herein have an EC₅₀ of less than 1 μM, less than 0.5 μM, or less than 0.1 μM. In some embodiments, the compounds provided herein have an EC₅₀ of 1 to 10 μM, 0.01 to 1 μM, or 0.01 to 10 μM.

In some embodiments, compounds provided herein have an AC₅₀ of less than 1 μM, less than 0.5 μM, or less than 0.1 μM. In some embodiments, the compounds provided herein have an AC₅₀ of 1 to 10 μM, 0.01 to 1 μM, or 0.01 to 10 μM. In some embodiments, the AC₅₀ value reflects the compound's ability to prevent calcium influx by inhibiting APOL1.

In some embodiments, the compounds provided herein inhibit a cation channel. In some embodiments, the compounds of the present disclosure inhibit a calcium channel. In some embodiments, the compounds of the present disclosure reduce calcium transport.

Provided herein is a method of treating an APOL1-mediated disease, disorder, or condition in an individual in need thereof, comprising administering to the individual a a compound of formula (A), or any variation or embodiment thereof, as described elsewhere herein, or a stereoisomer or tautomer thereof, or a pharmaceutically acceptable salt of any of the foregoing. Also provided herein is a method of treating an APOL1-mediated disease, disorder, or condition in an individual in need thereof, comprising administering to the individual a pharmaceutical composition comprising a compound of formula (A), or any variation or embodiment thereof, as described elsewhere herein, or a stereoisomer or tautomer thereof, or a pharmaceutically acceptable salt of any of the foregoing, and one or more pharmaceutically acceptable excipients.

Provided herein is a method of treating an APOL1-mediated disease, disorder, or condition in an individual in need thereof, comprising administering to the individual a therapeutically effective amount of a compound of formula (A), or any variation or embodiment thereof, as described elsewhere herein, or a stereoisomer or tautomer thereof, or a pharmaceutically acceptable salt of any of the foregoing. Also provided herein is a method of treating an APOL1-mediated disease, disorder, or condition in an individual in need thereof, comprising administering to the individual a pharmaceutical composition comprising a therapeutically effective amount of a compound of formula (A), or any variation or embodiment thereof, as described elsewhere herein, or a stereoisomer or tautomer thereof, or a pharmaceutically acceptable salt of any of the foregoing, and one or more pharmaceutically acceptable excipients.

Provided herein is a method of treating a kidney disease, disorder, or condition in an individual in need thereof, comprising administering to the individual a therapeutically effective amount of a compound of formula (A), or any variation or embodiment thereof, as described elsewhere herein, or a stereoisomer or tautomer thereof, or a pharmaceutically acceptable salt of any of the foregoing. Also provided herein is a method of treating a kidney disease, disorder, or condition in an individual in need thereof, comprising administering to the individual a pharmaceutical composition comprising a therapeutically effective amount of a compound of formula (A), or any variation or embodiment thereof, as described elsewhere herein, or a stereoisomer or tautomer thereof, or a pharmaceutically acceptable salt of any of the foregoing, and one or more pharmaceutically acceptable excipients.

In some embodiments, the individual has a chronic kidney disease. In some embodiments, the individual has hypertension-attributed kidney disease. In some embodiments, the kidney disease, disorder, or condition is an APOL1-mediated kidney disease, disorder, or condition. In some embodiments, the kidney disease, disorder, or condition is selected from the group consisting of focal segmental glomerulosclerosis (FSGS), hypertension-attributed kidney disease, viral nephropathy, COVID-19 associated nephropathy, human immunodeficiency virus-associated nephropathy (HIVAN), sickle-cell nephropathy, lupus nephritis, and diabetic kidney disease.

Also provided herein is a method of treating an APOL1-mediated disorder, such as preeclampsia and sepsis, comprising administering to an individual in need thereof a therapeutically effective amount of a compound of formula (A), or any variation or embodiment thereof, as described elsewhere herein, or a stereoisomer or tautomer thereof, or a pharmaceutically acceptable salt of any of the foregoing. In some embodiments, the individual is genetically predisposed to developing the APOL1-mediated disorder.

Also provided herein is a method of delaying development of progressive renal allograft loss in a kidney transplant recipient comprising administering to the kidney transplant recipient a therapeutically effective amount of a compound of formula (A), or any variation or embodiment thereof, as described elsewhere herein, or a stereoisomer or tautomer thereof, or a pharmaceutically acceptable salt of any of the foregoing. In some embodiments, the kidney transplant recipient receives a kidney from a high-risk APOL1 genotype donor. In some embodiments, the kidney transplant recipient is administered a therapeutically effective amount of the compound for a period of time before receiving the kidney transplant. In some embodiments, the kidney transplant recipient is administered a therapeutically effective amount of the compound subsequent to receiving the kidney transplant.

Provided herein is a method of treating a kidney disease, disorder, or condition in an individual in need thereof, comprising administering to the individual a therapeutically effective amount of a compound of formula (A), or any variation or embodiment thereof, as described elsewhere herein, or a stereoisomer or tautomer thereof, or a pharmaceutically acceptable salt of any of the foregoing, wherein the individual has an APOL1 mutation. Also provided herein is a method of treating a kidney disease, disorder, or condition in an individual in need thereof, comprising administering to the individual a pharmaceutical composition comprising a therapeutically effective amount of a compound of formula (A), or any variation or embodiment thereof, as described elsewhere herein, or a stereoisomer or tautomer thereof, or a pharmaceutically acceptable salt of any of the foregoing, and one or more pharmaceutically acceptable excipients, wherein the individual has an APOL1 mutation.

The compounds provided herein may also be used in a method of delaying the development of an APOL1-mediated disease, disorder, or condition, comprising administering a compound of formula (A), or any variation or embodiment thereof, as described elsewhere herein, or a stereoisomer or tautomer thereof, or a pharmaceutically acceptable salt of any of the foregoing, to an individual who is at risk of developing an APOL1-mediated disease, disorder, or condition. In some embodiments, the APOL1-mediated disease, disorder, or condition is preeclampsia or sepsis and the individual has two APOL1 risk alleles. In some embodiments, the APOL1-mediated disease, disorder, or condition is a chronic kidney disease and the individual has any binary combination of G1 and G2 APOL1 risk alleles. In some embodiments, the chronic kidney disease is focal segmental glomerulosclerosis (FSGS), hypertension-attributed kidney disease, human immunodeficiency virus-associated nephropathy (HIVAN), hypertension-attributed kidney disease, sickle cell nephropathy, viral nephropathy, COVID-19 associated nephropathy, lupus nephritis, diabetic kidney disease, or APOL1-associated nephropathy. The compounds as provided herein may also be used in a method of delaying the development of progressive renal allograft loss in an individual who has received a kidney transplantation from a high-risk APOL1 genotype donor.

In some embodiments, the individual has a gain-of-function mutation in APOL1. In some embodiments, the individual has an APOL1 risk allele. In some embodiments, the APOL1 risk allele is a missense variant. In some embodiments, the APOL1 risk allele is a G1 variant. In some embodiments, the G1 variant is G1^{G} (p.S342 G) or G1^{M} (p.I384 M). In some embodiments, the APOL1 risk allele is the G2 variant. In some embodiments, the G2 variant is NYK388-389K. In some embodiments, the APOL1 risk variant is a mutation in the serum resistance-associated (SRA) binding domain of the APOL1 protein.

Also provided herein is a method of inhibiting APOL1 in an individual comprising administering to the individual a therapeutically effective amount of a compound of formula (A), or any variation or embodiment thereof, as described elsewhere herein, or a stereoisomer or tautomer thereof, or a pharmaceutically acceptable salt of any of the foregoing.

Also provided herein is method of preventing kidney failure in an individual comprising administering a therapeutically effective amount of a compound of Formula (A), or any variation or embodiment thereof, as described elsewhere herein, or a stereoisomer or tautomer thereof, or a pharmaceutically acceptable salt of any of the foregoing to the individual. In some embodiments, the compound prevents tissue necrosis. In some embodiments, the compound prevents apoptosis. In some embodiments, the compound reduces inflammation.

In some embodiments, the compounds provided herein reduce or eliminate one or more symptoms of a kidney disease. In some embodiments, the compounds reduce nausea, vomiting, loss of appetite, fatigue and weakness, sleep problems, urinary frequency issues, muscle twinges and cramps, swelling, itching, chest pain, shortness of breath, and/or high blood pressure.

In some embodiments, the compounds provided herein reduce the rate of kidney damage and/or progression of kidney damage. In some embodiments, the compounds provided herein reduce the rate of kidney failure. In some embodiments, the compounds provided herein reverse kidney damage. In some embodiments, the compounds reduce the need for dialysis. In some embodiments, the compounds provided herein delay the need for dialysis at least one month, at least two months, at least three months, or at least one year.

In some embodiments, the compounds reduce the rate of or delay the need for a kidney transplant. For example, in some embodiments, the compounds provided herein delay the need for a kidney transplant at least one month, at least two months, at least three months, at least six months, or at least one year. In some embodiments, the compounds provided herein eliminate the need for a kidney transplant.

In some embodiments, the individual has stage 1, stage 2, stage 3A, stage 3B, stage 4, or stage 5 chronic kidney disease. In some embodiments, kidney function is evaluated using an estimated glomerular filtration rate (eGFR) kidney function test.

In some embodiments, the administration is oral administration.

### Kits

The present disclosure further provides kits for carrying out the methods of the invention. The kits may comprise a compound or pharmaceutically acceptable salt thereof as described herein and suitable packaging. The kits may comprise one or more containers comprising any compound described herein. In one aspect, a kit includes a compound of the disclosure or a pharmaceutically acceptable salt thereof, and a label and/or instructions for use of the compound in the treatment of a disease or disorder described herein. The kits may comprise a unit dosage form of the compound.

Provided herein are kits, comprising (i) a compound of formula (A), or any variation or embodiment thereof, as described elsewhere herein, or a stereoisomer or tautomer thereof, or a pharmaceutically acceptable salt of any of the foregoing, and (ii) instructions for use in treating an APOL1-mediated disease, disorder, or condition in an individual in need thereof. Also provided herein are kits, comprising (i) a pharmaceutical composition comprising a compound of formula (A), or any variation or embodiment thereof, as described elsewhere herein, or a stereoisomer or tautomer thereof, or a pharmaceutically acceptable salt of any of the foregoing, and one or more pharmaceutically acceptable excipients; and (ii) instructions for use in treating an APOL1-mediated disease, disorder, or condition in an individual in need thereof. Articles of manufacture are also provided, wherein the article of manufacture comprises a compound of formula (A), or any variation or embodiment thereof, as described elsewhere herein, or a stereoisomer or tautomer thereof, or a pharmaceutically acceptable salt of any of the foregoing, in a suitable container. Also provided herein are articles of manufacture, comprising a pharmaceutical composition comprising a compound of formula (A), or any variation or embodiment thereof, as described elsewhere herein, or a stereoisomer or tautomer thereof, or a pharmaceutically acceptable salt of any of the foregoing, in a suitable container. The container may be a vial, jar, ampoule, preloaded syringe, or intravenous bag.

### Methods of Assaying APOL1 Activity

Provided herein is a method of assessing APOL1 inhibition in a cell, comprising inducing APOL1 expression in a cell, contacting the cell with an APOL1 inhibitor, and measuring inhibition of calcium transport. In some embodiments, inducing APOL1 expression comprises contacting the cell with doxycycline. In some embodiments, the cell stably expresses a genetically encoded calcium indicator. In some embodiments, the genetically encoded calcium indicator comprises GCaMP6f. In some embodiments, the cell inducibly expresses APOL1 G2. In some embodiments, the cell stably expresses a genetically encoded calcium indicator and inducibly expresses APOL1 G2. In some embodiments, the APOL1 inhibitor is a compound of formula (A), or any variation or embodiment thereof, as described elsewhere herein, or a stereoisomer or tautomer thereof, or a pharmaceutically acceptable salt of any of the foregoing

Provided herein is a method of assessing rescue of HEK cell death caused by overexpression of APOL1, inducing APOL1 expression in a cell, contacting the cell with an APOL1 inhibitor, exposing the cell to a luminescence reagent, and measuring luminescence. In some embodiments, inducing APOL1 expression comprises contacting the cell with doxycycline. In some embodiments, the cell overexpresses APOL1G2. In some embodiments, the APOL1 inhibitor is a compound of formula (A), or any variation or embodiment thereof, as described elsewhere herein, or a stereoisomer or tautomer thereof, or a pharmaceutically acceptable salt of any of the foregoing.

### Methods of Preparing

The present disclosure further provides methods for preparing the compounds of present invention. In some aspect, provided herein are methods of preparing a compound of formula (A), or any embodiment or variation thereof, such as a compound of formula (I), (II), (III), (B), (B-2), (B-4), (C), (C-1), or (D), or a stereoisomer or tautomer thereof, or a pharmaceutically acceptable salt of any of the foregoing.

In some embodiments, a method for preparing a compound of formula (A), or a stereoisomer or tautomer thereof, or a pharmaceutically acceptable salt of any of the foregoing, comprises a step of reacting a compound of formula (A-I1):

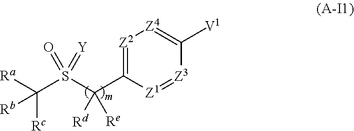

wherein, R^{a}, R^{b}, R^{c}, R^{d}, R^{e}, Z¹, Z², Z³, Z⁴, Y, and m, are as defined for a compound of formula (A); and
V¹ is selected from the group consisting of:

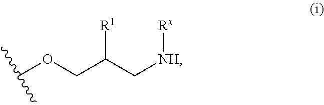

wherein
R^{x} and R¹ are taken together with the atoms to which they are attached to form a 3-8 membered heterocycle, wherein the 3-8 membered heterocycle is substituted with n independently selected R^{m} substituents, wherein n is an integer from 0-6, and R^{m} is C₁₋₆alkyl, or C(O)C₁₋₆alkyl, wherein the C₁₋₆alkyl is optionally substituted with one or more Rⁿ substituents, and Rⁿ is, independently at each occurrence, —OH;

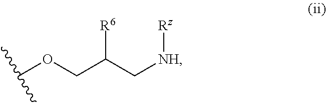

wherein
R^{z} and R⁶ are taken together with the atoms to which they are attached to form a 3-8 membered heterocycleheterocycle substituted with n independently selected R^{m} substituents, wherein n is an integer from 0-6, and R^{m} is C₁₋₆alkyl or C(O)C₁₋₆alkyl, wherein the C₁₋₆ alkyl is optionally substituted with one or more Rⁿ substituents, and Rⁿ is, independently at each occurrence, —OH;
with:
a compound of formula (A-I2):

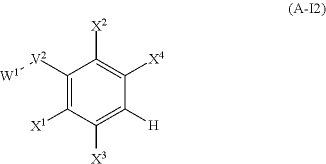

wherein X¹, X², X³, and X⁴ are as defined for a compound of formula (A);
the dashed line represents a single or double bond;
W¹ is oxo, halo or sulfonate ester; and
V² is selected from the group consisting of:

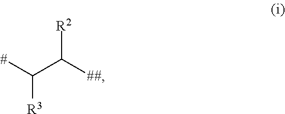

wherein
R² is H, —OH, or C₁₋₆alkyl, wherein the C₁₋₆alkyl of R² is optionally substituted with one or more OH; and
R³ is H or C₁₋₆alkyl, wherein the C₁₋₆alkyl of R³ is optionally substituted with one or more OH;
provided that when V² is (i), either:
(1) m is 1,
(2) at least one of Z¹, Z², Z³, and Z⁴ is —N— or —C(R^{f})—,
(3) R³ is other than H,
(4) at least one of R^{a}, R^{b}, and R^{c} is heterocycle, or
(5) at least one of R^{g}, Rⁱ, R^{j}, R^{k}, and Rⁿ is present; and

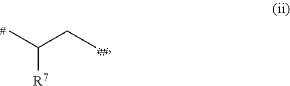

wherein
R⁷ is taken, together with one of X¹ and X² and the atoms to which they are attached, to form a C₄₋₈cycloalkyl;
wherein # denotes the point of attachment to W¹ and ## denotes the point of attachment to the remainder of the molecule;
to give a compound of formula (A) or a stereoisomer or tautomer thereof, or a pharmaceutically acceptable salt of any of the foregoing.

In some embodiments, the compound of formula (A) is prepared by a step comprising:
a) alkylation of an amine of formula (A-I1) with an alkyl halide, or sulfonate ester compound of formula (A-I2) in the presence of an inorganic or organic base; or
b) reductive amination of an aldehyde or ketone of formula (A-I2) with an amine of formula (A-I1) in the presence of a reducing agent.

In some embodiments, the compound of formula (A) is prepared by a step comprising alkylation of an amine of formula (A-I1) with an alkyl halide, or sulfonate ester compound of formula (A-I2) in the presence of an inorganic or organic base. In some embodiments, the inorganic base is selected from the group consisting of potassium carbonate, sodium carbonate, and sodium bicarbonate. In some embodiments, the organic base is a tertiary amine. In some embodiments, the organic base is selected from the group consisting of trimethylamine, triethylamine, and diisopropylethyamine.

In some embodiments, the sulfonate ester compound of formula (A-12) is a mesylate or a tosylate. In some embodiments, the sulfonate ester compound of formula (A-12) is a mesylate. In some embodiments, the sulfonate ester compound of formula (A-I2) is a tosylate.

In some embodiments, the compound of formula (A) is prepared by a step comprising reductive amination of an aldehyde or ketone of formula (A-I2) with an amine of formula (I-I1). In some embodiments, the reductive amination proceeds under the action of a reducing agent. In some embodiments, the reducing agent is sodium triacetoxyborohydride, or sodium cyanoborohidride.

In some embodiments, a method for preparing a compound of formula (A), or a stereoisomer or tautomer thereof, or a pharmaceutically acceptable salt of any of the foregoing, comprises a step of reacting a compound of formula (A-I3):

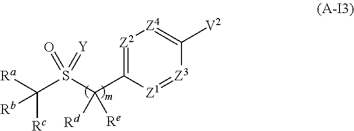

wherein, R^{a}, R^{b}, R^{c}, R^{d}, R^{e}, Z¹, Z², Z³, Z⁴, Y, and m, are as defined for a compound of formula (A); and
V² is halo or OH,
with:
a compound of formula (A-I4):

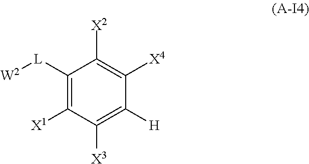

wherein, X¹, X², X³, X⁴, and L are as defined for a compound of formula (A); and
W² is H, or sulfamate;
to give a compound of formula (A) or a stereoisomer or tautomer thereof, or a pharmaceutically acceptable salt of any of the foregoing.

In some embodiments, the compound of formula (A) is prepared by a step comprising alkylation of an alcohol of formula (A-I4) with an alkyl halide compound of formula (A-I3) in the presence of an inorganic or organic base. In some embodiments, the inorganic base is selected from the group consisting of potassium carbonate, sodium carbonate, sodium bicarbonate and cesium carbonate. In some embodiments, the organic base is a tertiary amine. In some embodiments, the organic base is selected from the group consisting of trimethylamine, triethylamine, and diisopropylethyamine.

In some embodiments, the compound of formula (A) is prepared by a step comprising alkylation of an alcohol of formula (A-I3) with a sulfamate compound of formula (A-I4) in the presence of an inorganic or organic base. In some embodiments, the inorganic base is selected from the group consisting of potassium carbonate, sodium carbonate, and sodium bicarbonate. In some embodiments, the organic base is a tertiary amine. In some embodiments, the organic base is selected from the group consisting of trimethylamine, triethylamine and diisopropylethyamine.

In some embodiments, a method for preparing a compound of formula (A), or a stereoisomer or tautomer thereof, or a pharmaceutically acceptable salt of any of the foregoing, comprises a step of reacting a compound of formula (A-I5):

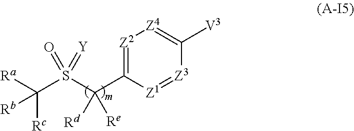

wherein, R^{a}, R^{b}, R^{c}, R^{d}, R^{e}, Z¹, Z², Z³, Z⁴, Y, and m, are as defined for a compound of formula (A); and
V³ is

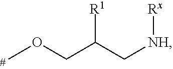

wherein
R^{x} and R¹ are taken together with the atoms to which they are attached to form a 3-8 membered heterocycle, wherein the 3-8 membered heterocycle is substituted with n independently selected R^{m} substituents, wherein n is an integer from 0-6, and R^{m} is C₁₋₆alkyl, or C(O)C₁₋₆alkyl, wherein the C₁₋₆alkyl is optionally substituted with one or more Rⁿ substituents, and Rⁿ is, independently at each occurrence, —OH;
with:
a compound of formula (A-I6):

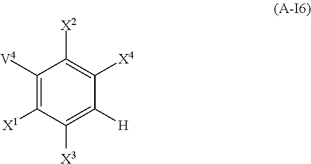

wherein X¹, X², X³, and X⁴ are as defined for a compound of formula (A); and
V⁴ is

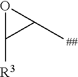

provided that when L is (i), either:
(1) m is 1,
(2) at least one of Z¹, Z², Z³, and Z⁴ is —N— or —C(R^{f})—,
(3) R³ is other than H,
(4) at least one of R^{a}, R^{b}, and R^{c} is heterocycle, or
(5) at least one of R^{g}, Rⁱ, R^{j}, R^{k}, and Rⁿ is present;
to give a compound of formula (A) or a stereoisomer or tautomer thereof, or a pharmaceutically acceptable salt of any of the foregoing.

In some embodiments, the compound of formula (A) is prepared by a step comprising alkylation of an epoxide compound of formula (A-I6) with an amine compound of formula (A-I5) in the presence of an inorganic or organic base. In some embodiments, the inorganic base is selected from the group consisting of potassium carbonate, sodium carbonate, and sodium bicarbonate. In some embodiments, the organic base is a tertiary amine. In some embodiments, the organic base is selected from the group consisting of trimethylamine, triethylamine and diisopropylethyamine.

### EXAMPLES

The following synthetic reaction schemes, which are detailed in the Schemes, General Procedures, and Examples, are merely illustrative of some of the methods by which the compounds of the present disclosure, or an embodiment or aspect thereof, can be synthesized. Various modifications to these synthetic reaction schemes can be made, as will be apparent to those of ordinary skill in the art.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Although certain exemplary embodiments are depicted and described herein, the compounds of the present disclosure, or any variation or embodiment thereof, may be prepared using appropriate starting materials according to the methods described generally herein and/or by methods available to one of ordinary skill in the art.

### SYNTHETIC EXAMPLES

As depicted in the Schemes, General Procedures, and Examples below, in certain exemplary embodiments, compounds of formula (A), or any variation or embodiment thereof, as described elsewhere herein, or a stereoisomer or tautomer thereof, or a pharmaceutically acceptable salt of any of the foregoing, are prepared according to the general procedures. The general methods below, and other methods known to synthetic chemists of ordinary skill in the art, can be applied to all formulae, variations, embodiments, and species described herein.

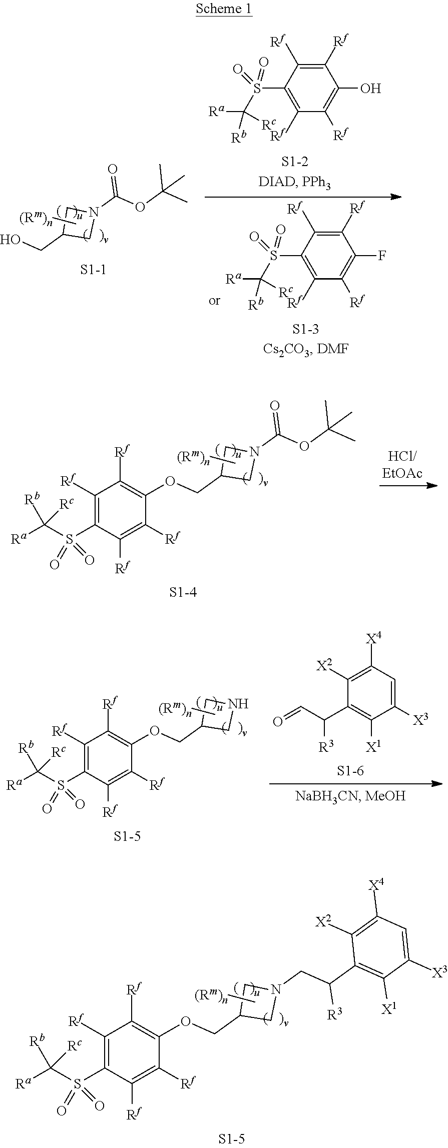

Compounds of formula S1-7 may be prepared by the general synthetic method shown in Scheme 1. It is to be understood that, where applicable, the moieties and variables depicted in Scheme 1 are as defined elsewhere herein for a compound of formula (A), or formula (A), or any variation or embodiment thereof, or a stereoisomer or tautomer thereof, or a pharmaceutically acceptable salt of any of the foregoing. In addition, with reference to Scheme 1, v is an integer from 0-5 and u is an integer from 0-5, provided that 1<(v+u)<6.

C—O bond formation may be accomplished through either a Mitsunobu reaction with phenols of formula S1-2 or an S_{N}Ar with aryl fluorides of formula S1-3 to provide compounds of formula S1-4. Deprotection of the N-tert-butyloxycarbonyl (Boc) group may proceed using a protic acid such as hydrochloric acid to give compounds of formula S1-5. Compounds of formula S1-7 can be prepared through reductive amination using an aldehyde of formula S1-6 and a hydride source such as NaBH₃CN. Chiral preparative SFC or HPLC separation may be utilized to provide two or more single stereoisomers.

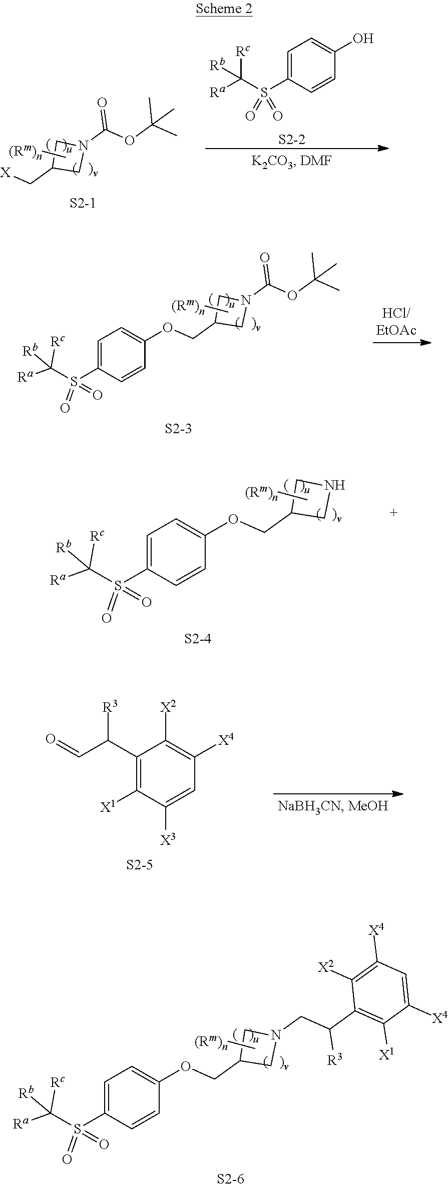

Compounds of formula S2-6 may be prepared by the alternative general synthetic method shown in Scheme 2. It is to be understood that, where applicable, the moieties and variables depicted in Scheme 2 are as defined elsewhere herein for a compound of formula (A), or formula (A), or any variation or embodiment thereof, or a stereoisomer or tautomer thereof, or a pharmaceutically acceptable salt of any of the foregoing. In addition, with reference to Scheme 2, v is an integer from 0-5 and u is an integer from 0-5, provided that 1<(v+u)<6.

C—O bond formation may be accomplished through an S_{N}2 reaction with alkyl halide or alkyl sulfonate ester such as alkyl bromide of formula S2-1 with phenols of formula S2-2 in the presence of bases such as K₂CO₃ to give compounds of formula S2-3. Deprotection of the N-tert-butyloxycarbonyl (Boc) group may proceed using a protic acid such as HCl to give compounds of formula S2-4. Compounds of formula S2-6 can be prepared through reductive amination using an aldehyde of formula S2-5 and a hydride source such as NaBH₃CN. Chiral preparative SFC or HPLC separation may be utilized to provide two or more single stereoisomers.

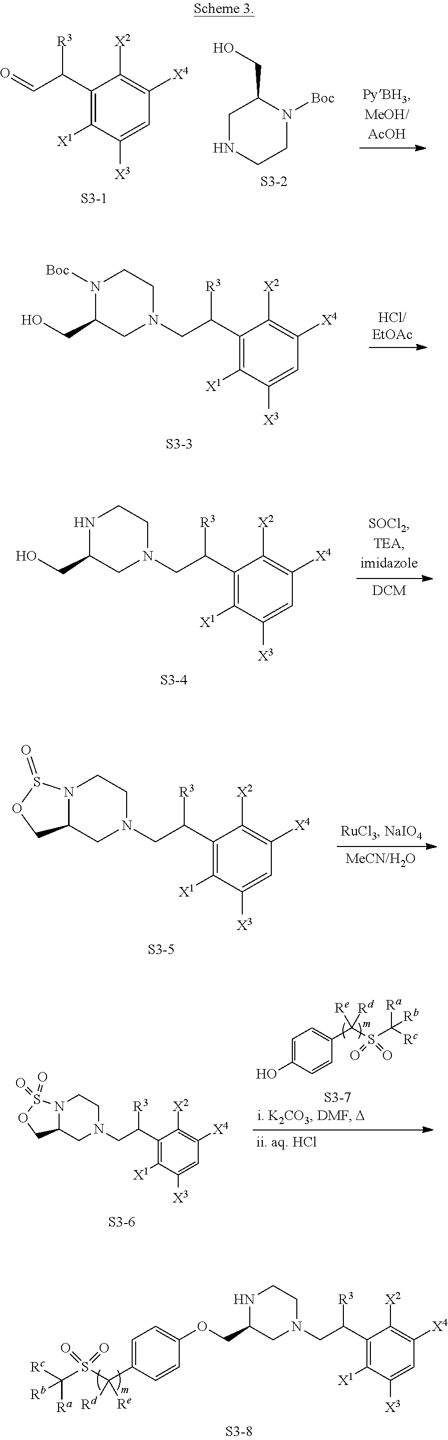

Compounds of formula S3-8 may be prepared according to Scheme 3. Reductive amination of a mono-protected piperazine such as S3-2 with an aldehyde such as S3-1 gives compound S3-3. Removal of the the N-tert-butyloxycarbonyl (Boc) group upon treatment with a protic acid such as HCl in a solvent such as MeOH gives S3-4. Treatment with thionyl chloride, triethylamine, and imidazole gives rise to S3-5. Oxidation to the oxathiazolidine-2,2-dioxide occurs on treatment with ruthenium (III) chloride and sodium periodate in a mixed solvent system of acetonitrile and water to give S3-6. Heating S3-6 with a phenol such as S3-7 and potassium carbonate in DMF, followed by treatment with aqueous HCl, gives compounds of formula S3-8. Chiral preparative SFC or HPLC separation may be utilized to provide two or more single stereoisomers of compounds derived from formula S3-8.

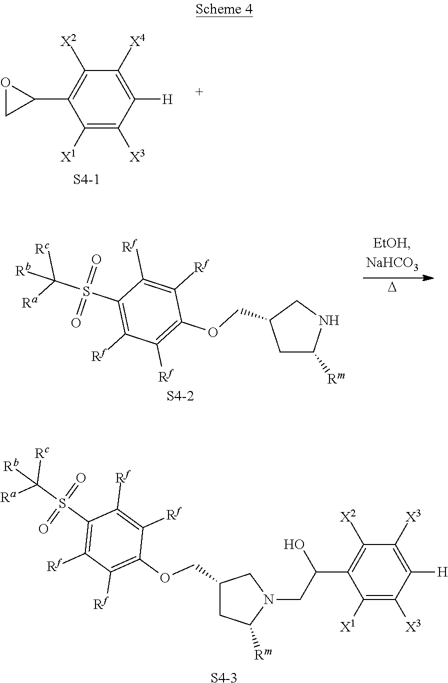

Scheme 4 depicts an approach to compounds of the formula S4-3. Coupling of an epoxide such as S4-1 with an amine such as S4-2 in ethanol solvent using sodium bicarbonate as base gives compounds of formula S4-3. Chiral preparative SFC or HPLC separation may be utilized to provide two or more single stereoisomers of compounds derived from formula S4-3.

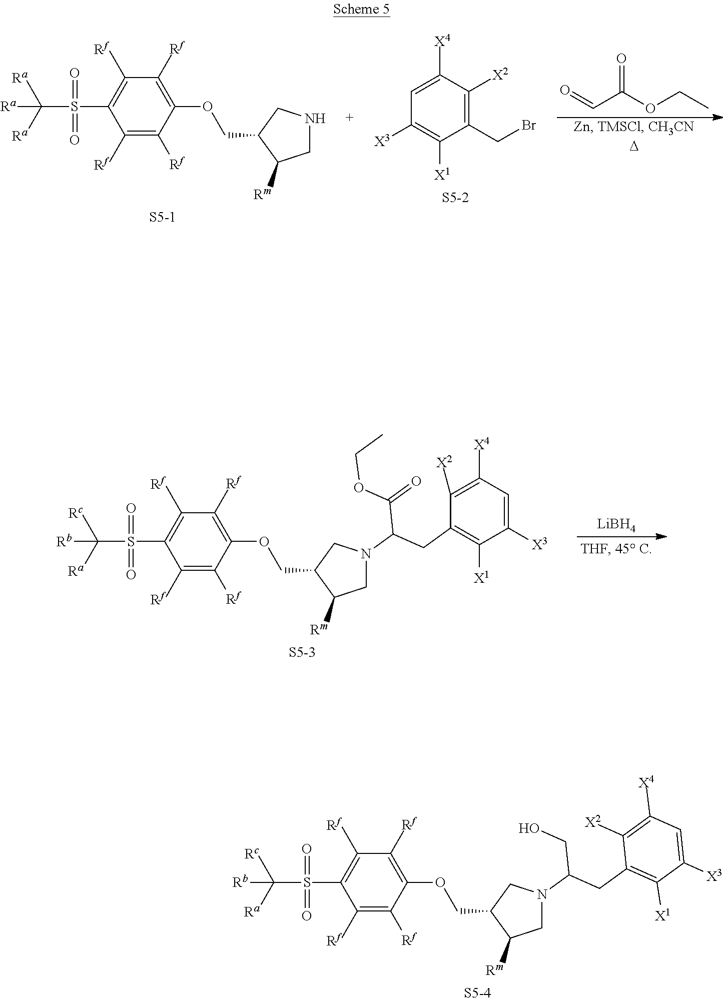

Scheme 5 depicts an approach to compounds of formula S5-4. Reaction of amine S5-1 with ethyl glyoxaldehyde generates an intermediate that can undergo reaction in situ with a benzyl zinc reagent derived from benzyl bromide S5-2. Reduction of ester S5-3 with lithium borohydride gives rise to compounds of formula S5-4. Chiral preparative SFC or HPLC separation may be utilized to provide two or more single stereoisomers of compounds derived from formula S5-4.

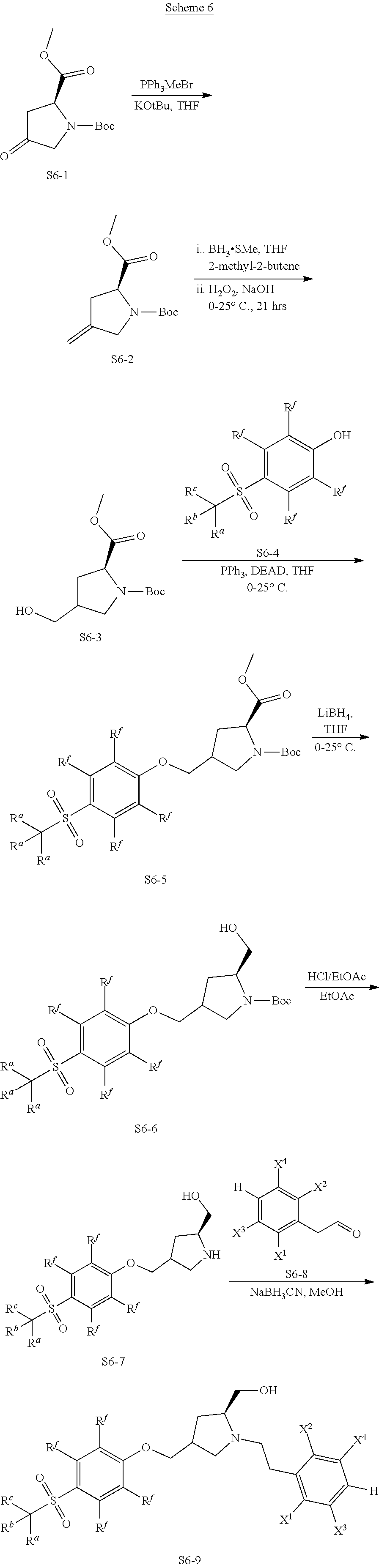

Scheme 6 depicts an approach to compounds of formula S6-9. Reaction of ketone S6-1 with methyl triphenylphosphonium bromide in the presence of a base such as potassium tert-butoxide in an aprotic solvent such as THE gives olefin S6-2. Hydroboration and oxidation then provides a primary alcohol such as S6-3. Mitsunobu reaction of S6-3 with phenol S6-4 generates S6-5. Reduction of S6-5 with lithium borohydride delivers S6-6. N-Boc removal with HCl in ethyl acetate gives S6-7, which can then undergo reductive amination with an aldehyde such as S6-8 to generate compounds of formula S6-9. Chiral preparative SFC or HPLC separation may be utilized to provide two or more single stereoisomers of compounds derived from formula S6-9.

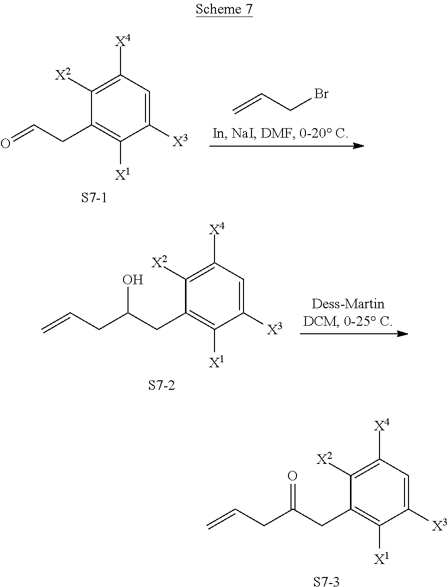

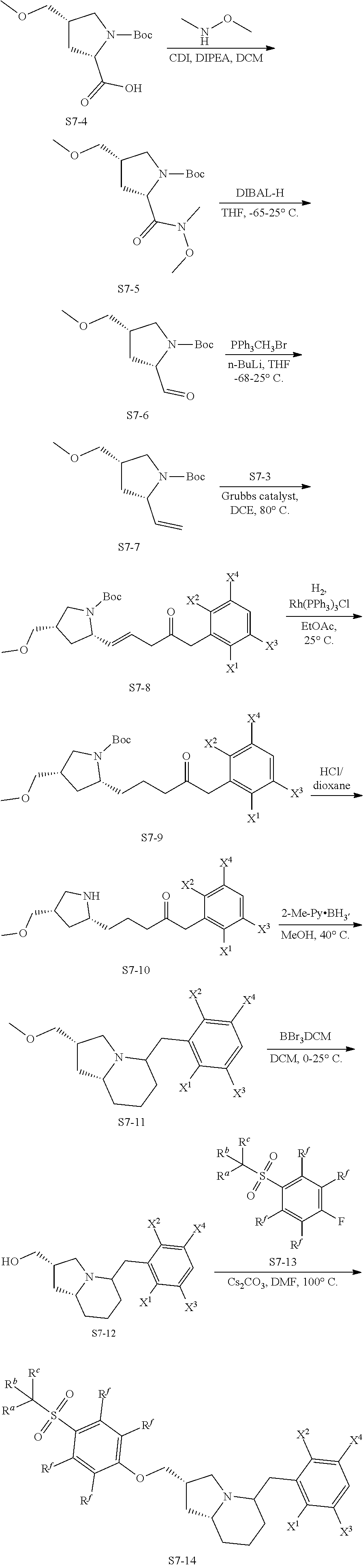

Scheme 7 depicts a synthetic approach to bicyclic amines of formula S7-14. Coupling of an allyl indium reagent generated from allyl bromide, indium metal, and sodium iodide in DMF with an aldehyde of formula S7-1 gives olefin S7-2. Oxidation with Dess-Martin periodinane gives rise to ketone S7-3. Separately, carboxylic acid S7-4 undergoes coupling with N,O-dimethyl hydroxylamine in the presence of CDI and a tertiary amine base such as DIPEA to give amide S7-5. Reduction of amide S7-5 with DIBAL-H generates aldehyde S7-6, which then undergoes a Wittig methylenation with methyl triphenylphosphonium bromide and n-butyllithium as base to give terminal olefin S7-7. Cross metathesis of S7-7 and S7-3 with the Gen II Grubbs catalyst (Benzylidene[1,3-bis(2,4,6-trimethylphenyl)-2-imidazolidinylidene]dichloro(tricyclohexylphosphine)ruthenium) in dicholoroethane gives S7-8. Hydrogenation of S7-8 with Wilkinson's catalyst (Tris(triphenylphosphine)rhodium(I) chloride) gives S7-9, which, after Boc group removal with a protic acid such as HCl in an aprotic solvent such as dioxane to provide amine S7-10, may then undergo intramolecular reductive amination under the action of 2-methylpyridine borane complex to give bicycle S7-11. Cleavage of the methyl ether to give alcohol S7-12 occurs upon reaction with a Lewis acid such as boron tribromide. S_{N}Ar reaction of alcohol S7-12 with a fluorobenzene such as S7-13 gives S7-14. Chiral preparative SFC or HPLC separation may be utilized to provide two or more single stereoisomers of compounds derived from formula S7-14.

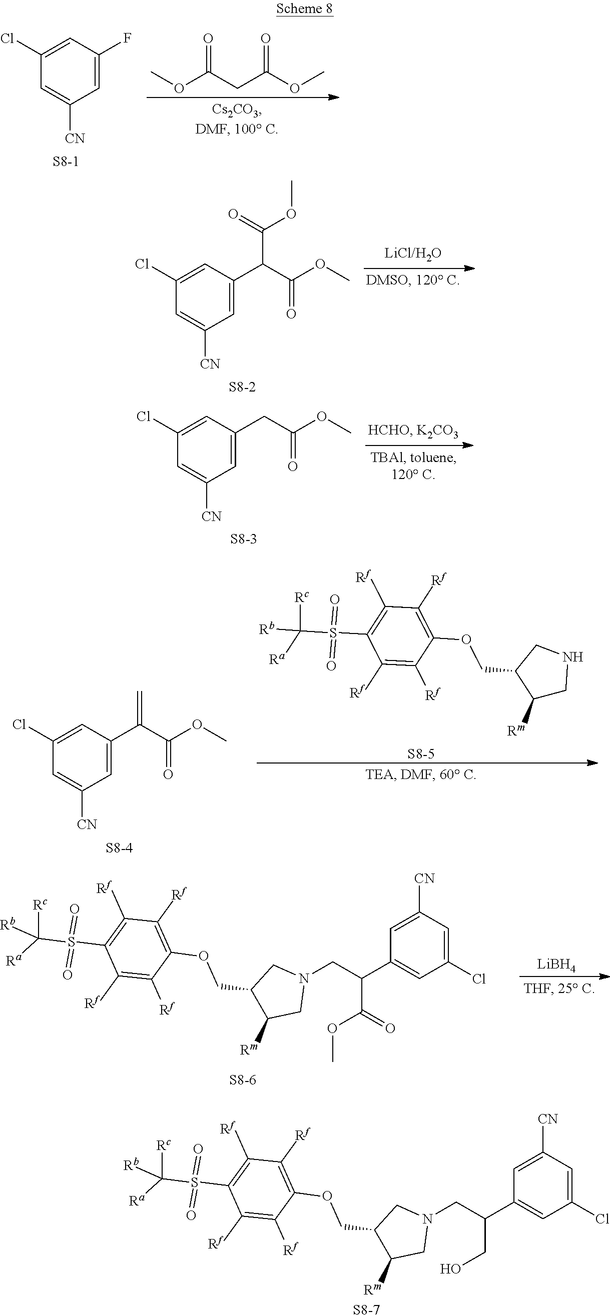

Scheme 8 depicts an approach to compounds of formula S8-7. S_{N}Ar reaction of benzene S8-1 with dimethyl malonate occurs upon heating with a base such as cesium carbonate in DMF. Heating S8-2 and LiCl in DMSO leads to decarbomethoxylation to provide S8-3. An aldol/elimination reaction with paraformaldehyde, potassium carbonate, and tetrabutylammonium iodide gives olefin S8-4. Reaction of S8-4 with amine S8-5 in the presence of a tertiary amine base such as triethylamine produces S8-6. Reduction of S8-6 with lithium borohydride gives S8-7. Chiral preparative SFC or HPLC separation may be utilized to provide two or more single stereoisomers of compounds derived from formula S8-7.

### Intermediate 1

### 2-bromo-5-(methylsulfonyl)pyrazine

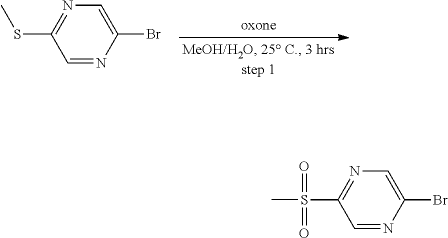

### Step 1: 2-bromo-5-(methylsulfonyl)pyrazine

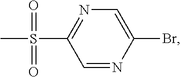

To a solution of 2-bromo-5-methylsulfanyl-pyrazine (1.00 g, 4.88 mmol) in MeOH (40 mL) at 0° C. was added a solution of Oxone (8.99 g, 14.63 mmol) in H₂O (6 mL). The mixture was stirred for 3 h at 25° C. The reaction mixture was filtered and concentrated to remove MeOH. The mixture was diluted with H₂O (20 mL) and extracted with EtOAc (10 mL×3). The combined organic layers were washed with brine (10 mL), dried over Na₂SO₄, filtered and concentrated under reduced pressure to give a residue. The residue was purified by flash silica gel chromatography (ISCO 12 g cartridge, 0-50% Ethyl acetate/Petroleum ether) to give 2-bromo-5-methylsulfonyl-pyrazine. MS=236.9/238.9 [M+H]⁺

### Intermediate 2

### 5-chloro-2-methyl-3-(2-oxoethyl)benzonitrile

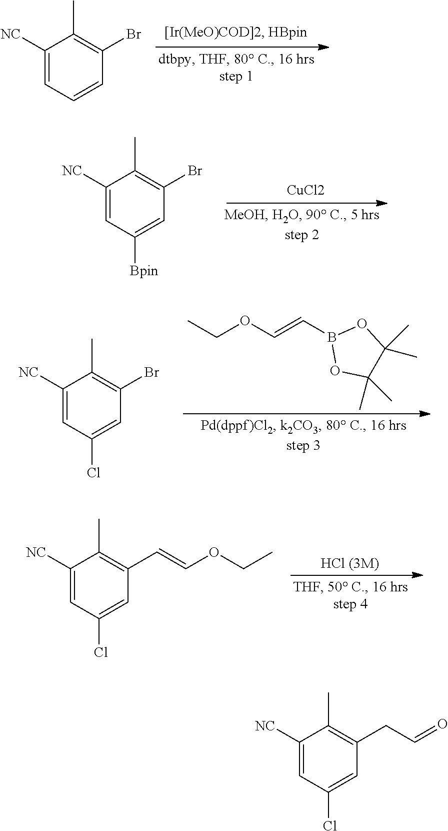

### Step 1: 3-bromo-2-methyl-5-(4,4,5,5-tetramethyl-1,3,2-dioxaborolan-2-yl)benzonitrile

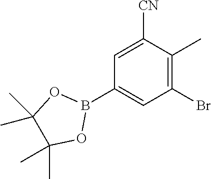

To a solution of 3-bromo-2-methyl-benzonitrile (5.00 g, 25.5 mmol) and 4,4,5,5-tetramethyl-1,3,2-dioxaborolane (32.6 g, 255 mmol) in THF (80 mL) was added 4-tert-butyl-2-(4-tert-butyl-2-pyridyl)pyridine (685 mg, 2.55 mmol) and [Ir(cod)(OMe)]₂ (845 mg, 1.28 mmol) at room temperature under N₂. Then the mixture was stirred at 80° C. for 8 h under N₂. The reaction mixture was diluted with water (100 mL) and extracted with ethyl acetate (100 mL×3). The combined organic layers were washed with brine (100 mL), dried over Na₂SO₄, filtered and concentrated under reduced pressure to give a residue. The residue was purified by flash silica gel chromatography (ISCO 80 g cartridge, 0-20% Ethyl acetate/Petroleum ether) to give 3-bromo-2-methyl-5-(4,4,5,5-tetramethyl-1,3,2-dioxaborolan-2-yl)benzonitrile. ¹H NMR (CDCl₃, 400 MHz) δ 8.15 (s, 1H), 7.98 (s, 1H), 2.65 (s, 3H), 1.35 (s, 12H).

### Step 2: 3-bromo-5-chloro-2-methylbenzonitrile

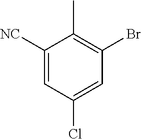

To solution of 3-bromo-2-methyl-5-(4,4,5,5-tetramethyl-1,3,2-dioxaborolan-2-yl)benzonitrile (3.1 g, 9.63 mmol) in MeOH (30 mL) and H₂O (10 mL) was added CuCl₂ (3.88 g, 28.9 mmol) at RT, then the mixture was stirred at 90° C. for 16 h. The reaction mixture was then diluted with water (50 mL) and extracted with ethyl acetate (30 mL×3). The combined organic layers were washed with brine (20 mL), dried over Na₂SO₄, filtered and concentrated under reduced pressure to give a residue. The residue was purified by flash silica gel chromatography (ISCO 20 g cartridge, 0-5% Ethyl acetate/Petroleum ether) to give 3-bromo-5-chloro-2-methyl-benzonitrile. ¹H NMR (CDCl₃, 400 MHz) δ 7.78 (s, 1H), 7.57 (s, 1H), 2.61 (s, 3H).

### Step 3: (E)-5-chloro-3-(2-ethoxyvinyl)-2-methylbenzonitrile

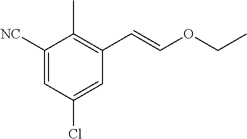

To a solution of 3-bromo-5-chloro-2-methyl-benzonitrile (300 mg, 1.30 mmol) and 2-[(E)-2-ethoxyvinyl]-4,4,5,5-tetramethyl-1,3,2-dioxaborolane (296 mg, 1.50 mmol) in 1,4-dioxane (5 mL) were added K₂CO₃ (360 mg, 2.60 mmol) and Pd(dppf)Cl₂ (95.2 mg, 130 umol) at room temperature under N₂. Then the mixture was stirred at 80° C. for 16 h. The reaction mixture was allowed to cool to RT, diluted with water (10 mL), and extracted with ethyl acetate (10 mL×3). The combined organic layers were washed with brine (20 mL), dried over Na₂SO₄, filtered and concentrated under reduced pressure to give a residue. The residue was purified by flash silica gel chromatography (ISCO 4 g cartridge, 0-5% Ethyl acetate/Petroleum ether) to give 5-chloro-3-[(E)-2-ethoxyvinyl]-2-methyl-benzonitrile. MS=222.1 [M+H]⁺

### Step 4: 5-chloro-2-methyl-3-(2-oxoethyl)benzonitrile

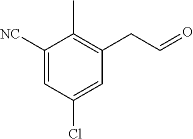

To a solution of 5-chloro-3-[(E)-2-ethoxyvinyl]-2-methyl-benzonitrile (1.5 g, 6.77 mmol) in THE (12 mL) was added aq. HCl (4 M, 12 mL), and then the mixture was stirred at 50° C. for 5 h. The reaction mixture was diluted with water (30 mL) and extracted with ethyl acetate (10 mL×3). The combined organic layers were washed with brine (30 mL), dried over Na₂SO₄, filtered and concentrated under reduced pressure to give a residue. The residue was purified by flash silica gel chromatography (ISCO 12 g cartridge, 0-50% Ethyl acetate/Petroleum ether) to give 5-chloro-2-methyl-3-(2-oxoethyl)benzonitrile. MS=194.0 [M+H]⁺.

### Intermediate 3

### 3-chloro-5-(2-oxopropyl)benzonitrile

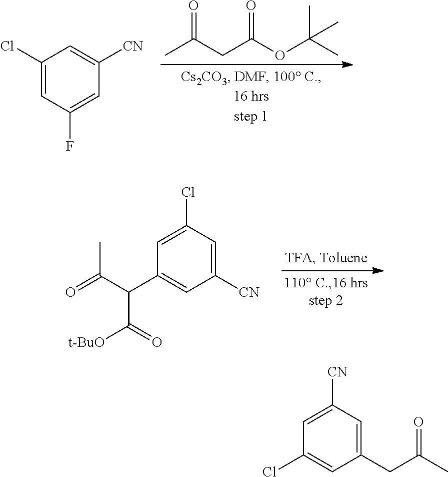

### Step 1: tert-butyl 2-(3-chloro-5-cyanophenyl)-3-oxobutanoate

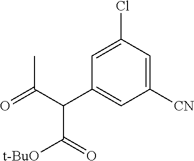 [SMILES: CC(=O)C(C(=O)OC(C)(C)C)c1cc(Cl)cc(C#N)c1]

To a solution of 3-chloro-5-fluoro-benzonitrile (5.00 g, 32.1 mmol) in DMF (50 mL) were added tert-butyl 3-oxobutanoate (7.63 g, 48.2 mmol), Cs₂CO₃ (26.2 g, 80.4 mmol). The mixture was stirred at 100° C. for 16 h. The reaction mixture was allowed to cool to RT, then was poured into water (200 mL) and extracted with ethyl acetate (100 mL×3). The combined organic layers were washed with brine (200 mL), dried over anhydrous Na₂SO₄, filtered and concentrated under reduced pressure to give a residue. The residue was purified by flash silica gel chromatography (ISCO 40 g cartridge, −0-20% Petroleum ether/Ethyl acetate-) to give tert-butyl 2-(3-chloro-5-cyano-phenyl)-3-oxo-butanoate as yellow oil. MS=292.2. [M−H]⁻

### Step 2: 3-chloro-5-(2-oxopropyl)benzonitrile

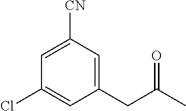

To a mixture of tert-butyl 2-(3-chloro-5-cyano-phenyl)-3-oxo-butanoate (3.00 g, 5.11 mmol, 50% purity) in toluene (30 mL) was added TFA (2.91 g, 25.5 mmol). The mixture was stirred at 110° C. for 16 h. The reaction mixture was allowed to cool to RT, then was concentrated under reduced pressure to give a residue. The crude product was purified by reversed-phase HPLC (Agela C18, 35%-65% H₂O:CH₃CN) to give 3-acetonyl-5-chloro-benzonitrile. MS=192.1 [M−H]⁻

### Intermediate 4

### 7-oxo-5,6,7,8-tetrahydronaphthalene-2-carbonitrile

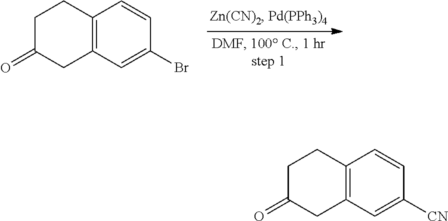 [SMILES: O=C1CCc2ccc(Br)cc2C1 ; N#Cc1ccc2c(c1)CC(=O)CC2]

### Step 1: 7-oxo-5,6,7,8-tetrahydronaphthalene-2-carbonitrile

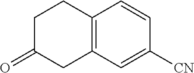 [SMILES: N#Cc1ccc2c(c1)CC(=O)CC2]

To a solution of 7-bromotetralin-2-one (1.5 g, 6.7 mmol) in DMF (20 mL) was added Pd(PPh₃)₄ (770 mg, 666 umol) and Zn(CN)₂ (1.66 g, 14.1 mmol). The mixture was stirred at 100° C. for 1 h in MW under N₂. The mixture was poured into water (50 mL) and extracted with EtOAc (30 mL×3). The combined organic layers were washed with brine (60 mL), dried over Na₂SO₄, filtered and concentrated under reduced pressure to give a residue. The residue was purified by flash silica gel chromatography (ISCO 20 g cartridge, 0-30% Petroleum ether/Ethyl acetate) to give 3-oxotetralin-6-carbonitrile. MS=172.1 [M+H]⁺

### Intermediate 5

### 7-oxo-5,6,7,8-tetrahydronaphthalene-2-carbonitrile

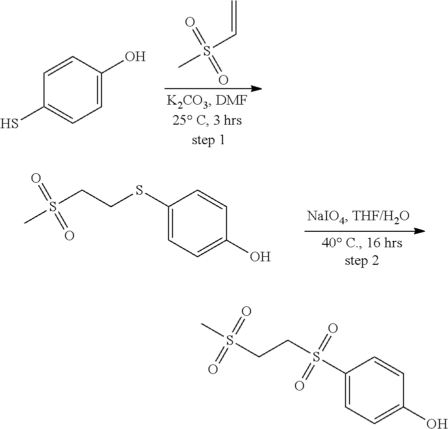

### Step 1: 4-((2-(methylsulfonyl)ethyl)thio)phenol

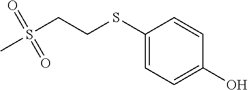

To a solution of 4-sulfanylphenol (4.75 g, 37.7 mmol) and 1-methylsulfonylethylene (4.00 g, 37.7 mmol) in DMF (40 mL) was added K₂CO₃ (7.81 g, 56.5 mmol). The mixture was stirred at room temperature for 3 h. The reaction mixture was diluted with H₂O (100 mL) and then extracted with EtOAc (100 mL×3). The combined organic layers were washed with brine (100 mL), dried over Na₂SO₄, filtered and concentrated under reduced pressure to give a residue. The residue was purified by flash silica gel chromatography (Biotage 40 g cartridge, 0-40% Ethyl acetate/Petroleum ether) to give 4-((2-(methylsulfonyl) ethyl) thio) phenol. MS=231.1 [M−H]⁻

### Step 2: 4-((2-(methylsulfonyl)ethyl)sulfonyl)phenol

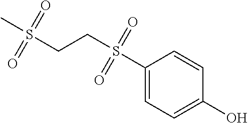 [SMILES: CS(=O)(=O)CCS(=O)(=O)c1ccc(O)cc1]

To a solution of 4-(2-methylsulfonylethylsulfanyl) phenol (3.0 g, 12.9 mmol) in THE (40 mL) at RT was added a mixture of NaIO₄ (5.5 g, 25.8 mmol) in H₂O (10 mL). The mixture was stirred at 40° C. for 16 h. The reaction mixture was diluted with sat. aq. Na₂SO₃ (20 mL), extracted with EtOAc (20 mL×3), dried over Na₂SO₄, filtered and concentrated under reduced pressure to give a residue. The residue was purified by flash silica gel chromatography (ISCO 40 g cartridge, 0-100% Ethyl acetate/Petroleum ether) to give 4-(2-methylsulfonylethylsulfonyl) phenol. MS=263.1 [M−H]⁻

### Intermediate 6

### 6-(methylsulfonyl)pyridin-3-ol

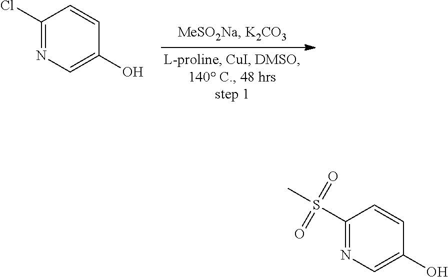

### Step 1: 6-(methylsulfonyl)pyridin-3-ol

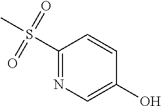

To a solution of 6-chloropyridin-3-ol (3.00 g, 23.2 mmol), MeSO₂Na (9.46 g, 92.6 mmol) in DMSO (30 mL) at RT were added L-proline (799 mg, 6.95 mmol), K₂CO₃ (960 mg, 6.95 mmol) and CuI (1.32 g, 6.95 mmol). The mixture was stirred at 140° C. for 48 h. The reaction mixture was filtered, and the filtrate was diluted with H₂O (50 mL) and extracted with EtOAc (30 mL×3). The combined organic layers were washed with H₂O (30 mL) and brine (30 mL), then dried over Na₂SO₄, filtered and concentrated under reduced pressure to give a residue. The residue was purified by flash silica gel chromatography (ISCO 40 g cartridge, 0-70% Ethyl acetate/Petroleum ether) to give 6-methylsulfonylpyridin-3-ol. MS=172.1 [M−H]⁻

### Intermediate 7

### 3-(oxiran-2-yl)benzonitrile

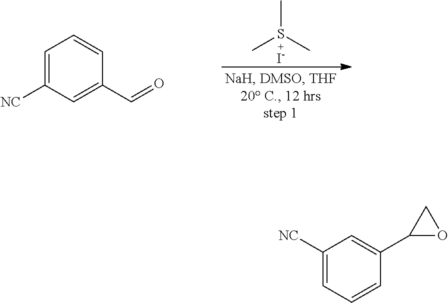

### Step 1: 3-(oxiran-2-yl)benzonitrile

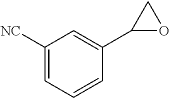 [SMILES: N#Cc1cccc(C2CO2)c1]

To a suspension of NaH (73 mg, 1.83 mmol, 60% by weight in mineral oil) in THE (5 mL) was added a solution of trimethylsulfonium iodide (374 mg, 1.83 mmol) in DMSO (3 mL) under N₂ atmosphere. After 10 min, a solution of 3-formylbenzonitrile (200 mg, 1.53 mmol) in THE (2 mL) was slowly added. The reaction mixture was degassed and purged with N₂ for 3 times, and then the mixture was stirred at room temperature for 12 h under N₂ atmosphere. The reaction mixture was quenched by the addition of H₂O (10 mL) and extracted with EtOAc (10 mL×3). The combined organic layers were washed with brine (10 mL×3), dried over Na₂SO₄, filtered and concentrated under reduced pressure to give a residue. The residue was purified by flash silica gel chromatography (Biotage, 4 g cartridge, 0-50% Ethyl acetate/Petroleum ether) to give 3-(oxiran-2-yl)benzonitrile. MS=146.1 [M+H]⁺

### Intermediate 8

### 3-chloro-5-(3-methyloxiran-2-yl)benzonitrile

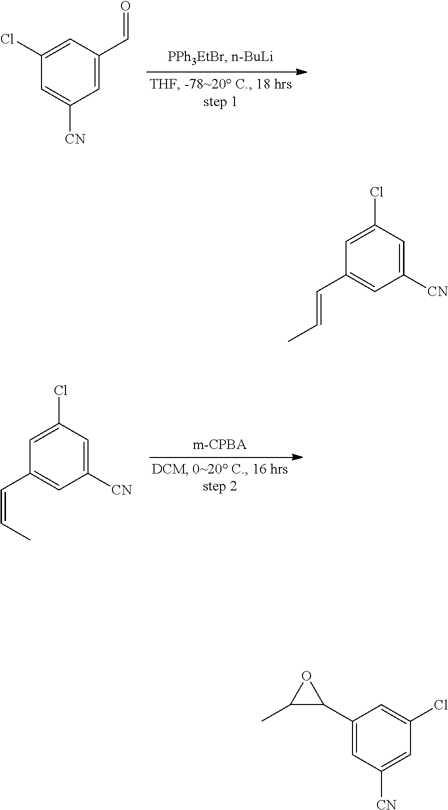

### Step 1: (Z)-3-chloro-5-(prop-1-en-1-yl)benzonitrile and (E)-3-chloro-5-(prop-1-en-1-yl)benzonitrile

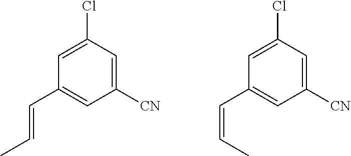

To a solution of bromo-ethyl-triphenyl-λ5-phosphane (16.8 g, 45.3 mmol) in THE (50 mL) at −78° C. was added n-BuLi (2.5M in hexane, 16.9 mL, 42.3 mmol) under N₂. The mixture was then stirred at room temperature for 2 h. The mixture was cooled to −78° C., then a solution of 3-chloro-5-formyl-benzonitrile (5.0 g, 30.2 mmol) in THE (50 mL) was added. The mixture was allowed to warm to room temperature and stirred for 4 h. The reaction mixture was quenched by addition sat. aq. NH₄Cl (100 mL), and then extracted with EtOAc (150 mL×3). The combined organic layers were washed with brine (200 mL), dried over Na₂SO₄, filtered and concentrated under reduced pressure to give a residue. The residue was purified by flash silica gel chromatography (Biotage 40 g cartridge, 0-5% EtOAc:Hexane) to give a mixture of (Z)-3-chloro-5-(prop-i-en-1-yl)benzonitrile and (E)-3-chloro-5-(prop-i-en-1-yl)benzonitrile. MS=178.1 [M+H]⁺

### Step 2: 3-chloro-5-(3-methyloxiran-2-yl)benzonitrile

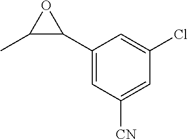

To a mixture of 3-chloro-5-[(E)-prop-i-enyl]benzonitrile and (Z)-3-chloro-5-(prop-i-en-1-yl)benzonitrile (5.0 g, 28.2 mmol) in DCM (100 mL) at 0° C. was added m-CPBA (7.43 g, 36.6 mmol, 85% by weight). The mixture was stirred at room temperature for 16 h. The reaction mixture was cooled 0° C. and quenched by addition sat. aq. Na₂SO₃ (100 mL), then was extracted with DCM (150 mL×3). The combined organic layers were washed with sat. aq. NaHCO₃ (150 mL) and brine (150 mL), dried over Na₂SO₄ filtered and concentrated under reduced pressure to give a residue. The residue was purified by flash silica gel chromatography (Biotage 40 g cartridge, 0-5% Ethyl acetate/Petroleum ether) to give 3-chloro-5-(3-methyloxiran-2-yl)benzonitrile. MS=194.1 [M+H]⁺

### Intermediate 9

### 4-(1-(methylsulfonyl)ethyl)phenol

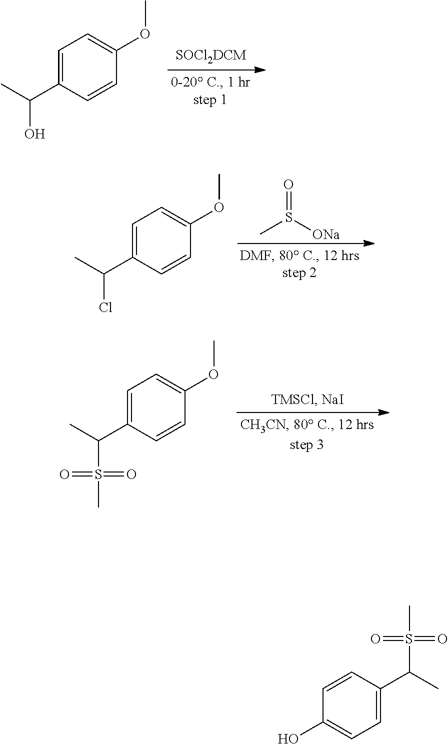

### Step 1: 1-(1-chloroethyl)-4-methoxybenzene

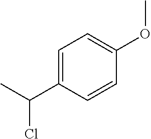

To a solution of SOCl₂ (2.81 g, 23.7 mmol) in dichloromethane (20 mL) at 0° C. was added a solution of 1-(4-methoxyphenyl)ethanol (3.00 g, 19.7 mmol) in dichloromethane (10 mL). The mixture was stirred at RT for 1 h, then was concentrated under reduced pressure to give 1-(1-chloroethyl)-4-methoxy-benzene.

### Step 2: 1-methoxy-4-(1-(methylsulfonyl)ethyl)benzene

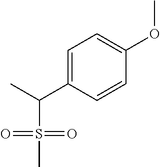

To a solution of methylsulfinyloxysodium (4.01 g, 39.3 mmol) in DMF (30 mL) at RT was added a solution of 1-(1-chloroethyl)-4-methoxy-benzene (3.35 g, 19.6 mmol) in DMF (30 mL). The mixture was stirred at 80° C. for 12 h. The reaction mixture was allowed to cool to RT, then was diluted with H₂O (100 mL) and extracted with ethyl acetate (100 mL×3). The combined organic layers were washed with brine (50 mL), dried over Na₂SO₄, filtered and concentrated under reduced pressure to give a residue. The residue was purified by flash silica gel chromatography (ISCO 40 g cartridge, 0-50% EtOAc/Hexane) to give 1-methoxy-4-(1-methylsulfonylethyl)benzene. MS=215.2 [M+H]⁺.

### Step 3: 4-(1-(methylsulfonyl)ethyl)phenol

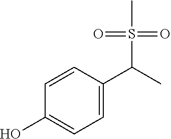

To a solution of 1-methoxy-4-(1-methylsulfonylethyl)benzene (1.1 g, 5.1 mmol) in MeCN (10 mL) at RT were added NaI (3.08 g, 20.5 mmol) and TMSCl (2.23 g, 20.5 mmol). The mixture was stirred at 80° C. for 12 h. The reaction mixture was diluted with H₂O (20 mL) and extracted with ethyl acetate (30 mL×2). The combined organic layers were washed with brine (15 mL), dried over Na₂SO₄, filtered and concentrated under reduced pressure to give a residue. The residue was purified by flash silica gel chromatography (ISCO 40 g cartridge, 0-50% EtOAc/Hexane) to give 4-(1-methylsulfonylethyl)phenol. MS=201.2 [M+H]⁺.

### Intermediate 10

### 4-((3-methoxycyclobutyl)sulfonyl)phenol

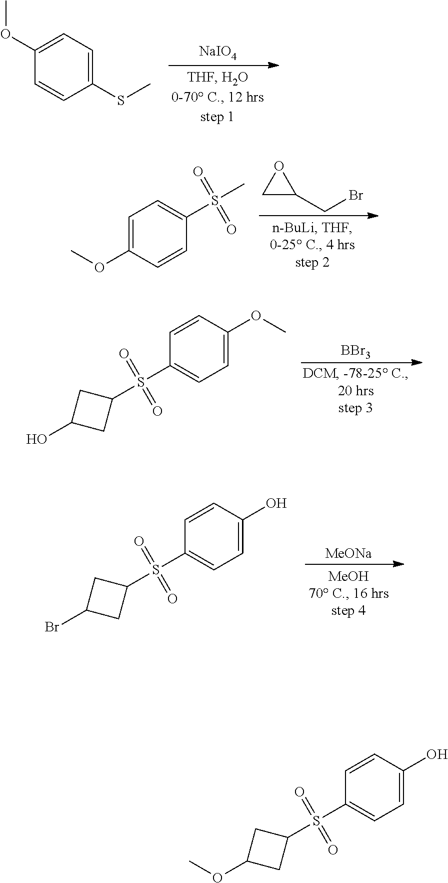

### Step 1:1-methoxy-4-(methylsulfonyl)benzene

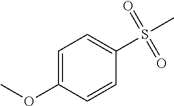

To a solution of 1-methoxy-4-methylsulfanyl-benzene (5.00 g, 32.4 mmol) in THF (30 mL) and H₂O (30 mL) at 0° C. was added NaIO₄ (20.80 g, 97.3 mmol) portionwise. The mixture was then stirred at 70° C. for 12 h. The reaction mixture was allowed to cool to RT and then was filtered. The filtrate was quenched by addition of sat. aq. Na₂SO₃ (40 mL), and then extracted with EtOAc (40 mL×3). The combined organic layers were dried over Na₂SO₄, filtered and concentrated under reduced pressure to give 1-methoxy-4-methylsulfonyl-benzene, which was used without further purification. MS=187.1 [M+H]⁺

### Step 2: 3-((4-methoxyphenyl)sulfonyl)cyclobutanol

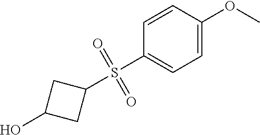 [SMILES: COc1ccc(S(=O)(=O)C2CC(O)C2)cc1]

A solution of 1-methoxy-4-methylsulfonyl-benzene (2.00 g, 10.7 mmol) in THE (50 mL) was degassed and purged with N₂ three times, then cooled to 0° C., n-BuLi (2.5 M in THF, 10.7 mL, 26.8 mmol) was added dropwise. The mixture was stirred at 0° C. for 1 h, then 2-(bromomethyl) oxirane (3.68 g, 26.9 mmol) was added dropwise at 0° C. The mixture was stirred at RT for 3 h. The reaction mixture was quenched by addition of sat. aq. NH₄Cl (20 mL), and extracted with EtOAc (20 mL×3). The combined layers were washed with brine (30 mL), dried over Na₂SO₄, filtered and concentrated under reduced pressure to give a residue. The residue was purified by flash silica gel chromatography (Biotage 20 g cartridge, 0-95% Ethyl acetate/Petroleum ether) to give 3-(4-methoxyphenyl)sulfonylcyclobutanol. MS=243.1 [M+H]⁺

### Step 3: 4-((3-hydroxycyclobutyl)sulfonyl)phenol

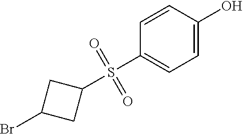 [SMILES: O=S(=O)(c1ccc(O)cc1)C1CC(Br)C1]

To a solution of 3-(4-methoxyphenyl)sulfonylcyclobutanol (0.85 g, 3.5 mmol) in DCM (15 mL) at −78° C. was dropwise added BBr₃ (1.01 mL 10.5 mmol) under N₂. The mixture was stirred at −78° C. for 1 h, then was allowed to warm to RT and stirred for 19 h. The reaction mixture was quenched by addition of MeOH (15 mL) and concentrated under reduced pressure to give a residue. The residue was purified by flash silica gel chromatography (Biotage 12 g cartridge, 0-65% Ethyl acetate/Petroleum ether) to give 4-(3-bromocyclobutyl)sulfonylphenol. MS=291.0/293.0 [M+H]⁺

### Step 4: 4-((3-methoxycyclobutyl)sulfonyl)phenol

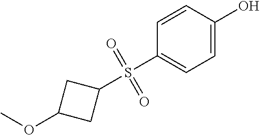 [SMILES: COC1CC(S(=O)(=O)c2ccc(O)cc2)C1]

To a solution of 4-(3-bromocyclobutyl)sulfonylphenol (1.65 g, 5.67 mmol) in MeOH (20 mL) at RT was added sodium methoxide (4.08 g, 323 mmol, 30% purity). The mixture was stirred at 70° C. for 16 h. The reaction mixture was allowed to cool to RT, then was quenched by addition of water (20 mL). The reaction mixture was adjusted to pH=5-6 by addition of aq. HCl (3 M) and extracted with EtOAc (30 mL×2). The combined organic layers were washed with brine (30 mL), dried over Na₂SO₄, filtered and concentrated under reduced pressure to give a residue. The crude product was triturated with (3:1 Petroleum ether:Ethyl acetate, 40 mL) and filtered to give 4-(3-methoxycyclobutyl)sulfonylphenol. MS=243.1 [M+H]⁺

### Intermediate 11

### 4-((2-methoxyethyl)sulfonyl)phenol

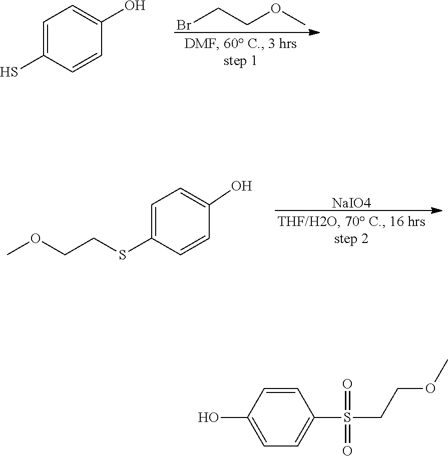

### Step 1: 4-((2-methoxyethyl)thio)phenol

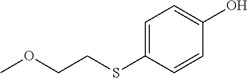

A mixture of 4-sulfanylphenol (1.00 g, 7.93 mmol), 1-bromo-2-methoxy-ethane (1.10 g, 7.93 mmol) and Cs₂CO₃ (2.58 g, 7.93 mmol) in DMF (12 mL) was stirred at 60° C. for 3 h. The reaction mixture was poured into ice water (30 mL) and extracted with EtOAc (30 mL×3). The combined organic layers were washed with brine (50 mL), dried over Na₂SO₄, filtered and concentrated under reduced pressure. The residue was purified by flash silica gel chromatography (Biotage 20 g cartridge, 0-20% EtOAc/Hexane) to give 4-(2-methoxyethylsulfanyl)phenol. MS=183.1 [M−H]⁻

### Step 2: 4-((2-methoxyethyl)sulfonyl)phenol

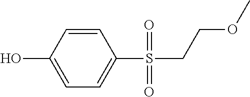

A mixture of 4-(2-methoxyethylsulfanyl)phenol (700 mg, 3.80 mmol) and NaIO₄ (2.44 g, 11.4 mmol) in THE (6 mL) and H₂O (6 mL) was stirred at 70° C. for 16 h. The mixture was filtered, quenched with sat. aq. Na₂S₂O₃ (15 mL) and extracted with EtOAc (20 mL×3). The combined organic layers were washed with brine (40 mL), dried over Na₂SO₄, filtered and the filtrate was concentrated under reduced pressure. The residue was purified by flash silica gel chromatography (Biotage 20 g cartridge, 0-70% EtOAc/Hexane) to give 4-(2-methoxyethylsulfonyl)phenol. MS=217.1 [M+H]⁺

### Intermediate 12

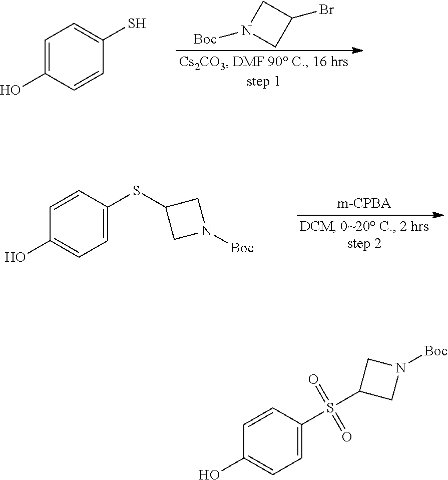 [SMILES: Oc1ccc(S)cc1 ; CC(C)(C)OC(=O)N1CC(Br)C1 ; CC(C)(C)OC(=O)N1CC(Sc2ccc(O)cc2)C1 ; CC(C)(C)OC(=O)N1CC(S(=O)(=O)c2ccc(O)cc2)C1]

### Step 1: tert-butyl 3-((4-hydroxyphenyl)thio)azetidine-1-carboxylate

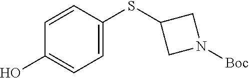 [SMILES: CC(C)(C)OC(=O)N1CC(Sc2ccc(O)cc2)C1]

To a solution of tert-butyl 3-bromoazetidine-1-carboxylate (1.87 g, 7.93 mmol) in DMF (30 mL) at RT were added Cs₂CO₃ (5.16 g, 15.85 mmol) and 4-sulfanylphenol (1.00 g, 7.93 mmol), and the resulting mixture was stirred at 90° C. for 16 h. The reaction mixture was poured into water (30 mL) and extracted with EtOAc (30 mL×3). The combined organic layers were washed with brine (50 mL), dried over Na₂SO₄, filtered and concentrated under reduced pressure to give a residue. The residue was purified by flash silica gel chromatography (Biotage 12 g cartridge, 0-100% Ethyl acetate/Petroleum ether) to give tert-butyl 3-(4-hydroxyphenyl)sulfanylazetidine-1-carboxylate. MS=282.1 [M+H]⁺

### Step 2: tert-butyl 3-((4-hydroxyphenyl)sulfonyl)azetidine-1-carboxylate

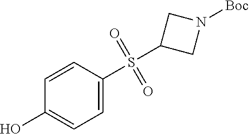 [SMILES: CC(C)(C)OC(=O)N1CC(S(=O)(=O)c2ccc(O)cc2)C1]

To a solution of tert-butyl 3-(4-hydroxyphenyl)sulfanylazetidine-1-carboxylate (50 mg, 178 umol) in DCM (2 mL) at 0° C. was added m-CPBA (46 mg, 267 μmol, 77% purity) at 0° C. The mixture was stirred at RT for 2 h. The reaction mixture was quenched by addition of sat. aq. Na₂SO₃ (5 mL), and then extracted with EtOAc (5 mL×3). The combined organic layers were washed with brine (5 mL×3), dried over Na₂SO₄, filtered and concentrated under reduced pressure to give a residue. The residue was purified by flash silica gel chromatography (Biotage 4 g cartridge, 0-50% EtOAc/Hexane) to give tert-butyl 3-(4-hydroxyphenyl)sulfonylazetidine-1-carboxylate. MS=258.0 [M−C₄H₈+H]⁺.

### Intermediate 13

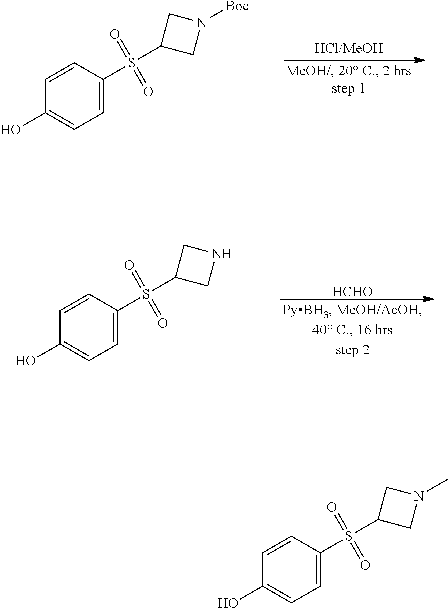 [SMILES: CC(C)(C)OC(=O)N1CC(S(=O)(=O)c2ccc(O)cc2)C1 ; O=S(=O)(c1ccc(O)cc1)C1CNC1 ; "HCHO" ; CN1CC(S(=O)(=O)c2ccc(O)cc2)C1]

### Step 1: 4-(azetidin-3-ylsulfonyl)phenol

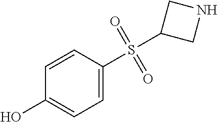 [SMILES: O=S(=O)(c1ccc(O)cc1)C1CNC1]

To a solution of tert-butyl 3-(4-hydroxyphenyl)sulfonylazetidine-1-carboxylate (0.5 g, 1.91 mmol) in MeOH (3 mL) was added HCl in MeOH (4 M, 3 mL). The reaction mixture was stirred at RT for 2 h. The reaction mixture was then concentrated under reduced pressure to give 4-(azetidin-3-ylsulfonyl)phenol HCl salt, which was taken to the next step without further purification. MS=214.1 [M+H]⁺

### Step 2: 4-((1-methylazetidin-3-yl)sulfonyl)phenol

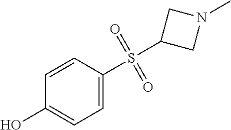

To a solution of 4-(azetidin-3-ylsulfonyl)phenol (400 mg, 1.60 mmol, HCl salt) in MeOH (5 mL) and AcOH (0.05 mL) were added HCHO (120 mg, 4.00 mmol) and borane-2-methylpyridine complex (205 mg, 1.90 mmol). The mixture was stirred at 40° C. for 16 h. The reaction mixture was quenched by addition of sat. aq. NaHCO₃ (10 mL), and then extracted with EtOAc (10 mL×3). The combined organic layers were washed with brine (10 mL), dried over Na₂SO₄, filtered and concentrated under reduced pressure to give a residue. The residue was purified by flash silica gel chromatography (Biotage 12 g cartridge, 0-100% EtOAc/Hexane) to give 4-(1-methylazetidin-3-yl)sulfonylphenol. MS=228.0 [M+H]⁺

### Intermediate 14

### 1-(3-((4-hydroxyphenyl)sulfonyl)azetidin-1-yl)ethan-1-one

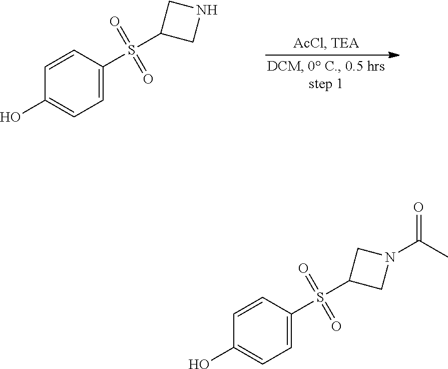 [SMILES: O=S(=O)(c1ccc(O)cc1)C1CNC1 ; CC(=O)N1CC(S(=O)(=O)c2ccc(O)cc2)C1]

### Step 1: 1-(3-((4-hydroxyphenyl)sulfonyl)azetidin-1-yl)ethan-1-one

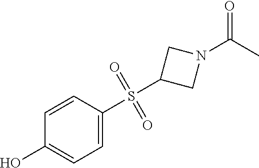 [SMILES: CC(=O)N1CC(S(=O)(=O)c2ccc(O)cc2)C1]

To a solution of 4-(azetidin-3-ylsulfonyl)phenol (300 mg, 1.20 mmol, HCl salt) in DCM (3 mL) at 0° C. were added TEA (365 mg, 3.60 mmol) and acetyl chloride (56.6 mg, 721 umol). The reaction was stirred at 0° C. for 0.5 h. The reaction mixture was then poured into water (3 mL) and extracted with DCM (3 mL×3). The combined organic layers were washed with brine (15 mL), dried over Na₂SO₄, filtered and concentrated under reduced pressure to give a residue. The residue was purified by flash silica gel chromatography (ISCO 4 g cartridge, 0-100% EtOAc/Hexane) to give 1-[3-(4-hydroxyphenyl) sulfonylazetidin-1-yl]ethan-1-one. MS=256.1 [M+H]⁺

### Intermediate 15

### 2-(bromomethyl)-4-chloro-1-methoxybenzene

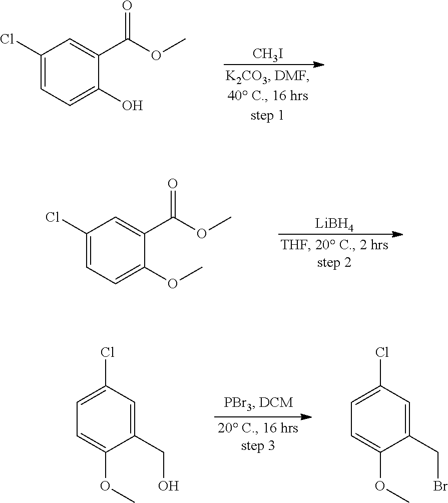

### Step 1: methyl 5-chloro-2-methoxybenzoate

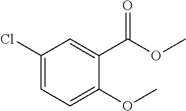

To a solution of methyl 5-chloro-2-hydroxy-benzoate (2.00 g, 10.7 mmol) in DMF (20 mL) were added K₂CO₃ (2.96 g, 21.4 mmol) and CH₃I (7.61 g, 53.6 mmol). The mixture was stirred at 40° C. for 16 h. The reaction mixture was then cooled to RT, poured into water (100 mL), and extracted with EtOAc (30 mL×3). The combined organic layers were washed with brine (80 mL), dried over Na₂SO₄, filtered and concentrated under reduced pressure to give the residue, which was purified by flash silica gel chromatography (ISCO 20 g cartridge, 0-10% EtOAc/Hexane) to give methyl 5-chloro-2-methoxy-benzoate. MS=201.0 [M+H]⁺

### Step 2: (5-chloro-2-methoxyphenyl)methanol

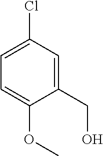

To a solution of methyl 5-chloro-2-methoxy-benzoate (1.5 g, 7.48 mmol) in THE (15 mL) at 0° C. was added LiBH₄ (2 M in THF, 7.5 mL). The reaction mixture was stirred at RT for 2 h. The reaction was quenched with water (20 mL) and extracted with EtOAc (20 mL×3). The combined organic layers were washed with brine (60 mL), dried over Na₂SO₄, filtered and concentrated to give a residue. The residue was purified by flash silica gel chromatography (ISCO 20 g cartridge, 0-50% EtOAc/Hexane) to give (5-chloro-2-methoxy-phenyl)methanol. ¹H NMR (CDCl₃, 400 MHz) δ 7.30 (d, J=2.8 Hz, 1H), 7.25-7.21 (m, 1H), 6.81 (d, J=8.8 Hz, 1H), 4.66 (s, 2H), 3.86 (s, 3H).

### Step 3: 2-(bromomethyl)-4-chloro-1-methoxybenzene

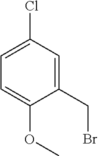

To a solution of (5-chloro-2-methoxy-phenyl)methanol (1.10 g, 6.37 mmol) in DCM (50 mL) at 0° C. was added PBr₃ (1.73 g, 6.37 mmol). The reaction mixture was then stirred at RT for 16 h. The mixture was concentrated, diluted with water (50 mL) and neutralized by addition of sat. aq. NaHCO₃ to pH=7-8, then extracted with DCM (50 mL×3). The combined organic layers were washed with brine (100 mL), dried over Na₂SO₄, filtered and concentrated to give 2-(bromomethyl)-4-chloro-1-methoxy-benzene. ¹H NMR (CDCl₃, 400 MHz) δ 7.27 (d, J=2.8 Hz, 1H), 7.21 (dd, J=5.6 Hz, 2.8 Hz, 1H), 6.77 (d, J=8.8 Hz, 1H), 4.45 (s, 2H), 3.85 (s, 3H).

### Intermediate 16

### 2-(bromomethyl)-4-chloro-1-(difluoromethoxy)benzene

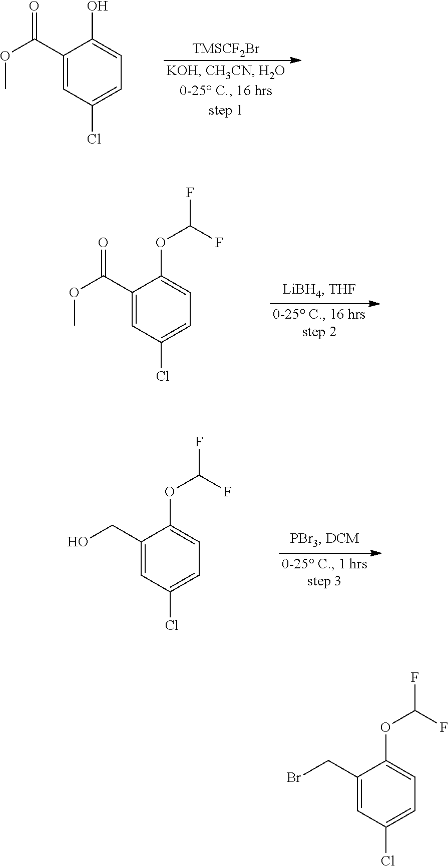

### Step 1: methyl 5-chloro-2-(difluoromethoxy)benzoate

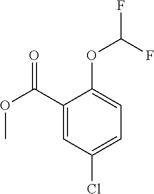

To a mixture of methyl 5-chloro-2-hydroxy-benzoate (1.5 g, 8.04 mmol) in MeCN (75 mL) and H₂O (37.5 mL) at 0° C. were added KOH (2.71 g, 48.2 mmol), [bromo(difluoro)methyl]-trimethylsilane (3.27 g, 16.1 mmol). The mixture was then stirred at RT for 16 h. The reaction mixture was poured into water (20 mL) and extracted with ethyl acetate (20 mL×3). The combined organic layers were washed with brine (25 mL), dried over Na₂SO₄, filtered and concentrated under reduced pressure to give a residue. The residue was purified by flash silica gel chromatography (ISCO 20 g cartridge, 0-20% EtOAc/Hexane) to give methyl 5-chloro-2-(difluoromethoxy)benzoate. ¹H NMR (DMSO-d₆, 400 MHz) δ 7.86 (d, J=2.8 Hz, 1H), 7.75 (dd, J=8.8 Hz, 2.8 Hz, 1H), 7.37 (d, J=9.2 Hz, 1H), 7.20 (t, J=73.6 Hz, 1H), 3.85 (s, 3H).

### Step 2: (5-chloro-2-(difluoromethoxy)phenyl)methanol

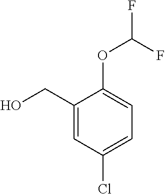 [SMILES: OCc1cc(Cl)ccc1OC(F)F]

To a mixture of methyl 5-chloro-2-(difluoromethoxy)benzoate (200 mg, 845 umol) in THE (5 mL) at 0° C. was added LiBH₄ (4 M in THF, 634 uL) dropwise under N₂. The mixture was then stirred at RT for 16 h under N₂. The reaction mixture was poured into saturated aqueous NH₄Cl (5 mL), then extracted with ethyl acetate (15 mL×3). The combined organic layers were washed with brine (5 mL), dried over Na₂SO₄, filtered and concentrated under reduced pressure to give [5-chloro-2-(difluoromethoxy)phenyl]methanol, which was used without further purification. MS=190.9 [M−H₂O]⁺.

### Step 3: 2-(bromomethyl)-4-chloro-1-(difluoromethoxy) benzene

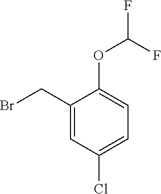 [SMILES: FC(F)Oc1ccc(Cl)cc1CBr]

To a solution of [5-chloro-2-(difluoromethoxy)phenyl]methanol (170 mg, 815 umol) in DCM (3 mL) at 0° C. was added PBr₃ (221 mg, 815 umol). The mixture was stirred at RT for 1 h. The reaction mixture was then poured into water (15 mL) and extracted with DCM (10 mL×3). The combined organic layers were washed with brine (5 mL), dried over anhydrous Na₂SO₄, filtered and concentrated under reduced pressure to give a residue. The residue was purified by flash silica gel chromatography (ISCO 4 g cartridge, 0-10% EtOAc/Hexane) to give 2-(bromomethyl)-4-chloro-1-(difluoromethoxy)benzene. ¹H NMR (DMSO-d₆, 400 MHz) δ 7.69 (d, J=2.4 Hz, 1H), 7.50 (dd, J=2.8 Hz, 8.0 Hz, 1H), 7.25 (d, J=8.8 Hz, 1H), 7.31 (t, J=73.2 Hz, 1H), 4.63 (s, 2H).

### Intermediate 17

### 2-(3, 5-dichlorophenyl) acetaldehyde

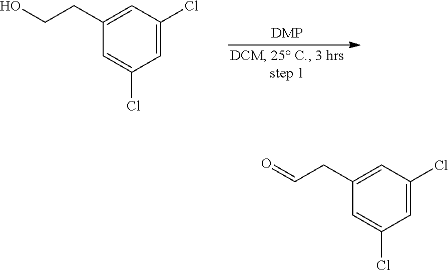 [SMILES: OCCc1cc(Cl)cc(Cl)c1 ; O=CCc1cc(Cl)cc(Cl)c1]

### Step 1: 2-(3, 5-dichlorophenyl) acetaldehyde

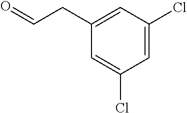 [SMILES: O=CCc1cc(Cl)cc(Cl)c1]

To a mixture of 2-(3,5-dichlorophenyl)ethanol (1.00 g, 5.23 mmol) in DCM (10 mL) was added DMP (2.66 g, 6.28 mmol). The mixture was then stirred at RT for 3 h under N₂. The reaction mixture was diluted with H₂O (5 mL) and extracted with EtOAc (8 mL×3). The combined organic layers were washed with brine (10 mL), dried over Na₂SO₄, filtered and concentrated under reduced pressure to give a residue. The residue was purified by flash silica gel chromatography (Biotage 12 g cartridge, 0-20% EtOAc/Hexane) to give 2-(3, 5-dichlorophenyl) acetaldehyde. ¹H NMR (DMSO-d₆, 400 MHz) 9.68 (s, 1H), 7.53 (s, 1H), 7.47 (s, 1H), 7.46 (s, 1H), 3.86 (s, 2H).

### Intermediate 18

### 3-chloro-5-(2-oxoethyl) benzonitrile

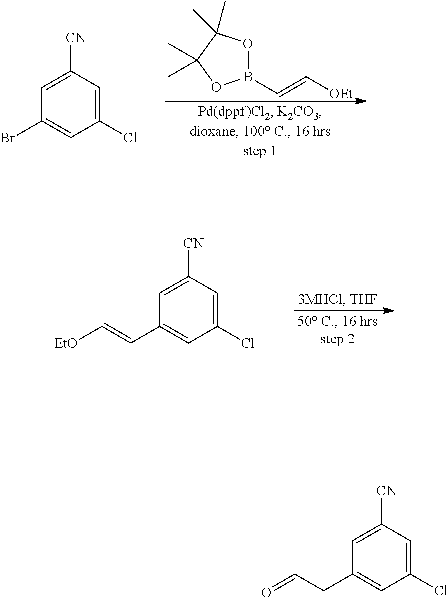

### Step 1: (E)-3-chloro-5-(2-ethoxyvinyl) benzonitrile

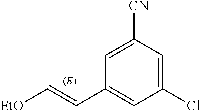 [SMILES: CCO/C=C/c1cc(Cl)cc(C#N)c1]

^{To} a solution of 2-[(E)-2-ethoxyvinyl]-4,4,5,5-tetramethyl-1,3,2-dioxaborolane (503 mg, 2.54 mmol) and 3-bromo-5-chloro-benzonitrile (500 mg, 2.31 mmol) in dioxane (10 mL) were added K₂CO₃ (958 mg, 6.93 mmol) and Pd(dppf)Cl₂ (169 mg, 231 umol) under N₂. The reaction mixture was then stirred at 100° C. for 16 h under N₂. The reaction mixture was diluted with H₂O (10 mL) and extracted with EtOAc (5 mL×3). The combined organic layers were washed with brine (10 mL), dried over Na₂SO₄, filtered and concentrated under reduced pressure to give (E)-3-chloro-5-(2-ethoxyvinyl)benzonitrile, which was taken to the next step without further purification. ¹H NMR (CDCl₃, 400 MHz) δ 7.40 (s, 1H), 7.36 (s, 1H), 7.35 (s, 1H), 7.05 (d, J=13.2 Hz, 1H), 5.73 (d, J=12.8 Hz, 1H), 3.94 (q, J=7.2 Hz, 2H), 1.37 (t, J=7.2 Hz, 3H).

### Step 2: 3-chloro-5-(2-oxoethyl) benzonitrile

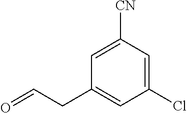 [SMILES: N#Cc1cc(Cl)cc(CC=O)c1]

To a solution of (E)-3-chloro-5-(2-ethoxyvinyl)benzonitrile (750 mg, 3.61 mmol) in THF (4 mL) was added HCl (3 M in H₂O, 4 mL). The reaction mixture was then stirred at 50° C. for 16 h. The reaction mixture was diluted with H₂O (10 mL) and extracted with EtOAc (5 mL×3). The combined organic layers were washed with brine (10 mL), dried over Na₂SO₄, filtered and concentrated under reduced pressure to give a residue. The residue was purified by flash silica gel chromatography (Biotage 4 g cartridge, 0-30% EtOAc/Hexane) to give 3-chloro-5-(2-oxoethyl)benzonitrile. ¹H NMR (CDCl₃, 400 MHz) δ 9.81 (s, 1H), 7.60 (s, 1H), 7.46 (s, 1H), 7.42 (s, 1H), 3.80 (s, 2H).

### Intermediate 19

### 3-chloro-5-(oxiran-2-yl) benzonitrile

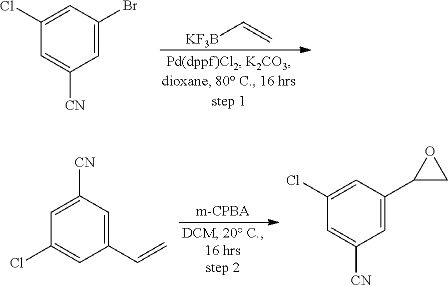

### Step 1: 3-chloro-5-vinylbenzonitrile

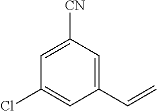

To a solution of 3-bromo-5-chloro-benzonitrile (2.50 g, 11.5 mmol), potassium vinyltrifluoroborate (3.09 g, 23.1 mmol) in dioxane (25 mL) and H₂O (2.5 mL) were added K₂CO₃ (3.19 g, 23.1 mmol) and Pd(dppf)Cl₂ (845 mg, 1.15 mmol). The mixture was purged with N₂ three times and then stirred at 80° C. for 16 h. The reaction mixture was poured into water (50 mL) and extracted with EtOAc (40 mL×3). The combined organic layers were washed with brine (100 mL), dried over Na₂SO₄, filtered and concentrated under reduced pressure to give a residue. The residue was purified by flash silica gel chromatography (BIOTAGE 20 g cartridge, 0-10% EtOAc/Hexane) to give 3-chloro-5-vinyl-benzonitrile. ¹H NMR (CDCl₃, 400 MHz) δ 7.60 (s, 1H), 7.56 (s, 1H), 7.52 (s, 1H), 6.69-6.62 (m, 1H), 5.85 (d, J=17.6 Hz, 1H), 5.47 (d, J=10.8 Hz, 1H).

### Step 2: 3-chloro-5-(oxiran-2-yl) benzonitrile

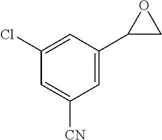 [SMILES: N#Cc1cc(Cl)cc(C2CO2)c1]

To a solution of 3-chloro-5-vinyl-benzonitrile (500 mg, 3.06 mmol) in DCM (5 mL) was added mCPBA (931 mg, 4.58 mmol, 85% purity) at 0° C., the reaction was stirred at RT for 16 h. The reaction mixture was then poured into sat. aq. Na₂SO₃ (20 mL) and extracted with EtOAc (30 mL×3). The combined organic layers were washed with brine (120 mL), dried over Na₂SO₄, filtered and concentrated under reduced pressure to give a residue. The residue was purified by flash silica gel chromatography (ISCO 12 g cartridge, 0-100% EtOAc/Hexane) to give 3-chloro-5-(oxiran-2-yl)benzonitrile.

### Intermediate 20

### 4-(3-(methylsulfonyl)oxetan-3-yl)phenol

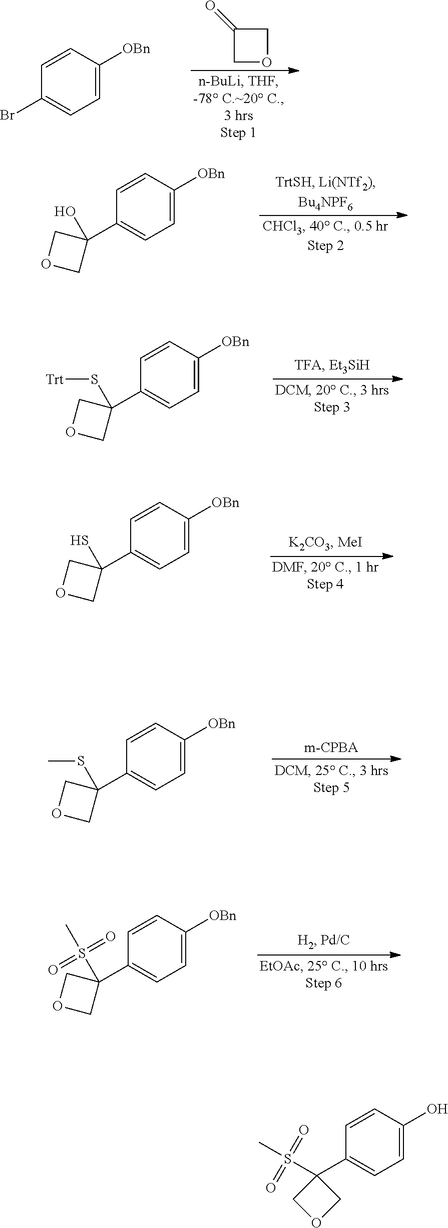

### Step 1: 3-(4-(benzyloxy)phenyl)oxetan-3-ol

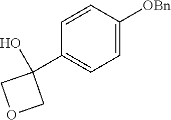 [SMILES: OC1(c2ccc(OCc3ccccc3)cc2)COC1]

To a solution of 1-(benzyloxy)-4-bromobenzene (3.00 g, 11.4 mmol) in THE (20 mL) was added n-BuLi (2.5 M in hexane, 7.30 mL) at −78° C. After 1 h at −78° C., oxetan-3-one (985 mg, 13.7 mmol) was added. The mixture was allowed to warm to RT and stirred for 3 h. The reaction mixture was cooled to 0° C., quenched by addition of H₂O (30 mL), and extracted with EtOAc (20 mL×3). The combined organic layers were dried over Na₂SO₄, filtered and concentrated under reduced pressure. The residue was purified by flash silica gel chromatography (Biotage 45 g cartridge, 0-50% EtOAc/Hexane) to give 3-(4-(benzyloxy)phenyl)oxetan-3-ol. MS=279.1 [M+Na]⁺

### Step 2: 3-(4-(benzyloxy)phenyl)-3-(tritylthio)oxetane

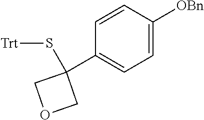

To a solution of 3-(4-(benzyloxy)phenyl)oxetan-3-ol (7.4 g, 28.9 mmol) and triphenylmethanethiol (39.9 g, 144.4 mmol) in CHCl₃ (80 mL) were added [bis(trifluoromethylsulfonyl)amino]lithium (911 mg, 3.18 mmol) and tetrabutylammonium hexafluorophosphate (615 mg, 1.59 mmol). The mixture was then stirred at 40° C. for 0.5 h. The reaction mixture was quenched by addition of H₂O (80 mL) and extracted with DCM (50 mL×3). The combined organic layers were dried over Na₂SO₄, filtered and concentrated under reduced pressure. The residue was purified by flash silica gel chromatography (Biotage 120 g cartridge, 0-40% EtOAc/Hexane) to give 3-(4-(benzyloxy)phenyl)-3-(tritylthio)oxetane. MS=537.3 [M+Na]⁺

### Step 3: 3-(4-(benzyloxy)phenyl)oxetane-3-thiol

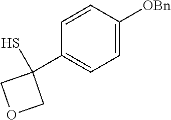 [SMILES: SC1(c2ccc(OCc3ccccc3)cc2)COC1]

To a solution of 3-(4-(benzyloxy)phenyl)-3-(tritylthio)oxetane (10.0 g, 19.4 mmol) in DCM (40 mL) were added TFA (810 mmol, 60.0 mL) and Et₃SiH (5.65 g, 48.6 mmol). The mixture was stirred at RT for 3 h. The reaction mixture was cooled to 0° C., quenched by addition of H₂O (80 mL), and extracted with DCM (50 mL×3). The combined organic layers were dried over Na₂SO₄, filtered and concentrated under reduced pressure to give 3-(4-(benzyloxy)phenyl)oxetane-3-thiol, which was taken to the next step without further purification. MS=273.1 [M+H]⁺

### Step 4: 3-(4-(benzyloxy)phenyl)-3-(methylthio)oxetane

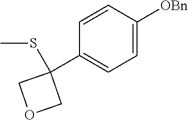 [SMILES: CSC1(c2ccc(OCc3ccccc3)cc2)COC1]

To a solution of 3-(4-(benzyloxy)phenyl)oxetane-3-thiol (5.00 g, 18.4 mmol) in DMF (40 mL) were added MeI (13.03 g, 91.8 mmol) and K₂CO₃ (3.81 g, 27.5 mmol). The mixture was stirred at RT for 1 h. The reaction mixture was cooled to 0° C., quenched by addition of H₂O (80 mL), extracted with EtOAc (50 mL×3). The combined organic layers were washed with brine (50 mL×2), dried over Na₂SO₄, filtered and concentrated under reduced pressure. The residue was purified by flash silica gel chromatography (Biotage 80 g cartridge, 0-30% EtOAc/Hexane) to give 3-(4-(benzyloxy)phenyl)-3-(methylthio)oxetane. ¹H NMR (CDCl₃, 400 MHz) δ 7.46-7.39 (m, 5H), 7.11 (d, J=6.8 Hz, 2H), 6.98 (d, J=8.8 Hz, 2H), 5.18 (d, J=6.4 Hz, 2H), 5.08 (s, 2H), 4.90 (d, J=6.4 Hz, 2H), 2.04 (s, 3H).

### Step 5: 3-(4-(benzyloxy)phenyl)-3-(methylsulfonyl)oxetane

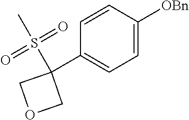 [SMILES: CS(=O)(=O)C1(c2ccc(OCc3ccccc3)cc2)COC1]

To a solution of 3-(4-(benzyloxy)phenyl)-3-(methylthio)oxetane (3.25 g, 11.35 mmol) in DCM (40 mL) was added mCPBA (6.91 g, 34.04 mmol, 85% purity) at 0° C. The mixture was then stirred at RT for 3 h. The reaction mixture was cooled to 0° C. and quenched by addition of sat. aq. Na₂SO₃ (20 mL). The mixture was then diluted with H₂O (60 mL) and extracted with DCM (40 mL×3). The combined organic layers were washed with sat. aq. NaHCO₃, dried over Na₂SO₄, filtered and concentrated under reduced pressure. The crude product was triturated with EtOAc at RT for 10 min to give 3-(4-(benzyloxy)phenyl)-3-(methylsulfonyl)oxetane, which was taken to the next step with out further purification. MS=336.2 [M+NH₄]⁺

### Step 6: 4-(3-(methylsulfonyl)oxetan-3-yl)phenol

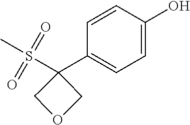

To a solution of 3-(4-(benzyloxy)phenyl)-3-(methylsulfonyl)oxetane (1.6 g, 5.03 mmol) in EtOAc (100 mL) was added Pd/C (2 g, 10% by weight) under N₂. The suspension was degassed under vacuum and purged with H₂ several times. The mixture was stirred under H₂ (50 Psi) at RT for 10 h. The reaction mixture was filtered and concentrated under reduced pressure to give 4-(3-(methylsulfonyl)oxetan-3-yl)phenol, which was taken to the next step with out further purification. MS=246.1 [M+NH₄]⁺

### Intermediate 21

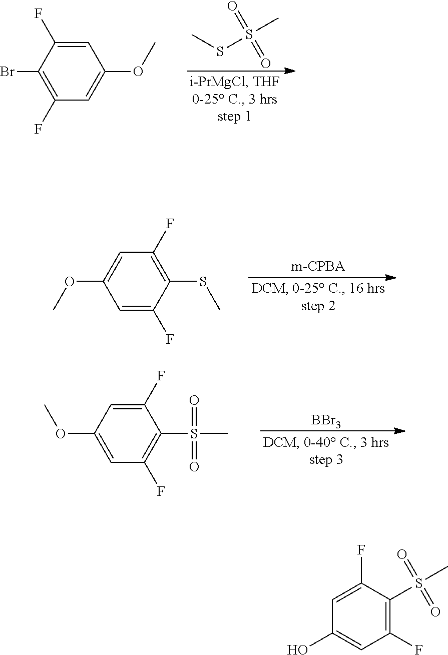

### Step 1: (2,6-difluoro-4-methoxyphenyl)(methyl)sulfane

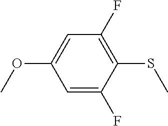

To a solution of 2-bromo-1, 3-difluoro-5-methoxy-benzene (20.0 g, 89.7 mmol) in THE (180 mL) at 0° C. under N₂ was added dropwise iPrMgCl (2 M in THF, 53.8 mL, 107.6 mmol) The mixture was stirred at 0° C. for 0.5 h. Methylsulfonylsulfanylmethane (16.98 g, 134.5 mmol) was then added dropwise at 0° C. under N₂. The mixture was warmed to RT and stirred for 2.5 h. The mixture was quenched by slow addition of sat. aq. NH₄Cl solution (300 mL), and then extracted with EtOAc (150 mL×3). The combined organic layers were washed with brine (100 mL), dried over Na₂SO₄, filtered and concentrated under reduced pressure to give 1, 3-difluoro-5-methoxy-2-methylsulfanyl-benzene, which was taken to the next step without further purification. ¹H NMR (CDCl₃, 400 MHz) δ 6.50 (s, 1H), 6.48 (s, 1H), 3.79 (s, 3H), 2.36 (s, 3H)

### Step 2: 1, 3-difluoro-5-methoxy-2-(methylsulfonyl) benzene

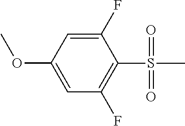

To a solution of 1, 3-difluoro-5-methoxy-2-methylsulfanyl-benzene (10.00 g, 52.57 mmol) in DCM (150 mL) was added m-CPBA (32.02 g, 157.7 mmol, 85% purity) at 0° C. The mixture was then stirred at RT for 16 h. The mixture was diluted with DCM (100 mL) and washed with saturated Na₂SO₃ solution (150 mL). The organic layer was washed with saturated Na₂CO₃ solution (100 mL), dried over Na₂SO₄, filtered and concentrated under reduced pressure. The residue was purified by flash silica gel chromatography (Biotage 80 g cartridge, 0-25% EtOAc/Hexane) to give 1,3-difluoro-5-methoxy-2-methylsulfonyl benzene. ¹H NMR (DMSO-d₆, 400 MHz) δ 6.99 (s, 1H), 6.96 (s, 1H), 3.87 (s, 3H), 3.34 (s, 3H)

### Step 3: 3, 5-difluoro-4-(methylsulfonyl) phenol

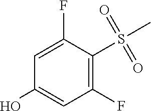

To a solution of 1,3-difluoro-5-methoxy-2-methylsulfonyl-benzene (5.5 g, 24.8 mmol) in DCM (60 mL) at 0° C. was added BBr₃ (24.8 g, 99.0 mmol) dropwise. The mixture was then stirred at 40° C. for 3 h. The mixture was slowly poured into H₂O (150 mL) with vigorous stirring. The mixture was then extracted with EtOAc (80 mL×2). The combined organic layers were dried over Na₂SO₄, filtered and concentrated under reduced pressure. The residue was purified by flash silica gel chromatography (Biotage 40 g cartridge, 0-40% EtOAc/Hexane) to give 3,5-difluoro-4-methylsulfonyl-phenol. MS=207.1 [M−H]⁻.

### Intermediate 22

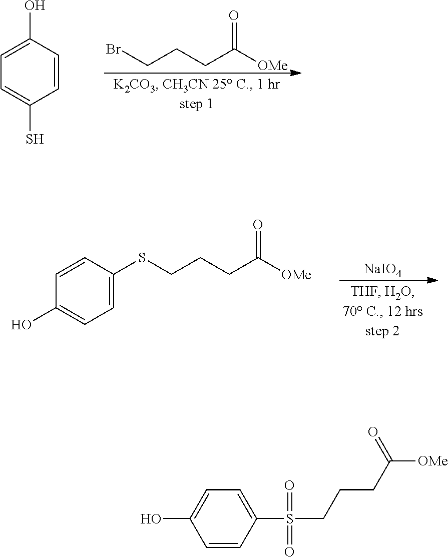 [SMILES: Oc1ccc(S)cc1 ; COC(=O)CCCBr ; COC(=O)CCCSc1ccc(O)cc1 ; "NaIO4" ; COC(=O)CCCS(=O)(=O)c1ccc(O)cc1]

### Step 1: methyl 4-((4-hydroxyphenyl)thio)butanoate

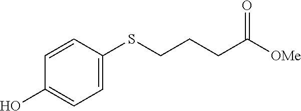 [SMILES: COC(=O)CCCSc1ccc(O)cc1]

A mixture of 4-sulfanylphenol (3.5 g, 27.74 mmol), methyl 4-bromobutanoate (6.03 g, 33.29 mmol), K₂CO₃ (7.67 g, 55.48 mmol) in CH₃CN (50 mL) was degassed and purged with N₂ for 3 times, then stirred at RT for 1 h under N₂. The mixture was diluted with H₂O (30 mL) and extracted with ethyl acetate (50 mL×3). The combined organic phase was washed with brine (50 mL×2), dried with anhydrous Na₂SO₄, filtered and concentrated under reduced pressure to give a residue. The residue was purified by flash silica gel chromatography (BIOTAGE 40 g cartridge, 0-30% EtOAc/Hexane) to give methyl 4-(4-hydroxyphenyl) sulfanylbutanoate. MS=227.1 [M+H]⁺.

### Step 2: methyl 4-((4-hydroxyphenyl) sulfonyl) butanoate

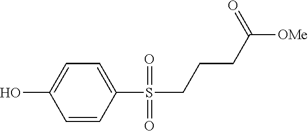 [SMILES: COC(=O)CCCS(=O)(=O)c1ccc(O)cc1]

To a mixture of methyl 4-(4-hydroxyphenyl)sulfanylbutanoate (8.0 g, 35.4 mmol) in THE (100 mL) and H₂O (100 mL) was added NaIO₄ (15.1 g, 70.7 mmol). The mixture was then stirred at 70° C. for 12 h under N₂. The mixture was diluted with H₂O (30 mL), then extracted with ethyl acetate (50 mL×3). The combined organic phase was washed with brine (50 mL×2), dried over anhydrous Na₂SO₄, filtered and concentrated under reduced pressure to give a residue. The residue was purified by flash silica gel chromatography (BIOTAGE 80 g cartridge, 0-30% EtOAc/Hexane) to give methyl 4-(4-hydroxyphenyl) sulfonylbutanoate. MS=259.1 [M+H]⁺.

### Intermediate 23

### 3-(bromomethyl)-5-(trifluoromethyl) benzonitrile

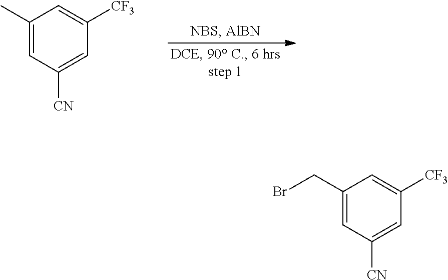

### Step 1: 3-(bromomethyl)-5-(trifluoromethyl) benzonitrile

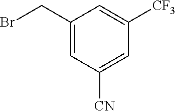 [SMILES: N#Cc1cc(CBr)cc(C(F)(F)F)c1]

A mixture of 3-methyl-5-(trifluoromethyl) benzonitrile (750 mg, 4.05 mmol), NBS (865 mg, 4.86 mmol) and AIBN (67 mg, 405 umol) in DCE (7.5 mL) was stirred at 90° C. for 6 h. The mixture was then diluted with DCM (20 mL) and washed with water (20 mL). The organic layer was concentrated under reduced pressure. The residue was purified twice by flash silica gel chromatography (Biotage 12 g cartridge, 0-1% EtOAc/Hexane) to give 3-(bromomethyl)-5-(trifluoro methyl)benzonitrile. MS=264.0/266.1 [M+H]⁺

### Intermediate 24

### 4-((1-hydroxy-2-methylpropan-2-yl) sulfonyl)phenol

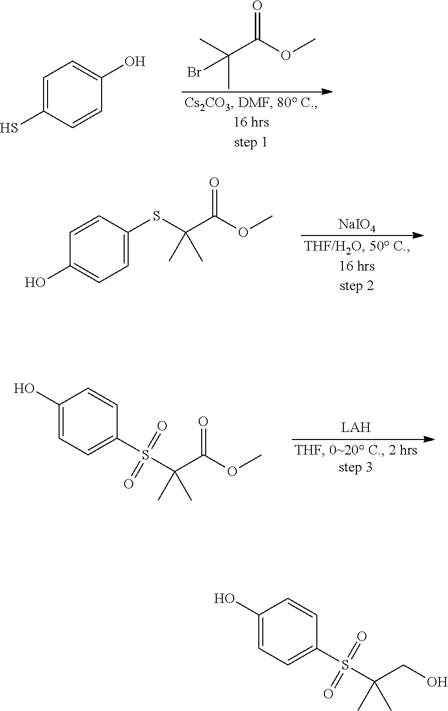

### Step 1: methyl 2-((4-hydroxyphenyl)thio)-2-methylpropanoate

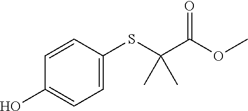

To a mixture of 4-sulfanylphenol (2.00 g, 15.9 mmol) and methyl 2-bromo-2-methyl-propanoate (2.40 g, 13.3 mmol) in DMF (30 mL) was added Cs₂CO₃ (7.20 g, 22.1 mmol). The mixture was then stirred at 80° C. for 16 h. The reaction mixture was diluted with sat. aq. NH₄Cl (30 mL), extracted with EtOAc (30 mL×3), dried over Na₂SO₄, filtered and concentrated under reduced pressure to give a residue. The residue was purified by flash silica gel chromatography (ISCO 12 g cartridge, 0-100% EtOAc/Hexane) to give methyl 2-(4-hydroxyphenyl) sulfanyl-2-methyl-propanoate. MS=225.2 [M−H]⁻.

### Step 2: methyl 2-((4-hydroxyphenyl) sulfonyl)-2-methylpropanoate

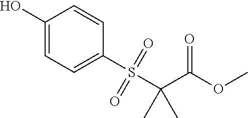 [SMILES: COC(=O)C(C)(C)S(=O)(=O)c1ccc(O)cc1]

To a solution of methyl 2-(4-hydroxyphenyl) sulfanyl-2-methyl-propanoate (2.30 g, 10.16 mmol) in THF (30 mL) was added a solution of NaIO₄ (6.52 g, 30.49 mmol) in H₂O (6 mL). The mixture was stirred at 50° C. for 16 h. The reaction mixture was quenched with sat. aq. Na₂SO₃ (15 mL), extracted with EtOAc (10 mL×3), dried over Na₂SO₄, filtered and concentrated under reduced pressure to give a residue. The residue was purified by flash silica gel chromatography (ISCO 20 g cartridge, 0-30% EtOAc/Hexane) to give methyl 2-(4-hydroxyphenyl) sulfonyl-2-methyl-propanoate. MS=257.1 [M−H]⁻.

### Step 3: 4-((1-hydroxy-2-methylpropan-2-yl) sulfonyl)phenol

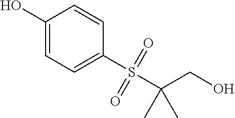

To a solution of methyl 2-(4-hydroxyphenyl) sulfonyl-2-methyl-propanoate (2.3 g, 8.90 mmol) in THF (30 mL) at 0° C. was added LiAlH₄ (507 mg, 13.36 mmol). The mixture was stirred at RT for 2 h. The mixture was then diluted with aq. NaOH (5 M, 2 mL), dried over Na₂SO₄, filtered and concentrated under reduced pressure to give a residue. The residue was purified by flash silica gel chromatography (ISCO 4 g cartridge, 0-100% EtOAc:Hexane to give 4-(2-hydroxy-1, 1-dimethyl-ethyl) sulfonylphenol. MS=229.0 [M−H]⁻.

### Intermediate 25

### 3-chloro-6-oxo-5, 6, 7, 8-tetrahydronaphthalene-1-carbonitrile

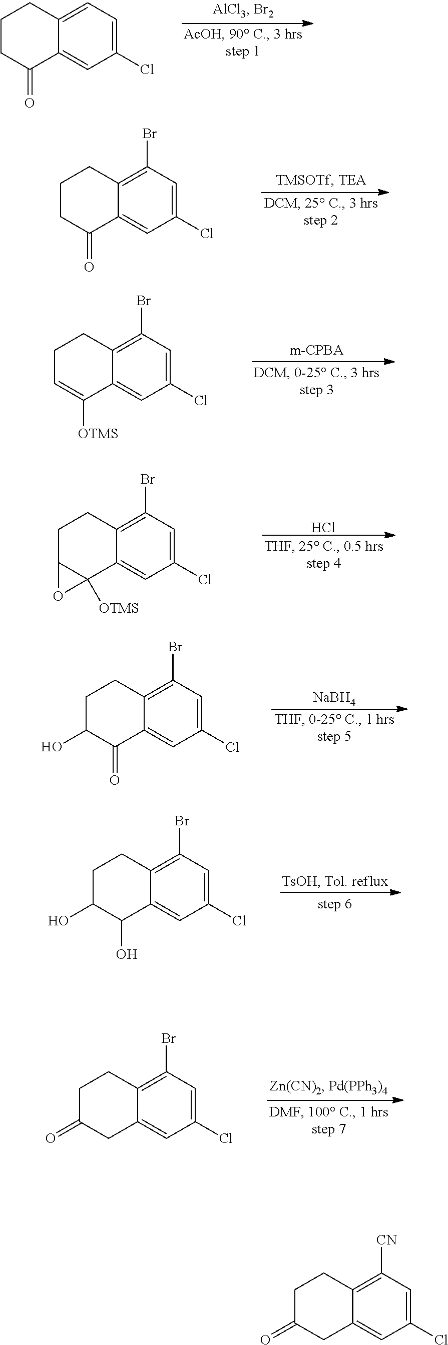

### Step 1: 5-bromo-7-chloro-3, 4-dihydronaphthalen-1(2H)-one

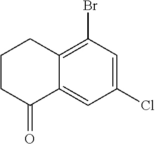 [SMILES: O=C1CCCc2c(Br)cc(Cl)cc21]

To a solution of AlCl₃ (23.99 g, 179.93 mmol) in DCE (400 mL) at RT was added 7-chlorotetralin-1-one (13.00 g, 71.97 mmol), followed by dropwise addition of Br₂ (12.65 g, 79.17 mmol). The reaction mixture was stirred at RT for 16 h. The reaction mixture was quenched by aq HCl (12 M, 30 mL) and ice (200 g). The mixture was then extracted with EtOAc (3×250 mL). The combined organic layers were washed with brine (200 mL), dried over Na₂SO₄, filtered and concentrated under reduced pressure to give a residue. The residue was purified by flash silica gel chromatography (Biotage 330 g cartridge, 0-6% EtOAc/Hexane) to give 5-bromo-7-chloro-3, 4-dihydronaphthalen-1(2H)-one. MS=259.0/261.0 [M+H]⁺.

### Step 2: ((5-bromo-7-chloro-3, 4-dihydronaphthalen-1-yl) oxy)trimethylsilane

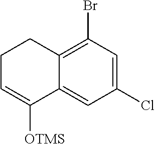

To a solution of 5-bromo-7-chloro-3,4-dihydronaphthalen-1(2H)-one (21.00 g, 80.92 mmol) in DCM (200 mL) were added TEA (16.38 g, 161.83 mmol) and trimethylsilyl trifluoromethanesulfonate (21.58 g, 97.10 mmol) at 0° C. The reaction mixture was then stirred at RT for 3 h. The reaction mixture was diluted with H₂O (200 mL) and extracted with EtOAc (3×150 mL). The combined organic layers were washed with brine (200 mL), dried over Na₂SO₄, filtered and concentrated under reduced pressure to give a residue. The residue was purified by flash silica gel chromatography (Biotage 200 g cartridge, 0-10% EtOAc/Hexane) to give ((5-bromo-7-chloro-3, 4-dihydronaphthalen-1-yl) oxy) trimethylsilane. MS=331.0/333.0 [M+H]⁺.

### Step 3: ((4-bromo-6-chloro-1a, 2, 3, 7b-tetrahydronaphtho [1,2-b]oxiren-7b yl) oxy) trimethylsilane

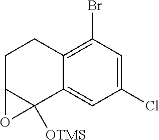

To a solution of ((5-bromo-7-chloro-3,4-dihydronaphthalen-1-yl)oxy)trimethylsilane (21.00 g, 63.31 mmol) in DCM (300 mL) was added m-CPBA (19.28 g, 94.96 mmol, 85% purity) portion wise at 0° C. The reaction mixture was then stirred at RT for 3 h. The reaction mixture was quenched by addition of sat. aq Na₂S₂O₃ (200 mL) and extracted with EtOAc (200 mL×3). The combined organic layers were washed with brine (200 mL), dried over Na₂SO₄, filtered and concentrated under reduced pressure to give ((4-bromo-6-chloro-1a,2,3,7b-tetrahydronaphtho[1,2-b]oxiren-7b-yl)oxy)trimethylsilane, which was taken to the next step without further purification.

### Step 4: 5-bromo-7-chloro-2-hydroxy-3, 4-dihydronaphthalen-1(2H)-one

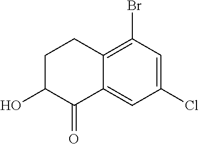 [SMILES: O=C1c2cc(Cl)cc(Br)c2CCC1O]

A mixture of ((4-bromo-6-chloro-1a,2,3,7b-tetrahydronaphtho[1,2-b]oxiren-7b-yl)oxy)trimethylsilane (19.0 g, 54.64 mmol) in THE (50 mL) and aq. HCl (12 M, 50 mL) was stirred at RT for 0.5 h. The reaction mixture was diluted with sat. aq. NaHCO₃ (100 mL) and extracted with EtOAc (3×50 mL). The combined organic layers were washed with brine (100 mL), dried over Na₂SO₄, filtered and concentrated under reduced pressure to give a residue. The residue was purified by flash silica gel chromatography (Biotage 220 g cartridge, 0-30% EtOAc/Hexane) to give 5-bromo-7-chloro-2-hydroxy-3,4-dihydronaphthalen-1(2H)-one. ¹H NMR (DMSO-d₆, 400 MHz) δ 8.01 (d, J=2.4 Hz, 1H), 7.80 (d, J=2.0 Hz, 1H), 4.40-4.33 (m, 1H), 3.01-2.92 (m, 2H), 2.29-2.26 (m, 1H), 1.98-1.95 (m, 1H).

### Step 5: 5-bromo-7-chloro-1, 2, 3, 4-tetrahydronaphthalene-1, 2-diol

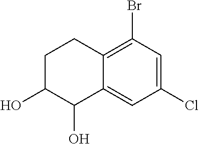 [SMILES: OC1CCc2c(Br)cc(Cl)cc2C1O]

To a solution of 5-bromo-7-chloro-2-hydroxy-3, 4-dihydronaphthalen-1(2H)-one (7.5 g, 27.22 mmol) in THE (100 mL) at 0° C. was added NaBH₄ (3.09 g, 81.66 mmol). The reaction mixture was then stirred at RT for 1 h. The reaction mixture was quenched by addition of sat. aq. NH₄Cl (100 mL) and extracted with EtOAc (100 mL×2). The combined organic layers were washed with brine (100 mL), dried over Na₂SO₄, filtered and concentrated under reduced pressure to give a residue. The residue was purified by flash silica gel chromatography (Biotage 80 g cartridge, 0-50% EtOAc/Hexane) to give 5-bromo-7-chloro-1, 2, 3, 4-tetrahydronaphthalene-1, 2-diol. ¹H NMR (DMSO-d₆, 400 MHz) δ 7.59 (d, J=2.0 Hz, 1H), 7.44 (d, J=2.0 Hz, 1H), 5.59 (d, J=5.6 Hz, 1H), 4.95 (d, J=3.6 Hz, 1H), 4.26 (t, J=6.0 Hz, 1H), 3.68-3.64 (m, 1H), 2.70-2.62 (m, 2H), 1.97-1.94 (m, 1H), 1.75-1.71 (m, 1H).

### Step 6: 5-bromo-7-chloro-3, 4-dihydronaphthalen-2(1H)-one

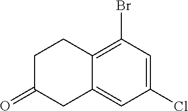 [SMILES: O=C1CCc2c(Br)cc(Cl)cc2C1]

To a solution of 5-bromo-7-chloro-1,2,3,4-tetrahydronaphthalene-1,2-diol (5.00 g, 18.02 mmol) in toluene (25 mL) was added 4-methylbenzenesulfonic acid hydrate (3.43 g, 18.02 mmol). The reaction mixture was stirred at 130° C. for 2 h to remove water by Dean-Stark trap. The reaction mixture was concentrated under reduced pressure to give a residue. The residue was diluted with H₂O (20 mL) and extracted with EtOAc (3×20 mL). The combined organic layers were washed with brine (20 mL), dried over Na₂SO₄, filtered and concentrated under reduced pressure to give a residue. The residue was purified by flash silica gel chromatography (Biotage 40 g cartridge, 0-30% EtOAc/Hexane) to give 5-bromo-7-chloro-3, 4-dihydronaphthalen-2(1H)-one. ¹H NMR (CDCl₃, 400 MHz) δ 7.50 (d, J=1.6 Hz, 1H), 7.09 (s, 1H), 3.57 (s, 2H), 3.20 (t, J=6.4 Hz, 2H), 2.56 (t, J=6.8 Hz, 2H)

### Step 7: 3-chloro-6-oxo-5, 6, 7, 8-tetrahydronaphthalene-1-carbonitrile

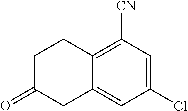 [SMILES: N#Cc1cc(Cl)cc2c1CCC(=O)C2]

A mixture of 5-bromo-7-chloro-3, 4-dihydronaphthalen-2(1H)-one (800 mg, 3.08 mmol), Pd(PPh₃)₄ (356 mg, 308 umol) and Zn(CN)₂ (723 mg, 6.17 mmol) in DMF (10 mL) was stirred at 100° C. for 1 h in a Microwave. The reaction mixture was diluted with H₂O (15 mL) and extracted with EtOAc (3×15 mL). The combined organic layers were washed with brine (15 mL), dried over Na₂SO₄, filtered and concentrated under reduced pressure to give a residue. The residue was purified by flash silica gel chromatography (Biotage 12 g cartridge, 0-100% EtOAc/Hexane) to give 3-chloro-6-oxo-5,6,7,8-tetrahydronaphthalene-1-carbonitrile. MS=206.10 [M+H]⁺.

### Intermediate 26

### 4-(1-(methylsulfonyl) cyclopropyl)phenol

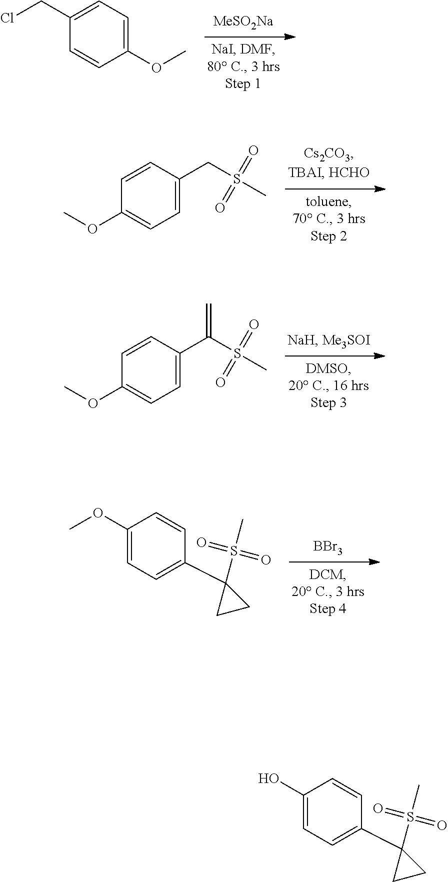

### Step 1: 1-methoxy-4-((methylsulfonyl) methyl) benzene

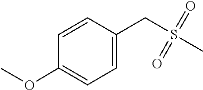

A mixture of 1-(chloromethyl)-4-methoxy-benzene (10.00 g, 63.85 mmol), sodium methanesulfinate (9.13 g, 89.4 mmol) and NaI (28.71 mg, 191.6 umol) in DMF (80 mL) was stirred at 80° C. for 3 h. The reaction mixture was cooled to 0° C., quenched by addition of H₂O (200 mL), and extracted with EtOAc (100 mL×3). The combined organic layers were washed with brine (100 mL×2), dried over Na₂SO₄, filtered and concentrated under reduced pressure. The crude product was triturated with EtOAc (30 mL) at RT for 10 min and filtered to give 1-methoxy-4-(methylsulfonylmethyl)benzene, which was taken to the next step without further purification. ¹H NMR (CDCl₃, 400 MHz) δ 7.33 (d, J=8.4 Hz, 2H), 6.94 (d, J=8.8 Hz, 2H), 4.20 (s, 2H), 3.83 (s, 3H), 2.74 (s, 3H).

### Step 2: 1-methoxy-4-(1-(methylsulfonyl) vinyl) benzene

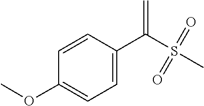

A mixture of 1-methoxy-4-(methylsulfonylmethyl)benzene (9.00 g, 44.9 mmol), Cs₂CO₃ (43.93 g, 135 mmol), TBAI (166 mg, 449 umol) and HCHO (13.49 g, 449.4 mmol) in toluene (150 mL) was stirred at 70° C. for 3 h. The reaction mixture was cooled to 0° C., quenched by addition of H₂O (200 mL), and extracted with EtOAc (100 mL×2). The combined organic layers were dried over Na₂SO₄, filtered and concentrated under reduced pressure. The residue was purified by flash silica gel chromatography (Biotage 120 g cartridge, 0-100% EtOAc/Hexane) to give 1-methoxy-4-(1-methylsulfonylvinyl)benzene. MS=213.1 [M+H]⁺

### Step 3: 1-methoxy-4-(1-(methylsulfonyl)cyclopropyl)benzene

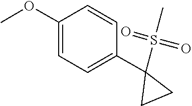

To a solution of trimethylsulfoxonium iodide (522 mg, 2.37 mmol) in DMSO (5 mL) was added NaH (84.4 mg, 2.11 mmol, 60% in mineral oil). The mixture was stirred at RT for 1 h, then a solution of 1-methoxy-4-(1-methylsulfonylvinyl) benzene (280 mg, 1.32 mmol) in DMSO (1 mL) was added. The resulting mixture was stirred at RT for 16 h. The reaction mixture was cooled to 0° C., quenched by addition of sat. aq. NH₄Cl (10 mL), and extracted with EtOAc (5 mL×3). The combined organic layers were washed with brine (5 mL×2), dried over Na₂SO₄, filtered and concentrated under reduced pressure. The residue was purified by flash silica gel chromatography (Biotage 4 g cartridge, 0-50% EtOAc/Hexane) to give 1-methoxy-4-(1-methylsulfonylcyclopropyl) benzene. ¹H NMR (DMSO-d₆, 400 MHz) δ 7.46 (d, J=8.4 Hz, 2H), 6.95 (d, J=8.8 Hz, 2H), 3.76 (s, 3H), 2.81 (s, 3H), 1.58 (dd, J=6.8 Hz, 4.8 Hz, 2H), 1.22 (dd, J=6.8 Hz, 4.8 Hz, 2H).

### Step 4: 4-(1-(methylsulfonyl) cyclopropyl)phenol

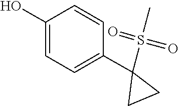

To a solution of 1-methoxy-4-(1-methylsulfonylcyclopropyl) benzene (1.20 g, 5.30 mmol) in DCM (20 mL) at 0° C. was added BBr₃ (3.99 g, 15.9 mmol). The mixture was stirred at RT for 3 h. The reaction mixture was cooled to 0° C., quenched by addition of H₂O (30 mL), adjusted to pH=8-9 with sat. aq. NaHCO₃, and then extracted with DCM (20 mL×3). The combined organic layers were dried over Na₂SO₄, filtered and concentrated under reduced pressure to give 4-(1-methylsulfonylcyclopropyl) phenol, which was taken to the next step without further purification. ¹H NMR (DMSO-d₆, 400 MHz) δ 9.66 (s, 1H), 7.33 (d, J=8.4 Hz, 2H), 6.76 (d, J=8.4 Hz, 2H), 2.79 (s, 3H), 1.55 (dd, J=6.8 Hz, 4.8 Hz, 2H), 1.18 (dd, J=6.8 Hz, 4.8 Hz, 2H).

### Intermediate 27

### allyl 3-((4-hydroxyphenyl) sulfonyl) azetidine-1-carboxylate

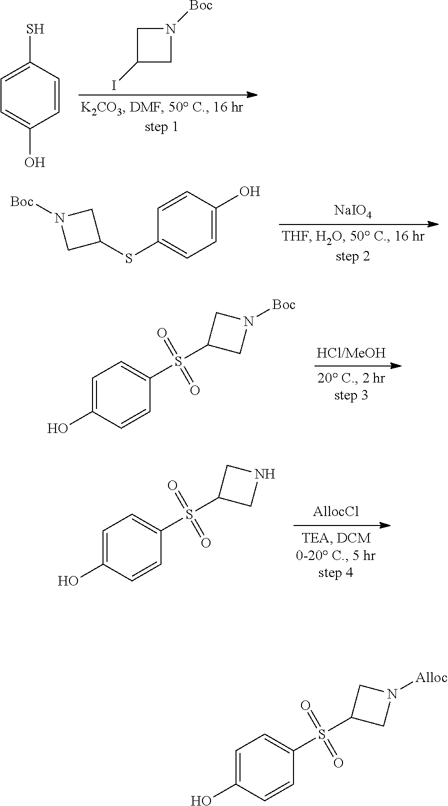

### Step 1: tert-butyl 3-((4-hydroxyphenyl) thio) azetidine-1-carboxylate

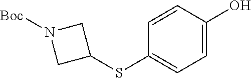 [SMILES: CC(C)(C)OC(=O)N1CC(Sc2ccc(O)cc2)C1]

To a solution of 4-sulfanylphenol (10.0 g, 79.25 mmol,) in DMF (100 mL) were added K₂CO₃ (10.9 g, 79.25 mmol) and tert-butyl 3-iodoazetidine-1-carboxylate (22.4 g, 79.25 mmol). The mixture was then stirred at 40° C. for 16 h. The reaction mixture was diluted with water (200 mL) and extracted with ethyl acetate (100 mL×3). The combined organic layers were washed with H₂O (80 mL) and brine (80 mL), dried over (Na₂SO₄), filtered and concentrated under reduced pressure to give a residue. The crude product was triturated with MTBE (100 mL) at RT for 5 h and filtered to give tert-butyl 3-(4-hydroxyphenyl)sulfanylazetidine-1-carboxylate. MS=226.1 [M−C₄H₈+H]⁺.

### Step 2: tert-butyl 3-((4-hydroxyphenyl) sulfonyl) azetidine-1-carboxylate

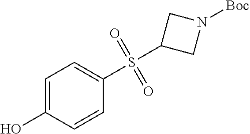 [SMILES: CC(C)(C)OC(=O)N1CC(S(=O)(=O)c2ccc(O)cc2)C1]

To a solution of tert-butyl 3-(4-hydroxyphenyl) sulfanylazetidine-1-carboxylate (6.5 g, 13.9 mmol) in THE (60 mL) and H₂O (20 mL) was added NaIO₄ (8.8 g, 41.58 mmol). The mixture was stirred at 50° C. for 16 h. The reaction mixture was cooled to RT, quenched with sat. aq. Na₂SO₃ (60 mL) and extracted with EtOAc (30 mL×3). The combined organic layers were washed with brine (30 mL), dried over Na₂SO₄, filtered and concentrated under reduced pressure to give a residue. The residue was purified by flash silica gel chromatography (ISCO 20 g cartridge, 0-100% EtOAc/Hexane) to give tert-butyl 3-(4-hydroxyphenyl) sulfonylazetidine-1-carboxylate. MS=312.2 [M−H]⁻.

### Step 3: 4-(azetidin-3-ylsulfonyl) phenol

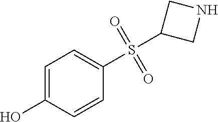 [SMILES: O=S(=O)(c1ccc(O)cc1)C1CNC1]

To a solution of tert-butyl 3-(4-hydroxyphenyl) sulfonylazetidine-1-carboxylate (1.5 g, 4.79 mmol) in MeOH (4 mL) was added HCl in MeOH (4 M, 12 mL). The mixture was stirred at RT for 2 h. The reaction mixture was concentrated under reduced pressure to give 4-(azetidin-3-ylsulfonyl) phenol HCl salt, which was used without further purification. MS=214.1 [M+H]⁺.

### Step 4: allyl 3-((4-hydroxyphenyl) sulfonyl) azetidine-1-carboxylate

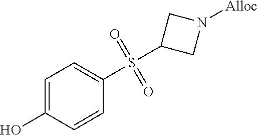

To a solution of 4-(azetidin-3-ylsulfonyl)phenol (1.10 g, 4.41 mmol, HCl salt) in DCM (10 mL) were added TEA (1.11 g, 11.0 mmol, 1.53 mL) and allyl chloroformate (584 mg, 4.85 mmol) at 0° C. The mixture was then stirred at RT for 3 h. The reaction mixture was diluted with H₂O (30 mL) and extracted with DCM (15 mL×3). The combined organic layers were washed with brine (5 mL), dried over Na₂SO₄, filtered and concentrated under reduced pressure to give a residue. The residue was purified by flash silica gel chromatography (ISCO 12 g cartridge, 0-100% EtOAc/Hexane) to give allyl 3-(4-hydroxyphenyl)sulfonylazetidine-1-carboxylate. MS=298.1 [M+H]⁺.

### Intermediate 28

### 4-(S-methyl-N-(2-(methylsulfonyl) ethyl) sulfonimidoyl) phenol

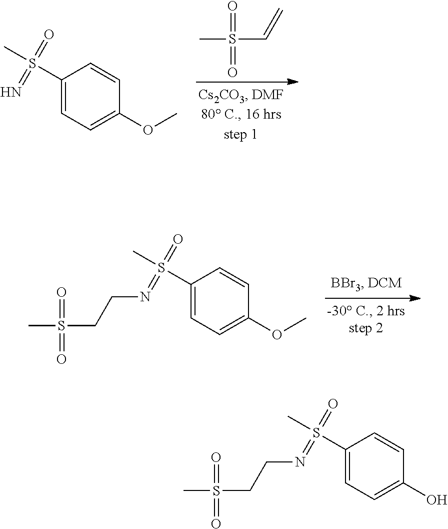

### Step 1: (4-methoxyphenyl)-methyl-(2-methylsulfonylethylimino)-oxo-λ6-sulfane

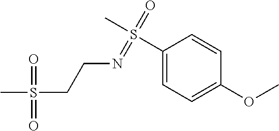

To a solution of imino-(4-methoxyphenyl)-methyl-oxo-λ6-sulfane (500 mg, 2.70 mmol) and 1-methylsulfonylethylene (287 mg, 2.70 mmol) in DMF (10 mL) was added Cs₂CO₃ (1.76 g, 5.40 mmol). The mixture was then stirred at 80° C. for 16 h. The reaction mixture was concentrated under reduced pressure. The residue was diluted with H₂O (15 mL) and extracted with EtOAc (5 mL×3), dried over Na₂SO₄, filtered and concentrated under reduced pressure to give (4-methoxyphenyl)-methyl-(2-methylsulfonylethylimino)-oxo-6-sulfane, which was taken to the next step without further purification. MS=292.0 [M+H]⁺.

### Step 2: 4-(S-methyl-N-(2-(methylsulfonyl) ethyl) sulfonimidoyl) phenol

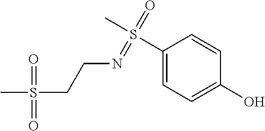 [SMILES: CS(=O)(=O)CCN=S(C)(=O)c1ccc(O)cc1]

To a solution of (4-methoxyphenyl)-methyl-(2-methylsulfonylethylimino)-oxo-λ6-sulfane (300 mg, 1.03 mmol) in DCM (5 mL) at −30° C. was added BBr₃ (1.29 g, 5.15 mmol). The mixture was stirred at −30° C. for 2 h. The reaction mixture was quenched with H₂O (15 mL), extracted with EtOAc (10 mL×3), dried over Na₂SO₄, filtered and concentrated under reduced pressure to give a residue. The residue was purified by reversed phase HPLC (Agela C18, 15-45% MeCN:H₂O) to give 4-[S-methyl-N-(2-methylsulfonylethyl) sulfonimidoyl]phenol. MS=278.0 [M+H]⁺.

### Intermediate 29

### 6-(N, S-dimethylsulfonimidoyl) pyridin-3-ol

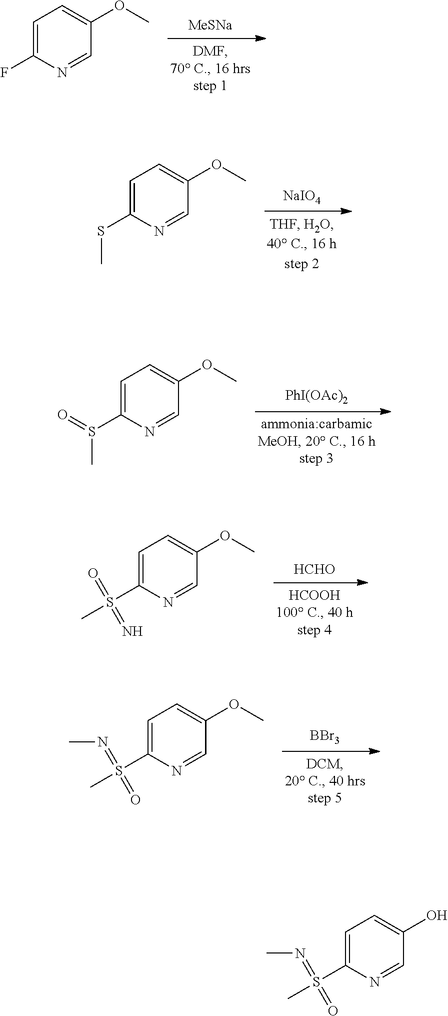

### Step 1: 5-methoxy-2-(methylthio) pyridine

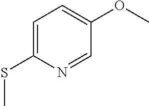

To a solution of 2-fluoro-5-methoxy-pyridine (9.00 g, 70.80 mmol) in DMF (80 mL) was added methylsulfanylsodium (7.44 g, 106.20 mmol). The mixture was stirred at 70° C. for 16 h. The mixture was poured into water (200 mL) and extracted with EtOAc (60 mL×3). The combined organic layers were washed with brine 100 mL, dried over Na₂SO₄, filtered and concentrated under reduced pressure to give a residue. The residue was purified by flash silica gel chromatography (ISCO 80 g cartridge, 0-25% EtOAc/Hexane) to give 5-methoxy-2-methylsulfanyl-pyridine. MS=156.1 [M+H]⁺.

### Step 2: 5-methoxy-2-(methylsulfinyl) pyridine

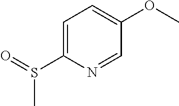

To a solution of 5-methoxy-2-methylsulfanyl-pyridine (8.50 g, 54.76 mmol) in THE (50 mL) and H₂O (50 mL) was added NaIO₄ (14.06 g, 65.71 mmol). The mixture was then stirred at 40° C. for 16 h. The mixture was diluted with water (50 mL), extracted with EtOAc (50 mL×3). The combined organic layers were washed with brine (100 mL×2), dried over Na₂SO₄, filtered and concentrated under reduced pressure to give a residue. The residue was purified by flash silica gel chromatography (ISCO 80 g cartridge, 0-40% EtOAc/Hexane) to give 5-methoxy-2-methylsulfinyl-pyridine. MS=172.1 [M+H]⁺.

### Step 3: 5-methoxy-2-(S-methylsulfonimidoyl) pyridine

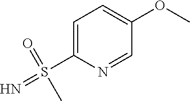

To a solution of 5-methoxy-2-methylsulfinyl-pyridine (2.00 g, 11.68 mmol) in MeOH (80 mL) were added iodosobenzene diacetate (15.05 g, 46.72 mmol) and ammonium carbamate (1.37 g, 17.52 mmol). The mixture was stirred at RT for 16 h. The reaction was concentrated under reduced pressure. The residue was diluted with sat. aq. NaHCO₃ (40 mL) and extracted with EtOAc (50 mL×3). The combined organic layers were washed with brine (50 mL), dried over Na₂SO₄, filtered and concentrated under reduced pressure to give crude 5-methoxy-2-(S-methylsulfonimidoyl) pyridine, which was taken to the next step without further purification. MS=187.1 [M+H]

### Step 4: (5-methoxy-2-pyridyl)-methyl-methylimino-oxo-)⁶-sulfane

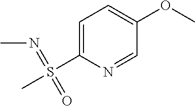

To a solution of 5-methoxy-2-(S-methylsulfonimidoyl) pyridine (1.50 g, 8.05 mmol) in HCOOH (15 mL) was added formaldehyde (16.35 g, 201.5 mmol, 37% in H₂O). The mixture was then stirred at 100° C. for 40 h. The reaction mixture was concentrated under reduced pressure. The residue was diluted with sat. aq. NaHCO₃ (30 mL) and extracted with EtOAc (30 mL×3). The combined organic layers were washed with brine (50 mL), dried over Na₂SO₄, filtered and concentrated under reduced pressure to give crude (5-methoxy-2-pyridyl)-methyl-methylimino-oxo-)⁶-sulfane, which was taken to the next step without further purification. MS=201.1 [M+H]⁺.

### Step 5: 6-(N, S-dimethylsulfonimidoyl) pyridin-3-ol

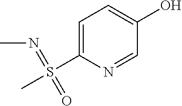

To a solution of (5-methoxy-2-pyridyl)-methyl-methylimino-oxo-λ⁶-sulfane (1.30 g, 6.49 mmol) in DCM (20 mL) at 0° C. was added BBr₃ (16.26 g, 64.92 mmol). The mixture was stirred at RT for 40 h. The mixture was poured into MeOH (100 mL), stirred for 10 min, and then concentrated under reduced pressure to give a residue. The residue was purified by preparative reverse phase HPLC (Phenomenex luna C18, 1-30% MeCN: 0.1% TFA in H₂O) to give 6-(N, S-dimethylsulfonimidoyl) pyridin-3-ol. MS=187.1 [M+H]⁺.

### Intermediate 30

### 5-hydroxy-2-(methylsulfonyl) benzonitrile

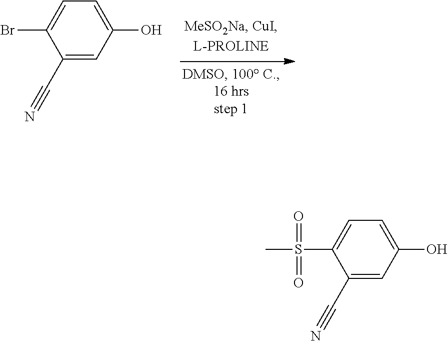

### Step 1: 5-hydroxy-2-(methylsulfonyl) benzonitrile

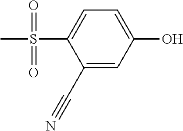

To a mixture of 2-bromo-5-hydroxy-benzonitrile (500 mg, 2.53 mmol) and sodium methanesulfinate (645 mg, 6.31 mmol) in DMSO (10 mL) were added CuI (72 mg, 378 umol), and L-proline (87 mg, 757 umol). The mixture was then stirred under N₂ at 100° C. for 16 h. The reaction mixture was diluted with H₂O (30 mL) and extracted with EtOAc (10 mL×3). The combined organic layers were washed with brine (10 mL×3), dried over Na₂SO₄, filtered and concentrated under reduced pressure to give a residue. The residue was purified by preparative SiO₂ TLC (3:1 Petroleum ether/Ethyl acetate) to give 5-hydroxy-2-methylsulfonyl-benzonitrile. MS=196.1 [M−H]⁻

### Intermediate 31

### 5-(2-oxoethyl) isophthalonitrile

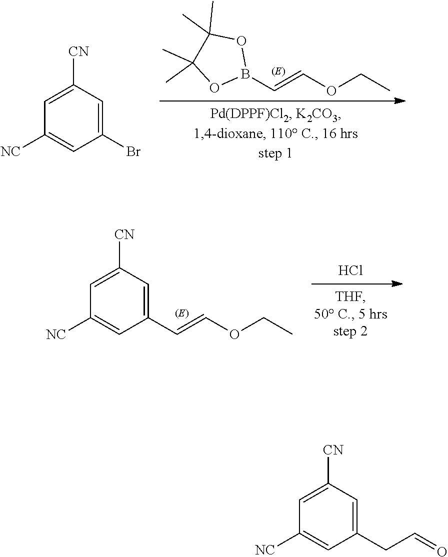

### Step 1: (E)-5-(2-ethoxyvinyl) isophthalonitrile

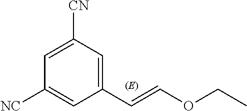

A mixture of 5-bromobenzene-1,3-dicarbonitrile (1.00 g, 4.83 mmol), 2-[(E)-2-ethoxyvinyl]-4,4,5,5-tetramethyl-1,3,2-dioxaborolane (1.05 g, 5.31 mmol), Pd(dppf)Cl₂ (353 mg, 483 umol) and K₂CO₃ (1.34 g, 9.66 mmol) in 1,4-dioxane (25 mL) was degassed and purged with N₂ three times. The mixture was then stirred at 110° C. for 13 h under N₂. The reaction mixture was cooled to RT and poured into H₂O (30 mL) and then extracted with EtOAc (30 mL×3). The combined organic layers were washed with brine (60 mL), dried over Na₂SO₄, concentrated under reduced pressure to give (E)-5-(2-ethoxyvinyl) isophthalonitrile, which was taken to the next step without further purification. MS=199.1 [M+H]⁺

### Step 2: 5-(2-oxoethyl) isophthalonitrile

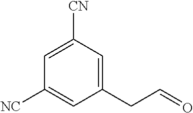 [SMILES: N#Cc1cc(C#N)cc(CC=O)c1]

To a solution of (E)-5-(2-ethoxyvinyl) isophthalonitrile (500 mg, 2.52 mmol) in THE (5 mL) was added aq. HCl (3 M, 5 mL). The reaction mixture was then stirred at 50° C. for 5 h. The reaction mixture was cooled to RT and extracted with EtOAc (3×10 mL). The combined organic layers were washed with brine (50 mL), dried over Na₂SO₄, filtered and concentrated under reduced pressure to give a residue. The residue was purified by flash silica gel chromatography (Biotage 12 g cartridge, 0-100% EtOAc/Hexane) to give 5-(2-oxoethyl)isophthalonitrile. ¹H NMR (400 MHz, CDCl₃) δ 9.86 (s, 1H), 7.90 (s, 1H), 7.81 (s, 1H), 7.74 (s, 1H), 3.49 (s, 2H)

### Intermediate 32

### 5-(oxiran-2-yl) isophthalonitrile

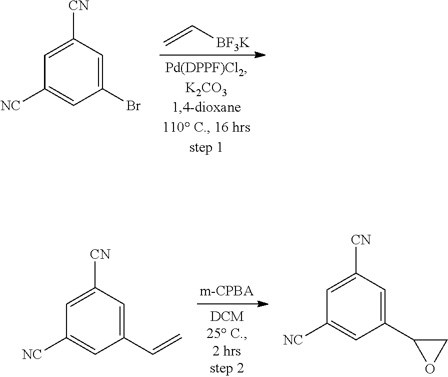

### Step 1: 5-vinylisophthalonitrile

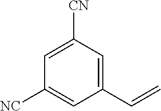

A mixture of 5-bromobenzene-1,3-dicarbonitrile (3.00 g, 14.49 mmol), potassium vinyltrifluoroborate (2.90 g, 21.74 mmol, K₂CO₃ (4.01 g, 29.0 mmol) and Pd(dppf)Cl₂ (1.10 g, 1.45 mmol) in 1,4-dioxane (60 mL) was degassed and purged with N₂ three times. The mixture was then stirred at 110° C. for 16 h under N₂. The reaction mixture was cooled to RT, poured into H₂O (40 mL), then extracted with EtOAc (40 mL×3). The combined organic layers were washed with brine (30 mL×3), dried over Na₂SO₄, filtered and concentrated under reduced pressure to give a residue. The residue was purified by flash silica gel chromatography (ISCO 40 g cartridge, 0-10% EtOAc/Hexane) to give 5-vinylbenzene-1, 3-dicarbonitrile. ¹H NMR (400 MHz, CDCl₃) δ 7.88-7.87 (m, 2H), 7.81 (s, 1H), 6.75-6.67 (m, 1H), 5.93 (d, J=17.6 Hz, 1H), 5.58 (d, J=11.2 Hz, 1H)

### Step 2: 5-(oxiran-2-yl) isophthalonitrile

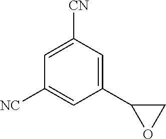 [SMILES: N#Cc1cc(C#N)cc(C2CO2)c1]

To a solution of 5-vinylbenzene-1, 3-dicarbonitrile (500 mg, 3.24 mmol) in DCM (6 mL) was added m-CPBA (987 mg, 4.86 mmol, 85% purity). The mixture was stirred at RT for 2 h under N₂. The reaction mixture was diluted with sat. aq. Na₂SO₃ (10 mL) and extracted with DCM (10 mL×3). The combined organic layers were washed with sat. aq. NaHCO₃ (10 mL) and brine (20 mL), dried over Na₂SO₄, filtered and concentrated under reduced pressure to give a residue. The residue was purified by flash silica gel chromatography (ISCO 12 g cartridge, 0-20% EtOAc/Hexane) to give 5-(oxiran-2-yl) benzene-1, 3-dicarbonitrile. ¹H NMR (400 MHz, CDCl₃) δ 7.69 (s, 1H), 7.62-7.61 (m, 2H), 3.77-3.75 (m, 1H), 3.07-3.05 (m, 1H), 2.58-2.56 (m, 1H).

### Intermediate 33

### 3-methyl-4-(methylsulfonyl)phenol

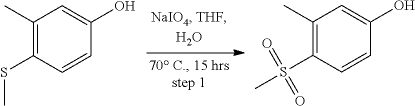

### Step 1: 3-methyl-4-(methylsulfonyl)phenol

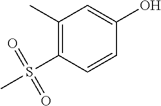

To a solution of 3-methyl-4-methylsulfanyl-phenol (5.00 g, 32.42 mmol) in THF (30 mL) and H₂O (30 mL) was added NaIO₄ (20.80 g, 97.26 mmol) in portions. The mixture was then stirred at 70° C. for 15 h. The reaction mixture was cooled to RT and filtered. The filtrate was cooled to 0° C. and quenched by addition of sat. aq. Na₂SO₃ (30 mL), then extracted with EtOAc (40 mL×3). The combined organic layers were dried over Na₂SO₄, filtered and concentrated under reduced pressure to give 3-methyl-4-methylsulfonyl-phenol, which was taken to the next step without further purification. MS=187.1 [M+H]⁺

### Intermediate 34

### 2-(2-bromo-5-chlorophenyl)acetaldehyde

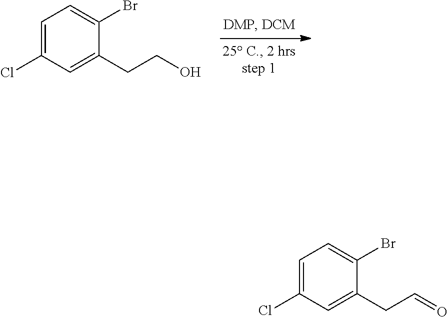 [SMILES: OCCc1cc(Cl)ccc1Br ; O=CCc1cc(Cl)ccc1Br]

### Step 1: 2-(2-bromo-5-chlorophenyl)acetaldehyde

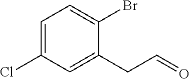 [SMILES: O=CCc1cc(Cl)ccc1Br]

To a mixture of 2-(2-bromo-5-chloro-phenyl)ethanol (1.00 g, 4.25 mmol) in DCM (10 mL) was added Dess-Martin reagent (2.16 g, 5.10 mmol) at 0° C. The mixture was then stirred at RT for 2 h. The reaction mixture was poured into sat. aq. NaHCO₃, and then extracted with DCM (15 mL×3). The combined organic layers were washed with sat. aq. NaHCO₃ (20 mL×2), sat. aq. Na₂SO₃ (20 mL×2), brine (10 mL), dried over Na₂SO₄, filtered and concentrated under reduced pressure to give a residue. The residue was purified by flash silica gel chromatography (ISCO 20 g cartridge, 0-20% EtOAc/Hexane) to give 2-(2-bromo-5-chloro-phenyl)acetaldehyde. ¹H NMR (400 MHz, DMSO-d₆) δ 9.70 (s, 1H), 7.66 (d, J=8.8 Hz, 1H), 7.51 (d, J=2.4 Hz, 1H), 7.32 (d, J=8.4 Hz, 2.4 Hz, 1H), 3.98 (s, 2H).

### Intermediate 35

### 3-(bromomethyl)-5-chlorobenzonitrile

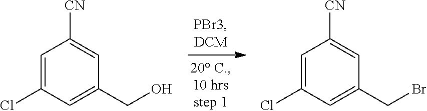 [SMILES: N#Cc1cc(Cl)cc(CO)c1 ; N#Cc1cc(Cl)cc(CBr)c1]

### Step 1: 3-(bromomethyl)-5-chlorobenzonitrile

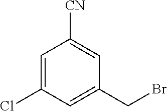 [SMILES: N#Cc1cc(Cl)cc(CBr)c1]

To a solution of 3-chloro-5-(hydroxymethyl)benzonitrile (500 mg, 2.98 mmol) in DCM (10 mL) was added PBr₃ (807 mg, 2.98 mmol, 1) at 0° C. The reaction mixture was then stirred at RT for 10 h, then the reaction mixture was concentrated. The residue was diluted with water (10 mL), neutralized by addition of sat. aq. NaHCO₃ to pH=7 and extracted with EtOAc (10 mL×3). The combined organic layers were washed with brine (20 mL), dried over Na₂SO₄, filtered and concentrated to give 3-(bromomethyl)-5-chloro-benzonitrile, which was taken to the next step without further purification. ¹H NMR (400 MHz, CDCl₃) δ 7.63 (s, 1H), 7.58 (s, 2H), 4.43 (s, 2H).

### Intermediate 36

### 4-((2-hydroxyethyl)sulfonyl)phenol

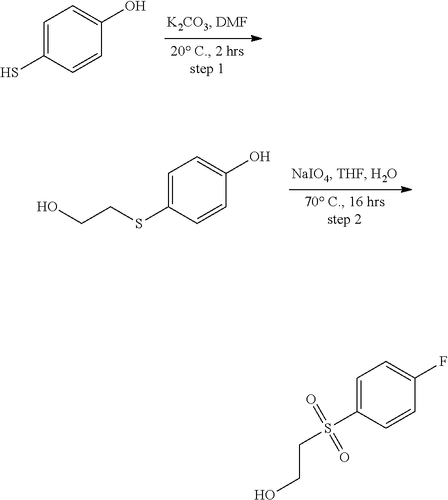 [SMILES: Oc1ccc(S)cc1 ; OCCSc1ccc(O)cc1 ; O=S(=O)(CCO)c1ccc(F)cc1]

### Step 1: 4-((2-hydroxyethyl)thio)phenol

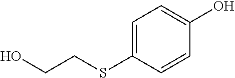 [SMILES: OCCSc1ccc(O)cc1]

To a solution of 4-sulfanylphenol (2.00 g, 15.85 mmol) in DMF (20 mL) was added K₂CO₃ (2.41 g, 17.4 mmol) and 2-bromoethanol (2.18 g, 17.44 mmol). The reaction mixture was then stirred at RT for 2 h. The reaction mixture was poured into water (30 mL) and extracted with EtOAc (30 mL×3). The combined organic layers were washed with brine (50 mL), dried over Na₂SO₄, filtered and concentrated under reduced pressure to give a residue. The residue was purified by flash silica gel chromatography (ISCO 20 g cartridge, 0-100% EtOAc/Hexane) to give 4-(2-hydroxyethylsulfanyl)phenol. MS=169.1 [M−H]⁻

### Step 2: 4-((2-hydroxyethyl)sulfonyl)phenol

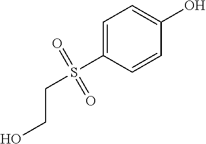 [SMILES: O=S(=O)(CCO)c1ccc(O)cc1]

To a solution of 4-(2-hydroxyethylsulfanyl)phenol (300 mg, 1.76 mmol) in THF (3 mL) and H₂O (3 mL) was added NaIO₄ (377 mg, 1.76 mmol). The mixture was then stirred at 70° C. for 16 h. The reaction mixture was quenched by addition of sat. aq. Na₂SO₃ solution (5 mL) at 0° C., and then extracted with ethyl acetate (5 mL×3). The combined organic layers were washed with brine (20 mL), dried over Na₂SO₄, filtered and concentrated under reduced pressure to give a residue. The residue was purified by preparative SiO₂ TLC (1:2 PE/EtOAc) to give 4-(2-hydroxyethylsulfonyl)phenol. MS=201.1 [M−H]⁻

### Intermediate 37

### 3-((4-fluorophenyl sulfonyl-3-methyloxetane

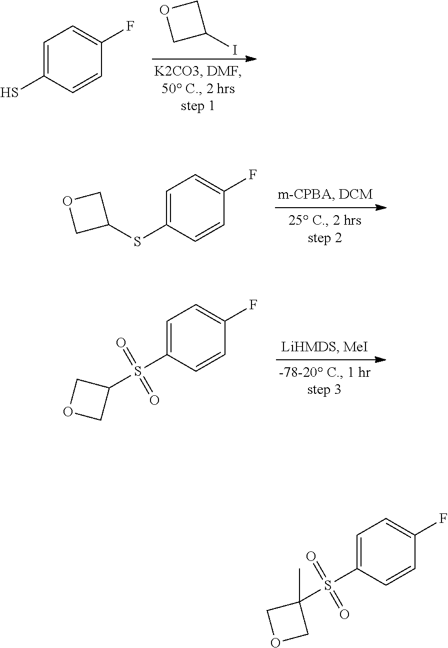

### Step 1: 3-((4-fluorophenyl)thio)oxetane

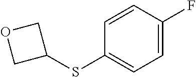 [SMILES: Fc1ccc(SC2COC2)cc1]

To a solution of 4-fluorobenzenethiol (3.00 g, 23.41 mmol) and 3-iodooxetane (4.74 g, 25.75 mmol) in DMF (30 mL) was added K₂CO₃ (6.47 g, 46.81 mmol). The mixture was then stirred at 50° C. for 2 h. The residue was poured into ice-water (150 mL) and stirred for 5 min. The aqueous phase was extracted with ethyl acetate (50 mL×3). The combined organic phase was washed with brine (10 mL), dried with anhydrous Na₂SO₄, filtered and concentrated under reduced pressure. The residue was purified by flash silica gel chromatography (ISCO 40 g cartridge, 0-10% EtOAc/Hexane) to give 3-(4-fluorophenyl)sulfanyloxetane. ¹H NMR (400 MHz, DMSO-d₆) δ 7.26-7.23 (m, 2H), 7.00-6.96 (m, 2H), 4.97-4.92 (m, 2H), 4.60-4.57 (m, 2H), 4.38-4.31 (m, 1H).

### Step 2: 3-((4-fluorophenyl)sulfonyl)oxetane

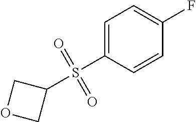 [SMILES: O=S(=O)(c1ccc(F)cc1)C1COC1]

To a solution of 3-(4-fluorophenyl)sulfanyloxetane (2.90 g, 15.74 mmol) in DCM (20 mL) was added m-CPBA (7.99 g, 39.35 mmol, 85% purity). The mixture was stirred at RT for 2 h. The reaction mixture was quenched by addition of H₂O (20 mL), and then extracted with DCM (20 mL×3). The combined organic layers were washed with sat. aq. Na₂SO₃ (60 mL) and brine (60 mL), dried over Na₂SO₄, filtered and concentrated under reduced pressure to give a residue. The residue was purified by flash silica gel chromatography (ISCO 40 g cartridge, 0-50% EtOAc/Hexane) to give 3-(4-fluorophenyl)sulfonyloxetane. ¹H NMR (400 MHz, DMSO-d₆) δ 8.03-7.99 (m, 2H), 7.55-7.51 (m, 2H), 4.93-4.90 (m, 1H), 4.78-4.70 (m, 4H).

### Step 3: 3-((4-fluorophenyl)sulfonyl)-3-methyloxetane

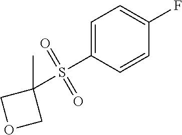

To a solution of 3-(4-fluorophenyl)sulfonyloxetane (3.50 g, 16.2 mmol) in THE (30 mL) at −78° C. was added LiHMDS (1 M in THF, 32.37 mL) dropwise. The mixture was stirred at this temperature for 1 h, then MeI (3.45 g, 24.28 mmol) was added dropwise at −78° C. The resulting mixture was stirred at RT for 1 h. The reaction mixture was quenched by addition of sat. aq. NH₄Cl solution (50 mL) and extracted with EtOAc (50 mL×3). The combined organic layers were washed with brine (100 mL), dried over Na₂SO₄, filtered and concentrated under reduced pressure to give a residue. The residue was purified by flash silica gel chromatography (ISCO 40 g cartridge, 0-50% EtOAc:Hexane) to give 3-(4-fluorophenyl)sulfonyl-3-methyl-oxetane. ¹H NMR (400 MHz, DMSO-d₆) δ 8.03-7.99 (m, 2H), 7.55-7.51 (m, 2H), 4.95 (d, J=7.2 Hz, 2H), 4.46 (d, J=7.2 Hz, 2H), 1.53 (s, 3H).

### Intermediate 38

### 4-((3-(methylsulfonyl)propyl)sulfonyl)phenol

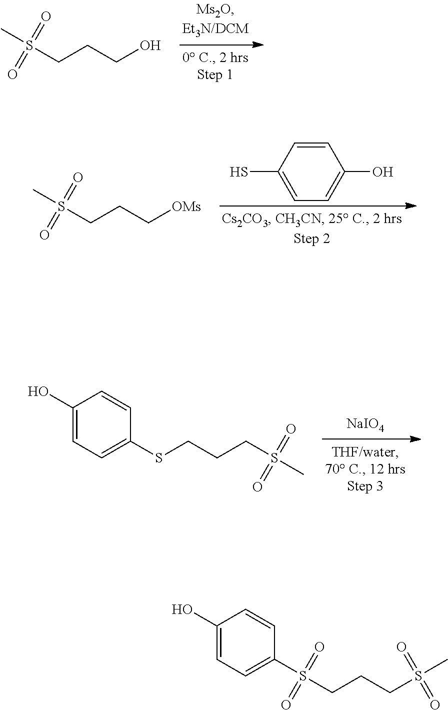

### Step 1: 3-(methylsulfonyl)propyl methanesulfonate

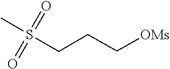

To a solution of 3-methylsulfonylpropan-1-ol (500 mg, 3.62 mmol) in DCM (5 mL) at 0° C. were added Et₃N (732 mg, 7.24 mmol) and methylsulfonyl methanesulfonate (945 mg, 5.43 mmol). The mixture was stirred at 0° C. for 2 h. The reaction mixture was diluted with H₂O (20 mL) and extracted with EtOAc (10 mL×3). The combined organic layers were washed with brine (30 mL), dried over Na₂SO₄, filtered and concentrated under reduced pressure to give crude 3-(methylsulfonyl)propyl methanesulfonate, which was taken to the next step without further purification. ¹H NMR (400 MHz, DMSO-d₆) δ 4.30 (t, J=6.4 Hz, 2H), 3.23-3.01 (m, 5H), 3.01 (s, 3H), 2.13-2.08 (m, 2H).

### Step 2: 4-((3-(methylsulfonyl)propyl)thio)phenol

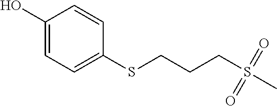

To a mixture of 3-(methylsulfonyl)propyl methanesulfonate (390 mg, 1.80 mmol) and 4-sulfanylphenol (318 mg, 2.52 mmol) in CH₃CN (5 mL) was added Cs₂CO₃ (705 mg, 2.16 mmol). The mixture was then stirred at RT for 2 h. The reaction mixture was then diluted with H₂O (20 mL) and extracted with EtOAc (20 mL×3). The combined organic layers were washed with brine (40 mL), dried over Na₂SO₄, filtered and concentrated under reduced pressure to give a residue. The residue was purified by flash silica gel chromatography (ISCO 12 g cartridge, 0-50% EtOAc/Hexane) to give 4-(3-methylsulfonylpropylsulfanyl)phenol. MS=245.1 [M−H]⁻

### Step 3: 4-((3-(methylsulfonyl)propyl)sulfonyl)phenol

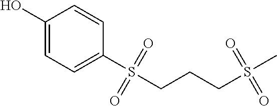 [SMILES: CS(=O)(=O)CCCS(=O)(=O)c1ccc(O)cc1]

To a solution of 4-(3-methylsulfonylpropylsulfanyl)phenol (370 mg, 1.50 mmol) in THE (2 mL) and H₂O (2 mL) was added NaIO₄ (964 mg, 4.51 mmol) at 0° C. The mixture was then stirred at 70° C. for 12 h. The reaction mixture was quenched by addition of sat. aq. Na₂SO₃ (10 mL), and then diluted with H₂O (5 mL) and extracted with ethyl acetate (15 mL×3). The combined organic layers were washed with brine (30 mL), dried over Na₂SO₄, filtered and concentrated under reduced pressure to give a residue. The residue was purified by flash silica gel chromatography (ISCO 12 g cartridge, 0-50% EtOAc/Hexane) to give 4-(3-methylsulfonylpropylsulfonyl)phenol. MS=277.1 [M−H]⁻

### Intermediate 39

### 4-(methylsulfonyl)-3-(trifluoromethyl)phenol

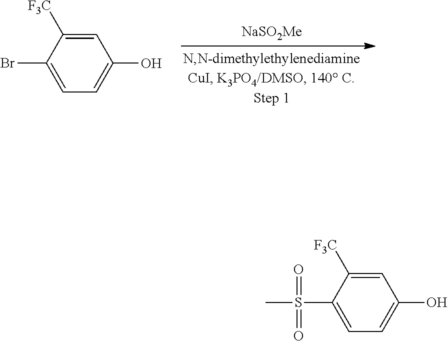

### Step 1: 4-(methylsulfonyl)-3-(trifluoromethyl)phenol

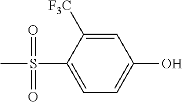

To a solution of 4-bromo-3-(trifluoromethyl)phenol (1.00 g, 4.15 mmol) and sodium methanesulfinate (4.24 g, 41.5 mmol) in DMSO (10 mL) were added CuI (79 mg, 415 umol), N,N-dimethylethane-1,2-diamine (73 mg, 829 umol) and K₃PO₄ (176 mg, 829 umol). The mixture was then stirred at 140° C. for 16 h. The reaction mixture was poured into water (20 mL) and extracted with EtOAc (20 mL×3). The combined organic layers were washed with brine (50 mL), dried over Na₂SO₄, filtered and concentrated under reduced pressure to give a residue. The residue was purified by flash silica gel chromatography (Biotage 12 g cartridge, 0-100% EtOAc/Hexane) to give 4-methylsulfonyl-3-(trifluoromethyl)phenol. MS=239.1 [M−H]⁻

### Intermediate 40

### 3-(2-oxoethyl)benzonitrile

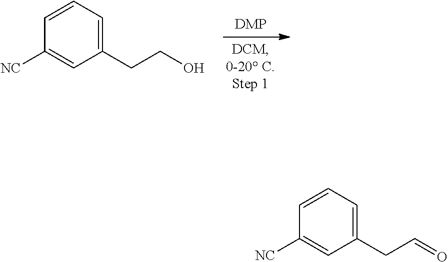 [SMILES: N#Cc1cccc(CCO)c1 ; N#Cc1cccc(CC=O)c1]

### Step 1: 3-(2-oxoethyl)benzonitrile

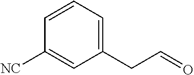 [SMILES: N#Cc1cccc(CC=O)c1]

To a solution of 3-(2-hydroxyethyl)benzonitrile (3.00 g, 20.4 mmol) in DCM (30 mL) was added DMP (8.65 g, 20.38 mmol) at 0° C. The reaction mixture was then stirred at RT for 2 h. The reaction was diluted with water (30 mL), adjusted pH to 7-8 with sat. aq. NaHCO₃, and then extracted with DCM (30 mL×3). The combined organic layers were washed with brine (100 mL), dried over Na₂SO₄, filtered and the filtrate was concentrated to give a residue. The residue was purified by flash silica gel chromatography (Biotage 20 g cartridge, 0-100% EtOAc/Hexane) to give 3-(2-oxoethyl)benzonitrile. ¹H NMR (400 MHz, CDCl₃) δ 9.80 (t, J=2.0 Hz, 1H), 7.63-7.52 (m, 4H), 3.92-3.88 (m, 2H).

### Intermediate 41

### N-(2-((4-hydroxyphenyl)sulfonyl)ethyl)-N-methylacetamide

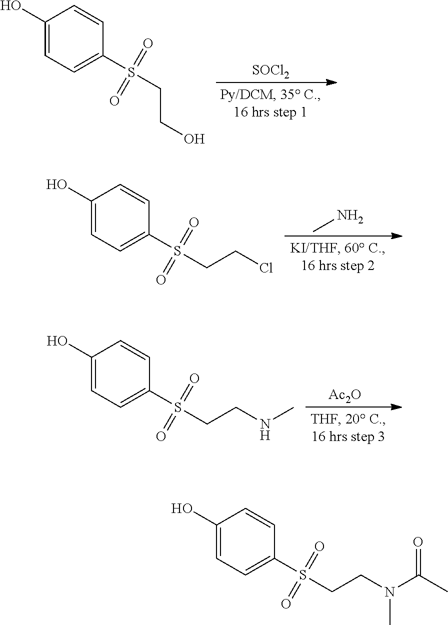

### Step 1: 4-((2-chloroethyl)sulfonyl)phenol

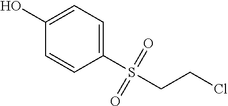 [SMILES: O=S(=O)(CCCl)c1ccc(O)cc1]

To a mixture of 4-(2-hydroxyethylsulfonyl)phenol0 (1.1 g, 5.44 mmol) and pyridine (860 mg, 10.9 mmol) in DCM (10 mL) was added SOCl₂ (1.94 g, 16.32 mmol) at 0° C. The mixture was then stirred at 35° C. for 16 h. The reaction mixture was cooled to RT and H₂O (20 mL) was added. The mixture was extracted with DCM (20 mL×3). The combined organic layers were washed with brine (60 mL), dried over Na₂SO₄, filtered and the filtrate was concentrated under reduced pressure. The residue was purified by flash silica gel chromatography (Biotage 20 g cartridge, 0-100% EtOAc/Hexane) to give 4-(2-chloroethylsulfonyl)phenol. ¹H NMR (400 MHz, CDCl₃) δ 7.84-7.80 (m, 2H), 7.02-6.98 (m, 2H), 5.55 (br s, 1H), 3.77-3.74 (m, 2H), 3.53-3.49 (m, 2H).

### Step 2: 4-((2-(methylamino)ethyl)sulfonyl)phenol

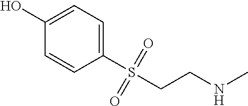

A mixture of 4-(2-chloroethylsulfonyl)phenol (750 mg, 3.40 mmol), methylamine (2 M in THF, 22.1 mL) and KI (225 mg, 1.36 mmol) in THF (2 mL) was stirred at 60° C. for 16 h under N₂. The mixture was filtered and the filtrate was concentrated under reduced pressure to give 4-((2 (methylamino)ethyl)sulfonyl)phenol, which was taken to the next step without further purification. MS=216.2 [M+H]⁺

### Step 3: N-(2-((4-hydroxyphenyl)sulfonyl)ethyl)-N-methylacetamide

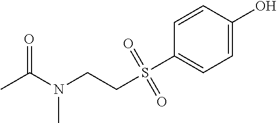 [SMILES: CC(=O)N(C)CCS(=O)(=O)c1ccc(O)cc1]

A mixture of 4-[2-(methylamino)ethylsulfonyl]phenol (1.1 g, 5.11 mmol) and Ac₂O (521 mg, 5.11 mmol) in THE (10 mL) was stirred at RT for 16 h. The mixture was filtered and the filtered cake was further purified by flash silica gel chromatography (Biotage 20 g cartridge, 0-50% EtOAc/Hexane) to N-(2-((4-hydroxyphenyl)sulfonyl)ethyl)-N-methylacetamide. MS=258.2 [M+H]⁺

### Intermediate 42

### tert-butyl (3S,4S)-3-methyl-4-(((methylsulfonyl)oxy)methyl)pyrrolidine-1-carboxylate

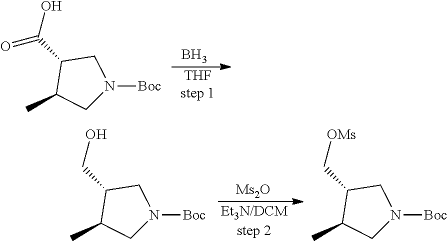

### Step 1: tert-butyl (3S,4S)-3-(hydroxymethyl)-4-methylpyrrolidine-1-carboxylate

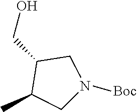

To a solution of (3S,4S)-1-tert-butoxycarbonyl-4-methyl-pyrrolidine-3-carboxylic acid (5.00 g, 21.81 mmol) in THE (50 mL) was added BH₃ (10 M in Me₂S, 10.90 mL, 109 mmol) at 0° C. Then the reaction was stirred at RT for 16 h. The reaction mixture was quenched by addition of MeOH (40 mL) and stirred for 0.5 h. The mixture was then diluted with water (100 mL) and extracted with EtOAc (60 mL×3). The combined organic layers were washed with brine (100 mL), dried over Na₂SO₄, filtered and the filtrate was concentrated to give tert-butyl (3S,4S)-3-(hydroxymethyl)-4-methyl-pyrrolidine-1-carboxylate. MS=160.2 [M−C₄H₈+H]⁺.

### Step 2: tert-butyl (3S,4S)-3-methyl-4-(((methylsulfonyl)oxy)methyl)pyrrolidine-1-carboxylate

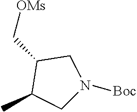 [SMILES: C[C@@H]1CN(C(=O)OC(C)(C)C)C[C@H]1COS(C)(=O)=O]

To a solution of tert-butyl (3S,4S)-3-(hydroxymethyl)-4-methyl-pyrrolidine-1-carboxylate (200 mg, 929 umol) in DCM (3 mL) were added Et₃N (188 mg, 1.86 mmol) and methylsulfonyl methanesulfonate (161 mg, 929 umol) at 0° C. and then the mixture was stirred at RT for 2 h. The reaction mixture was then concentrated under reduced pressure. The residue was diluted with sat. aq. NH₄Cl (5 mL), extracted with EtOAc (5 mL×3), dried over Na₂SO₄, filtered and concentrated under reduced pressure to give tert-butyl (3S,4S)-3-methyl-4-(((methylsulfonyl)oxy)methyl)pyrrolidine-1-carboxylate. ¹H NMR (400 MHz, CDCl₃) δ 4.32-4.28 (m, 1H), 4.16-4.14 (m, 1H), 3.65-3.61 (m, 3H), 3.20-3.18 (m, 1H), 3.03 (s, 3H), 2.97-2.95 (m, 1H), 2.33-2.20 (m, 4H), 1.46 (s, 9H).

### Intermediate 43

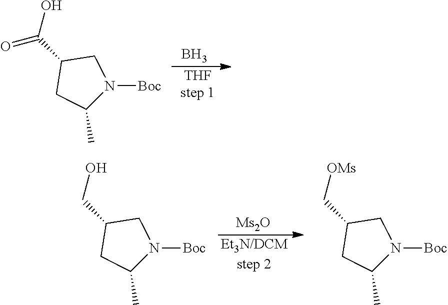

### Step 1: tert-butyl (2R,4S)-4-(hydroxymethyl)-2-methylpyrrolidine-1-carboxylate

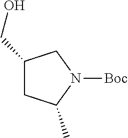

To a solution of (3S,5R)-1-tert-butoxycarbonyl-5-methyl-pyrrolidine-3-carboxylic acid (5.00 g, 21.81 mmol) in THE (30 mL) was added BH₃ (1 M in THF, 34.9 mL, 34.9 mmol) at 0° C. The mixture was stirred at RT for 2 h under N₂. The reaction mixture was quenched by addition of MeOH (30 mL) and stirred at 0° C. for 20 min. Then mixture was diluted with water (60 mL) and extracted with ethyl acetate (30 mL×3). The combined organic layers were washed with sat. aq. NaHCO₃ (60 mL) and brine (60 mL), dried over Na₂SO₄, filtered and concentrated under reduced pressure to give tert-butyl (2R,4S)-4-(hydroxymethyl)-2-methyl-pyrrolidine-1-carboxylate, which was used in the next step without further purification. MS=160.2 [M−C₄H₈+H]⁺.

### Step 2: tert-butyl (2R,4S)-2-methyl-4-(((methylsulfonyl)oxy)methyl)pyrrolidine-1-carboxylate

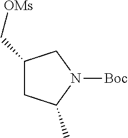 [SMILES: C[C@@H]1C[C@H](COS(C)(=O)=O)CN1C(=O)OC(C)(C)C]

To a solution of tert-butyl (2R,4S)-4-(hydroxymethyl)-2-methyl-pyrrolidine-1-carboxylate (3.50 g, 16.26 mmol) in DCM (50 mL) were added Et₃N (4.94 g, 48.77 mmol) and methylsulfonyl methanesulfonate (3.40 g, 19.51 mmol). The reaction mixture was stirred at RT for 2 h. The reaction was concentrated under reduced pressure and then the residue was diluted with sat. aq. NH₄Cl (30 mL) and extracted with EtOAc (30 mL×3). The combined organic layers were washed with brine (100 mL), dried over Na₂SO₄, filtered and the filtrate was concentrated to give tert-butyl (2R,4S)-2-methyl-4-(methylsulfonyloxymethyl)pyrrolidine-1-carboxylate, which was used in the next step without further purification. ¹H NMR (400 MHz, DMSO-d₆) δ 4.24-4.14 (m, 2H), 3.73-3.71 (m, 1H), 3.63-3.58 (m, 1H), 3.18 (s, 3H), 2.96-2.92 (m, 1H), 2.25-2.23 (m, 1H), 2.21-2.18 (m, 1H), 1.86 (d, J=3.2 Hz, 3H), 1.39 (s, 9H), 1.32-1.27 (m, 1H).

### Example 1

### 3-{2-[(2R,4S)-4-{[(5-methanesulfonylpyrazin-2-yl)oxy]methyl}-2-methylpyrrolidin-1-yl]ethyl}(Compound 1)

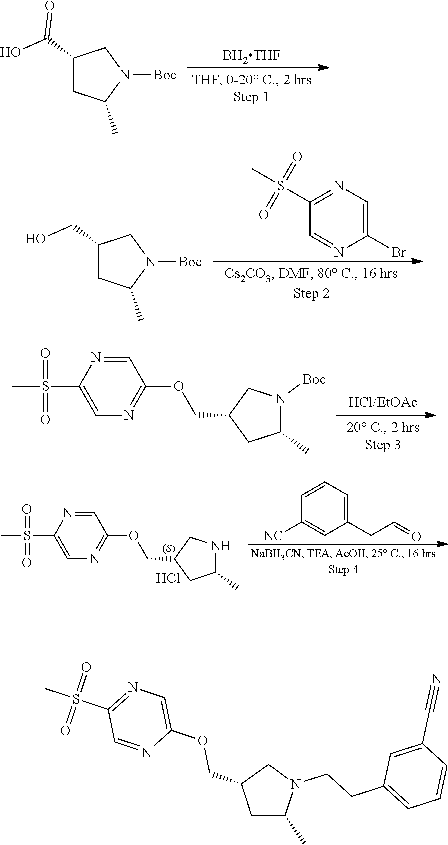

### Step 1: (2R,4S)-tert-butyl 4-(hydroxymethyl)-2-methylpyrrolidine-1-carboxylate

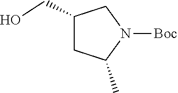

To a solution of (3S,5R)-1-(tert-butoxycarbonhylpyrrolidine-3-carboxylic acid (250 mg, 1.09 mmol) in THE (5 mL) at 0° C. was added BH₃·THF (1 M in THF, 1.74 mL), and the mixture was stirred at RT for 2 h. The reaction mixture was then cooled to 0° C. and quenched by addition of aq. HCl (0.5 M, 2 mL), then stirred at 0° C. for 20 min. The mixture was then diluted with water (10 mL) and extracted with ethyl acetate (3 mL×3). The combined organic layers were washed with sat. aq. NaHCO₃ (3 mL) and brine (3 mL), dried over Na₂SO₄, filtered and concentrated under reduced pressure to give (2R,4S)-tert-butyl 4-(hydroxymethyl)-2-methylpyrrolidine-1-carboxylate, which was used without further purification. MS=160.1 [M−C₄H₈+H]⁺.

### Step 2: (2R,4S)-tert-butyl 2-methyl-4-(((5-(methylsulfonyl)pyrazin-2-yl)oxy)methyl)pyrrolidine-1-carboxylate

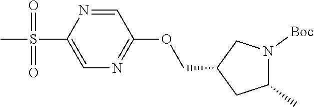 [SMILES: C[C@@H]1C[C@H](COc2cnc(S(C)(=O)=O)cn2)CN1C(=O)OC(C)(C)C]

To a mixture of (2R,4S)-tert-butyl 4-(hydroxymethyl)-2-methyl-pyrrolidine-1-carboxylate (50 mg, 232 umol) and 2-bromo-5-(methylsulfonyl)pyrazine (110 mg, 464 umol) in DMF (2 mL) was added Cs₂CO₃ (151 mg, 464 umol). The mixture was stirred at 80° C. for 16 h. The reaction mixture was diluted with H₂O (10 mL) and extracted with EtOAc (5 mL×3). The combined organic layers were washed with H₂O (5 mL×2) and brine (5 mL), dried over Na₂SO₄, filtered and concentrated under reduced pressure to give a residue. The residue was purified by preparative TLC SiO₂ (EtOAc:PE=1:1) to give (2R,4S)-tert-butyl 2-methyl-4-(((5-(methylsulfonyl)pyrazin-2-yl)oxy)methyl)pyrrolidine-1-carboxylate. MS=316.3 [M−C₄H₈+H]⁺.

### Step 3: 2-(((3S,5R)-5-methylpyrrolidin-3-yl)methoxy)-5-(methylsulfonyl)pyrazine

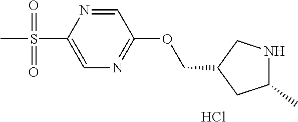

To a solution of (2R,4S)-tert-butyl 2-methyl-4-(((5-(methylsulfonyl)pyrazin-2-yl)oxy)methyl)pyrrolidine-1-carboxylate (100 mg, 269 umol) in EtOAc (1 mL) was added HCl in EtOAc (4 M, 3 mL). The mixture was stirred at RT for 2 h. The reaction mixture was then concentrated under reduced pressure to give 2-(((3S,5R)-5-methylpyrrolidin-3-yl)methoxy)-5-(methylsulfonyl)pyrazine HCL salt. MS=272.2 [M+H]⁺.

### Step 4: 3-{2-[(2R,4S)-4-{[(5-methanesulfonylpyrazin-2-yl)oxy]methyl}-2-methylpyrrolidin-1-yl]ethyl}benzonitrile

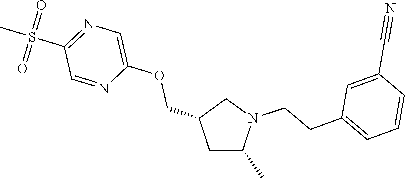 [SMILES: C[C@@H]1C[C@H](COc2cnc(S(C)(=O)=O)cn2)CN1CCc1cccc(C#N)c1]

To a mixture of 2-(((3S,5R)-5-methylpyrrolidin-3-yl)methoxy)-5-(methylsulfonyl)pyrazine (100 mg, 324 umol, HCl salt) in MeOH (4 mL) were added TEA (32 mg, 325 umol), 3-(2-oxoethyl)benzonitrile (61 mg, 422 umol) and AcOH (19 mg, 325 umol). The mixture was stirred at RT for 2 h, followed by addition of NaBH₃CN (40 mg, 650 umol). The mixture was stirred at RT for 14 h. The reaction mixture was then diluted with H₂O (10 mL) and extracted with EtOAc (5 mL×3). The combined organic layers were washed with brine (5 mL), dried over Na₂SO₄, filtered and concentrated under reduced pressure to give a residue. The residue was purified by preparative reverse phase IPLC (Waters Xbridge BEH C18, 40-70% MeCN:10 mM N₄HCO₃ in H₂O) to give 3-(2-((2R,4S)-2-methyl-4-(((5-(methylsulfonyl)pyrazin-2-yl)oxy)methyl)pyrrolidin-1-yl)ethyl)benzonitrile (Compound 1) ¹H NMR (DMSO-d₆, 400 MHz) δ 8.77 (d, J=1.2 Hz, 1H), 8.47 (d, J=1.2 Hz, 1H), 7.70-7.62 (m, 1H), 7.62-7.57 (m, 2H), 7.46-7.42 (m, 1H), 4.23 (d, J=7.6 Hz, 2H), 3.25 (s, 3H), 3.09-3.06 (m, 1H), 3.00-2.93 (m, 1H), 2.83-2.71 (m, 2H), 2.38-2.20 (m, 4H), 2.15-2.09 (m, 1H), 1.08-1.02 (m, 1H), 0.96 (d, J=6.0 Hz, 3H). MS=401.2 [M+H]⁺.

The following compounds in Table 2 were prepared according to procedures similar to those described for Example 1 using the appropriate starting materials.

TABLE 2

| | | | | | Chiral |
|---|---|---|---|---|---|
| | | | Exact | Chiral | purification |
| | | | Mass | purification | Elution |
| # | Structure | IUPAC Name | [M + H]⁺ | column | order |
| 2 | 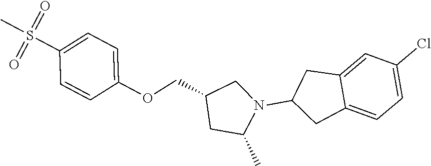 [SMILES: C[C@@H]1C[C@H](COc2ccc(S(C)(=O)=O)cc2)CN1C1Cc2ccc(Cl)cc2C1] | (2R,4S)-1-[5- chloro-2,3- dihydro-1H-inden- 2-yl]-4-[(4- methanesulfonyl- phenoxy)methyl]-2- methylpyrrolidine | Calc'd 420.1 Found 420.2 | n/a | |
| 3 | 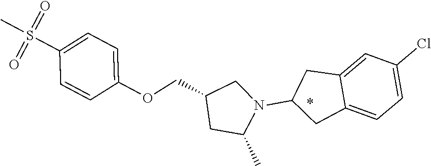 [SMILES: C[C@@H]1C[C@H](COc2ccc(S(C)(=O)=O)cc2)CN1C1Cc2ccc(Cl)cc2C1] | (2R,4S)-1-[(2R or 2S)-5-chloro-2,3- dihydro-1H-inden- 2-yl]-4-[(4- methanesulfonyl- phenoxy)methyl]-2- methylpyrrolidine | Calc'd 420.1 Found 420.2 | DAICEL CHIRALCEL OD | 1st |
| 4 | 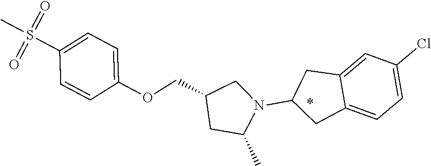 [SMILES: C[C@@H]1C[C@H](COc2ccc(S(C)(=O)=O)cc2)CN1C1Cc2ccc(Cl)cc2C1] | (2R,4S)-1-[(2S or 2R)-5-chloro-2,3- dihydro-1H-inden- 2-yl]-4-[(4- methanesulfonyl- phenoxy)methyl]-2- methylpyrrolidine | Calc'd 420.1 Found 420.2 | DAICEL CHIRALCEL OD | 2nd |
| 5 | 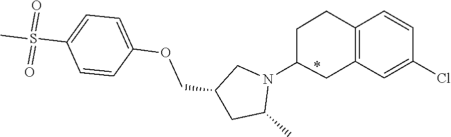 [SMILES: C[C@@H]1C[C@H](COc2ccc(S(C)(=O)=O)cc2)CN1C1CCc2ccc(Cl)cc2C1] | (2R,4S)-1-[(2S or 2R)-7-chloro- 1,2,3,4- tetrahydro- naphthalen-2- yl]-4-[(4- methanesulfonyl- phenoxy)methyl]-2- methylpyrrolidine | Calc'd 434.1 Found 434.2 | DAICEL CHIRALPAK IG | 1st |
| 6 | 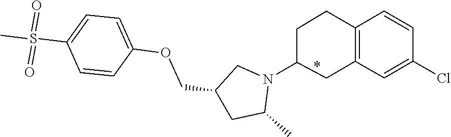 [SMILES: C[C@@H]1C[C@H](COc2ccc(S(C)(=O)=O)cc2)CN1C1CCc2ccc(Cl)cc2C1] | (2R,4S)-1-[(2R or 2S)-7-chloro- 1,2,3,4- tetrahydro- naphthalen-2- yl]-4-[(4- methanesulfonyl- phenoxy)methyl]-2- methylpyrrolidine | Calc'd 434.1 Found 434.2 | DAICEL CHIRALPAK IG | 2nd |
| 7 | 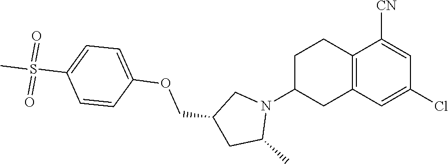 [SMILES: C[C@@H]1C[C@H](COc2ccc(S(C)(=O)=O)cc2)CN1C1CCc2c(C#N)cc(Cl)cc2C1] | 3-chloro-6- [(2R,4S)-4-[(4- methanesulfonyl- phenoxy)methyl]-2- methylpyrrolidin- 1-yl]-5,6,7,8- tetrahydro- naphthalene- 1-carbonitrile | Calc'd 459.1 Found 459.1 | n/a | |
| 8 | 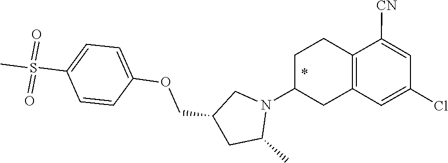 [SMILES: C[C@@H]1C[C@H](COc2ccc(S(C)(=O)=O)cc2)CN1C1CCc2c(C#N)cc(Cl)cc2C1] | (6R or 6S)-3- chloro-6-[(2R,4S)- 4-[(4- methanesulfonyl- phenoxy)methyl]-2- methylpyrrolidin- 1-yl]-5,6,7,8- tetrahydro- naphthalene- 1-carbonitrile | Calc'd 459.1 Found 459.1 | DAICEL CHIRALPAK AD | 1st |
| 9 | 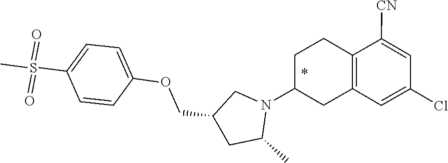 [SMILES: C[C@@H]1C[C@H](COc2ccc(S(C)(=O)=O)cc2)CN1C1CCc2c(C#N)cc(Cl)cc2C1] | (6S or 6R)-3- chloro-6-[(2R,4S)- 4-[(4- methanesulfonyl- phenoxy)methyl]-2- methylpyrrolidin- 1-yl]-5,6,7,8- tetrahydronaphthal ene-1-carbonitrile | Calc'd 459.1 Found 459.1 | DAICEL CHIRALPAK AD | 2nd |
| 10 | 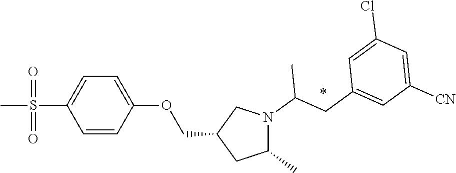 [SMILES: CC(Cc1cc(Cl)cc(C#N)c1)N1C[C@@H](COc2ccc(S(C)(=O)=O)cc2)C[C@H]1C] | 3-chloro-5-[(2S or 2R)-2-[(2R,4S)-4- [(4- methanesulfonyl- phenoxy)methyl]-2- methylpyrrolidin- 1-yl]propyl] benzonitrile | Calc'd 447.1 Found 447.0 | prep- HPLC Waters Xbridge BEH C18 | 1st |
| 11 | 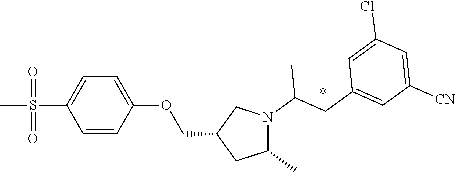 [SMILES: CC(Cc1cc(Cl)cc(C#N)c1)N1C[C@@H](COc2ccc(S(C)(=O)=O)cc2)C[C@H]1C] | 3-chloro-5-[(2R or 2S)-2-[(2R,4S)-4- [(4- methanesulfonyl- phenoxy)methyl]-2- methylpyrrolidin- 1-yl]propyl] benzonitrile | Calc'd 447.1 Found 447.0 | prep- HPLC Waters Xbridge BEH C18 | 2nd |
| 12 | 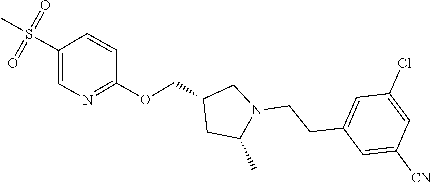 [SMILES: C[C@@H]1C[C@H](COc2ccc(S(C)(=O)=O)cn2)CN1CCc1cc(Cl)cc(C#N)c1] | 3-chloro-5-{2- [(2R,4S)-4-{[(5- methanesulfonyl- pyridin-2- yl)oxy]methyl}-2- methylpyrrolidin- 1-yl]ethyl} benzonitrile | Calc'd 434.1 Found 434.1 | n/a | |
| 13 | 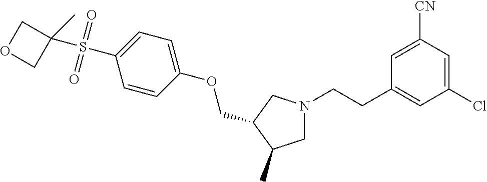 [SMILES: C[C@@H]1CN(CCc2cc(Cl)cc(C#N)c2)C[C@H]1COc1ccc(S(=O)(=O)C2(C)COC2)cc1] | 3-chloro-5-{2- [(3S,4S)-3-methyl- 4-({4-[(3- methyloxetan-3- yl)sulfonyl] phenoxy}methyl) pyrrolidin-1- yl]ethyl} benzonitrile | Calc'd 489.1 Found 489.2 | n/a | |
| 14 | 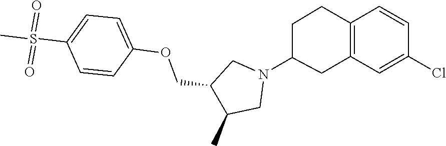 [SMILES: C[C@@H]1CN(C2CCc3ccc(Cl)cc3C2)C[C@H]1COc1ccc(S(C)(=O)=O)cc1] | (3S,4S)-1-[7- chloro-1,2,3,4- tetrahydro- naphthalen- 2-yl]-3-[(4- methanesulfonyl- phenoxy) methyl]-4- methylpyrrolidine | Calc'd 434.2 Found 434.2 | n/a | |
| 15 | 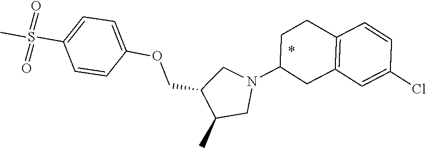 [SMILES: C[C@@H]1CN(C2CCc3ccc(Cl)cc3C2)C[C@H]1COc1ccc(S(C)(=O)=O)cc1] | (3S,4S)-1-[(2S or 2R)-7-chloro- 1,2,3,4- tetrahydro- naphthalen-2- yl]-3-[(4- methanesulfonyl- phenoxy) methyl]-4- methylpyrrolidine | Calc'd 434.2 Found 434.1 | DAICEL CHIRALPAK AD-H | 1st |
| 16 | 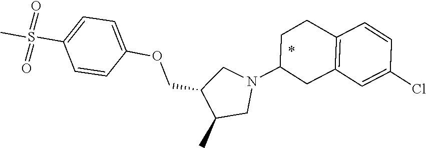 [SMILES: C[C@@H]1CN(C2CCc3ccc(Cl)cc3C2)C[C@H]1COc1ccc(S(C)(=O)=O)cc1] | (3S,4S)-1-[(2R or 2S)-7-chloro- 1,2,3,4- tetrahydro- naphthalen-2- yl]-3-[(4- methanesulfonyl- phenoxy) methyl]-4- methylpyrrolidine | Calc'd 434.2 Found 434.1 | DAICEL CHIRALPAK AD-H | 2nd |
| 17 | 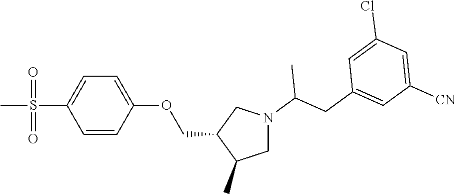 [SMILES: CC(Cc1cc(Cl)cc(C#N)c1)N1C[C@@H](COc2ccc(S(C)(=O)=O)cc2)[C@H](C)C1] | 3-chloro-5-[2- [(3S,4S)-3-[(4- methanesulfonyl- phenoxy) methyl]-4- methylpyrrolidin- 1-yl]propyl] benzonitrile | Calc'd 447.2 Found 447.1 | n/a | |
| 18 | 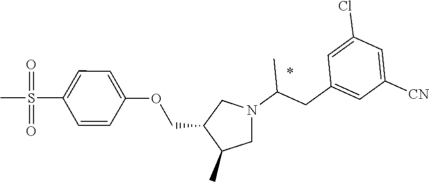 [SMILES: CC(Cc1cc(Cl)cc(C#N)c1)N1C[C@@H](COc2ccc(S(C)(=O)=O)cc2)[C@H](C)C1] | 3-chloro-5-[(2S or 2R)-2-[(3S,4S)-3- [(4- methanesulfonyl- phenoxy) methyl]-4- methylpyrrolidin- 1-yl]propyl] benzonitrile | Calc'd 447.2 Found 447.1 | DAICEL CHIRALPAK AD | 1st |
| 19 | 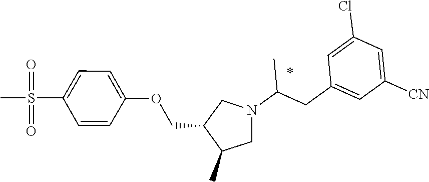 [SMILES: CC(Cc1cc(Cl)cc(C#N)c1)N1C[C@@H](COc2ccc(S(C)(=O)=O)cc2)[C@H](C)C1] | 3-chloro-5-[(2R or 2S)-2-[(3S,4S)- 3-[(4- methanesulfonyl- phenoxy) methyl]-4- methylpyrrolidin- 1-yl]propyl] benzonitrile | Calc'd 447.2 Found 447.1 | DAICEL CHIRALPAK AD | 2nd |
| 20 | 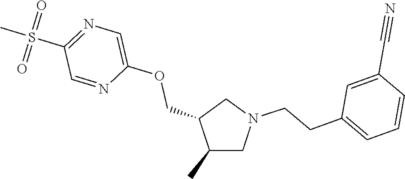 [SMILES: C[C@@H]1CN(CCc2cccc(C#N)c2)C[C@H]1COc1cnc(S(C)(=O)=O)cn1] | 3-{2-[(35,4S)-3- {[(5- methanesulfonyl pyrazin-2- yl)oxy]methyl}-4- methylpyrrolidin- 1-yl]ethyl} benzonitrile | Calc'd 401.2 Found 401.2 | n/a | |
| 21 | 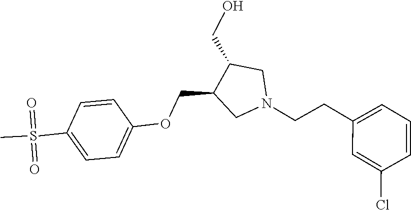 [SMILES: CS(=O)(=O)c1ccc(OC[C@@H]2CN(CCc3cccc(Cl)c3)C[C@H]2CO)cc1] | [(3S,4S)-1-[2-(3- chlorophenyl) ethyl 1-4-[(4- methanesulfonyl- phenoxy)methyl] pyrrolidin-3- yl]methanol | Calc'd 424.1 Found 424.1 | n/a | |
| 22 | 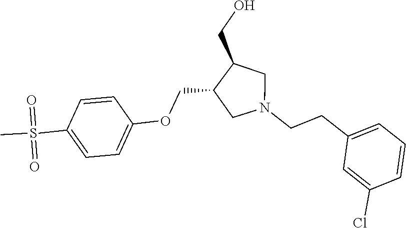 [SMILES: CS(=O)(=O)c1ccc(OC[C@H]2CN(CCc3cccc(Cl)c3)C[C@@H]2CO)cc1] | [(3R,4R)-1-[2-(3- chlorophenyl) ethyl]-4-[(4- methanesulfonyl- phenoxy)methyl] pyrrolidin-3- yl]methanol | Calc'd 424.1 Found 424.2 | n/a | |
| 23 | 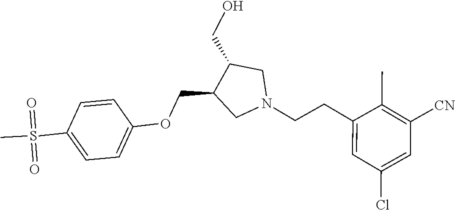 [SMILES: Cc1c(C#N)cc(Cl)cc1CCN1C[C@@H](CO)[C@H](COc2ccc(S(C)(=O)=O)cc2)C1] | 5-chloro-3-{2- [(3S,4S)-3- (hydroxymethyl)- 4-[(4- methanesulfonyl- phenoxy)methyl] pyrrolidin-1- yl]ethyl}-2- methylbenzonitrile | Calc'd 463.1 Found 463.0 | n/a | |
| 24 | 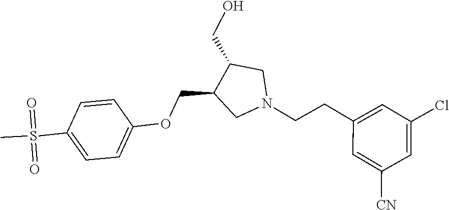 [SMILES: CS(=O)(=O)c1ccc(OC[C@@H]2CN(CCc3cc(Cl)cc(C#N)c3)C[C@H]2CO)cc1] | 3-chloro-5-{2- [(3S,4S)-3- (hydroxymethyl)- 4-[(4- methanesulfonyl phenoxy)methyl] pyrrolidin-1- yl]ethyl} benzonitrile | Calc'd 449.1 Found 449.1 | n/a | |
| 25 | 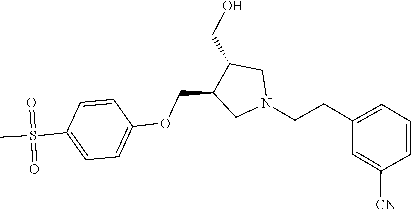 [SMILES: CS(=O)(=O)c1ccc(OC[C@@H]2CN(CCc3cccc(C#N)c3)C[C@H]2CO)cc1] | 3-{2-[(3S,4S)-3- (hydroxymethyl)- 4-[(4- methanesulfonyl- phenoxy)methyl] pyrrolidin-1- ylethyl} benzonitrile | Calc'd 415.2 Found 415.1 | n/a | |
| 26 | 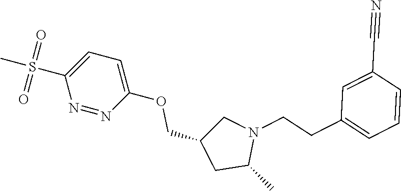 [SMILES: C[C@@H]1C[C@H](COc2ccc(S(C)(=O)=O)nn2)CN1CCc1cccc(C#N)c1] | 3-{2-[(2R,4S)-4- {[(6- methanesulfonyl- pyridazin-3- yl)oxy]methyl}-2- methylpyrrolidin- 1-yl]ethyl} benzonitrile | Calc'd 401.2 Found 401.2 | n/a | |

### Example 2

### 7-[(2R,4S)-4-{[4-(azetidine-3-sulfonyl)phenoxy]methyl}-2-methylpyrrolidin-1-yl]-5,6,7,8-tetrahydronaphthalene-2-carbonitrile (Compound 27), (7R or 7S)-7-[(2R,4S)-4-{[4-(azetidine-3-sulfonyl)phenoxy]methyl}-2-methylpyrrolidin-1-yl]-5,6,7,8-tetrahydronaphthalene-2-carbonitrile (Compound 28) and (7S or 7R)-7-[(2R,4S)-4-{[4-(azetidine-3-sulfonyl)phenoxy]methyl}-2-methylpyrrolidin-1-yl]-5,6,7,8-tetrahydronaphthalene-2-carbonitrile (Compound 29)

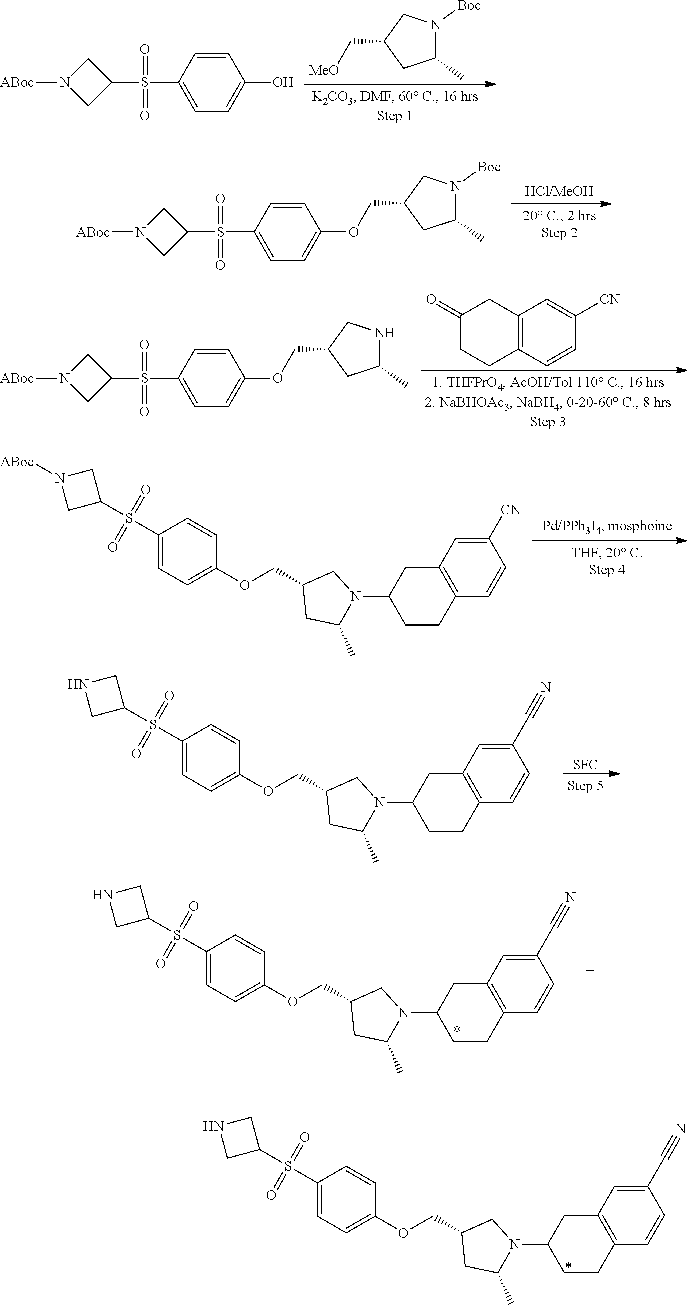

### Step 1: (2R,4S)-tert-butyl 4-((4-((1-((allyloxy)carbonyl)azetidin-3-yl)sulfonyl)phenoxy)methyl)-2-methylpyrrolidine-1-carboxylate

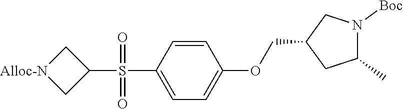

To a solution of allyl 3-((4-hydroxyphenyl)sulfonyl)azetidine-1-carboxylate (400 mg, 1.35 mmol) in DMF (4 mL) were added K₂CO₃ (372 mg, 2.69 mmol) and tert-butyl (2R,4S)-2-methyl-4-(methylsulfonyloxymethyl)pyrrolidine-1-carboxylate (434 mg, 1.48 mmol). The mixture was stirred at 80° C. for 16 h. The reaction mixture was partitioned between water (15 mL) and EtOAc (10 mL). The organic phase was separated, washed with brine (10 mL), dried over Na₂SO₄, filtered and concentrated under reduced pressure to give (2R,4S)-tert-butyl 4-((4-((1-((allyloxy)carbonyl)azetidin-3-yl)sulfonyl)phenoxy)methyl)-2-methylpyrrolidine-1-carboxylate, which was used without further purification. MS=395.1 [M−C₅H₉O₂+H]⁺

### Step 2: allyl 3-((4-(((3S,5R)-5-methylpyrrolidin-3-yl)methoxy)phenyl)sulfonyl)azetidine-1-carboxylate

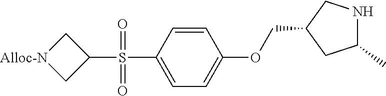

To a solution of (2R,4S)-tert-butyl 4-((4-((1-((allyloxy)carbonyl)azetidin-3-yl)sulfonyl)phenoxy)methyl)-2-methylpyrrolidine-1-carboxylate (0.73 g, 1.48 mmol) in MeOH (2 mL) was added HCl in MeOH (4 M, 10 mL). The mixture was stirred at RT for 2 h. MeOH was removed under reduced pressure to give allyl 3-((4-(((3S,5R)-5-methylpyrrolidin-3-yl)methoxy)phenyl)sulfonyl)azetidine-1-carboxylate, which was used without further purification. MS=395.2 [M+H]⁺.

### Step 3: allyl 3-((4-(((3S,5R)-1-(7-cyano-1,2,3,4-tetrahydronaphthalen-2-yl)-5-methylpyrrolidin-3-yl)methoxy)phenyl)sulfonyl)azetidine-1-carboxylate

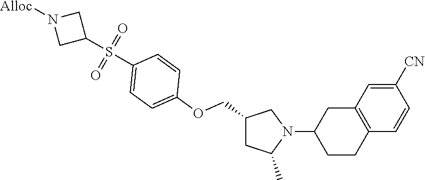

To a solution of allyl 3-((4-(((3S,5R)-5-methylpyrrolidin-3-yl)methoxy)phenyl)sulfonyl)azetidine-1-carboxylate (400 mg, 928 μmol, HCl salt), 7-oxo-5,6,7,8-tetrahydronaphthalene-2-carbonitrile (191 mg, 1.11 mmol), and AcOH (0.05 mL) in toluene (5 mL) was added Ti(i-PrO)₄ (1.32 g, 4.64 mmol). The mixture was stirred at 110° C. for 16 h. The mixture was then cooled to 0° C. and NaBH₄ (105 mg, 2.78 mmol) was added. The resulting mixture was stirred at RT for 4 h. The mixture was cooled to 0° C., then diluted with sat. aq. NH₄Cl (80 mL) and extracted with DCM/i-PrOH (3:1, 25 mL×3). The combined organic layers were washed with brine (25 mL), dried over Na₂SO₄, filtered and concentrated under reduced pressure to give a residue. The residue was purified by flash silica gel chromatography (Biotage 4 g cartridge, 0-100% Ethyl acetate/Petroleum ether) to give allyl 3-((4-(((3S,5R)-1-(7-cyano-1,2,3,4-tetrahydronaphthalen-2-yl)-5-methylpyrrolidin-3-yl)methoxy)phenyl)sulfonyl)azetidine-1-carboxylate. MS=550.3 [M+H]⁺

### Step 4: 7-((2R,4S)-4-((4-(azetidin-3-ylsulfonyl)phenoxy)methyl)-2-methylpyrrolidin-1-yl)-5,6,7,8-tetrahydronaphthalene-2-carbonitrile

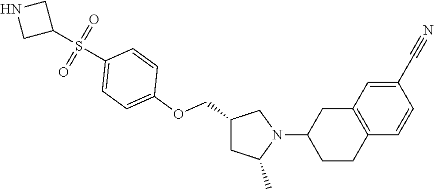 [SMILES: C[C@@H]1C[C@H](COc2ccc(S(=O)(=O)C3CNC3)cc2)CN1C1CCc2ccc(C#N)cc2C1]

To a solution of allyl 3-((4-(((3S,5R)-1-(7-cyano-1,2,3,4-tetrahydronaphthalen-2-yl)-5-methylpyrrolidin-3-yl)methoxy)phenyl)sulfonyl)azetidine-1-carboxylate (350 mg, 637 μmol) in THE (2 mL) was added Pd(PPh₃)₄ (147 mg, 127 μmol) and morpholine (222 mg, 2.55 mmol). The reaction mixture was stirred at RT for 2 h. The reaction mixture was filtered and concentrated under reduced pressure to give a residue. The residue was purified by preparative reverse phase HPLC (Phenomenex Luna C18, 1-20% MeCN:0.04% HCl in H₂O) to give 7-[(2R,4S)-4-{[4-(azetidine-3-sulfonyl)phenoxy]methyl}-2-methylpyrrolidin-1-yl]-5,6,7,8-tetrahydronaphthalene-2-carbonitrile (Compound 27). ¹H NMR (DMSO-d₆, 400 MHz) δ 11.65-11.47 (m, 1H), 9.73 (br s, 1H), 9.45 (br s, 1H), 7.91-7.88 (m, 2H), 7.70-7.61 (m, 2H), 7.45-7.34 (m, 1H), 7.25 (d, J=8.8 Hz, 2H), 4.69-4.61 (m, 1H), 4.32-4.11 (m, 6H), 3.84-3.69 (m, 2H), 3.59-3.50 (m, 1H), 3.42-3.25 (m, 2H), 3.16-2.89 (m, 4H), 2.45-2.38 (m, 2H), 1.96-1.77 (m, 1H), 1.71-1.63 (m, 1H), 1.52-1.49 (m, 3H). MS=466.3 [M+H]⁺

### Step 5: (7R or 7S)-7-[(2R,4S)-4-{[4-(azetidine-3-sulfonyl)phenoxy]methyl}-2-methylpyrrolidin-1-yl]-5,6,7,8-tetrahydronaphthalene-2-carbonitrile and (7S or 7R)-7-[(2R,4S)-4-{[4-(azetidine-3-sulfonyl)phenoxy]methyl}-2-methylpyrrolidin-1-yl]-5,6,7,8-tetrahydronaphthalene-2-carbonitrile

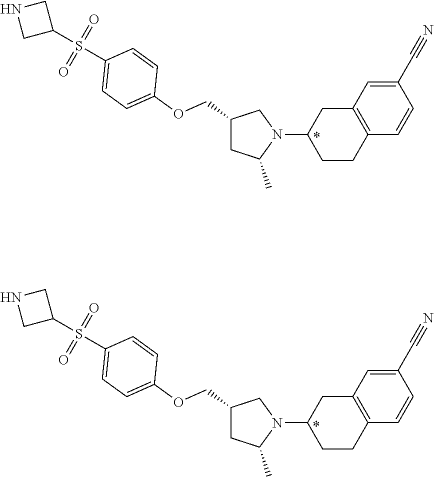 [SMILES: C[C@@H]1C[C@H](COc2ccc(S(=O)(=O)C3CNC3)cc2)CN1C1CCc2ccc(C#N)cc2C1 ; C[C@@H]1C[C@H](COc2ccc(S(=O)(=O)C3CNC3)cc2)CN1C1CCc2ccc(C#N)cc2C1]

7-[(2R,4S)-4-{[4-(azetidine-3-sulfonyl)phenoxy]methyl}-2-methylpyrrolidin-1-yl]-5,6,7,8-tetrahydronaphthalene-2-carbonitrile (95 mg, 189 μmol, HCl salt) was further purified by preparative chiral SFC (DAICEL CHIRALPAK AD, 50% ethanol with 0.1% NH₄OH in CO₂). The first eluting isomer (Compound 28): ¹H NMR (DMSO-d₆, 400 MHz) δ 7.76 (d, J=8.8 Hz, 2H), 7.57 (s, 1H), 7.51 (d, J=8.0 Hz, 1H), 7.27 (d, J=8.0 Hz, 1H), 7.16 (d, J=8.8 Hz, 2H), 4.47-4.39 (m, 1H), 4.02-3.94 (m, 2H), 3.71 (t, J=7.2 Hz, 2H), 3.50-3.47 (m, 4H), 3.06-2.99 (m, 1H), 2.97-2.86 (m, 3H), 2.84-2.77 (m, 2H), 2.75-2.68 (m, 2H), 2.17-2.08 (m, 1H), 1.93-1.90 (m, 1H), 1.70-1.60 (m, 1H), 1.16-1.09 (m, 1H), 1.05 (d, J=6.0 Hz, 3H). MS=466.3 [M+H]⁺. The second eluting isomer (Compound 29): ¹H NMR (400 MHz, CD₃CN) δ 7.79 (d, J=8.4 Hz, 2H), 7.44-7.40 (m, 2H), 7.23 (d, J=7.6 Hz, 1H), 7.09 (d, J=8.4 Hz, 2H), 4.24-4.22 (m, 1H), 4.03-3.95 (m, 2H), 3.83 (t, J=8.0 Hz, 2H), 3.60 (t, J=8.8 Hz, 2H), 3.38-3.21 (m, 1H), 3.10-2.99 (m, 3H), 2.94 (s, 1H), 2.86-2.79 (m, 5H), 2.52-2.46 (m, 1H), 2.04-2.01 (m, 1H), 1.67-1.57 (m, 1H), 1.22-1.15 (m, 1H), 1.06 (d, J=6.0 Hz, 3H). MS=466.3 [M+H]⁺

### Example 3 (Compound 30)

### 3-{2-[(2R,4S)-4-{[(6-methanesulfonyl-5-methylpyridin-3-yl)oxy]methyl}-2-methylpyrrolidin-1-yl]ethyl}benzonitrile

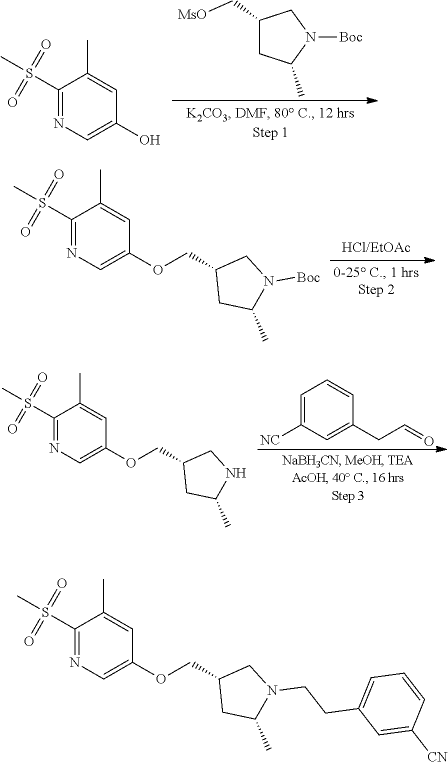

### Step 1: (2R,4S)-tert-butyl 2-methyl-4-(((5-methyl-6-(methylsulfonyl)pyridin-3-yl)oxy)methyl)pyrrolidine-1-carboxylate

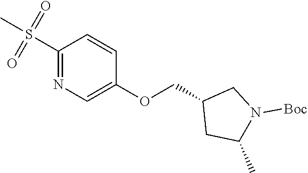 [SMILES: C[C@@H]1C[C@H](COc2ccc(S(C)(=O)=O)nc2)CN1C(=O)OC(C)(C)C]

To a solution of 5-methyl-6-(methylsulfonyl)pyridin-3-ol (500 mg, 2.67 mmol) and (2R,4S)-tert-butyl 2-methyl-4-(((methylsulfonyl)oxy)methyl)pyrrolidine-1-carboxylate (940 mg, 3.20 mmol) in DMF (5 mL) at RT was added K₂CO₃ (738 mg, 5.34 mmol). The mixture was stirred at 80° C. for 12 h. The reaction mixture was allowed to cool to RT, then was diluted with H₂O (20 mL) and extracted with EtOAc (20 mL×3). The combined organic layers were washed with brine (20 mL), dried over Na₂SO₄, filtered and concentrated under reduced pressure to give (2R,4S)-tert-butyl 2-methyl-4-(((5-methyl-6-(methylsulfonyl)pyridin-3-yl)oxy)methyl)pyrrolidine-1-carboxylate, which was used without further purification. MS=329.2 [M−C₄H₈+H]⁺.

### Step 2: 3-methyl-5-(((3S,5R)-5-methylpyrrolidin-3-yl)methoxy)-2-(methylsulfonyl)pyridine

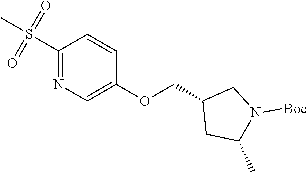 [SMILES: C[C@@H]1C[C@H](COc2ccc(S(C)(=O)=O)nc2)CN1C(=O)OC(C)(C)C]

To a 0° C. solution of (2R,4S)-tert-butyl 2-methyl-4-(((5-methyl-6-(methylsulfonyl)pyridin-3-yl)oxy)methyl)pyrrolidine-1-carboxylate (1.50 g, 3.90 mmol) in EtOAc (1 mL) was added HCl in EtOAc (4 M, 20 mL). The mixture was stirred at RT for 1 h, and the reaction was concentrated under reduced pressure to give a solid. The solid was washed with MTBE (10 mL×3) to give 3-methyl-5-(((3S,5R)-5-methylpyrrolidin-3-yl)methoxy)-2-(methylsulfonyl)pyridine, which was used without further purification. MS=285.1 [M+H]⁺.

### Step 3: 3-{2-[(2R,4S)-4-{[(6-methanesulfonyl-5-methylpyridin-3-yl)oxy]methyl}-2-methylpyrrolidin-1-yl]ethyl}benzonitrile

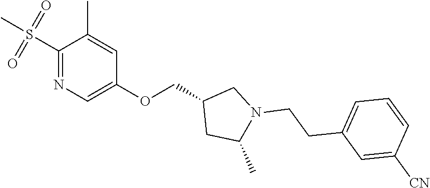 [SMILES: Cc1cc(OC[C@H]2C[C@@H](C)N(CCc3cccc(C#N)c3)C2)cnc1S(C)(=O)=O]

To a solution of 3-methyl-5-(((3S,5R)-5-methylpyrrolidin-3-yl)methoxy)-2-(methylsulfonyl)pyridine (150 mg, 467 μmol, HCl salt), TEA (94.6 mg, 935 μmol) and 3-(2-oxoethyl) benzonitrile (67.8 mg, 467 μmol) in MeOH (1 mL) at RT were added AcOH (28 mg, 467 μmol) and NaBH₃CN (117 mg, 1.87 mmol). The mixture was stirred at 40° C. for 16 h. The reaction mixture was diluted with H₂O (10 mL) and extracted with EtOAc (10 mL×3). The combined organic layers were washed with brine (30 mL), dried over Na₂SO₄, filtered and concentrated under reduced pressure to give a residue. The residue was purified by preparative reverse phase HPLC (Waters Xbridge BEH C18, 20-55% MeCN:10 mM NH₄HCO₃ in H₂O) to give 3-{2-[(2R,4S)-4-{[(6-methanesulfonyl-5-methylpyridin-3-yl)oxy]methyl}-2-methylpyrrolidin-1-yl]ethyl}benzonitrile (Compound 30). ¹H NMR (DMSO-d₆, 400 MHz) δ 8.18 (d, J=4.0 Hz, 1H), 7.70 (s, 1H), 7.61-7.56 (m, 2H), 7.50 (d, J=4.0 Hz, 1H), 7.42 (t, J=8.0 Hz, 1H), 3.94 (d, J=8.0 Hz, 2H), 3.30 (s, 3H), 3.06-3.02 (m, 1H), 2.98-2.93 (m, 1H), 2.83-2.78 (m, 1H), 2.76-2.69 (m, 1H), 2.58 (s, 3H), 2.52-2.51 (m, 1H), 2.37-2.32 (m, 1H), 2.30-2.23 (m. 2H), 2.15-2.07 (m, 1H). 1.07-1.02 (m, 1H), 0.98 (d, J=6.0 Hz, 3H). MS=414.2 [M+H]⁺.

### Example 4 (Compounds 31, 32, 33)

### 7-[(2R,4S)-4-{[4-(2-methanesulfonylethanesulfonyl)phenoxy]methyl}-2-methylpyrrolidin-1-yl]-5,6,7,8-tetrahydronaphthalene-2-carbonitrile (Compound 31), (7R or 7S)-7-[(2R,4S)-4-{[4-(2-methanesulfonylethanesulfonyl)phenoxy]methyl}-2-methylpyrrolidin-1-yl]-5,6,7,8-tetrahydronaphthalene-2-carbonitrile (Compound 32) and (7S or 7R)-7-[(2R,4S)-4-{[4-(2-methanesulfonylethanesulfonyl)phenoxy]methyl}-2-methylpyrrolidin-1-yl]-5,6,7,8-tetrahydronaphthalene-2-carbonitrile (Compound 33)

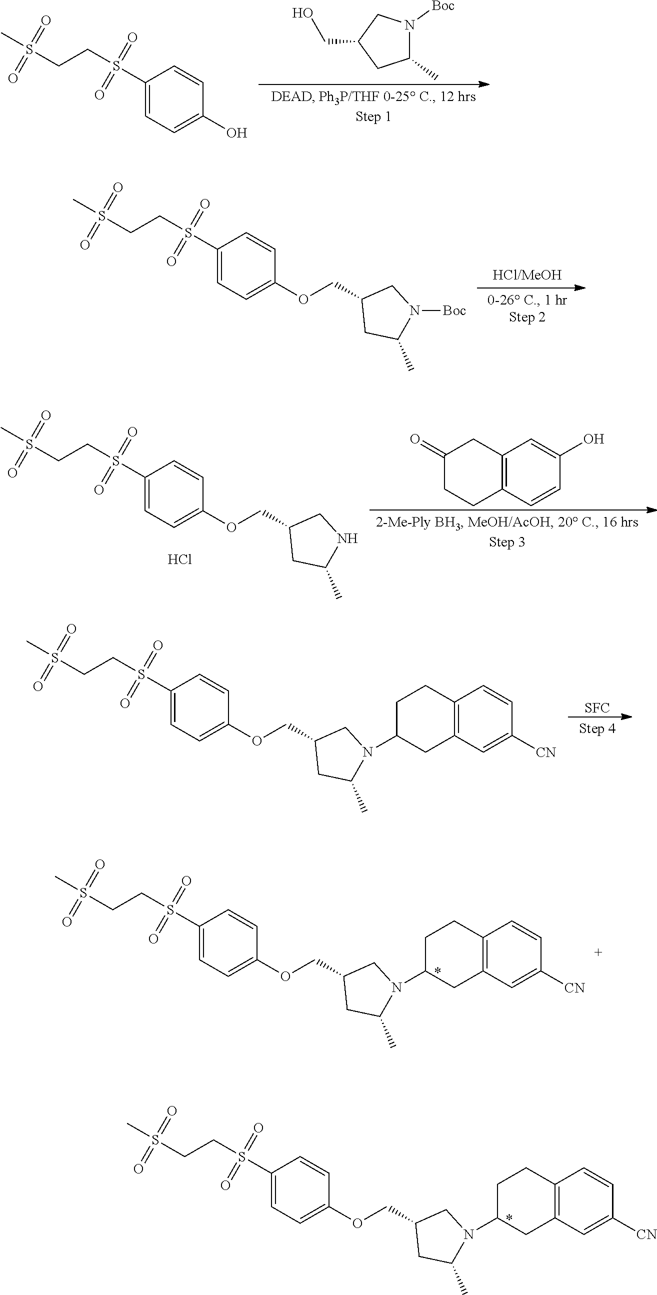

### Step 1: (2R,4S)-tert-butyl 2-methyl-4-((4-((2-(methylsulfonyl)ethyl)sulfonyl)phenoxy)methyl)pyrrolidine-1-carboxylate

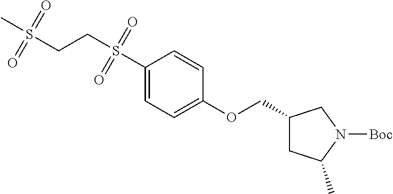 [SMILES: C[C@@H]1C[C@H](COc2ccc(S(=O)(=O)CCS(C)(=O)=O)cc2)CN1C(=O)OC(C)(C)C]

To a 0° C. solution of 4-((2-(methylsulfonyl)ethyl)sulfonyl)phenol (1.50 g, 3.97 mmol), (2R,4S)-tert-butyl 4-(hydroxymethyl)-2-methylpyrrolidine-1-carboxylate (855 mg, 3.97 mmol) and PPh₃ (2.08 g, 7.94 mmol) in THE (10 mL) was added DEAD (1.38 g, 7.94 mmol). The mixture was stirred at RT for 12 h, whereupon the reaction mixture was diluted with H₂O (20 mL) and extracted with EtOAc (20 mL×3). The combined organic layers were washed with brine (50 mL), dried over Na₂SO₄, filtered and concentrated under reduced pressure to give a residue. The residue was purified by flash silica gel chromatography (Biotage 12 g cartridge, 0-80% EtOAc/Hexane) to give (2R,4S)-tert-butyl 2-methyl-4-((4-((2-(methylsulfonyl)ethyl)sulfonyl)phenoxy)methyl)pyrrolidine-1-carboxylate. MS=362.1 [M−C₅H₉O₂+H]⁺.

### Step 2: (2R,4S)-2-methyl-4-((4-((2-(methylsulfonyl)ethyl)sulfonyl)phenoxy)methyl)pyrrolidine

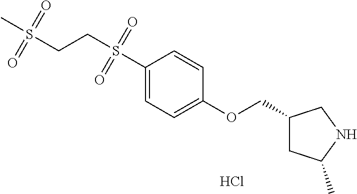 [SMILES: C[C@@H]1C[C@H](COc2ccc(S(=O)(=O)CCS(C)(=O)=O)cc2)CN1 ; Cl]

To a solution of (2R,4S)-tert-butyl 2-methyl-4-((4-((2-(methylsulfonyl)ethyl)sulfonyl)phenoxy)methyl)pyrrolidine-1-carboxylate (900 mg, 1.95 mmol) in MeOH (1 mL) at 0° C. was added HCl in MeOH (4 M, 20 mL). The mixture was stirred at RT for 1 h. The reaction mixture was filtered and concentrated under reduced pressure to give (2R,4S)-2-methyl-4-((4-((2-(methylsulfonyl)ethyl)sulfonyl)phenoxy)methyl)pyrrolidine, which was used without further purification. MS=362.1 [M+H]⁺.

### Step 3: 7-[(2R,4S)-4-{[4-(2-methanesulfonylethanesulfonyl)phenoxy]methyl}-2-methylpyrrolidin-1-yl]-5,6,7,8-tetrahydronaphthalene-2-carbonitrile

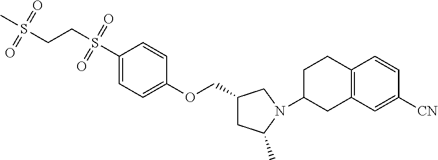 [SMILES: C[C@@H]1C[C@H](COc2ccc(S(=O)(=O)CCS(C)(=O)=O)cc2)CN1C1CCc2ccc(C#N)cc2C1]

To a mixture of (2R,4S)-2-methyl-4-((4-((2-(methylsulfonyl)ethyl)sulfonyl)phenoxy)methyl)pyrrolidine (130 mg, 360 μmol) and 7-oxo-5,6,7,8-tetrahydronaphthalene-2-carbonitrile (68 mg, 396 μmol) in MeOH (4 mL) and AcOH (0.02 mL) was added 2-methylpyridine borane complex (46 mg, 432 μmol). The mixture was stirred at RT for 16 h. The reaction mixture was concentrated under reduced pressure and the residue was diluted with water (10 mL) and extracted with DCM/i-PrOH (3:1, 10 mL×3). The combined organic layers were washed with brine (10 mL), dried over Na₂SO₄, filtered and concentrated under reduced pressure to give a residue. The residue was purified by preparative reverse phase HPLC (Waters Xbridge BEH C18, 30-55% MeCN:10 mM NH₄HCO₃ in H₂O) to give 7-[(2R,4S)-4-{[4-(2-methanesulfonylethanesulfonyl)phenoxy]methyl}-2-methylpyrrolidin-1-yl]-5,6,7,8-tetrahydronaphthalene-2-carbonitrile (Compound 31). ¹H NMR (DMSO-d₆, 400 MHz) δ 7.87 (d, J=8.8 Hz, 2H), 7.57 (s, 1H), 7.52 (dd, J=8.0, 2.0 Hz, 1H), 7.26 (d, J=8.0 Hz, 1H), 7.20 (d, J=9.2 Hz, 2H), 4.04-3.97 (m, 2H), 3.67-3.63 (m, 2H), 3.40-3.36 (m, 2H), 3.06 (s, 3H), 3.04-2.69 (m, 9H), 2.18-2.11 (m, 1H), 1.94-1.91 (m, 1H), 1.71-1.61 (m, 1H), 1.17-1.10 (m, 1H), 1.05 (d, J=6.0 Hz, 3H), MS=517.2 [M+H]⁺.

### Step 4: (7R or 7S)-7-[(2R,4S)-4-{[4-(2-methanesulfonylethanesulfonyl)phenoxy]methyl}-2-methylpyrrolidin-1-yl]-5,6,7,8-tetrahydronaphthalene-2-carbonitrile and (7S or 7R)-7-[(2R,4S)-4-{[4-(2-methanesulfonylethanesulfonyl)phenoxy]methyl}-2-methylpyrrolidin-1-yl]-5,6,7,8-tetrahydronaphthalene-2-carbonitrile

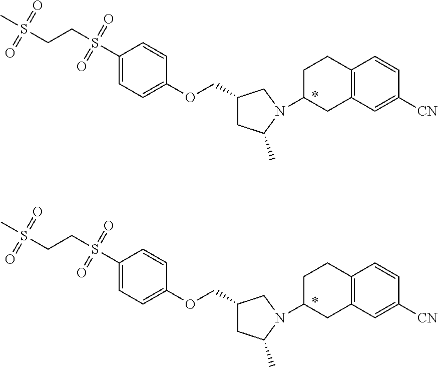 [SMILES: C[C@@H]1C[C@H](COc2ccc(S(=O)(=O)CCS(C)(=O)=O)cc2)CN1C1CCc2ccc(C#N)cc2C1 ; C[C@@H]1C[C@H](COc2ccc(S(=O)(=O)CCS(C)(=O)=O)cc2)CN1C1CCc2ccc(C#N)cc2C1]

7-[(2R,4S)-4-{[4-(2-methanesulfonylethanesulfonyl)phenoxy]methyl}-2-methylpyrrolidin-1-yl]-5,6,7,8-tetrahydronaphthalene-2-carbonitrile (60 mg, 116 μmol) was further purified by preparative chiral SFC (DAICEL CHIRALPAK AD, 55% IPA with 0.1% NH₄OH in CO₂). The first eluting isomer of the title compound:

(Compound 32): ¹H NMR (DMSO-d₆, 400 MHz) δ 7.85 (d, J=8.8 Hz, 2H), 7.57 (s, 1H), 7.51 (dd, J=8.0, 2.0 Hz, 1H), 7.27 (d, J=7.6 Hz, 1H), 7.20 (d, J=9.2 Hz, 2H), 4.04-3.97 (m, 2H), 3.67-3.63 (m, 2H), 3.40-3.36 (m, 2H), 3.06 (s, 3H), 3.04-3.02 (m, 1H), 2.96-2.88 (m, 3H), 2.85-2.81 (m, 2H), 2.79-2.66 (m, 3H), 2.18-2.11 (m, 1H), 1.94-1.91 (m, 1H), 1.71-1.61 (m, 1H), 1.17-1.10 (m, 1H), 1.05 (d, J=6.0 Hz, 3H), MS=517.2 [M+H]⁺. The second eluting isomer of the title compound:

(Compound 33): ¹H NMR (DMSO-d₆, 400 MHz) δ 7.86 (d, J=8.8 Hz, 2H), 7.55 (s, 1H), 7.51 (d, J=8.0 Hz, 1H), 7.27 (d, J=8.0 Hz, 1H), 7.21 (d, J=8.8 Hz, 2H), 4.02-4.00 (m, 2H), 3.68-3.63 (m, 2H), 3.07 (s, 3H), 3.00-2.92 (m, 5H), 2.85-2.80 (m, 4H), 2.80-2.76 (m, 2H), 2.20-2.12 (m, 1H), 1.98-1.95 (m, 1H), 1.62-1.52 (m, 1H), 1.18-1.12 (m, 1H), 1.04 (d, J=6.0 Hz, 3H). MS=517.2 [M+H]⁺.

### Example 5 (Compound 34, 35, 36)

### 7-[(2R,4S)-4-{[4-(3-methanesulfonylpropanesulfonyl)phenoxy]methyl}-2-methylpyrrolidin-1-yl]-5,6,7,8-tetrahydronaphthalene-2-carbonitrile (Compound 34), (7R or 7S)-7-[(2R,4S)-4-{[4-(3-methanesulfonylpropanesulfonyl)phenoxy]methyl}-2-methylpyrrolidin-1-yl]-5,6,7,8-tetrahydronaphthalene-2-carbonitrile (Compound 35), and (7S or 7R)-7-[(2R,4S)-4-{[4-(3-methanesulfonylpropanesulfonyl)phenoxy]methyl}-2-methylpyrrolidin-1-yl]-5,6,7,8-tetrahydronaphthalene-2-carbonitrile (Compound 36)

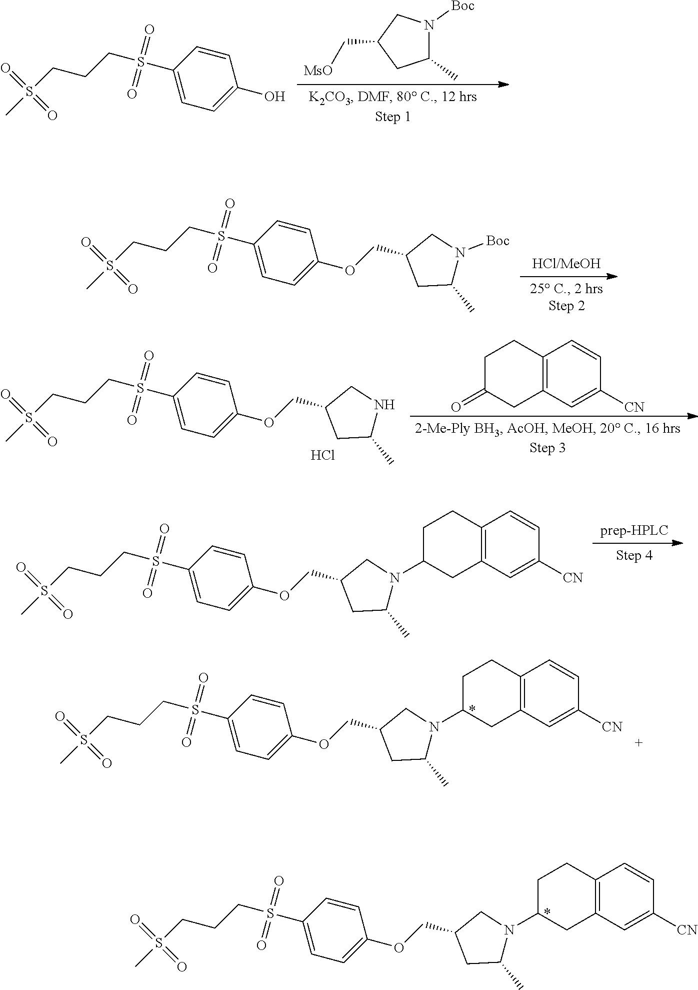

### Step 1: (2R,4S)-tert-butyl 2-methyl-4-((4-((3-(methylsulfonyl)propyl)sulfonyl)phenoxy)methyl)pyrrolidine-1-carboxylate

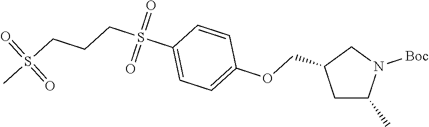 [SMILES: C[C@@H]1C[C@H](COc2ccc(S(=O)(=O)CCCS(C)(=O)=O)cc2)CN1C(=O)OC(C)(C)C]

To a solution of (2R,4S)-tert-butyl 2-methyl-4-(((methylsulfonyl)oxy)methyl)pyrrolidine-1-carboxylate (340 mg, 1.16 mmol) in DMF (5 mL) were added K₂CO₃ (320 mg, 2.32 mmol) and 4-((3-(methylsulfonyl)propyl)sulfonyl)phenol (387 mg, 1.39 mmol). The mixture was stirred at 80° C. for 12 h. The reaction mixture was allowed to cool to RT, then was diluted with H₂O (10 mL) and extracted with ethyl acetate (10 mL×2). The combined organic layers were washed with brine (10 mL), dried over Na₂SO₄, filtered and concentrated under reduced pressure to give a residue. The residue was purified by flash silica gel chromatography (Biotage12 cartridge, 0-60% EtOAc/Hexane) to give (2R,4S)-tert-butyl 2-methyl-4-((4-((3-(methylsulfonyl)propyl)sulfonyl)phenoxy)methyl)pyrrolidine-1-carboxylate. MS=420.2 [M−C₄H₈+H]⁺.

### Step 2: (2R,4S)-2-methyl-4-((4-((3-(methylsulfonyl)propyl)sulfonyl)phenoxy)methyl)pyrrolidine

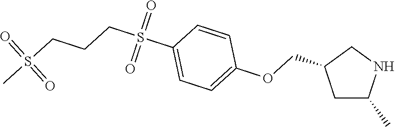

To a solution of (2R,4S)-tert-butyl 2-methyl-4-((4-((3-(methylsulfonyl)propyl)sulfonyl)phenoxy)methyl)pyrrolidine-1-carboxylate (200 mg, 420 μmol) in MeOH (1 mL) was added HCl in MeOH (4 M, 1.00 mL). The mixture was stirred at RT for 2 h. The reaction mixture was concentrated under reduced pressure to give (2R, 4S)-2-methyl-4-((4-((3-(methylsulfonyl)propyl)sulfonyl)phenoxy)methyl)pyrrolidine HCl salt, which was used without further purification. MS=376.2 [M+H]⁺.

### Step 3: 7-[(2R,4S)-4-{[4-(3-methanesulfonylpropanesulfonyl)phenoxy]methyl}-2-methylpyrrolidin-1-yl]-5,6,7,8-tetrahydronaphthalene-2-carbonitrile

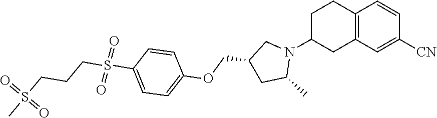 [SMILES: C[C@@H]1C[C@H](COc2ccc(S(=O)(=O)CCCS(C)(=O)=O)cc2)CN1C1CCc2ccc(C#N)cc2C1]

To a solution of (2R,4S)-2-methyl-4-((4-((3-(methylsulfonyl)propyl)sulfonyl)phenoxy)methyl)pyrrolidine (200 mg, 485 umol) and 7-oxo-5,6,7,8-tetrahydronaphthalene-2-carbonitrile (100 mg, 582 umol) in MeOH (3 mL) and AcOH (0.3 mL) was added 2-methylpyridine borane complex (104 mg, 970 umol). The mixture was stirred at RT for 16 h. The mixture was concentrated under reduced pressure. The residue was purified by preparative reverse phase HPLC (Phenomenex C18, 35-55% MeCN:10 mM NH₄HCO₃ in H₂O) to give 7-[(2R,4S)-4-{[4-(3-methanesulfonylpropanesulfonyl)phenoxy]methyl}-2-methylpyrrolidin-1-yl]-5,6,7,8-tetrahydronaphthalene-2-carbonitrile (Compound 34). ¹H NMR (DMSO-d₆, 400 MHz) δ 7.81 (d, J=8.4 Hz, 2H), 7.61-7.55 (m, 1H), 7.52 (d, J=8.0 Hz, 1H), 7.27 (d, J=8.0 Hz, 1H), 7.20 (d, J=8.8 Hz, 2H), 4.07-3.95 (m, 2H), 3.41 (t, J=7.6 Hz, 2H), 3.21 (t, J=7.6 Hz, 2H), 2.97 (s, 3H), 3.10-2.66 (m, 8H), 3.10-2.65 (m, 1H), 2.22-2.10 (m, 1H), 2.02-1.87 (m, 3H), 1.74-1.53 (m, 1H), 1.20-1.09 (m, 1H), 1.06 (d, J=6.0 Hz, 3H). MS=531.2 [M+H]⁺.

### Step 4: (7R or 7S)-7-[(2R,4S)-4-{[4-(3-methanesulfonylpropanesulfonyl)phenoxy]methyl}-2-methylpyrrolidin-1-yl]-5,6,7,8-tetrahydronaphthalene-2-carbonitrile and (7S or 7R)-7-[(2R,4S)-4-{[4-(3-methanesulfonylpropanesulfonyl)phenoxy]methyl}-2-methylpyrrolidin-1-yl]-5,6,7,8-tetrahydronaphthalene-2-carbonitrile

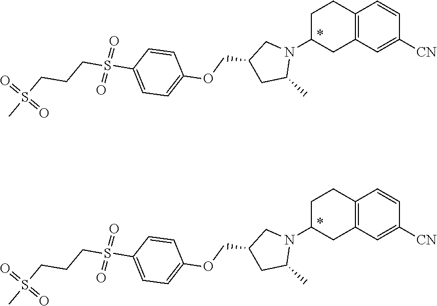 [SMILES: C[C@@H]1C[C@H](COc2ccc(S(=O)(=O)CCCS(C)(=O)=O)cc2)CN1C1CCc2ccc(C#N)cc2C1 ; C[C@@H]1C[C@H](COc2ccc(S(=O)(=O)CCCS(C)(=O)=O)cc2)CN1C1CCc2ccc(C#N)cc2C1]

7-[(2R,4S)-4-{[4-(3-methanesulfonylpropanesulfonyl)phenoxy]methyl}-2-methylpyrrolidin-1-yl]-5,6,7,8-tetrahydronaphthalene-2-carbonitrile (150 mg, 282 umol) was further purified by preparative reverse phase HPLC (Waters Xbridge BEH C18, 30-55% MeCN:10 mM NH₄HCO₃ in H₂O). The first eluting isomer of the title compound:

(Compound 35): ¹H NMR (DMSO-d₆, 400 MHz) δ 7.81 (d, J=8.4 Hz, 2H), 7.59 (s, 1H), 7.52 (d, J=8.0 Hz, 1H), 7.28 (d, J=8.0 Hz, 1H), 7.21 (d, J=8.8 Hz, 2H), 4.07-3.95 (m, 2H), 3.41 (t, J=7.6 Hz, 2H), 3.22 (t, J=8.0 Hz, 2H), 2.97 (s, 3H), 3.10-2.66 (m, 8H), 3.10-2.65 (m, 1H), 2.22-2.10 (m, 1H), 2.02-1.87 (m, 3H), 1.74-1.53 (m, 1H), 1.20-1.09 (m, 1H), 1.06 (d, J=6.0 Hz, 3H), MS=531.2 [M+H]⁺.

The second eluting isomer of the title compound:

(Compound 36): ¹H NMR (DMSO-d₆, 400 MHz) δ 7.82 (d, J=8.8 Hz, 2H), 7.56 (s, 1H), 7.52 (d, J=8.0 Hz, 1H), 7.28 (d, J=8.0 Hz, 1H), 7.21 (d, J=8.8 Hz, 2H), 4.06-3.96 (m, 2H), 3.44-3.38 (m, 2H), 3.25-3.18 (m, 2H), 3.07-2.73 (m, 10H), 2.22-2.12 (m, 1H), 2.01-1.90 (m, 3H), 1.65-1.52 (m, 1H), 1.20-1.11 (m, 1H), 1.05 (d, J=6.0 Hz, 3H), MS=531.2 [M+H]⁺.

The following compounds in Table 3 were prepared according to procedures similar to steps described for Compounds 27-36 using the appropriate starting materials.

TABLE 3

| | | | Exact | | |
|---|---|---|---|---|---|
| | | | Mass | Chiral | Elution |
| # | Structure | IUPAC Name | [M + H]⁺ | column | order |
| 37 | 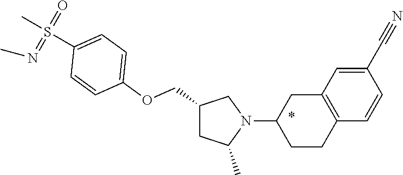 [SMILES: CN=S(C)(=O)c1ccc(OC[C@H]2C[C@@H](C)N(C3CCc4ccc(C#N)cc4C3)C2)cc1] | (7R or 7S)-7-[(2R,4S)- 2-methyl-4-({4- [methyl(methylimino) oxo-26- sulfanyl]phenoxy} methyl)pyrrolidin- 1-yl]-5,6,7,8- tetrahydronaphthalene- 2-carbonitrile | Calc'd 438.2 Found 438.3 | prep- HPLC Phenomenex C18 | 1st |
| 38 | 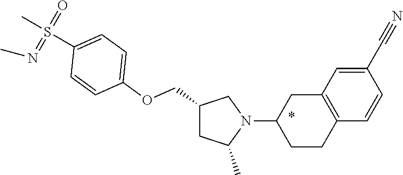 [SMILES: CN=S(C)(=O)c1ccc(OC[C@H]2C[C@@H](C)N(C3CCc4ccc(C#N)cc4C3)C2)cc1] | (7S or 7R)-7-[(2R,4S)- 2-methyl-4-({4- [methyl(methylimino) oxo-26- sulfanyl]phenoxy} methyl)pyrrolidin- 1-yl]-5,6,7,8- tetrahydronaphthalene- 2-carbonitrile | Calc'd 438.2 Found 438.3 | prep- HPLC Phenomenex C18 | 2nd |
| 39 | 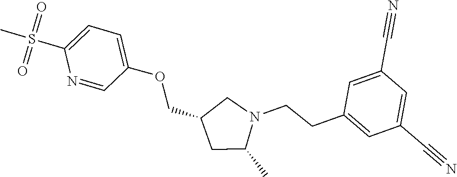 [SMILES: C[C@@H]1C[C@H](COc2ccc(S(C)(=O)=O)nc2)CN1CCc1cc(C#N)cc(C#N)c1] | 5-{2-[(2R,4S)-4-{[(6- methanesulfonyl- pyridin-3-yl)oxy] methyl}-2- methylpyrrolidin-1- yl]ethyl}benzene-1,3- dicarbonitrile | Calc'd 425.2 Found 425.2 | n/a | |
| 40 | 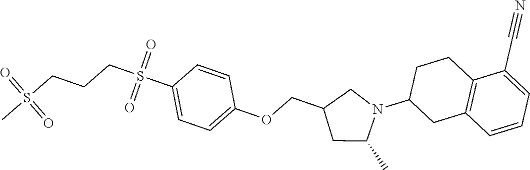 [SMILES: C[C@@H]1CC(COc2ccc(S(=O)(=O)CCCS(C)(=O)=O)cc2)CN1C1CCc2c(C#N)cccc2C1] | 6-[(2R,4S)-4-{[4-(3- methanesulfonyl- propanesulfonyl) phenoxy]methyl}-2- methylpyrrolidin-1- yl]-5,6,7,8- tetrahydronaphthalene- 1-carbonitrile | Calc'd 531.2 Found 531.3 | n/a | |
| 41 | 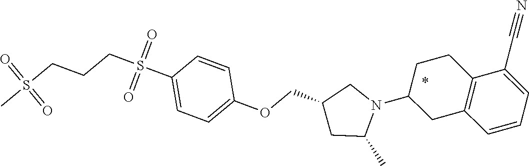 [SMILES: C[C@@H]1C[C@H](COc2ccc(S(=O)(=O)CCCS(C)(=O)=O)cc2)CN1C1CCc2c(C#N)cccc2C1] | (6S or 6R)-6-[(2R,4S)- 4-{[4-(3- methanesulfonyl- propanesulfonyl) phenoxy]methyl}-2- methylpyrrolidin-1- yl]-5,6,7,8- tetrahydro- naphthalene-1- carbonitrile | Calc'd 531.2 Found 531.3 | Chiral SFC DAICEL CHIRALCEL OJ | 1st |
| 42 | 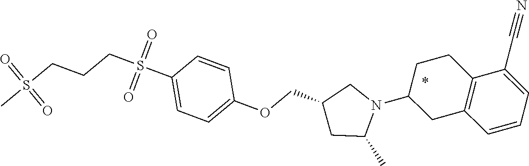 [SMILES: C[C@@H]1C[C@H](COc2ccc(S(=O)(=O)CCCS(C)(=O)=O)cc2)CN1C1CCc2c(C#N)cccc2C1] | (6R or 6S)-6-[(2R,4S)- 4-{[4-(3- methanesulfonyl- propanesulfonyl) phenoxy]methyl}-2- methylpyrrolidin-1- yl]-5,6,7,8- tetrahydronaphthalene- 1-carbonitrile | Calc'd 531.2 Found 531.2 | Chiral SFC DAICEL CHIRALCEL OJ | 2nd |
| 43 | 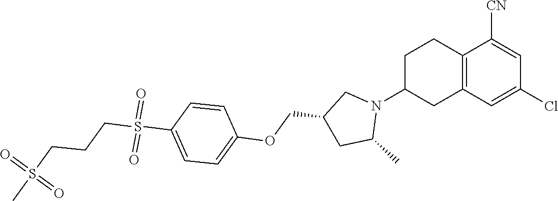 [SMILES: C[C@@H]1C[C@H](COc2ccc(S(=O)(=O)CCCS(C)(=O)=O)cc2)CN1C1CCc2c(C#N)cc(Cl)cc2C1] | 3-chloro-6-[(2R,4S)-4- {[4-(3- methanesulfonyl propanesulfonyl) phenoxy]methyl}-2- methylpyrrolidin-1- yl]-5,6,7,8- tetrahydronaphthalene- 1-carbonitrile | Calc'd 565.2 Found 565.2 | n/a | |
| 44 | 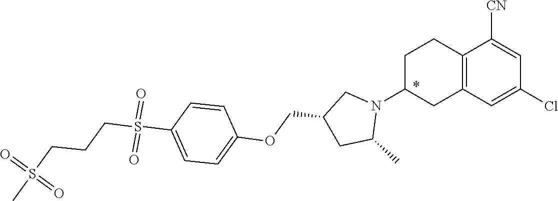 [SMILES: C[C@@H]1C[C@H](COc2ccc(S(=O)(=O)CCCS(C)(=O)=O)cc2)CN1C1CCc2c(C#N)cc(Cl)cc2C1] | (6R or 6S)-3-chloro-6- [(2R,4S)-4-{[4-(3- methanesulfonyl propanesulfonyl) phenoxy]methyl}-2- methylpyrrolidin-1- yl]-5,6,7,8- tetrahydronaphthalene- 1-carbonitrile | Calc'd 565.2 Found 565.1 | Chiral SFC DAICEL CHIRALPAK AD | 1st |
| 45 | 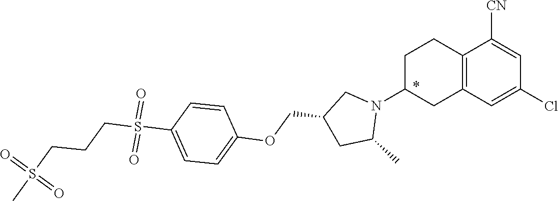 [SMILES: C[C@@H]1C[C@H](COc2ccc(S(=O)(=O)CCCS(C)(=O)=O)cc2)CN1C1CCc2c(C#N)cc(Cl)cc2C1] | (6S or 6R)-3-chloro-6- [(2R,4S)-4-{[4-(3- methanesulfonyl- propanesulfonyl) phenoxy]methyl}-2- methylpyrrolidin-1- yl]-5,6,7,8- tetrahydronaphthalene- 1-carbonitrile | Calc'd 565.2 Found 565.1 | Chiral SFC DAICEL CHIRALPAK AD | 2nd |
| 46 | 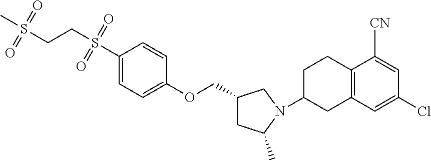 [SMILES: C[C@@H]1C[C@H](COc2ccc(S(=O)(=O)CCS(C)(=O)=O)cc2)CN1C1CCc2c(C#N)cc(Cl)cc2C1] | 3-chloro-6-[(2R,4S)-4- {[4-(2- methanesulfonyl- ethanesulfonyl) phenoxy]methyl}-2- methylpyrrolidin-1- yl]-5,6,7,8- tetrahydronaphthalene- 1-carbonitrile | Calc'd 551.1 Found 551.2 | n/a | |
| 47 | 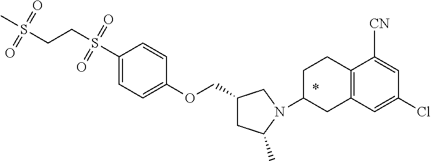 [SMILES: C[C@@H]1C[C@H](COc2ccc(S(=O)(=O)CCS(C)(=O)=O)cc2)CN1C1CCc2c(C#N)cc(Cl)cc2C1] | (6S or 6R)-3-chloro-6- [(2R,4S)-4-{[4-(2- methanesulfonyl- ethanesulfonyl) phenoxy]methyl}-2- methylpyrrolidin-1- yl]-5,6,7,8- tetrahydronaphthalene- 1-carbonitrile | Calc'd 551.1 Found 551.2 | DAICEL CHIRA LCEL OJ | 1st |
| 48 | 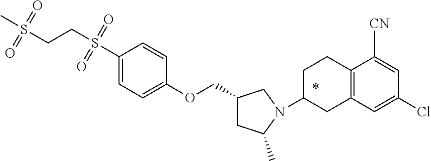 [SMILES: C[C@@H]1C[C@H](COc2ccc(S(=O)(=O)CCS(C)(=O)=O)cc2)CN1C1CCc2c(C#N)cc(Cl)cc2C1] | (6R or 6S)-3-chloro-6- [(2R,4S)-4-{[4-(2- methanesulfonyl- ethanesulfonyl) phenoxy]methyl}-2- methylpyrrolidin-1- yl]-5,6,7,8- tetrahydronaphthalene- 1-carbonitrile | Calc'd 551.1 Found 551.1 | DAICEL CHIRALCEL OJ | 2nd |
| 49 | 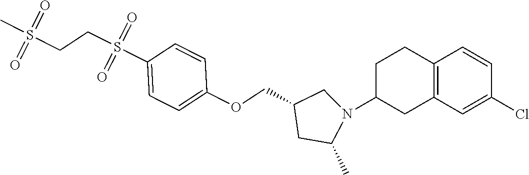 [SMILES: C[C@@H]1C[C@H](COc2ccc(S(=O)(=O)CCS(C)(=O)=O)cc2)CN1C1CCc2ccc(Cl)cc2C1] | (2R,4S)-1-[7-chloro- 1,2,3,4- tetrahydronaphthalen- 2-yl]-4-{[4-(2- methanesulfonyl- ethanesulfonyl) phenoxy]methyl}-2- methylpyrrolidine | Calc'd 526.1 Found 526.2 | n/a | |
| 50 | 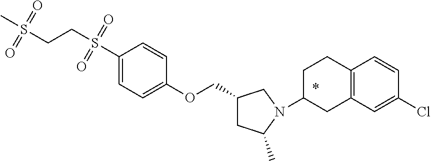 [SMILES: C[C@@H]1C[C@H](COc2ccc(S(=O)(=O)CCS(C)(=O)=O)cc2)CN1C1CCc2ccc(Cl)cc2C1] | (2R,4S)-1-[(2S or 2R)- 7-chloro-1,2,3,4- tetrahydronaphthalen- 2-yl]-4-{[4-(2- methanesulfonyl- ethanesulfonyl) phenoxy]methyl}-2- methylpyrrolidine | Calc'd 526.1 Found 526.2 | DAICEL CHIRALPAK AD | 1st |
| 51 | 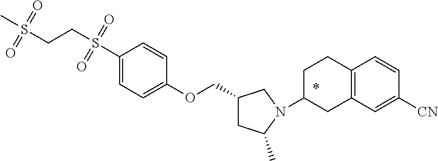 [SMILES: C[C@@H]1C[C@H](COc2ccc(S(=O)(=O)CCS(C)(=O)=O)cc2)CN1C1CCc2ccc(C#N)cc2C1] | (2R,4S)-1-[(2R or 2S)- 7-chloro-1,2,3,4- tetrahydronaphthalen- 2-yl]-4-{[4-(2- methanesulfonyl- ethanesulfonyl) phenoxy]methyl}-2- methylpyrrolidine | Calc'd 526.1 Found 526.1 | DAICEL CHIRALPAK AD | 2nd |
| 52 | 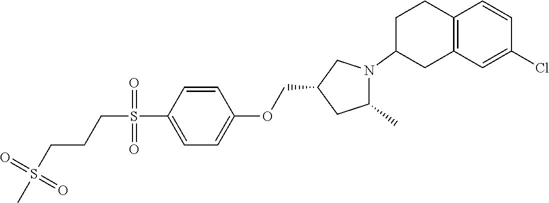 [SMILES: C[C@@H]1C[C@H](COc2ccc(S(=O)(=O)CCCS(C)(=O)=O)cc2)CN1C1CCc2ccc(Cl)cc2C1] | (2R,4S)-1-[7-chloro- 1,2,3,4- tetrahydronaphthalen- 2-yl]-4-{[4-(3- methanesulfonyl- propanesulfonyl) phenoxy]methyl}-2- methylpyrrolidine | Calc'd 540.2 Found 540.2 | n/a | |
| 53 | 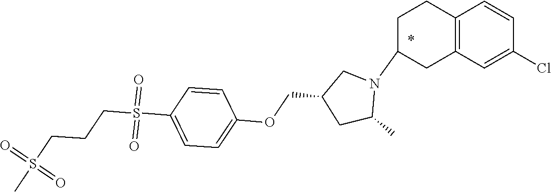 [SMILES: C[C@@H]1C[C@H](COc2ccc(S(=O)(=O)CCCS(C)(=O)=O)cc2)CN1C1CCc2ccc(Cl)cc2C1] | (2R,4S)-1-[(2R or 2S)- 7-chloro-1,2,3,4- tetrahydronaphthalen- 2-yl]-4-{[4-(3- methanesulfonyl- propanesulfonyl) phenoxy]methyl}-2- methylpyrrolidine | Calc'd 540.2 Found 540.1 | DAICEL CHIRALPAK AD | |
| 54 | 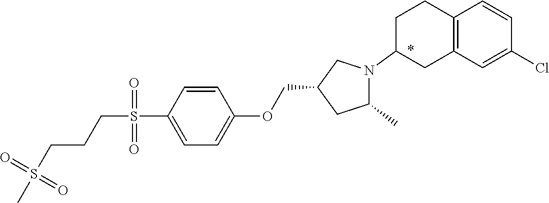 [SMILES: C[C@@H]1C[C@H](COc2ccc(S(=O)(=O)CCCS(C)(=O)=O)cc2)CN1C1CCc2ccc(Cl)cc2C1] | (2R,4S)-1-[(2S or 2R)- 7-chloro-1,2,3,4- tetrahydronaphthalen- 2-yl]-4-{[4-(3- methanesulfonyl- propanesulfonyl) phenoxy]methyl}-2- methylpyrrolidine | Calc'd 540.2 Found 540.1 | DAICEL CHIRALPAK AD | |
| 55 | 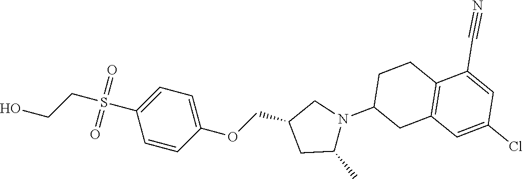 [SMILES: C[C@@H]1C[C@H](COc2ccc(S(=O)(=O)CCO)cc2)CN1C1CCc2c(C#N)cc(Cl)cc2C1] | 3-chloro-6-[(2R,4S)-4- {[4-(2- hydroxyethane- sulfonyl)phenoxy] methyl}-2- methylpyrrolidin-1- yl]-5,6,7,8- tetrahydronaphthalene- 1-carbonitrile | Calc'd 489.2 Found 489.2 | n/a | |
| 56 | 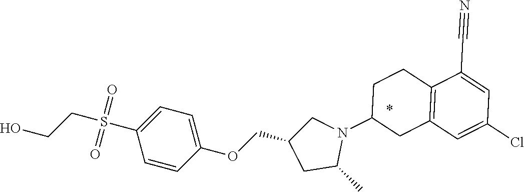 [SMILES: C[C@@H]1C[C@H](COc2ccc(S(=O)(=O)CCO)cc2)CN1C1CCc2c(C#N)cc(Cl)cc2C1] | (6R or 6S)-3-chloro-6- [(2R,4S)-4-{[4-(2- hydroxyethane- sulfony1)phenoxy] methyl}-2- methylpyrrolidin-1- yl]-5,6,7,8- tetrahydronaphthalene- 1-carbonitrile | Calc'd 489.2 Found 489.2 | DAICEL CHIRALPAK AD | 1st |
| 57 | 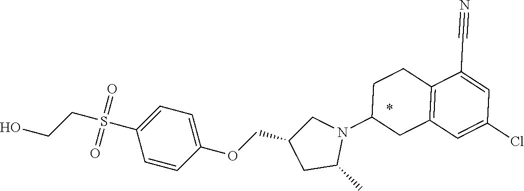 [SMILES: C[C@@H]1C[C@H](COc2ccc(S(=O)(=O)CCO)cc2)CN1C1CCc2c(C#N)cc(Cl)cc2C1] | (6S or 6R)-3-chloro-6- [(2R,4S)-4-{[4-(2- hydroxyethane- sulfony1)phenoxy] methyl}-2- methylpyrrolidin-1- yl]-5,6,7,8- tetrahydronaphthalene- 1-carbonitrile | Calc'd 489.2 Found 489.2 | DAICEL CHIRALPAK AD | 2nd |
| 58 | 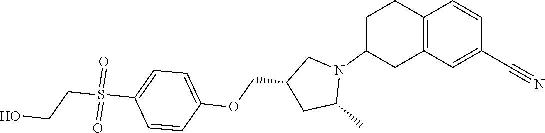 [SMILES: C[C@@H]1C[C@H](COc2ccc(S(=O)(=O)CCO)cc2)CN1C1CCc2ccc(C#N)cc2C1] | 7-[(2R,4S)-4-{[4-(2- hydroxyethane- sulfony1)phenoxy] methyl}-2- methylpyrrolidin-1- yl]-5,6,7,8- tetrahydronaphthalene- 2-carbonitrile | Calc'd 455.2 Found 455.2 | n/a | |
| 59 | 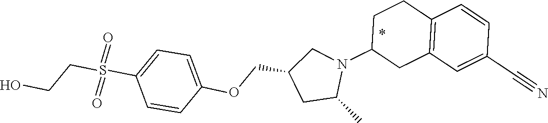 [SMILES: C[C@@H]1C[C@H](COc2ccc(S(=O)(=O)CCO)cc2)CN1C1CCc2ccc(C#N)cc2C1] | (7R or 7S)-7-[(2R,4S)- 4-{[4-(2- hydroxyethane- sulfony1)phenoxy] methyl}-2- methylpyrrolidin-1- yl]-5,6,7,8- tetrahydronaphthalene- 2-carbonitrile | Calc'd 455.2 Found 455.2 | prep- HPLC Waters Xbridge Prep OBD C18 | 1st |
| 60 | 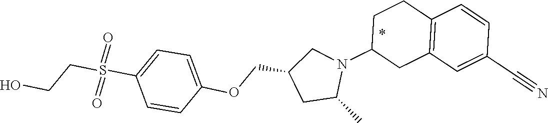 [SMILES: C[C@@H]1C[C@H](COc2ccc(S(=O)(=O)CCO)cc2)CN1C1CCc2ccc(C#N)cc2C1] | (7S or 7R)-7-[(2R,4S)- 4-{[4-(2- hydroxyethane- sulfony1)phenoxy] methyl}-2- methylpyrrolidin-1- yl]-5,6,7,8- tetrahydronaphthalene- 2-carbonitrile | Calc'd 455.2 Found 455.1 | prep- HPLC Waters Xbridge Prep OBD C18 | 2nd |
| 61 | 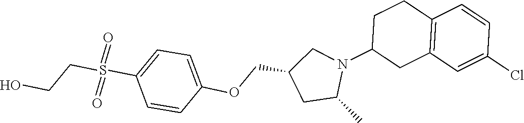 [SMILES: C[C@@H]1C[C@H](COc2ccc(S(=O)(=O)CCO)cc2)CN1C1CCc2ccc(Cl)cc2C1] | 2-(4-{[(3S,5R)-1-[7- chloro-1,2,3,4- tetrahydronaphthalen- 2-yl]-5- methylpyrrolidin-3- yl]methoxy} benzenesulfonyl) ethan-1-ol | Calc'd 464.2 Found 464.1 | n/a | |
| 62 | 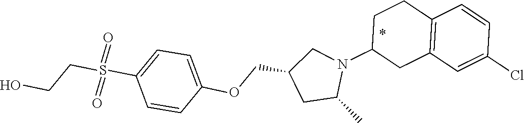 [SMILES: C[C@@H]1C[C@H](COc2ccc(S(=O)(=O)CCO)cc2)CN1C1CCc2ccc(Cl)cc2C1] | 2-(4-{[(35,5R)-1-[(2R or 2S)-7-chloro- 1,2,3,4- tetrahydronaphthalen- 2-yl]-5- methylpyrrolidin-3- yl]methoxy} benzenesulfonyl) ethan-1-ol | Calc'd 464.2 Found 464.2 | DAICEL CHIRALPAK AD | 1st |
| 63 | 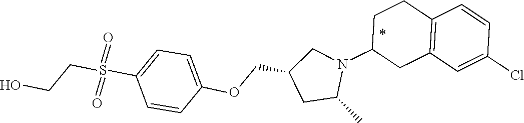 [SMILES: C[C@@H]1C[C@H](COc2ccc(S(=O)(=O)CCO)cc2)CN1C1CCc2ccc(Cl)cc2C1] | 2-(4-{[(35,5R)-1-[(2S or 2R)-7-chloro- 1,2,3,4- tetrahydronaphthalen- 2-yl]-5- methylpyrrolidin-3- yl]methoxy} benzenesulfonyl) ethan-1-ol | Calc'd 464.2 Found 464.1 | DAICEL CHIRALPAK AD | 2nd |
| 64 | 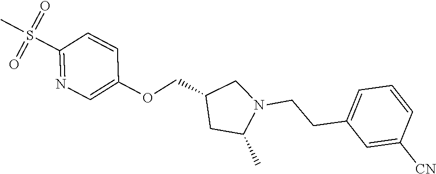 [SMILES: C[C@@H]1C[C@H](COc2ccc(S(C)(=O)=O)nc2)CN1CCc1cccc(C#N)c1] | 3-{2-[(2R,4S)-4-{[(6- methanesulfonyl- pyridin-3-yl)oxy] methyl}-2- methylpyrrolidin-1- yl]ethyl}benzonitrile | Calc'd 400.2 Found 400.1 | n/a | |
| 65 | 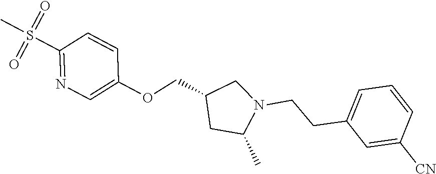 [SMILES: C[C@@H]1C[C@H](COc2ccc(S(C)(=O)=O)nc2)CN1CCc1cccc(C#N)c1] | 3-chloro-5-{2- [(2R,4S)-4-{[(6- methanesulfonyl- pyridin-3-yl)oxy] methyl}-2- methylpyrrolidin-1- yl]ethyl}benzonitrile | Calc'd 434.1 Found 434.2 | n/a | |
| 66 | 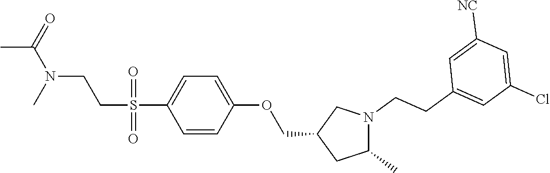 [SMILES: CC(=O)N(C)CCS(=O)(=O)c1ccc(OC[C@H]2C[C@@H](C)N(CCc3cc(Cl)cc(C#N)c3)C2)cc1] | N-[2-(4-{[(3S,5R)-1- [2-(3-chloro-5- cyanophenyl)ethyl]-5- methylpyrrolidin-3- yl]methoxy} benzenesulfonyl) ethyl]-N- methylacetamide | Calc'd 518.2 Found 518.2 | n/a | |
| 67 | 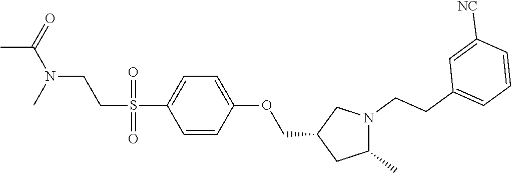 [SMILES: CC(=O)N(C)CCS(=O)(=O)c1ccc(OC[C@H]2C[C@@H](C)N(CCc3cccc(C#N)c3)C2)cc1] | N-[2-(4-{[(35,5R)-1- [2-(3- cyanophenyl)ethyl]-5- methylpyrrolidin-3- yl]methoxy} benzenesulfonyl) ethyl]-N- methylacetamide | Calc'd 484.2 Found 484.2 | n/a | |
| 68 | 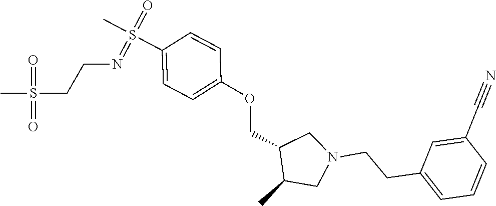 [SMILES: C[C@@H]1CN(CCc2cccc(C#N)c2)C[C@H]1COc1ccc(S(C)(=O)=NCCS(C)(=O)=O)cc1] | 3-{2-[(35,4S)-3-[(4- {[(2- methanesulfonylethyl) imino](methyl)oxo-λ⁶- sulfanyl}phenoxy) methyl]-4- methylpyrrolidin-1- yl]ethyl} benzonitrile | Calc'd 504.2 Found 504.3 | n/a | |
| 69 | 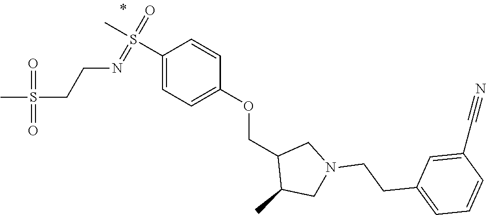 [SMILES: C[C@@H]1CN(CCc2cccc(C#N)c2)CC1COc1ccc(S(C)(=O)=NCCS(C)(=O)=O)cc1] | 3-{2-[(35,4S)-3-[(4- {[(2- methanesulfonylethyl) imino](methyl)oxo-λ⁶- sulfanyl}phenoxy) methyl]-4- methylpyrrolidin-1- yl]ethyl} benzonitrile | Calc'd 504.2 Found 504.3 | DAICEL CHIRALPAK IG | 1st |
| 70 | 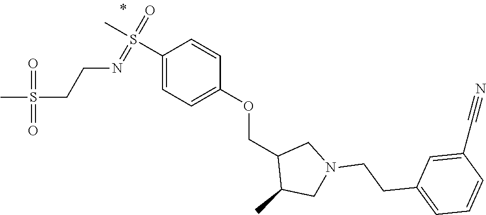 [SMILES: C[C@@H]1CN(CCc2cccc(C#N)c2)CC1COc1ccc(S(C)(=O)=NCCS(C)(=O)=O)cc1] | 3-{2-[(3S,4S)-3-[(4- {[(2- methanesulfonylethyl) imino](methyl)oxo-λ⁶- sulfanyl}phenoxy) methyl]-4- methylpyrrolidin-1- yl]ethyl}benzonitrile | Calc'd 504.2 Found 504.3 | DAICEL CHIRALPAK IG | 2nd |
| 71 | 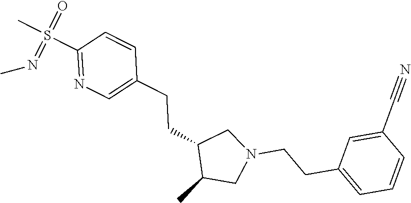 [SMILES: CN=S(C)(=O)c1ccc(CC[C@@H]2CN(CCc3cccc(C#N)c3)C[C@H]2C)cn1] | 3-{2-[(3S,4S)-3- methyl-4-[{{6- [methyl(methylimino) oxo-26- sulfanyl]pyridin-3- yl}oxy)methyl] pyrrolidin-1- yl]ethyl}benzonitrile | Calc'd 413.2 Found 413.3 | n/a | |
| 72 | 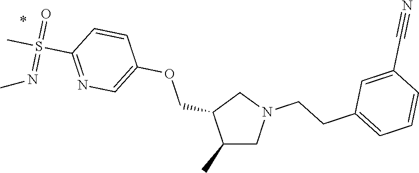 [SMILES: CN=S(C)(=O)c1ccc(OC[C@@H]2CN(CCc3cccc(C#N)c3)C[C@H]2C)cn1] | 3-{2-[(35,4S)-3- methyl-4-[{{6- [methyl(methylimino) oxo-λ⁶- sulfanyl]pyridin-3- yl}oxy)methyl] pyrrolidin-1- yl]ethyl}benzonitrile | Calc'd 413.2 Found 413.2 | Phenomenex- Cellulose-2 | 1st |
| 73 | 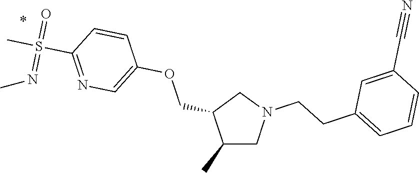 [SMILES: CN=S(C)(=O)c1ccc(OC[C@@H]2CN(CCc3cccc(C#N)c3)C[C@H]2C)cn1] | 3-{2-[(3S,4S)-3- methyl-4-[{{6- [methyl(methylimino) xox-λ⁶- sulfanyl]pyridin-3- yl}oxy)methyl] pyrrolidin-1- yl]ethyl}benzonitrile | Calc'd 413.2 Found 413.2 | Phenomenex- Cellulose-2 | 2nd |
| 74 | 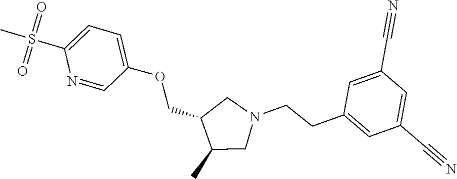 [SMILES: C[C@@H]1CN(CCc2cc(C#N)cc(C#N)c2)C[C@H]1COc1ccc(S(C)(=O)=O)nc1] | 5-{2-[(35,4S)-3-{[(6- methanesulfonyl- pyridin-3-yl)oxy] methyl}-4- methylpyrrolidin-1- yl]ethyl}benzene-1,3- dicarbonitrile | Calc'd 425.2 Found 425.2 | n/a | |
| 75 | 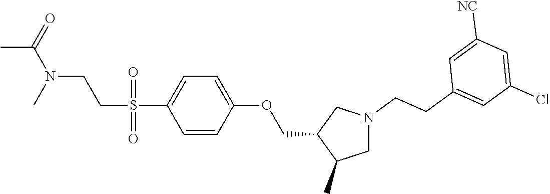 [SMILES: CC(=O)N(C)CCS(=O)(=O)c1ccc(OC[C@@H]2CN(CCc3cc(Cl)cc(C#N)c3)C[C@H]2C)cc1] | N-[2-(4-{[(35,4S)-1- [2-(3-chloro-5- cyanophenyl)ethyl]-4- methylpyrrolidin-3- yl]methoxy}benzenes ulfonyl)ethyl]-N- methylacetamide | Calc'd 518.2 Found 518.1 | n/a | |
| 76 | 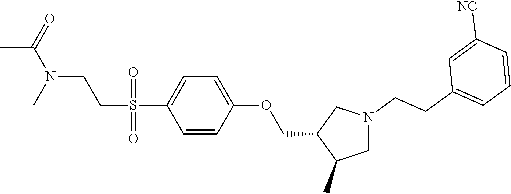 [SMILES: CC(=O)N(C)CCS(=O)(=O)c1ccc(OC[C@@H]2CN(CCc3cccc(C#N)c3)C[C@H]2C)cc1] | N-[2-(4-{[(35,4S)-1- [2-(3- cyanophenyl)ethyl]-4- methylpyrrolidin-3- yl]methoxy} benzenesulfonyl) ethyl]-N- methylacetamide | Calc'd 484.2 Found 484.4 | n/a | |
| 77 | 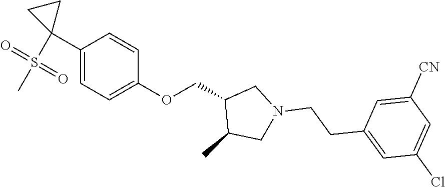 [SMILES: C[C@@H]1CN(CCc2cc(Cl)cc(C#N)c2)C[C@H]1COc1ccc(C2(S(C)(=O)=O)CC2)cc1] | 3-chloro-5-{2- [(3S,4S)-3-{[4-(1- methanesulfonyl- cyclopropyl) phenoxy] methyl}-4- methylpyrrolidin-1- yl]ethyl}benzonitrile | Calc'd 473.2 Found 473.1 | n/a | |
| 78 | 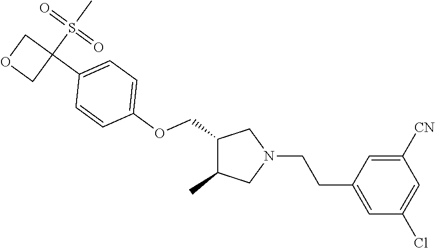 [SMILES: C[C@@H]1CN(CCc2cc(Cl)cc(C#N)c2)C[C@H]1COc1ccc(C2(S(C)(=O)=O)COC2)cc1] | 3-chloro-5-{2- [(35,4S)-3-{[4-(3- methanesulfonyl- oxetan-3- yl)phenoxy]methyl}- 4-methylpyrrolidin-1- yl]ethyl}benzonitrile | Calc'd 489.2 Found 489.2 | n/a | |
| 79 | 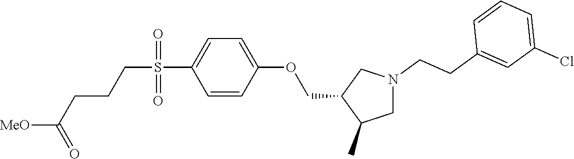 [SMILES: COC(=O)CCCS(=O)(=O)c1ccc(OC[C@@H]2CN(CCc3cccc(Cl)c3)C[C@H]2C)cc1] | methyl 4-(4-{[(3S,4S)- 1-[2-(3- chlorophenyl)ethyl]-4- methylpyrrolidin-3- yl]methoxy} benzenesulfonyl) butanoate | Calc'd 494.2 Found 494.2 | n/a | |
| 80 | 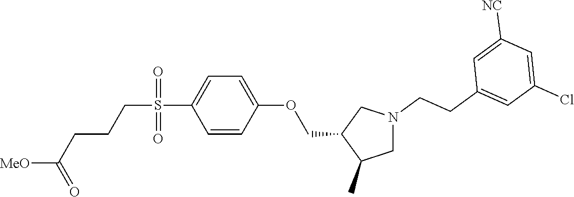 [SMILES: COC(=O)CCCS(=O)(=O)c1ccc(OC[C@@H]2CN(CCc3cc(Cl)cc(C#N)c3)C[C@H]2C)cc1] | methyl 4-(4-{[(3S,4S)- 1-[2-(3-chloro-5- cyanophenyl)ethyl]-4- methylpyrrolidin-3- yl]methoxy} benzenesulfonyl) butanoate | Calc'd 519.2 Found 519.3 | n/a | |
| 81 | 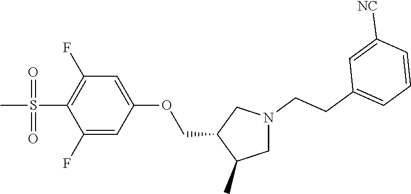 [SMILES: C[C@@H]1CN(CCc2cccc(C#N)c2)C[C@H]1COc1cc(F)c(S(C)(=O)=O)c(F)c1] | 3-{2-[(3S,4S)-3-[(3,5- difluoro-4- methanesulfonyl- phenoxy)methyl]-4- methylpyrrolidin-1- yl]ethyl}benzonitrile | Calc'd 435.2 Found 435.2 | n/a | |
| 82 | 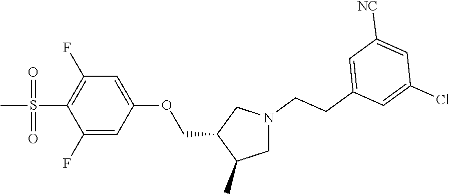 [SMILES: C[C@@H]1CN(CCc2cc(Cl)cc(C#N)c2)C[C@H]1COc1cc(F)c(S(C)(=O)=O)c(F)c1] | 3-chloro-5-{2- [(3S,4S)-3-[(3,5- difluoro-4- methanesulfonyl- phenoxy)methyl]-4- methylpyrrolidin-1- yl]ethyl}benzonitrile | Calc'd 469.1 Found 469.2 | n/a | |
| 83 | 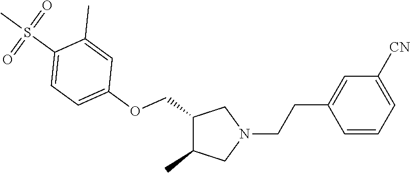 [SMILES: Cc1cc(OC[C@@H]2CN(CCc3cccc(C#N)c3)C[C@H]2C)ccc1S(C)(=O)=O] | 3-{2-[(3S,4S)-3-[(4- methanesulfonyl-3- methylphenoxy) methy1]-4- methylpyrrolidin- 1-yl]ethyl} benzonitrile | Calc'd 413.2 Found 413.2 | n/a | |
| 84 | 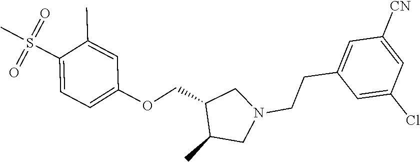 [SMILES: Cc1cc(OC[C@@H]2CN(CCc3cc(Cl)cc(C#N)c3)C[C@H]2C)ccc1S(C)(=O)=O] | 3-chloro-5-{2- [(3S,4S)-3-[(4- methanesulfonyl-3- methylphenoxy) methy1]-4- methylpyrrolidin- 1-yl]ethyl} benzonitrile | Calc'd 447.2 Found 447.1 | n/a | |
| 85 | 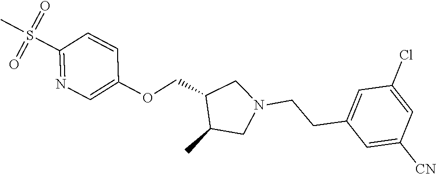 [SMILES: C[C@@H]1CN(CCc2cc(Cl)cc(C#N)c2)C[C@H]1COc1ccc(S(C)(=O)=O)nc1] | 3-chloro-5-{2- [(3S,4S)-3-{[(6- methanesulfonyl- pyridin-3-yl)oxy] methyl}-4- methylpyrrolidin-1- yl]ethyl}benzonitrile | Calc'd 434.1 Found 434.2 | n/a | |
| 86 | 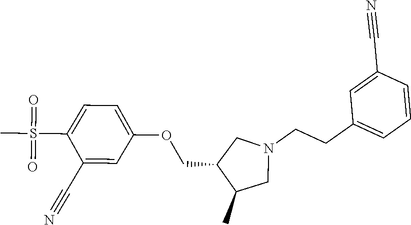 [SMILES: C[C@@H]1CN(CCc2cccc(C#N)c2)C[C@H]1COc1ccc(S(C)(=O)=O)c(C#N)c1] | 5-{[(3S,4S)-1-[2-(3- cyanophenyl)ethyl]-4- methylpyrrolidin-3- yl]methoxy}-2- methanesulfonyl- benzonitrile | Calc'd 424.2 Found 424.2 | n/a | |
| 87 | 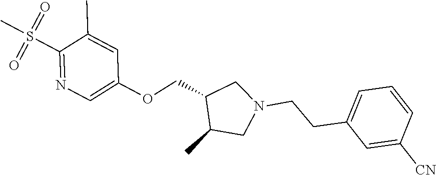 [SMILES: Cc1cc(OC[C@@H]2CN(CCc3cccc(C#N)c3)C[C@H]2C)cnc1S(C)(=O)=O] | 3-{2-[(3S,4S)-3-{[(6- methanesulfonyl-5- methylpyridin-3- yl)oxy]methyl}-4- methylpyrrolidin-1- ylethyl} benzonitrile | Calc'd 414.2 Found 414.0 | n/a | |
| 88 | 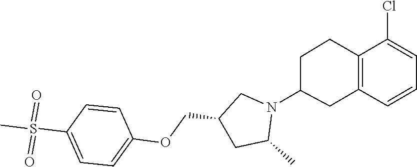 [SMILES: C[C@@H]1C[C@H](COc2ccc(S(C)(=O)=O)cc2)CN1C1CCc2c(Cl)cccc2C1] | (2R,4S)-1-(5-chloro- 1,2,3,4- tetrahydronaphthalen- 2-yl)-4-[(4- methanesulfonyl- phenoxy)methyl]-2- methylpyrrolidine | Calc'd 434.2 Found 434.1 | n/a | |
| 89 | 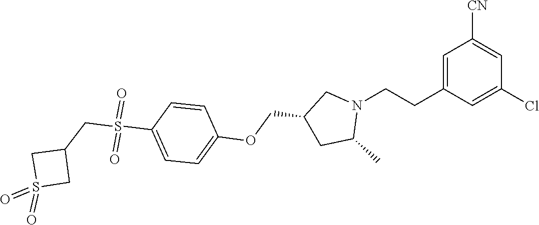 [SMILES: C[C@@H]1C[C@H](COc2ccc(S(=O)(=O)CC3CS(=O)(=O)C3)cc2)CN1CCc1cc(Cl)cc(C#N)c1] | 3-chloro-5-{2- [(3S,4S)-3-({4-[(1,1- dioxo-126-thietan-3- yl)methanesulfonyl] phenoxy}methyl)-4- methylpyrrolidin-1- yl]ethyl}benzonitrile | Calc'd 537.1 Found 537.1 | n/a | |
| 90 | 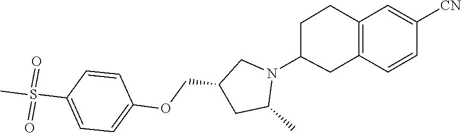 [SMILES: C[C@@H]1C[C@H](COc2ccc(S(C)(=O)=O)cc2)CN1C1CCc2cc(C#N)ccc2C1] | 7-[(2R,4S)-4-[(4- methanesulfonyl phenoxy)methyl]-2- methylpyrrolidin-1- yl]-5,6,7,8- tetrahydronaphthalene- 2-carbonitrile | Calc'd 425.2 Found 425.2 | n/a | |

### Example 6

### 3-((R) or (S))-1-hydroxy-2-((2R,4S)-2-methyl-4-(((6-(methylsulfonyl)pyridin-3-yl)oxy)methyl)pyrrolidin-1-yl)ethyl)benzonitrile (Compound 93) and 3-((S) or (R))-1-hydroxy-2-((2R,4S)-2-methyl-4-(((6-(methylsulfonyl)pyridin-3-yl)oxy)methyl)pyrrolidin-1-yl)ethyl)benzonitrile (Compound 94)

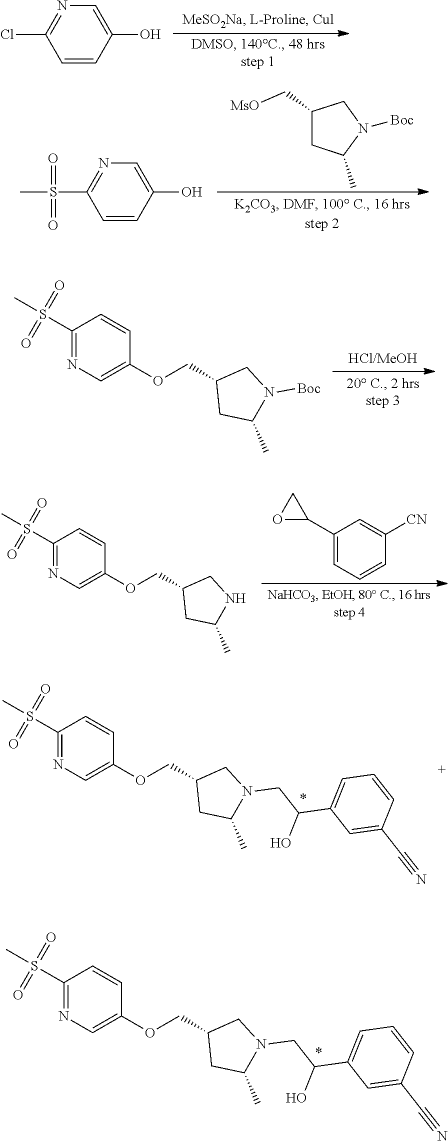

### Step 1: 6-(methylsulfonyl)pyridin-3-ol

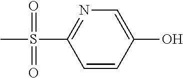

To a mixture of 6-chloropyridin-3-ol (5.00 g, 38.6 mmol) and sodium methanesulfinate (15.76 g, 154.4 mmol) in DMSO (50 mL) were added (2S)-pyrrolidine-2-carboxylic acid (1.33 g, 11.6 mmol), CuI (2.21 g, 11.6 mmol) and K₂CO₃ (1.60 g, 11.6 mmol). The mixture was purged with N₂ three times and stirred at 140° C. for 48 h under N₂. The reaction mixture was allowed to cool to RT, then was quenched by addition of water (150 mL) and extracted with EtOAc (6×50 mL). The combined organic layers were washed with brine (50 mL), dried over Na₂SO₄, filtered and concentrated under reduced pressure. The residue was purified by flash silica gel chromatography (Biotage 40 g cartridge, 0-80% ethyl acetate/petroleum ether) to provide 6-methylsulfonylpyridin-3-ol: MS=174.1 [M+H]⁺.

### Step 2: (2R,4S)-tert-butyl 2-methyl-4-(((6-(methylsulfonyl)pyridin-3-yl)oxy)methyl)pyrrolidine-1-carboxylate

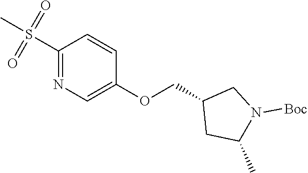 [SMILES: C[C@@H]1C[C@H](COc2ccc(S(C)(=O)=O)nc2)CN1C(=O)OC(C)(C)C]

To a solution of 6-methylsulfonylpyridin-3-ol (250 mg, 1.44 mmol) in DMF (5 mL) were added tert-butyl (2R,4S)-2-methyl-4-(methylsulfonyloxymethyl)pyrrolidine-1-carboxylate (423 mg, 1.44 mmol) and K₂CO₃ (399 mg, 2.89 mmol). The mixture was then heated to 100° C. and allowed to stir for 16 h. The reaction mixture was then allowed to cool to RT, diluted with water (20 mL) and extracted with EtOAc (3×20 mL). The combined organic layers were washed with brine (20 mL), dried over Na₂SO₄, filtered and concentrated under reduced pressure. The residue was purified by flash silica gel chromatography (Biotage 12 g cartridge, 0-75% ethyl acetate/petroleum ether) to provide (2R,4S)-tert-butyl 2-methyl-4-(((6-(methylsulfonyl)pyridin-3-yl)oxy)methyl)pyrrolidine-1-carboxylate: MS=315.0 [M−C₄H₈+H]⁺.

### Step 3: 5-(((3S,5R)-5-methylpyrrolidin-3-yl)methoxy)-2-(methylsulfonyl)pyridine

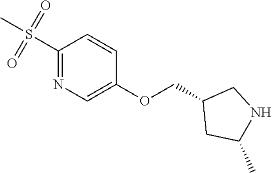 [SMILES: C[C@@H]1C[C@H](COc2ccc(S(C)(=O)=O)nc2)CN1]

A solution of (2R,4S)-tert-butyl 2-methyl-4-(((6-(methylsulfonyl)pyridin-3-yl)oxy)methyl)pyrrolidine-1-carboxylate (500 mg, 1.35 mmol) in HCl/MeOH (20 mL, 4.0 M, 80 mmol) was stirred at RT for 2 h. The reaction mixture was concentrated under reduced pressure to provide 5-[[(3S,5R)-5-methylpyrrolidin-3-yl]methoxy]-2-methylsulfonyl-pyridine (HCl salt), which was used without further purification: MS=271.1 [M+H]⁺.

### Step 4: 3-((R) or (S))-1-hydroxy-2-((2R,4S)-2-methyl-4-(((6-(methylsulfonyl)pyridin-3-yl)oxy)methyl)pyrrolidin-1-yl)ethyl)benzonitrile (Compound 93) and 3-((S) or (R)-1-hydroxy-2-((2R,4S)-2-methyl-4-(((6-(methylsulfonyl)pyridin-3-yl)oxy)methyl)pyrrolidin-1-yl)ethyl)benzonitrile (Compound 94)

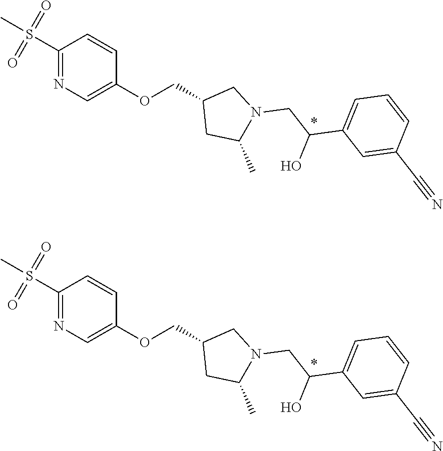 [SMILES: C[C@@H]1C[C@H](COc2ccc(S(C)(=O)=O)nc2)CN1CC(O)c1cccc(C#N)c1 ; C[C@@H]1C[C@H](COc2ccc(S(C)(=O)=O)nc2)CN1CC(O)c1cccc(C#N)c1]

To a solution of 5-[[(3S,5R)-5-methylpyrrolidin-3-yl]methoxy]-2-methylsulfonyl-pyridine (320 mg, 1.04 mmol, HCl salt) in EtOH (4 mL) were added NaHCO₃ (349 mg, 4.16 mmol) and 3-(oxiran-2-yl)benzonitrile (151 mg, 1.04 mmol) at RT. The mixture was then heated to 80° C. and allowed to stir for 16 h. The reaction mixture was concentrated under reduced pressure. Purification by reverse phase HPLC (Phenomenex C18, 20-50% CH₃CN/H₂O (0.1% NH₄HCO₃) to give the first eluting isomer, 3-((R) or (S))-1-hydroxy-2-((2R,4S)-2-methyl-4-(((6-(methylsulfonyl)pyridin-3-yl)oxy)methyl)pyrrolidin-1-yl)ethyl)benzonitrile (Compound 93): ¹H NMR (400 MHz, DMSO-d₆) δ 8.45 (d, J=2.8 Hz, 1H), 8.00 (d, J=8.8 Hz, 1H), 7.72 (s, 1H), 7.66-7.63 (m, 3H), 7.45-7.41 (m, 1H), 5.41 (s, 1H), 4.66 (t, J=6.4 Hz, 1H), 3.93 (d, J=7.6 Hz, 2H), 3.21 (s, 3H), 3.15-3.12 (m, 1H), 2.77-2.75 (m, 1H), 2.41-2.36 (m, 5H), 2.06-2.02 (m, 1H), 1.00-0.93 (m, 1H), 0.82 (d, J=6.0 Hz, 3H) MS=416.1 [M+H]⁺. The second eluting isomer 3-((S) or (R))-1-hydroxy-2-((2R,4S)-2-methyl-4-(((6-(methylsulfonyl) pyridin-3-yl)oxy)methyl)pyrrolidin-1-yl)ethyl)benzonitrile: (Compound 94): ¹H NMR (400 MHz, DMSO-d₆) δ 8.43 (d, J=2.8 Hz, 1H), 7.99 (d, J=8.8 Hz, 1H), 7.76 (s, 1H), 7.69-7.63 (m, 3H), 7.48-7.46 (m, 1H), 5.15 (s, 1H), 4.71-4.68 (m, 1H), 3.98 (d, J=7.6 Hz, 2H), 3.21 (s, 3H), 2.97 (d, J=7.2 Hz, 1H), 2.85-2.79 (m, 1H), 2.44-2.48 (m, 1H), 2.33-2.44 (m, 2H), 2.28-2.30 (m, 1H), 2.12-2.10 (m, 1H), 1.05-1.00 (m, 1H), 0.98 (d, J=6.0 Hz, 3H); MS=416.1 [M+H]⁺.

The following compounds in Table 4 were prepared according to procedures similar to step 1 described for Compounds 93-94 using the appropriate starting materials.

TABLE 4

| | | | Exact | | |
|---|---|---|---|---|---|
| | | | Mass | Chiral | Elution |
| # | Structure | IUPAC Name | [M + H]⁺ | column | order |
| 95 | 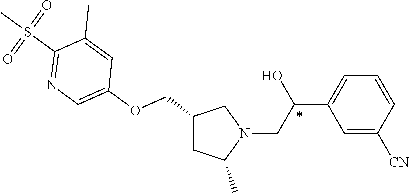 [SMILES: Cc1cc(OC[C@H]2C[C@@H](C)N(CC(O)c3cccc(C#N)c3)C2)cnc1S(C)(=O)=O] | 3-[(1R or 1S)-1- hydroxy-2-[(2R,4S)- 4-{[(6- methanesulfonyl-5- methylpyridin-3- yl)oxy]methyl}-2- methylpyrrolidin-1- yl]ethyl]benzonitrile | Calc'd 430.1 Found 430.2 | prep- HPLC Phenomenex Luna C18 | 3rd |
| 96 | 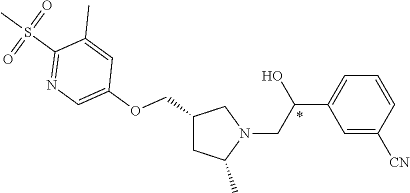 [SMILES: Cc1cc(OC[C@H]2C[C@@H](C)N(CC(O)c3cccc(C#N)c3)C2)cnc1S(C)(=O)=O] | 3-[(1S or 1R)-1- hydroxy-2-[(2R,4S)- 4-{[(6- methanesulfonyl-5- methylpyridin-3- yl)oxy]methyl}-2- methylpyrrolidin-1- yl]ethyl]benzonitrile | Calc'd 430.1 Found 430.2 | prep- HPLC Phenomenex Luna C18 | 4th |
| 97 | 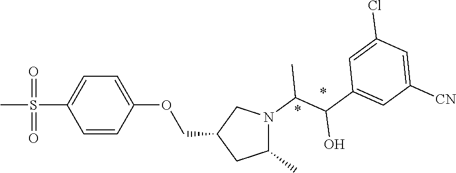 [SMILES: CC(C(O)c1cc(Cl)cc(C#N)c1)N1C[C@@H](COc2ccc(S(C)(=O)=O)cc2)C[C@H]1C] | 3-chloro-5-[(1S,2S or 1R,2R or 1R,2S or 1S,2R)-1-hydroxy-2- [(2R,4S)-4-[(4- methanesulfonyl- phenoxy)methyl]-2- methylpyrrolidin-1- yl]propyl]benzonitrile | Calc'd 463.1 Found 463.2 | prep- HPLC Phenomenex C18 | 1st |
| 98 | 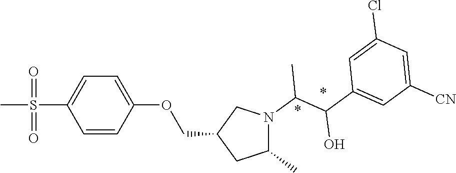 [SMILES: CC(C(O)c1cc(Cl)cc(C#N)c1)N1C[C@@H](COc2ccc(S(C)(=O)=O)cc2)C[C@H]1C] | 3-chloro-5-[(1R,2S or 1S,2R or 1S,2S or 1R,2R)-1-hydroxy-2- [(2R,4S)-4-[(4- methanesulfonyl- phenoxy)methyl]-2- methylpyrrolidin-1- yl]propyl]benzonitrile | Calc'd 463.1 Found 463.2 | prep- HPLC (column: Phenomenex C18 | 4th |
| 99 | 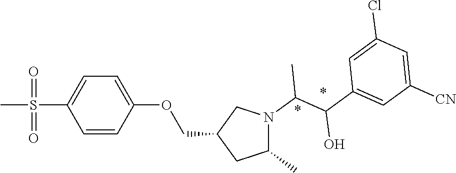 [SMILES: CC(C(O)c1cc(Cl)cc(C#N)c1)N1C[C@@H](COc2ccc(S(C)(=O)=O)cc2)C[C@H]1C] | 3-chloro-5-[(1S,2R or 1R,2S or 1S,2S or 1R,2R)-1-hydroxy-2- [(2R,4S)-4-[(4- methanesulfonyl- phenoxy)methyl]-2- methylpyrrolidin-1- yl]propyl]benzonitrile | Calc'd 463.1 Found 463.2 | prep- HPLC (column: Phenomenex C18 | 5th |
| 100 | 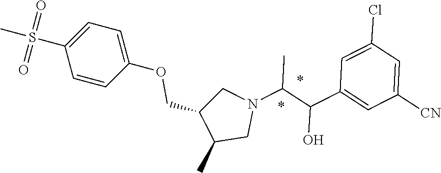 [SMILES: CC(C(O)c1cc(Cl)cc(C#N)c1)N1C[C@@H](COc2ccc(S(C)(=O)=O)cc2)[C@H](C)C1] | 3-chloro-5-((1R,2S or 1S,2R or 1R,2R or 1S,2S)-1-hydroxy-2- ((3S,4S)-3-methyl-4- ((4- (methylsulfonyl) phenoxy)methyl) pyrrolidin-1- yl)propyl)benzonitrile | Calc'd 463.1 Found 463.1 | Chiral SFC DAICEL CHIRAL- PAK AD | 1st |
| 101 | 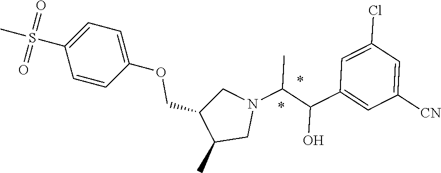 [SMILES: CC(C(O)c1cc(Cl)cc(C#N)c1)N1C[C@@H](COc2ccc(S(C)(=O)=O)cc2)[C@H](C)C1] | 3-chloro-5-((1S,2R or 1R,2R or 1S,2S or 1R,2S)-1-hydroxy-2- ((3S,4S)-3-methyl-4- ((4- (methylsulfonyl) phenoxy)methyl) pyrrolidin-1- yl)propyl)benzonitrile | Calc'd 463.1 Found 463.1 | Chiral SFC DAICEL CHIRAL- PAK AD | 2nd |
| 102 | 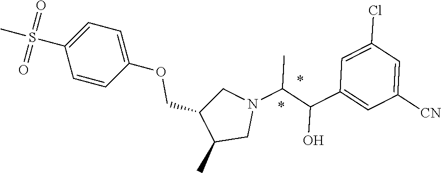 [SMILES: CC(C(O)c1cc(Cl)cc(C#N)c1)N1C[C@@H](COc2ccc(S(C)(=O)=O)cc2)[C@H](C)C1] | 3-chloro-5-((1R,2R or 1S,2S or 1R,2S or 1S,2R)-1-hydroxy-2- ((3S,4S)-3-methyl-4- ((4- (methylsulfonyl) phenoxy)methyl) pyrrolidin-1-yl) propyl)benzonitrile (Second Eluting Isomer) | Calc'd 463.1 Found 463.2 | DAICEL CHIRAL- PAK AD | 3rd |
| 103 | 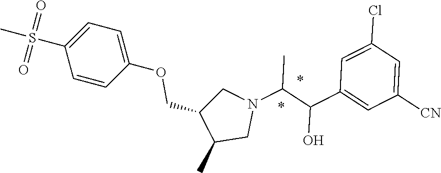 [SMILES: CC(C(O)c1cc(Cl)cc(C#N)c1)N1C[C@@H](COc2ccc(S(C)(=O)=O)cc2)[C@H](C)C1] | 3-chloro-5-((1S,2S or 1R,2S or 1S,2R or 1R,2R)-1-hydroxy- 2-((3S,4S)-3-methyl- 4-((4- (methylsulfonyl) phenoxy)methyl) pyrrolidin-1- yl)propyl)benzonitrile (Third Eluting Isomer) | Calc'd 463.1 Found 463.2 | DAICEL CHIRAL- PAK AD | 2^{nd} |
| 104 | 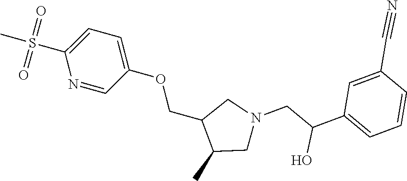 [SMILES: C[C@@H]1CN(CC(O)c2cccc(C#N)c2)CC1COc1ccc(S(C)(=O)=O)nc1] | 3-{1-hydroxy-2- [(3S,4S)-3-{[(6- methanesulfonylpyridin- 3-yl)oxy]methyl}-4- methylpyrrolidin-1- yl]ethyl}benzonitrile | Calc'd 416.2 Found 416.3 | n/a | |
| 105 | 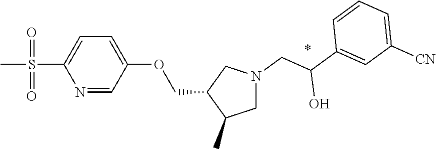 [SMILES: C[C@@H]1CN(CC(O)c2cccc(C#N)c2)C[C@H]1COc1ccc(S(C)(=O)=O)nc1] | 3-[(1S or 1R)-1- hydroxy-2-[(3S,4S)- 3-{[(6- methanesulfonylpyridin- 3-yl)oxy]methyl}-4- methylpyrrolidin-1- yl]ethyl]benzonitrile | Calc'd 416.2 Found 416.0 | DAICEL CHIRAL- PAK AD | 1st |
| 106 | 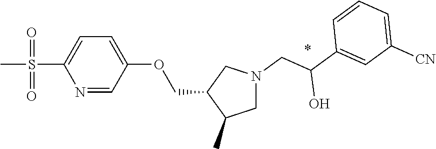 [SMILES: C[C@@H]1CN(CC(O)c2cccc(C#N)c2)C[C@H]1COc1ccc(S(C)(=O)=O)nc1] | 3-[(1R or 1S)-1- hydroxy-2-[(3S,4S)- 3-{[(6- methanesulfonylpyridin- 3-yl)oxy]methyl}-4- methylpyrrolidin-1- yl]ethyl]benzonitrile | Calc'd 416.2 Found 416.0 | Chiral SFC DAICEL CHIRAL- PAK AD | 2nd |

### Example 7 (Compound 107)

### (3S)-1-[2-(3-chlorophenyl)ethyl]-3-{[4-(3-methanesulfonyloxetan-3-yl)phenoxy]methyl}piperazine

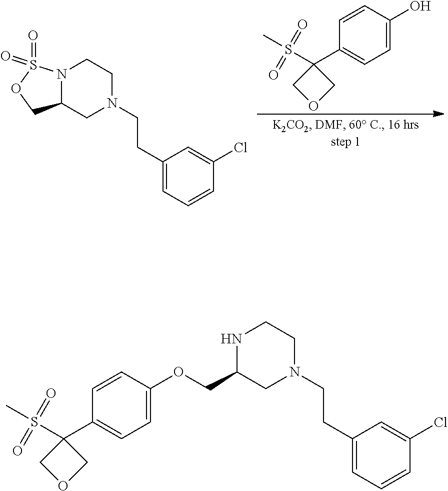 [SMILES: O=S1(=O)OC[C@@H]2CN(CCc3cccc(Cl)c3)CCN21 ; CS(=O)(=O)C1(c2ccc(O)cc2)COC1 ; CS(=O)(=O)C1(c2ccc(OC[C@@H]3CN(CCc4cccc(Cl)c4)CCN3)cc2)COC1]

### Step 1: (3S)-1-[2-(3-chlorophenyl)ethyl]-3-{[4-(3-methanesulfonyloxetan-3-yl)phenoxy]methyl}piperazine

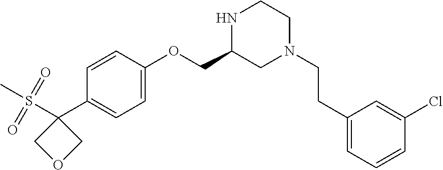 [SMILES: CS(=O)(=O)C1(c2ccc(OC[C@@H]3CN(CCc4cccc(Cl)c4)CCN3)cc2)COC1]

To a solution of 4-(3-(methylsulfonyl)oxetan-3-yl)phenol (80 mg, 350 umol) and (S)-5-(3-chlorophenethyl)hexahydro-[1,2,3]oxathiazolo[3,4-a]pyrazine 1,1-dioxide (111 mg, 350 umol) in DMF (1 mL) was added K₂CO₃ (96 mg, 701 umol). The mixture was stirred at 60° C. for 16 h. Then the mixture was allowed to cool to RT and adjusted to pH=3-4 with aq. HCl (3M), then was stirred at RT for 1 h. The reaction mixture was cooled to 0° C., and then quenched by addition of H₂O (10 mL), and extracted with EtOAc (5 mL×3). The combined organic layers were washed with brine (5 mL×2), dried over Na₂SO₄, filtered and concentrated under reduced pressure. The residue was purified by preparative reverse phase HPLC (Phenomenex luna C18, 14-34% MeCN:0.04% HCl in H₂O) to give (3S)-1-[2-(3-chlorophenyl)ethyl]-3-{[4-(3-methanesulfonyloxetan-3-yl)phenoxy]methyl}piperazine (Compound 107). ¹H NMR (DMSO-d₆, 400 MHz) δ 7.42-7.26 (m, 6H), 7.15 (d, J=8.8 Hz, 2H), 5.33 (d, J=7.6 Hz, 2H), 5.13 (d, J=7.6 Hz, 2H), 4.42-4.38 (m, 1H), 4.33-4.29 (m, 1H), 4.14-4.10 (m, 1H), 3.84-3.82 (m, 1H), 3.73-3.69 (m, 2H), 3.58-3.55 (m, 1H), 3.37-3.34 (m, 2H), 3.26-3.22 (m, 2H), 3.11-3.07 (m, 2H), 2.81 (s, 3H). MS=465.2 [M+H]⁺

The following compounds in Table 5 were prepared according to procedures similar to those described for Compound 107 using the appropriate starting materials.

TABLE 5

| | | | Exact | | |
|---|---|---|---|---|---|
| | | | Mass | Chiral | Elution |
| # | Structure | IUPAC Name | [M + H]⁺ | Column | Order |
| 108 | 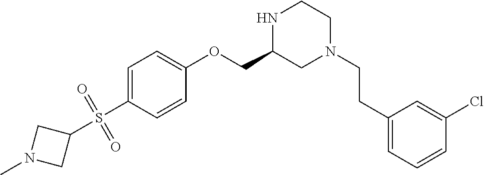 [SMILES: CN1CC(S(=O)(=O)c2ccc(OC[C@@H]3CN(CCc4cccc(Cl)c4)CCN3)cc2)C1] | (3S)-1-[2-(3- chlorophenyl) ethyl]-3-({4-[(1- methylazetidin- 3-yl)sulfonyl] phenoxy}methyl) piperazine | Calc'd 464.2 Found 464.2 | | N/A |
| 109 | 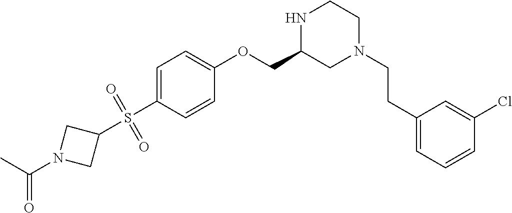 [SMILES: CC(=O)N1CC(S(=O)(=O)c2ccc(OC[C@@H]3CN(CCc4cccc(Cl)c4)CCN3)cc2)C1] | 1-[3-(4-{[(2S)-4- [2-(3- chlorophenyl) ethyl]piperazin-2- yl]methoxy} benzenesulfonyl) azetidin-1-yl] ethan-1-one | Calc'd 492.2 Found 492.3 | | N/A |
| 110 | 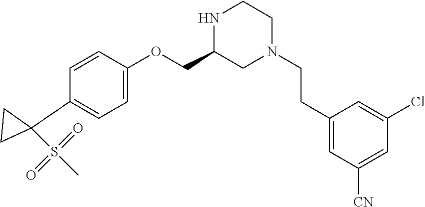 [SMILES: CS(=O)(=O)C1(c2ccc(OC[C@@H]3CN(CCc4cc(Cl)cc(C#N)c4)CCN3)cc2)CC1] | 3-chloro-{2- [(3S)-3-{[4-(1- methanesulfonyl cyclopropyl) phenoxy]methyl} piperazin-1- yl]ethyl} benzonitrile | Calc'd 474.2 Found 474.1 | | N/A |
| 111 | 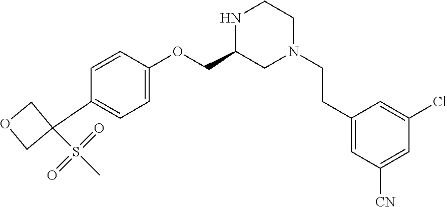 [SMILES: CS(=O)(=O)C1(c2ccc(OC[C@@H]3CN(CCc4cc(Cl)cc(C#N)c4)CCN3)cc2)COC1] | 3-chloro-5-{2- [(3S)-3-{[4-(3- methanesulfonyl oxetan-3- yl)phenoxy] methyl}piperazin- 1-yl]ethyl} benzonitrile | Calc'd 490.2 Found 490.2 | | N/A |
| 112 | 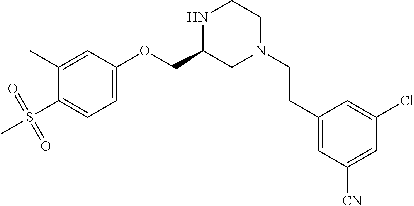 [SMILES: Cc1cc(OC[C@@H]2CN(CCc3cc(Cl)cc(C#N)c3)CCN2)ccc1S(C)(=O)=O] | 3-chloro-5-{2- [(3S)-3-[(4- methanesulfonyl- 3-methylphenoxy) methyl]piperazin- 1-yl]ethyl} benzonitrile | Calc'd 448.1 Found 448.1 | | N/A |
| 113 | 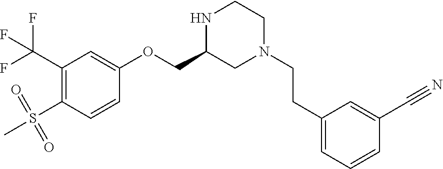 [SMILES: CS(=O)(=O)c1ccc(OC[C@@H]2CN(CCc3cccc(C#N)c3)CCN2)cc1C(F)(F)F] | 3-{2-[(3S)-3-{[4- methanesulfonyl- 3-(trifluoromethyl) phenoxy]methyl} piperazin-1-yl] ethyl}benzonitrile | Calc'd 468.2 Found 468.2 | | N/A |
| 114 | 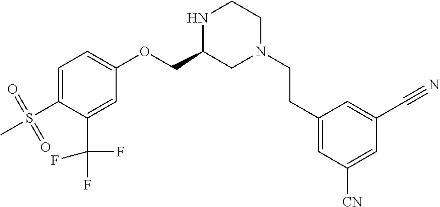 [SMILES: CS(=O)(=O)c1ccc(OC[C@@H]2CN(CCc3cc(C#N)cc(C#N)c3)CCN2)cc1C(F)(F)F] | 3-chloro-5-{2- [(3S)-3-{[4- methanesulfonyl- 3-(trifluoromethyl) phenoxy]methyl} piperazin-1-yl] ethyl}benzonitrile | Calc'd 502.1 Found 502.2 | | N/A |
| 140 | 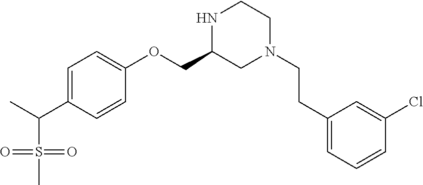 [SMILES: CC(c1ccc(OC[C@@H]2CN(CCc3cccc(Cl)c3)CCN2)cc1)S(C)(=O)=O] | (3S)-1-[2-(3- chlorophenyl) ethyl]-3-{[4-(1- methanesulfonyl ethyl)phenoxy] methyl}piperazine | Calc'd 437.2 Found 437.2 | | N/A |
| 141 | 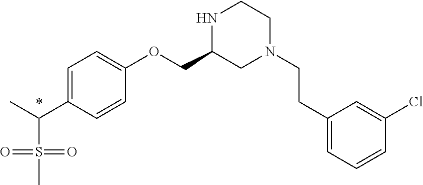 [SMILES: CC(c1ccc(OC[C@@H]2CN(CCc3cccc(Cl)c3)CCN2)cc1)S(C)(=O)=O] | (3S)-1-[2-(3- chlorophenyl) ethyl]-3-({4- [(1R) or (1S)-1- methanesulfonyl ethyl]phenoxy} methyl)piperazine | Calc'd 437.2 Found 437.2 | DAICEL CHIRAL- PAK AD | 2nd |
| 142 | 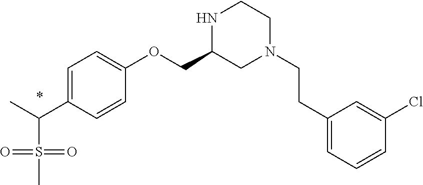 [SMILES: CC(c1ccc(OC[C@@H]2CN(CCc3cccc(Cl)c3)CCN2)cc1)S(C)(=O)=O] | (3S)-1-[2-(3- chlorophenyl) ethyl]-3-({4-[(1S) or (1R)-1- methanesulfonyl ethyl]phenoxy} methyl)piperazine | Calc'd 437.2 Found 437.2 | DAICEL CHIRAL- PAK AD | 1st |

### Example 8 (Compound 115, 116, 117)

### 3-(3-chlorophenyl)-2-[(3S,4S)-3-[(4-methanesulfonylphenoxy)methyl]-4-methylpyrrolidin-1-yl]propan-1-ol (Compound 115), (2R or 2S)-3-(3-chlorophenyl)-2-[(3S,4S)-3-[(4-methanesulfonylphenoxy)methyl]-4-methylpyrrolidin-1-yl]propan-1-ol (Compound 116) and (2S or 2R)-3-(3-chlorophenyl)-2-[(3S,4S)-3-[(4-methanesulfonylphenoxy)methyl]-4-methylpyrrolidin-1-yl]propan-1-ol (Compound 117)

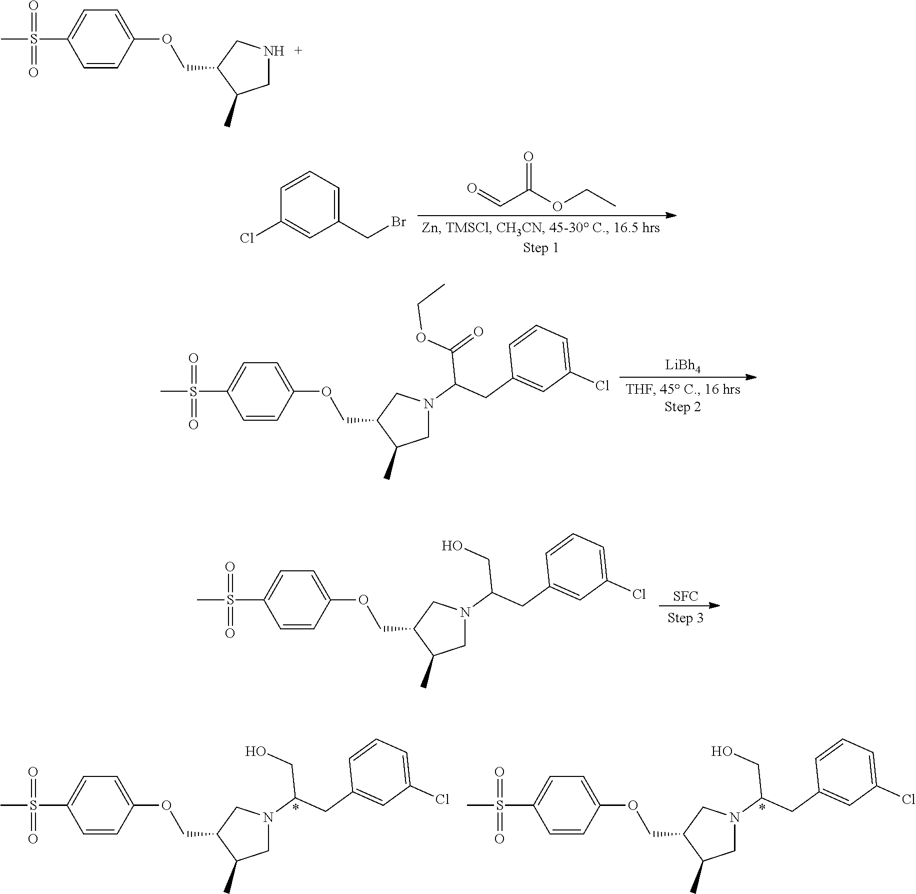

### Step 1: ethyl 3-(3-chlorophenyl)-2-((3S,4S)-3-methyl-4-((4-(methylsulfonyl)phenoxy)methyl)pyrrolidin-1-yl)propanoate

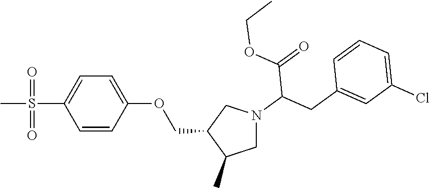 [SMILES: CCOC(=O)C(Cc1cccc(Cl)c1)N1C[C@@H](COc2ccc(S(C)(=O)=O)cc2)[C@H](C)C1]

To a suspension of Zn (303 mg, 4.64 mmol) in MeCN (8 mL) were added TMSCl (201 mg, 1.86 mmol) and three drops of 1-(bromomethyl)-3-chloro-benzene (in 0.1 mL of MeCN), the mixture was stirred at 45° C. for 0.5 h. Then 1-(bromomethyl)-3-chloro-benzene (286 mg, 1.39 mmol), (3S,4S)-3-methyl-4-((4-(methylsulfonyl)phenoxy)methyl)pyrrolidine (250 mg, 928 umol) and ethyl 2-oxoacetate (284 mg, 1.39 mmol, 50% purity) were added to the mixture. The reaction mixture was then stirred at 30° C. for 16 h. The reaction was quenched by addition of sat. aq. NH₄Cl (aq, 15 mL) and extracted with EtOAc (10 mL×3). The combined organic layers were washed with brine (10 mL), dried over Na₂SO₄, filtered and concentrated under reduced pressure to give a residue. The residue was purified by preparative reverse phase HPLC (Kromasil C18, 50-70% MeCN:10 mM NH₄HCO₃ in H₂O) to give ethyl 3-(3-chlorophenyl)-2-((3S,4S)-3-methyl-4-((4-(methylsulfonyl)phenoxy)methyl)pyrrolidin-1-yl)propanoate. MS=480.2 [M+H]⁺

### Step 2: 3-(3-chlorophenyl)-2-[(3S,4S)-3-[(4-methanesulfonylphenoxy)methyl]-4-methylpyrrolidin-1-yl]propan-1-ol

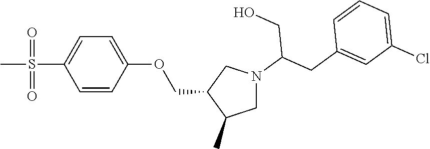 [SMILES: C[C@@H]1CN(C(CO)Cc2cccc(Cl)c2)C[C@H]1COc1ccc(S(C)(=O)=O)cc1]

To a solution of ethyl 3-(3-chlorophenyl)-2-((3S,4S)-3-methyl-4-((4-(methylsulfonyl) phenoxy) methyl)pyrrolidin-1-yl)propanoate (70 mg, 146 umol) in THE (2 mL) was added LiBH₄ (4 M in THF, 547 uL) at 0° C. under N₂. The reaction mixture was then stirred at 45° C. for 16 h. The reaction was quenched with sat. aq. NH₄Cl (10 mL) and extracted with EtOAc (5 mL×3). The combined organic layers were washed with brine (10 mL), dried over Na₂SO₄, filtered and concentrated under reduced pressure to give a residue. The residue was purified by preparative reverse phase HPLC (Phenomenex luna C18, 22-38% MeCN:0.04% HCl in H₂O) to give 3-(3-chlorophenyl)-2-[(3S,4S)-3-[(4-methanesulfonylphenoxy)methyl]-4-methylpyrrolidin-1-yl]propan-1-ol (Compound 115). ¹H NMR (DMSO-d₆, 400 MHz) δ 10.45 (s, 1H), 7.87 (d, J=8.8 Hz, 2H), 7.47 (s, 1H), 7.46-7.31 (m, 3H), 7.20-7.17 (m, 2H), 4.25-4.16 (m, 1H), 4.14-4.12 (m, 1H), 3.79-3.64 (m, 4H), 3.54-3.50 (m, 2H) 3.38-3.35 (m, 1H), 3.30-3.20 (m, 1H), 3.16 (s, 3H), 2.94-2.90 (m. 2H), 2.25-2.23 (m, 1H), 2.35 (s, 1H), 1.16 (d, J=6.0 Hz, 3H). MS=438.2 [M+H]⁺

### Step 3: (2R or 2S)-3-(3-chlorophenyl)-2-[(3S,4S)-3-[(4-methanesulfonylphenoxy)methyl]-4-methylpyrrolidin-1-yl]propan-1-ol and (2S or 2R)-3-(3-chlorophenyl)-2-[(3S,4S)-3-[(4-methanesulfonylphenoxy)methyl]-4-methylpyrrolidin-1-yl]propan-1-ol

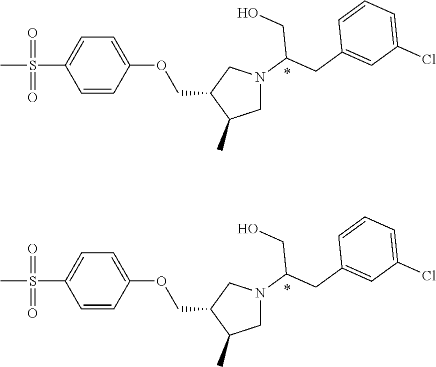 [SMILES: C[C@@H]1CN(C(CO)Cc2cccc(Cl)c2)C[C@H]1COc1ccc(S(C)(=O)=O)cc1 ; C[C@@H]1CN(C(CO)Cc2cccc(Cl)c2)C[C@H]1COc1ccc(S(C)(=O)=O)cc1]

3-(3-chlorophenyl)-2-((3S,4S)-3-methyl-4-((4-(methylsulfonyl)phenoxy)methyl)pyrrolidin-1-yl)propan-1-ol (30 mg, 69 umol) was further purified by preparative chiral SFC (Phenomenex-Cellulose-2, 50% ethanol with CO₂). The first eluting isomer of the the title compound (Compound 116): ¹H NMR (DMSO-d₆, 400 MHz) δ 7.83 (d, J=8.8 Hz, 2H), 7.28-7.13 (m, 6H), 4.43 (t, J=4.8 Hz, 1H), 4.04-3.96 (m, 2H), 3.37-3.36 (m, 2H), 3.15 (s, 3H), 2.95 (t, J=2.0 Hz, 1H), 2.85-2.75 (m, 2H), 2.70-2.65 (m, 2H), 2.55-2.50 (m, 1H), 2.25 (t, J=2.0 Hz, 1H), 2.05-1.95 (m, 1H), 1.90-1.80 (m, 1H), 1.05 (d, J=6.8 Hz, 3H). MS=438.3[M+H]⁺. The second eluting isomer of the the title compound (Compound 117): ¹H NMR (DMSO-d₆, 400 MHz) δ 7.83 (d, J=8.8 Hz, 2H), 7.28-7.12 (m, 6H), 4.01-3.94 (m, 2H), 3.35-3.32 (m, 2H), 3.12 (s, 3H), 2.96 (t, J=2.0 Hz, 1H), 2.85-2.75 (m, 2H), 2.70-2.67 (m, 2H), 2.66-2.59 (m, 1H), 2.24 (t, J=4.0 Hz, 1H), 2.22-2.00 (m, 1H), 1.87-1.86 (m, 1H), 1.03 (d, J=6.8 Hz, 3H). MS=438.3 [M+H]⁺.

The following compounds in Table 6 were prepared according to procedures similar to steps 1 to step 3 described for Compounds 115-117 using the appropriate starting materials.

TABLE 6

| | | | Exact | Step 3 | Step 3 |
|---|---|---|---|---|---|
| | | | Mass | Chrial | Elution |
| # | Structure | IUPAC Name | [M + H]⁺ | column | order |
| 118 | 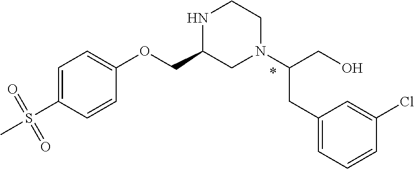 [SMILES: CS(=O)(=O)c1ccc(OC[C@@H]2CN(C(CO)Cc3cccc(Cl)c3)CCN2)cc1] | (2R or 2S)-3-(3- chlorophenyl)-2- [(3S)-3-[(4- methanesulfonyl- phenoxy)methyl] piperazin-1-yl] propan-1-ol | Calc'd 439.1 Found 439.1 | DAICEL CHIRALPAK AD | 1st |
| 119 | 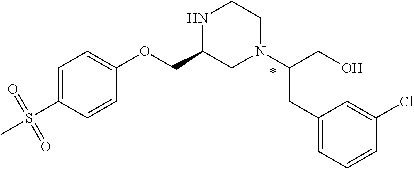 [SMILES: CS(=O)(=O)c1ccc(OC[C@@H]2CN(C(CO)Cc3cccc(Cl)c3)CCN2)cc1] | (2S or 2R)-3-(3- chlorophenyl)-2- [(3S)-3-[(4- methanesulfonyl- phenoxy)methyl] piperazin-1-yl] propan-1-ol | Calc'd 439.1 Found 439.2 | DAICEL CHIRALPAK AD | 2nd |
| 120 | 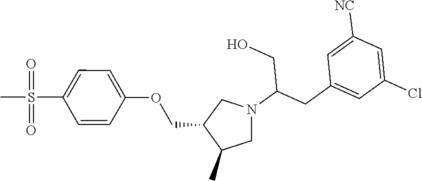 [SMILES: C[C@@H]1CN(C(CO)Cc2cc(Cl)cc(C#N)c2)C[C@H]1COc1ccc(S(C)(=O)=O)cc1] | 3-chloro-5-[3- hydroxy-2-[(3S, 4S)-3-[(4- methanesulfonyl- phenoxy)methyl]-4- methylpyrrolidin-1- yl]propyl]benzonitrile | Calc'd 463.1 Found 463.1 | n/a | |
| 121 | 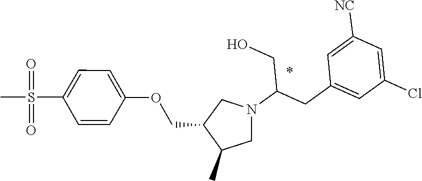 [SMILES: C[C@@H]1CN(C(CO)Cc2cc(Cl)cc(C#N)c2)C[C@H]1COc1ccc(S(C)(=O)=O)cc1] | 3-chloro-5-[(2R or 2S)-3-hydroxy-2- [(3S,4S)-3-[(4- methanesulfonyl- phenoxy)methyl]-4- methylpyrrolidin-1- yl]propyl]benzonitrile | Calc'd 433.1 Found 463.1 | DAICEL CHIRALPAK AD | 1st |
| 122 | 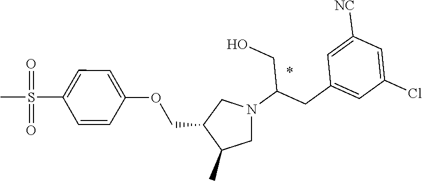 [SMILES: C[C@@H]1CN(C(CO)Cc2cc(Cl)cc(C#N)c2)C[C@H]1COc1ccc(S(C)(=O)=O)cc1] | 3-chloro-5-[(2S or 2R)-3-hydroxy-2- [(3S,4S)-3-[(4- methanesulfonyl- phenoxy)methyl]-4- methylpyrrolidin- 1-yl]propyl] benzonitrile | Calc'd 433.1 Found 463.1 | DAICEL CHIRALPAK AD | 2nd |

### Example 9 (Compounds 123 and 124)

### [(2S,4S or 4R)-1-[2-(3-chlorophenyl)ethyl]-4-[(4-methanesulfonylphenoxy)methyl]pyrrolidin-2-yl]methanol (Compound 124) and [(2S,4R or 4S)-1-[2-(3-chlorophenyl)ethyl]-4-[(4-methanesulfonylphenoxy)methyl]pyrrolidin-2-yl]methanol (Compound 123)

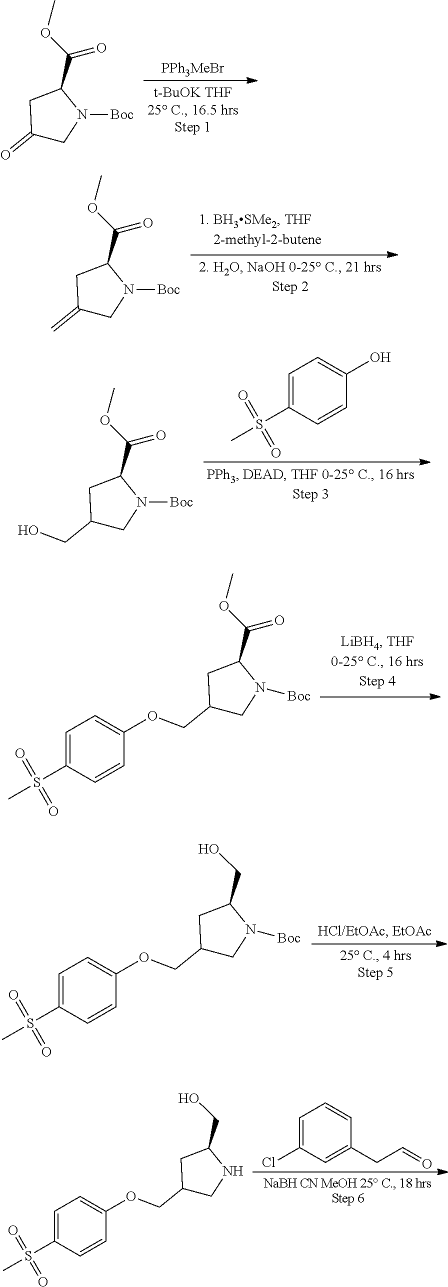

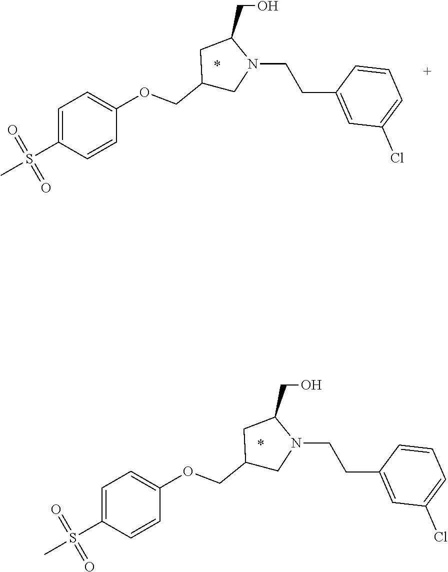 [SMILES: CS(=O)(=O)c1ccc(OCC2C[C@@H](CO)N(CCc3cccc(Cl)c3)C2)cc1 ; CS(=O)(=O)c1ccc(OCC2C[C@@H](CO)N(CCc3cccc(Cl)c3)C2)cc1]

### Step 1: (S)-1-tert-butyl 2-methyl 4-methylenepyrrolidine-1,2-dicarboxylate

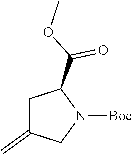 [SMILES: C=C1C[C@@H](C(=O)OC)N(C(=O)OC(C)(C)C)C1]

To a mixture of bromo(methyl)triphenylphosphorane (11.0 g, 30.83 mmol) in THE (50 mL) was added t-BuOK (3.23 g, 28.78 mmol). The mixture was stirred at RT for 0.5 h, and then (S)-1-tert-butyl 2-methyl 4-oxopyrrolidine-1,2-dicarboxylate (5.0 g, 20.55 mmol) in THE (25 mL) was added to the mixture dropwise. The mixture was stirred at room temperature for 16 h, then pentane (50 mL) was then added to the mixture. The resulting mixture was filtered, and the combined filtrates were concentrated in vacuum. The residue was purified by flash silica gel chromatography (Biotage 40 g cartridge, 0-20% EtOAc/Petroleum ether) to give (S)-1-tert-butyl 2-methyl 4-methylenepyrrolidine-1,2-dicarboxylate. ¹H NMR (CDCl₃, 400 MHz) δ 5.02-4.99 (m, 2H), 4.51-4.37 (dd, J=9.6 Hz, 3.2 Hz, 1H), 4.06 (dd, J=14.8 Hz, 2H), 3.72 (s, 3H), 3.01-2.94 (m, 1H), 2.62 (d, J=12.0 Hz, 1H), 1.4-1.42 (m, 9H).

### Step 2: (2S)-1-tert-butyl 2-methyl 4-(hydroxymethyl)pyrrolidine-1,2-dicarboxylate

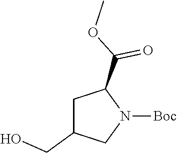 [SMILES: COC(=O)[C@@H]1CC(CO)CN1C(=O)OC(C)(C)C]

To a mixture of 2-methylbut-2-ene (1.71 g, 24.40 mmol) in THE (20 mL) was added BH₃-Me₂S (10 M in DMS, 1.22 mL) at 0° C. The mixture was stirred at 0° C. for 2 h, and then a solution of (S)-1-tert-butyl 2-methyl 4-methylenepyrrolidine-1,2-dicarboxylate (1.47 g, 6.10 mmol) in THE (2 mL) was added. The mixture was stirred at RT for 16 h. NaOH (3 M in H₂O, 2.38 mL) was then added, followed by addition of H₂O₂ (2.81 g, 24.74 mmol, 30% w/w) dropwise. The mixture was stirred at RT for 3 h and then was poured to water (5 mL). The aqueous phase was extracted with EtOAc (10 mL×3). The combined organic phase was washed with sat. aq. Na₂SO₃ (5 mL), then was washed with brine (5 mL), dried over anhydrous Na₂SO₄, filtered and concentrated under reduced pressure. The residue was purified by flash silica gel chromatography (Biotage 12 g cartridge, 0-100% EtOAc/Petroleum ether) to give (2S)-1-tert-butyl 2-methyl 4-(hydroxymethyl)pyrrolidine-1,2-dicarboxylate. MS=160.2 [M−C₅H₉O₂+H]⁺.

### Step 3: (2S)-1-tert-butyl 2-methyl 4-((4-(methylsulfonyl)phenoxy)methyl)pyrrolidine-1,2-dicarboxylate

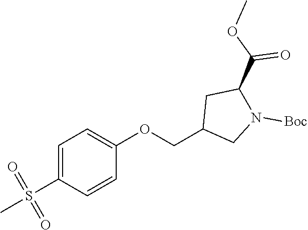 [SMILES: COC(=O)[C@@H]1CC(COc2ccc(S(C)(=O)=O)cc2)CN1C(=O)OC(C)(C)C]

To a mixture of (2S)-1-tert-butyl 2-methyl 4-(hydroxymethyl)pyrrolidine-1,2-dicarboxylate (1.2 g, 4.63 mmol) in THE (40 mL) were added 4-methylsulfonylphenol (1.20 g, 6.94 mmol), DEAD (1.21 g, 6.94 mmol), PPh₃ (1.82 g, 6.94 mmol) at 0° C. The mixture was stirred at RT for 16 h. The reaction mixture was poured into water (40 mL) and the aqueous phase was extracted with ethyl acetate (30 mL×3). The combined organic phase was washed with brine (30 mL), dried with anhydrous Na₂SO₄, filtered and concentrated under reduced pressure. The residue was poured into a mixture EtOAc (50 mL) and ZnCl₂ (1.3 g). The mixture was stirred at RT for 4 h, then the mixture was filtered, and the combined filtrates were concentrated in vacuum. The residue was purified by flash silica gel chromatography (Biotage 40 g cartridge, 0-50% EtOAc/Petroleum ether) to give (2S)-1-tert-butyl 2-methyl 4-((4-(methylsulfonyl)phenoxy)methyl)pyrrolidine-1,2-dicarboxylate. MS=314.0 [M−C₅H₉O₂+H]⁺.

### Step 4: (2S)-tert-butyl 2-(hydroxymethyl)-4-((4-(methylsulfonyl)phenoxy)methyl)pyrrolidine-1-carboxylate

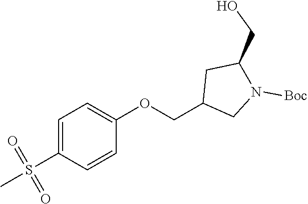 [SMILES: CC(C)(C)OC(=O)N1CC(COc2ccc(S(C)(=O)=O)cc2)C[C@H]1CO]

To a mixture of (2S)-1-tert-butyl 2-methyl 4-((4-(methylsulfonyl)phenoxy)methyl)pyrrolidine-1,2-dicarboxylate (415 mg, 1.00 mmol) in THE (3 mL) was added LiBH₄ (4 M in THF, 1.25 mL) dropwise at 0° C. under N₂. The mixture was stirred at RT for 16 h. The reaction mixture was poured into sat. aq. NH₄Cl (5 mL). The aqueous phase was extracted with EtOAc (15 mL×3). The combined organic phase was washed with brine (30 mL×1), dried over anhydrous Na₂SO₄, filtered and concentrated under reduced pressure. The residue was purified by flash silica gel chromatography (Biotage 4 g cartridge, 0-100% EtOAc/Hexane) to give (2S)-tert-butyl 2-(hydroxymethyl)-4-((4-(methylsulfonyl)phenoxy)methyl)pyrrolidine-1-carboxylate. MS=286.1 [M−C₅H₉O₂+H]⁺.

### Step 5: ((2S)-4-((4-(methylsulfonyl)phenoxy)methyl)pyrrolidin-2-yl)methanol

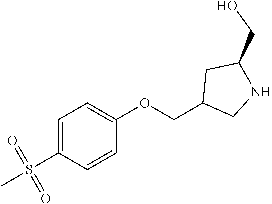 [SMILES: CS(=O)(=O)c1ccc(OCC2CN[C@H](CO)C2)cc1]

To a mixture of (2S)-tert-butyl 2-(hydroxymethyl)-4-((4-(methylsulfonyl)phenoxy)methyl)pyrrolidine-1-carboxylate (300 mg, 778 umol) in EtOAc (3 mL) was added HCl in EtOAc (4 M, 12 mL). The mixture was stirred at RT for 4 h. The reaction was concentrated under reduced pressure to give ((2S)-1-(3-chlorophenethyl)-4-((4-(methylsulfonyl)phenoxy)methyl)pyrrolidin-2-yl)methanol, which was taken forward without further purification. MS=286.1 [M+H]⁺

### Step 6: [(2S,4S or 4R)-1-[2-(3-chlorophenyl)ethyl]-4-[(4-methanesulfonylphenoxy)methyl]pyrrolidin-2-yl]methanol and [(2S,4R or 4S)-1-[2-(3-chlorophenyl)ethyl]-4-[(4-methanesulfonylphenoxy)methyl]pyrrolidin-2-yl]methanol

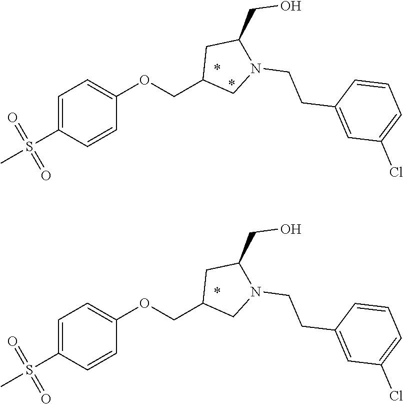 [SMILES: CS(=O)(=O)c1ccc(OCC2C[C@@H](CO)N(CCc3cccc(Cl)c3)C2)cc1 ; CS(=O)(=O)c1ccc(OCC2C[C@@H](CO)N(CCc3cccc(Cl)c3)C2)cc1]

To a mixture of ((2S)-1-(3-chlorophenethyl)-4-((4-(methylsulfonyl)phenoxy)methyl)pyrrolidin-2-yl)methanol (250 mg, 776 umol) in MeOH (2 mL) at RT was added Et₃N (235 mg, 2.33 mmol). Then AcOH (46.7 mg, 776 umol) and 2-(3-chlorophenyl)acetaldehyde (144 mg, 932 umol) were added and the mixture was stirred at RT for 16 h. NaBH₃CN (73 mg, 1.17 mmol) was then added, and the resulting mixture was stirred at RT for 2 h. The reaction mixture was adjusted to pH=7 by addition of sat. aq. NaHCO₃, then extracted with EtOAc (15 mL×3). The combined organic layers were washed with brine (30 mL), dried over anhydrous Na₂SO₄, filtered and concentrated under reduced pressure. The residue was purified by preparative reverse phase HPLC (Waters Xbridge Prep OBD C18, 30-60% MeCN:10 mM NH₄HCO₃ in H₂O). The first eluting isomer was further purified by preparative reverse phase HPLC (Phenomenex luna C18, 17-34% MeCN:0.04% HCl in H₂O) to give [(2S,4S or 4R)-1-[2-(3-chlorophenyl)ethyl]-4-[(4-methanesulfonylphenoxy)methyl]pyrrolidin-2-yl]methanol (Compound 123). ¹H NMR (DMSO-d₆, 400 MHz) δ 10.03 (s, 1H), 7.88-7.86 (m, 2H), 7.43 (s, 1H), 7.40-7.33 (m, 2H), 7.28 (d, J=15.2 Hz, 1H), 7.17 (d, J=8.8 Hz, 2H), 4.17 (d, J=6.8 Hz, 2H), 3.88-3.84 (m, 1H), 3.79-3.75 (m, 1H), 3.65-3.61 (m, 2H), 3.53-3.52 (m, 2H), 3.43-3.30 (m, 2H), 3.17 (s, 3H), 3.14-3.03 (m, 2H), 3.00-2.90 (m, 1H), 2.35-2.25 (m, 1H), 1.71-1.66 (m, 1H). MS=424.2 [M+H]⁺. The second eluting isomer was further purified by preparative reverse phase HPLC (:Phenomenex luna C18, 17-34% MeCN:0.04% HCl in H₂O) to give [(2S,4R or 4S)-1-[2-(3-chlorophenyl)ethyl]-4-[(4-methanesulfonylphenoxy)methyl]pyrrolidin-2-yl]methanol (Compound 124). ¹H NMR (DMSO-d₆, 400 MHz) δ 9.68-9.65 (m, 1H), 7.88 (d, J=8.8 Hz, 2H), 7.43 (s, 1H), 7.41-7.33 (m, 2H), 7.29-7.27 (m, 1H), 7.19 (d, J=8.8 Hz, 2H), 5.56-5.52 (m, 1H), 4.22-4.18 (m, 1H), 4.14-4.10 (m, 1H), 3.86-3.81 (m, 2H), 3.72-3.60 (m, 3H), 3.41-3.27 (m, 2H), 3.17 (s, 3H), 3.10-2.98 (m, 2H), 2.83-2.75 (m, 1H), 2.03 (t, J=8.0 Hz, 2H). MS=424.2 [M+H]⁺.

### Example 10 (Compound 125)

### 3-{[(2S,5R or 5S,8aR)-2-[(4-methanesulfonylphenoxy)methyl]-octahydroindolizin-5-yl]methyl}-5-chlorobenzonitrile

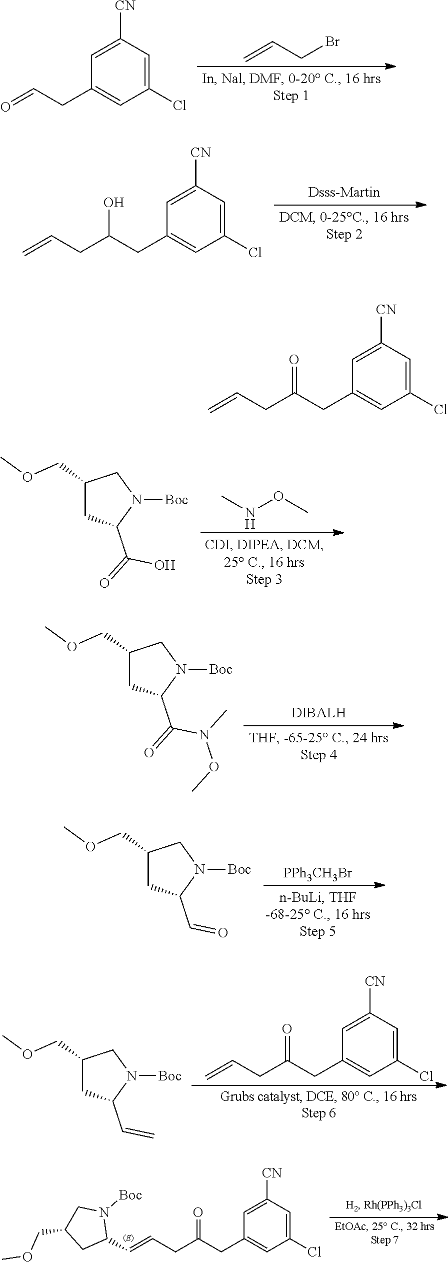

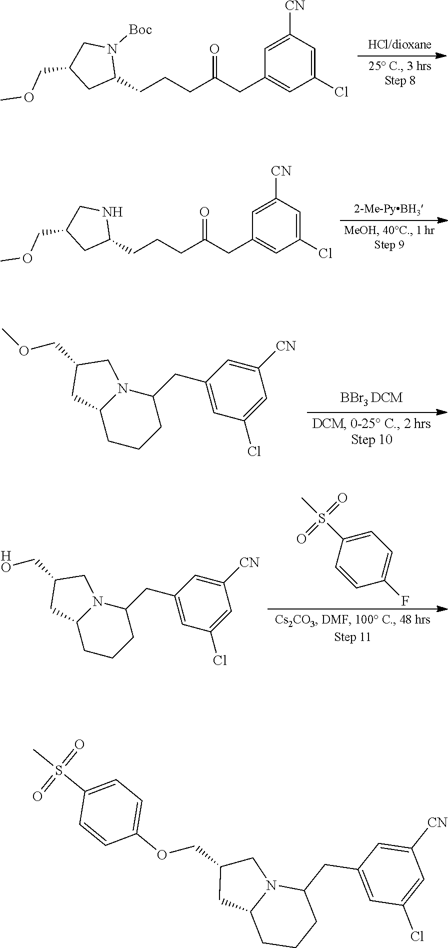

### Step 1: 3-chloro-5-(2-hydroxypent-4-en-1-yl)benzonitrile

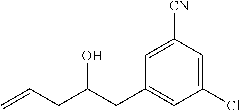

To a solution of 3-chloro-5-(2-oxoethyl)benzonitrile (5.00 g, 27.84 mmol), 3-bromoprop-1-ene (4.04 g, 33.41 mmol) and NaI (6.26 g, 41.76 mmol) in DMF (50 mL) was added In (3.36 g, 29.23 mmol) at 0° C. The mixture was then stirred at RT for 16 h. The reaction mixture was quenched by addition of H₂O (100 mL) and extracted with EtOAc (50 mL×3). The combined organic layers were washed with brine (200 mL×1), dried over Na₂SO₄, filtered and concentrated under reduced pressure. The residue was purified by flash silica gel chromatography (Biotage 40 g cartridge, 0-30% EtOAc/Hexane to give 3-chloro-5-(2-hydroxypent-4-en-1-yl)benzonitrile. ¹H NMR (CDCl₃, 400 MHz) δ 7.51-7.49 (m, 2H), 7.45 (s, 1H), 5.86-5.78 (m, 1H), 5.22-5.16 (m, 2H), 3.92-3.85 (m, 1H), 2.86-2.70 (m, 2H), 2.34-2.32 (m, 1H), 2.22-2.19 (m, 1H), 1.66-1.65 (m, 1H).

### Step 2: 3-chloro-5-(2-oxopent-4-en-1-yl)benzonitrile

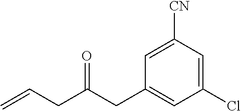

To a solution of 3-chloro-5-(2-hydroxypent-4-en-1-yl)benzonitrile (400 mg, 1.80 mmol) in DCM (10 mL) was added DMP (1.15 g, 2.71 mmol) at 0° C. The mixture was then stirred at RT for 16 h. The reaction mixture was filtered, and the filtrate was concentrated under reduced pressure to give a residue. The residue was purified by flash silica gel chromatography (Biotage 12 g cartridge, 0-30% EtOAc/Hexane to give 3-chloro-5-(2-oxopent-4-en-1-yl)benzonitrile. MS=220.1 [M+H]⁺.

### Step 3: (2S,4S)-tert-butyl 2-(methoxy(methyl)carbamoyl)-4-(methoxymethyl)pyrrolidine-1-carboxylate

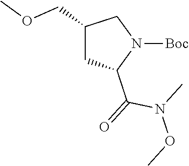

To a solution of (2S,4S)-1-(tert-butoxycarbonyl)-4-(methoxymethyl)pyrrolidine-2-carboxylic acid (20.00 g, 77.13 mmol) in DCM (100 mL) at RT was added CDI (15.00 g, 92.56 mmol). The resulting mixture was stirred for 0.5 h. N-methoxymethanamine hydrochloride (9.03 g, 92.56 mmol) and DIEA (12.00 g, 92.56 mmol) were then added. The reaction mixture was stirred for 15.5 h. The reaction mixture was diluted with H₂O (100 mL) and extracted with EtOAc (50 mL×3). The combined organic layers were washed with brine (50 mL), dried over Na₂SO₄, filtered and concentrated under reduced pressure. The residue was purified by flash silica gel chromatography (Biotage 300 g cartridge, 0-100% EtOAc/Hexane) to give (2S,4S)-tert-butyl 2-(methoxy(methyl)carbamoyl)-4-(methoxymethyl)pyrrolidine-1-carboxylate. MS=303.1 (M+H)+.

### Step 4: (2S,4S)-tert-butyl 2-formyl-4-(methoxymethyl)pyrrolidine-1-carboxylate

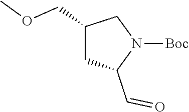 [SMILES: COC[C@H]1C[C@@H](C=O)N(C(=O)OC(C)(C)C)C1]

To a solution of (2S,4S)-tert-butyl 2-(methoxy(methyl)carbamoyl)-4-(methoxymethyl)pyrrolidine-1-carboxylate (12.00 g, 39.69 mmol) in THE (200 mL) was added diisobutylaluminum hydride (1 M in toluene, 43.66 mL) dropwise at −65° C. under N₂. The reaction mixture was then stirred at RT for 24 h. After being cooled to 0° C., the reaction mixture was quenched by addition of H₂O (100 mL), followed by 15 mL of 15% aq. NaOH solution. The resulting mixture was stirred at RT for 10 min. The solid was removed by filtration. The filtrate was concentrated. The residue was purified by flash silica gel chromatography (Biotage; 80 g cartridge, 0-50% EtOAc/Hexane) to give (2S,4S)-tert-butyl 2-formyl-4-(methoxymethyl)pyrrolidine-1-carboxylate. MS=244.1 (M+H)+.

### Step 5: (2S,4S)-tert-butyl 4-(methoxymethyl)-2-vinylpyrrolidine-1-carboxylate

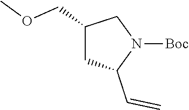 [SMILES: C=C[C@@H]1C[C@H](COC)CN1C(=O)OC(C)(C)C]

To a solution of methyl(triphenyl)phosphonium bromide (10.28 g, 28.77 mmol) in THF (50 mL) at −78° C. was added a solution of butyllithium in THE (2.5 M, 11.51 mL) dropwise. The mixture was stirred at −78° C. for 1 h. (2S,4S)-tert-butyl 2-formyl-4-(methoxymethyl)pyrrolidine-1-carboxylate (3.5o g, 14.39 mmol) was then added at −78° C. The resulting mixture was stirred at RT for 15 h. The reaction mixture was quenched by sat. aq. NH₄Cl (150 mL) and extracted with EtOAc (50 mL×3). The combined organic layers were washed with brine (50 mL×1), dried over Na₂SO₄, filtered and concentrated under reduced pressure. The residue was purified by flash silica gel chromatography (Biotage 20 g cartridge, 0-50% EtOAc/Hexane) to give (2S,4S)-tert-butyl 4-(methoxymethyl)-2-vinylpyrrolidine-1-carboxylate. ¹H NMR (CDCl₃, 400 MHz) δ 5.75 (br s, 1H), 5.05 (br s, 1H), 4.45-4.10 (m, 1H), 3.85-3.45 (m, 1H), 3.35 (s, 5H), 3.20-3.00 (m, 1H), 2.60-2.17 (m, 2H), 1.80 (s, 1H), 1.49-1.43 (m, 10H).

### Step 6: (2S,4S)-tert-butyl 2-((E)-5-(3-chloro-5-cyanophenyl)-4-oxopent-1-en-1-yl)-4-(methoxymethyl)pyrrolidine-1-carboxylate

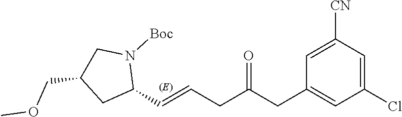 [SMILES: COC[C@H]1C[C@@H](/C=C/CC(=O)Cc2cc(Cl)cc(C#N)c2)N(C(=O)OC(C)(C)C)C1]

Twenty reactions were carried out in parallel.

To a solution of (2S,4S)-tert-butyl 4-(methoxymethyl)-2-vinylpyrrolidine-1-carboxylate (200 mg, 828 umol) and 3-chloro-5-(2-oxopent-4-en-1-yl)benzonitrile (182 mg, 828 umol) in DCE (15 mL) was added [1,3-bis(2,4,6-trimethylphenyl)imidazolidin-2-ylidene]-dichloro-[(2-isopropoxyphenyl) methylene]ruthenium (25 mg, 41 umol). The reaction mixture was stirred at 80° C. for 16 h under N₂. All 20 reaction mixtures were combined, filtered and concentrated under reduced pressure. The residue was purified by flash silica gel chromatography (Biotage 40 g cartridge, 0-50% EtOAc/Hexane) to give (2S,4S)-tert-butyl 2-((E)-5-(3-chloro-5-cyanophenyl)-4-oxopent-1-en-1-yl)-4-(methoxymethyl)pyrrolidine-1-carboxylate. MS=333.1 [M−C₅H₉O₂+H]⁺

### Step 7: (2R,4S)-tert-butyl 2-(5-(3-chloro-5-cyanophenyl)-4-oxopentyl)-4-(methoxymethyl)pyrrolidine-1-carboxylate

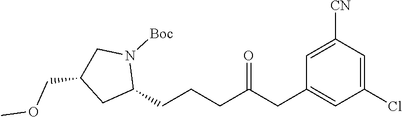 [SMILES: COC[C@H]1C[C@@H](CCCC(=O)Cc2cc(Cl)cc(C#N)c2)N(C(=O)OC(C)(C)C)C1]

To a solution of (2S,4S)-tert-butyl 2-((E)-5-(3-chloro-5-cyanophenyl)-4-oxopent-1-en-1-yl)-4-(methoxymethyl)pyrrolidine-1-carboxylate (1.00 g, 2.31 mmol) in EtOAc (20 mL) at RT was added chlororhodium triphenylphosphane (641 mg, 692 umol). The reaction mixture was stirred under H₂ (15 psi) at RT for 32 h. The reaction mixture was then filtered and concentrated under reduced pressure. The residue was purified by flash silica gel chromatography (Biotage 20 g cartridge, 0-50% EtOAc/Hexane) to give (2R,4S)-tert-butyl 2-(5-(3-chloro-5-cyanophenyl)-4-oxopentyl)-4-(methoxymethyl)pyrrolidine-1-carboxylate. MS=335.1 [M−C₅H₉O₂+H]⁺

### Step 8: 3-chloro-5-(5-((2R,4S)-4-(methoxymethyl)pyrrolidin-2-yl)-2-oxopentyl)benzonitrile

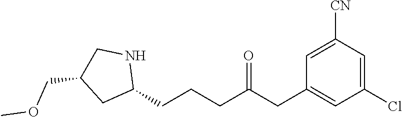 [SMILES: COC[C@@H]1CN[C@H](CCCC(=O)Cc2cc(Cl)cc(C#N)c2)C1]

A solution of (2R,4S)-tert-butyl 2-(5-(3-chloro-5-cyanophenyl)-4-oxopentyl)-4-(methoxymethyl)pyrrolidine-1-carboxylate (600 mg, 1.38 mmol) in HCl in dioxane (4 M, 20 mL) was stirred at RT for 3 h. The reaction mixture was then concentrated under reduced pressure to give 3-chloro-5-(5-((2R,4S)-4-(methoxymethyl)pyrrolidin-2-yl)-2-oxopentyl)benzonitrile HCl salt, which was used without further purification. MS=335.1 [M+H]⁺.

### Step 9: 3-chloro-5-(((2S,8aR)-2-(methoxymethyl)octahydroindolizin-5-yl)methyl)benzonitrile

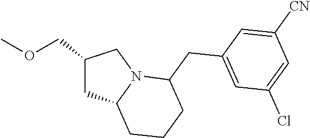 [SMILES: COC[C@H]1C[C@H]2CCCC(Cc3cc(Cl)cc(C#N)c3)N2C1]

To a solution of 3-chloro-5-(5-((2R,4S)-4-(methoxymethyl)pyrrolidin-2-yl)-2-oxopentyl)benzonitrile (350 mg, 1.05 mmol, HCl salt) in MeOH (3 mL) was added TEA to adjust pH to 7. 2-methylpyridine borane complex (223 mg, 2.09 mmol) and AcOH (0.3 mL) were then added. The resulting mixture was stirred at 40° C. for 1 h. The reaction mixture was diluted with H₂O (10 mL) and extracted with EtOAc (10 mL×3). The combined organic layers were washed with brine (15 mL×1), dried over Na₂SO₄, filtered and concentrated under reduced pressure. The residue was purified by flash silica gel chromatography (Biotage 12 g cartridge, 0-100% EtOAc/Hexane) to give 3-chloro-5-(((2S,8aR)-2-(methoxymethyl)octahydroindolizin-5-yl)methyl)benzonitrile. MS=319.1 [M+H]⁺.

### Step 10: 3-chloro-5-(((2S,8aR)-2-(hydroxymethyl)octahydroindolizin-5-yl)methyl)benzonitrile

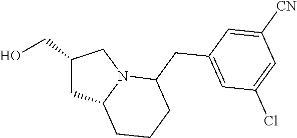 [SMILES: N#Cc1cc(Cl)cc(CC2CCC[C@@H]3C[C@H](CO)CN23)c1]

To a solution of 3-chloro-5-(((2S,8aR)-2-(methoxymethyl)octahydroindolizin-5-yl)methyl)benzonitrile (130 mg, 407 umol) in DCM (5 mL) was added BBr₃ (2 M in DCM, 2.04 mL) at 0° C. The reaction mixture was then stirred at RT for 2 h. The reaction was quenched by addition of H₂O (10 mL) and stirred for 10 min. The mixture was extracted with 10:1 DCM/i-PrOH (5 mL×3). The combined organic layers were washed with brine (10 mL), dried over Na₂SO₄, filtered and concentrated under reduced pressure to give 3-chloro-5-(((2S,8aR)-2-(hydroxymethyl)octahydroindolizin-5-yl)methyl)benzonitrile, which was taken to the next step without further purification. MS=305.1 [M+H]⁺.

### Step 11: 3-{1[(2S,5R or 5S,8aR)-2-[(4-methanesulfonylphenoxy)methyl]-octahydroindolizin-5-yl]methyl}-5-chlorobenzonitrile

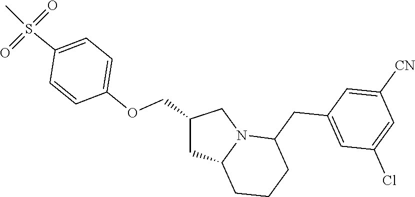 [SMILES: CS(=O)(=O)c1ccc(OC[C@H]2C[C@H]3CCCC(Cc4cc(Cl)cc(C#N)c4)N3C2)cc1]

To a solution of 3-chloro-5-(((2S,8aR)-2-(hydroxymethyl)octahydroindolizin-5-yl)methyl)benzonitrile (100 mg, 328 umol) and 1-fluoro-4-methylsulfonyl-benzene (171 mg, 984 umol) in DMF (10 mL) was added Cs₂CO₃ (855 mg, 2.62 mmol). The reaction mixture was then stirred at 100° C. for 48 h. The reaction mixture was allowed to cool to RT, then quenched by addition of aq. sat. NH₄Cl and extracted with EtOAc (10 mL×3). The combined organic layers were washed with brine (20 mL), dried over Na₂SO₄, filtered and concentrated under reduced pressure. The residue was purified by preparative reverse phase HPLC (Phenomenex Luna, 5-30% MeCN:0.04% HCl in H₂O) and was further purified by prep-TLC (PE:EA=3:1) to give 3-{[(2S,5R or 5S,8aR)-2-[(4-methanesulfonylphenoxy)methyl]-octahydroindolizin-5-yl]methyl}-5-chlorobenzonitrile (Compound 125). ¹H NMR (MeOD, 400 MHz) δ 7.91 (d, J=8.8 Hz, 2H), 7.77 (s, 1H), 7.71 (s, 1H), 7.67 (s, 1H), 7.19 (d, J=8.8 Hz, 2H), 4.21-4.13 (m, 2H), 3.75-3.74 (m, 1H), 3.56-3.43 (m, 5H), 3.09 (s, 3H), 2.80-2.79 (m, 1H), 2.60-2.49 (m, 1H), 2.25-2.15 (m, 1H), 2.00-1.86 (m, 1H), 1.82-1.73 (m, 1H), 1.67-1.53 (m, 4H). MS=459.2 [M+H]⁺.

### Example 11 (Compounds 126-128)

### 3-chloro-5-(1-hydroxy-3-((3S,4S)-3-methyl-4-((4-(methylsulfonyl)phenoxy)methyl)pyrrolidin-1-yl)propan-2-yl)benzonitrile (Compound 126) and 3-chloro-5-[(2R or 2S)-1-hydroxy-3-[(3S,4S)-3-[(4-methanesulfonylphenoxy)methyl]-4-methylpyrrolidin-1-yl]propan-2-yl]benzonitrile (Compound 127) and 3-chloro-5-[(2S or 2R)-1-hydroxy-3-[(3S,4S)-3-[(4-methanesulfonylphenoxy)methyl]-4-methylpyrrolidin-1-yl]propan-2-yl]benzonitrile (Compound 128)

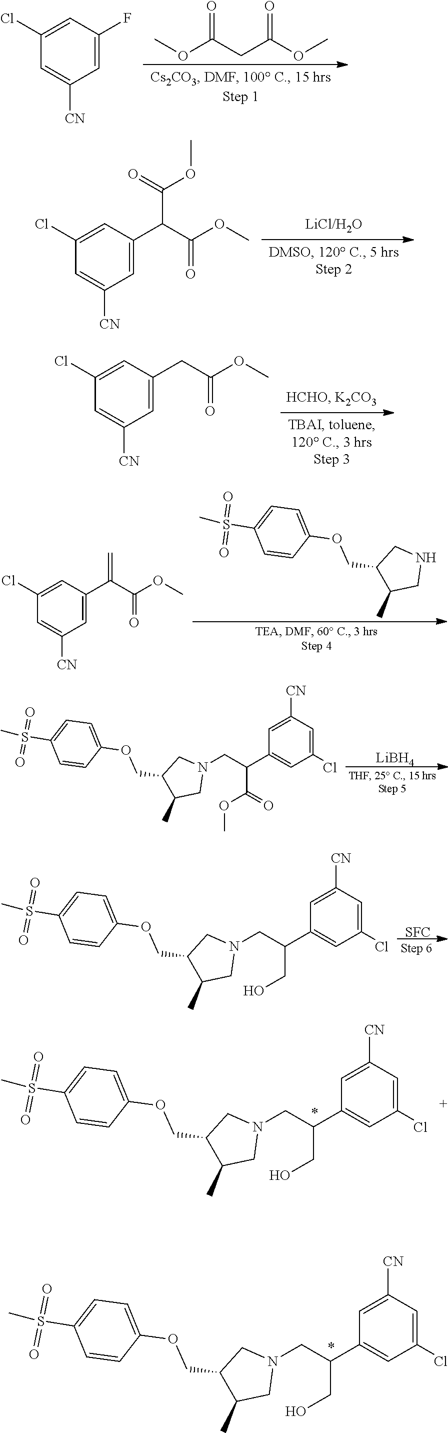

### Step 1: dimethyl 2-(3-chloro-5-cyanophenyl)malonate

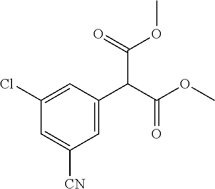 [SMILES: COC(=O)C(C(=O)OC)c1cc(Cl)cc(C#N)c1]

A mixture of 3-chloro-5-fluoro-benzonitrile (3.00 g, 19.29 mmol), dimethyl propanedioate (3.82 g, 28.93 mmol, 3.32 mL), Cs₂CO₃ (15.7 g, 48.21 mmol) in DMF (20 mL) was degassed and purged with N₂ for 3 times, and then the mixture was stirred at 100° C. for 15 h under N₂. The reaction mixture was cooled to 0° C. and quenched by addition ice-water (50 mL). The pH of the mixture was adjusted to 4-5 by addition of 4 N aq. HCl and extracted with EtOAc (20 mL×3). The combined organic layers were washed with brine (50 mL×2), dried over Na₂SO₄, filtered and concentrated under reduced pressure. The residue was purified by flash silica gel chromatography (Biotage 20 g cartridge, 0-10% Ethyl acetate/Petroleum ether) to give dimethyl 2-(3-chloro-5-cyanophenyl)malonate. MS=265.9 [M−H]⁻.

### Step 2: methyl 2-(3-chloro-5-cyanophenyl)acetate

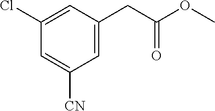

To a solution of dimethyl 2-(3-chloro-5-cyanophenyl)malonate (5.00 g, 18.68 mmol) in DMSO (50 mL) were added LiCl (1.98 g, 46.70 mmol) and H₂O (337 uL, 18.68 mmol). The mixture was stirred at 120° C. for 5 h. The reaction mixture was cooled to 0° C., quenched by addition of water (20 mL), then extracted with EtOAc (20 mL×3). The combined organic layers were washed with brine (50 mL×2), dried over Na₂SO₄, filtered and concentrated under reduced pressure. The residue was purified by flash silica gel chromatography (Biotage 40 g cartridge, 0-10% Ethyl acetate/Petroleum ether) to give methyl 2-(3-chloro-5-cyanophenyl)acetate. ¹H NMR (DMSO-d₆, 400 MHz) δ 7.96 (s, 1H), 7.77 (s, 2H), 3.83 (s, 2H), 3.64 (s, 3H).

### Step 3: methyl 2-(3-chloro-5-cyanophenyl)acrylate

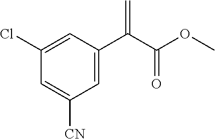

A mixture of methyl 2-(3-chloro-5-cyanophenyl)acetate (900 mg, 4.29 mmol), HCHO (1.29 g, 42.93 mmol), K₂CO₃ (1.78 g, 12.88 mmol), TBAI (15.86 mg, 42.93 umol) in toluene (20 mL) was degassed and purged with N₂ for 3 times, and then the mixture was stirred at 110° C. for 3 h under N₂. The mixture was filtered, and the filtrate was concentrated under reduced pressure. The residue was purified by flash silica gel chromatography (Biotag; 12 g cartridge, 0-10% Ethyl acetate/Petroleum ether) to give methyl 2-(3-chloro-5-cyanophenyl)acrylate. ¹H NMR (DMSO-d₆, 400 MHz) δ 8.04 (t, J=0.8 Hz, 1H), 7.93 (t, J=0.8 Hz, 1H), 7.89 (t, J=0.8 Hz, 1H), 6.45 (s, 1H), 6.28 (s, 1H), 3.77 (s, 3H).

### Step 4: methyl 2-(3-chloro-5-cyanophenyl)-3-((3S,4S)-3-methyl-4-((4-(methylsulfonyl)phenoxy)methyl)pyrrolidin-1-yl)propanoate

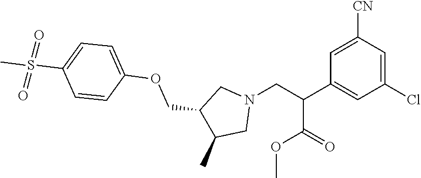 [SMILES: COC(=O)C(CN1C[C@@H](COc2ccc(S(C)(=O)=O)cc2)[C@H](C)C1)c1cc(Cl)cc(C#N)c1]

A mixture of methyl 2-(3-chloro-5-cyanophenyl)acrylate (220 mg, 993 umol), (3S,4S)-3-methyl-4-((4-(methylsulfonyl)phenoxy)methyl)pyrrolidine (267 mg, 993 umol) and TEA (201 mg, 1.99 mmol) in DMF (2 mL) was degassed and purged with N₂ three times, and then the mixture was stirred at 60° C. for 3 h under N₂ atmosphere. The reaction mixture was poured to ice-water (10 mL), the solid was precipitated, the mixture was filtered, and the solid was washed with water. The solid was dried in vacuo to give methyl 2-(3-chloro-5-cyanophenyl)-3-((3S,4S)-3-methyl-4-((4-(methylsulfonyl)phenoxy)methyl)pyrrolidin-1-yl)propanoate. MS=491.2 [M+H]⁺.

### Step 5: 3-chloro-5-(1-hydroxy-3-((3S,4S)-3-methyl-4-((4-(methylsulfonyl)phenoxy)methyl)pyrrolidin-1-yl)propan-2-yl)benzonitrile

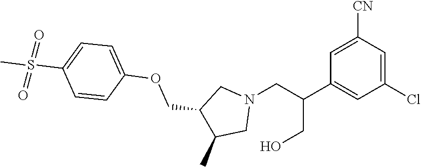 [SMILES: C[C@@H]1CN(CC(CO)c2cc(Cl)cc(C#N)c2)C[C@H]1COc1ccc(S(C)(=O)=O)cc1]

To a solution of methyl 2-(3-chloro-5-cyanophenyl)-3-((3S,4S)-3-methyl-4-((4-(methylsulfonyl)phenoxy)methyl)pyrrolidin-1-yl)propanoate (240 mg, 488.80 umol) in THE (10 mL) at 0° C. was added LiBH₄ (2 M in THF, 1.05 mL, 2.10 mmol). The mixture was stirred at RT for 15 h. The reaction mixture was cooled to 0° C., quenched by addition of water (10 mL), adjusted pH to 4-5 with 4 N aq. HCl, then extracted with EtOAc (10 mL×2). The combined organic layers were washed with brine (20 mL×2), dried over Na₂SO₄, filtered and concentrated under reduced pressure. The residue was purified by preparative reverse phase HPLC (Waters Xbridge BEH C18. mobile phase: [water (10 mM NH₄HCO₃)-ACN]; B %: 35%-65% MeCN:10 mM NH₄HCO₃ in H₂O—) to give 3-chloro-5-(1-hydroxy-3-((3S,4S)-3-methyl-4-((4-(methylsulfonyl)phenoxy)methyl)pyrrolidin-1-yl)propan-2-yl)benzonitrile (Compound 126). MS=463.0 [M+H]⁺.

### Step 6: 3-chloro-5-[(2R or 2S)-1-hydroxy-3-[(3S,4S)-3-[(4-methanesulfonylphenoxy)methyl]-4-methylpyrrolidin-1-yl]propan-2-yl]benzonitrile (Compound 127) and 3-chloro-5-[(2S or 2R)-1-hydroxy-3-[(3S,4S)-3-[(4-methanesulfonylphenoxy)methyl]-4-methylpyrrolidin-1-yl]propan-2-yl]benzonitrile (Compound 128)

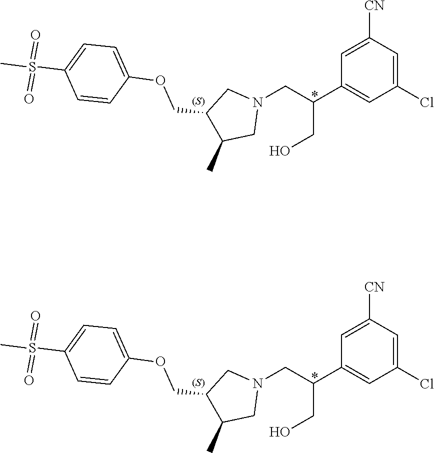

3-chloro-5-(1-hydroxy-3-((3S,4S)-3-methyl-4-((4-(methylsulfonyl)phenoxy)methyl)pyrrolidin-1-yl)propan-2-yl)benzonitrile (40 mg, 86.4 umol) was separated by chiral SFC (DAICEL CHIRALPAK AD, 55% ethanol with 0.1% NH₄OH in CO₂) The first eluting isomer of the title compound (Compound 127): ¹H NMR (DMSO-d₆, 400 MHz) δ 7.86-7.79 (m, 3H), 7.72-7.66 (m, 2H), 7.12-7.09 (m, 2H), 4.75 (brs, 1H), 3.95-3.92 (m, 1H), 3.87-3.85 (m, 1H), 3.69-3.59 (m, 2H), 3.46-3.38 (m, 2H), 3.15 (s, 3H), 3.05-2.95 (m, 1H), 2.80-2.76 (m, 1H), 2.67-2.62 (m, 2H), 2.06-1.99 (m, 2H), 1.87-1.83 (m, 1H), 1.00 (s, 3H). MS=463.1 [M+H]⁺. The second eluting isomer of the title compound (Compound 128): ¹H NMR (DMSO-d₆, 400 MHz) δ 7.84-7.79 (m, 3H), 7.72-7.68 (m, 2H), 7.13-7.10 (m, 2H), 4.74 (br s, 1H), 4.00-3.95 (m, 1H), 3.93-3.85 (m, 1H), 3.68-3.60 (m, 1H), 3.58-3.50 (m, 1H), 3.45-3.39 (m, 2H), 3.14 (s, 3H), 3.03-2.97 (m, 1H), 2.80-2.73 (m, 2H), 2.59-2.53 (m, 1H), 2.43-2.31 (m, 1H), 2.06-1.99 (m, 2H), 1.90-1.80 (m, 1H), 1.00 (s, 3H). MS=463.1 [M+H]⁺.

The following compounds in Table 7 were prepared according to procedures similar to steps 1 to step 6 described for Compounds 126-128 using the appropriate starting materials.

TABLE 7

| | | | Exact | | Step 6 |
|---|---|---|---|---|---|
| | | | Mass | Step 6 | sElution |
| # | Structure | IUPAC Name | [M + H]⁺ | column | order |
| 129 | 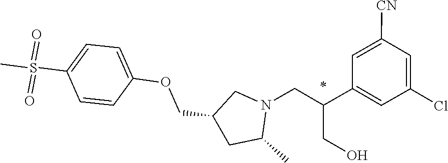 [SMILES: C[C@@H]1C[C@H](COc2ccc(S(C)(=O)=O)cc2)CN1CC(CO)c1cc(Cl)cc(C#N)c1] | 3-chloro-5-[(2R or 2S)- 1-hydroxy-3-[(2R,4S)-4- [(4-methanesulfonyl- phenoxy)methyl]-2- methylpyrrolidin-1-yl] propan-2-yl]benzonitrile | Calc'd 463.1 Found 463.1 | prep- HPLC Waters Xbridge BEH C18 | 1st |
| 130 | 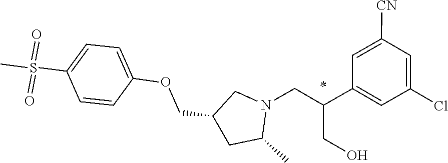 [SMILES: C[C@@H]1C[C@H](COc2ccc(S(C)(=O)=O)cc2)CN1CC(CO)c1cc(Cl)cc(C#N)c1] | 3-chloro-5-[(2S or 2R)-1-hydroxy-3- [(2R,4S)-4-[(4- methanesulfonyl- phenoxy)methyl]-2- methylpyrrolidin-1- yl]propan-2-yl] benzonitrile | Calc'd 463.1 Found 463.1 | prep- HPLC Waters Xbridge BEH C18 | 2nd |

### Example 12 (Compounds 131-133)

### (3R)-1-(7-chloro-1,2,3,4-tetrahydronaphthalen-2-yl)-3-[(4-methanesulfonylphenoxy)methyl]piperidine (Compounds 131) and (3R)-1-[(2S or 2R)-7-chloro-1,2,3,4-tetrahydronaphthalen-2-yl]-3-[(4-methanesulfonylphenoxy)methyl]piperidine and (3R)-1-[(2R or 2S)-7-chloro-1,2,3,4-tetrahydronaphthalen-2-yl]-3-[(4-methanesulfonylphenoxy)methyl]piperidine

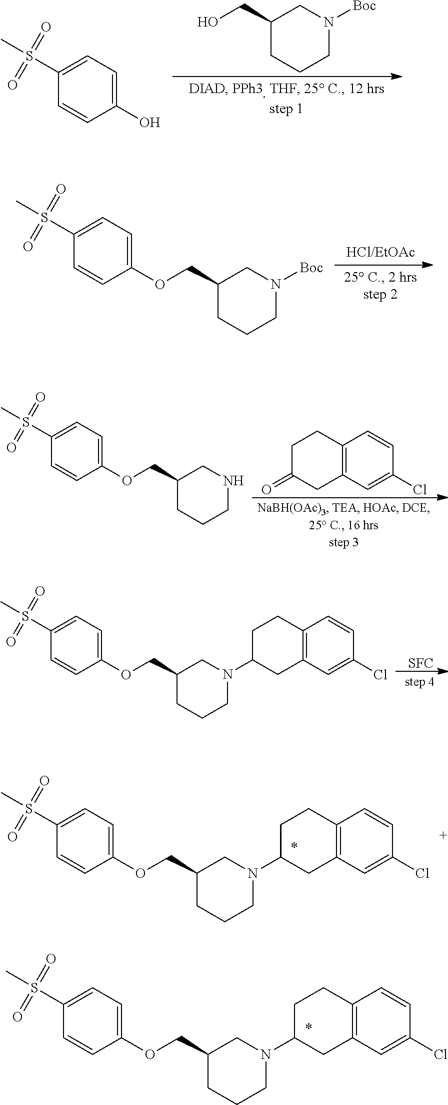

### Step 1: tert-butyl (R)-3-((4-(methylsulfonyl)phenoxy)methyl)piperidine-1-carboxylate

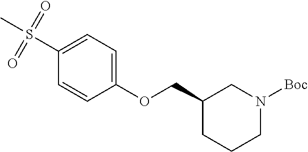 [SMILES: CC(C)(C)OC(=O)N1CCC[C@@H](COc2ccc(S(C)(=O)=O)cc2)C1]

To a mixture of tert-butyl (3R)-3-(hydroxymethyl)piperidine-1-carboxylate (10.0 g, 46.5 mmol), 4-methylsulfonylphenol (8.00 g, 46.5 mmol), PPh₃ (24.37 g, 92.9 mmol) in THF (150 mL) at 0° C. was added DIAD (18.79 g, 92.9 mmol). The mixture was stirred at RT for 12 h under N₂, then concentrated under reduced pressure. The residue was purified by column chromatography (Biotage 12 g cartridge, 0-50% Ethyl acetate/Petroleum ether gradient) to give tert-butyl (3R)-3-[(4-methylsulfonylphenoxy)methyl]piperidine-1-carboxylate. MS=314.1 [M−C₄H₈+H]⁺.

### Step 2: (R)-3-((4-(methylsulfonyl)phenoxy)methyl)piperidine

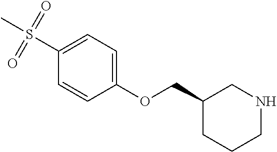 [SMILES: CS(=O)(=O)c1ccc(OC[C@@H]2CCCNC2)cc1]

To a solution of tert-butyl (3R)-3-[(4-methylsulfonylphenoxy)methyl]piperidine-1-carboxylate (12 g, 32.5 mmol) in EtOAc (50 mL) was added HCl in EtOAc (4 M, 100 mL). The mixture was stirred at RT for 2 h. The reaction mixture was filtered to give (3R)-3-[(4-methylsulfonylphenoxy)methyl]piperidine. MS=270.2 [M+H]⁺

### Step 3: (3R)-1-(7-chloro-1,2,3,4-tetrahydronaphthalen-2-yl)-3-[(4-methanesulfonylphenoxy)methyl]piperidine

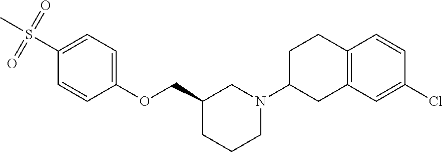 [SMILES: CS(=O)(=O)c1ccc(OC[C@@H]2CCCN(C3CCc4ccc(Cl)cc4C3)C2)cc1]

To a solution of (3R)-3-[(4-methylsulfonylphenoxy)methyl]piperidine (150 mg, 490 umol) in DCE (4 mL) were added TEA (68 uL, 49 umol), HOAc (28 uL, 49 umol) and 7-chlorotetralin-2-one (88.6 mg, 490 umol) The mixture was stirred RT for 1 h. Then NaBH(OAc)₃ (312 mg, 1.47 mmol) was added, and the mixture was stirred at RT for 15 h. The reaction mixture was quenched with water (15 mL) and extracted with EtOAc (10 mL×3). The combined organic layers were washed with brine (8 mL), dried over Na₂SO₄, filtered and concentrated under reduced pressure to give a residue. The residue was purified by preparative reverse phase HPLC (Waters Xbridge C18, 10 mM NH₄HCO₃)-40%-80% MeCN:10 mM NH₄HCO₃ in H₂O) to give (3R)-1-(7-chloro-1,2,3,4-tetrahydronaphthalen-2-yl)-3-[(4-methanesulfonylphenoxy)methyl]piperidine (Compound 131). ¹H NMR (DMSO-d₆, 400 MHz) δ 7.82 (d, J=7.6 Hz, 2H), 7.17-7.06 (m, 5H), 3.97 (d, J=6.0 Hz, 2H), 3.15 (s, 3H), 3.00-2.93 (m, 1H), 2.84-2.70 (m, 6H), 2.30-2.10 (m, 2H), 2.02-1.94 (m, 2H), 1.77-1.66 (m, 2H), 1.61-1.44 (m, 2H), 1.18-1.12 (m, 1H). MS=434.2 [M+H]⁺

### Step 4: (3R)-1-[(2S or 2R)-7-chloro-1,2,3,4-tetrahydronaphthalen-2-yl]-3-[(4-methanesulfonylphenoxy)methyl]piperidine and (3R)-1-[(2R or 2S)-7-chloro-1,2,3,4-tetrahydronaphthalen-2-yl]-3-[(4-methanesulfonylphenoxy)methyl]piperidine

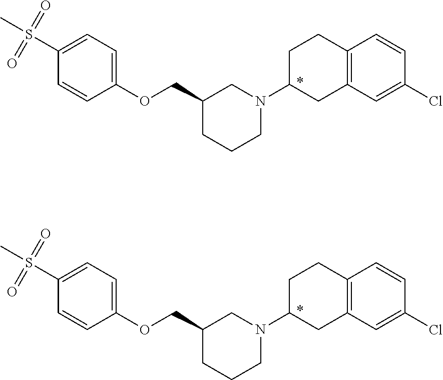 [SMILES: CS(=O)(=O)c1ccc(OC[C@@H]2CCCN(C3CCc4ccc(Cl)cc4C3)C2)cc1 ; CS(=O)(=O)c1ccc(OC[C@@H]2CCCN(C3CCc4ccc(Cl)cc4C3)C2)cc1]

(3R)-1-[7-chloro-1,2,3,4-tetrahydronaphthalen-2-yl]-3-[(4-methanesulfonylphenoxy)methyl]piperidine (0.067 g, 154 umol) was separated by preparative chiral SFC (DAICEL CHIRALPAK AD, 60% ethanol with 0.1% NH₄OH in CO₂). The first eluting isomer of the title compound (Compound 132): ¹H NMR (DMSO-d₆, 400 MHz) δ 7.82 (d, J=8.8 Hz, 2H), 7.16-7.12 (m, 3H), 7.10-7.06 (m, 2H), 3.98-3.97 (m, 2H), 3.15 (s, 3H), 2.99-2.97 (m, 1H), 2.83-2.67 (m, 6H), 2.23-2.32 (m, 1H), 2.15-2.12 (m, 1H), 2.02-1.92 (m, 2H), 1.78-1.66 (m, 2H), 1.61-1.38 (m, 2H), 1.23-1.10 (m, 1H). MS=434.3 [M+H]⁺. The second eluting isomer of the title compound (Compound 133): ¹H NMR (DMSO-d₆, 400 MHz) δ 7.82 (d, J=8.8 Hz, 2H), 7.17-7.15 (m, 3H), 7.12-7.06 (m, 2H), 3.99-3.95 (m, 2H), 3.15 (s, 3H), 2.96-2.91 (m, 1H), 2.85-2.67 (m, 6H), 2.30-2.16 (m, 2H), 2.08-1.91 (m, 2H), 1.78-1.67 (m, 2H), 1.60-1.44 (m, 2H), 1.26-1.08 (m, 1H). MS=434.3 [M+H]⁺

The following compounds in Table 8 were prepared according to procedures similar to procedures described for Compounds 131-133 using the appropriate starting materials.

TABLE 8

| | | | Exact | | |
|---|---|---|---|---|---|
| | | | Mass | Step 4 | Elution |
| # | Structure | IUPAC Name | [M + H]⁺ | column | order |
| 134 | 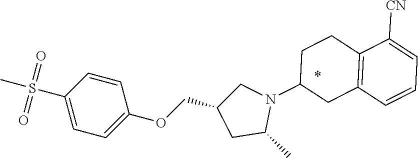 [SMILES: C[C@@H]1C[C@H](COc2ccc(S(C)(=O)=O)cc2)CN1C1CCc2c(C#N)cccc2C1] | (6R or 6S)-6- [(2R,4S)-4-[(4- methanesulfonyl- phenoxy)methyl]-2- methylpyrrolidin- 1-yl]-5,6,7,8- tetrahydronaphthalene- 1-carbonitrile | Calc'd 425.2 Found 425.2 | DAICEL CHIRAL- PAK AD | 1st |
| 135 | 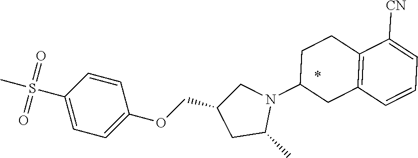 [SMILES: C[C@@H]1C[C@H](COc2ccc(S(C)(=O)=O)cc2)CN1C1CCc2c(C#N)cccc2C1] | (6S or 6S)-6- [(2R,4S)-4-[(4- methanesulfonyl- phenoxy)methyl]-2- methylpyrrolidin-1- yl]-5,6,7,8- tetrahydronaphthalene- 1-carbonitrile | Calc'd 425.2 Found 425.0 | DAICEL CHIRAL- PAK AD | 2nd |
| 136 | 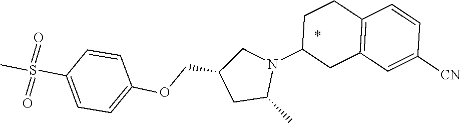 [SMILES: C[C@@H]1C[C@H](COc2ccc(S(C)(=O)=O)cc2)CN1C1CCc2ccc(C#N)cc2C1] | (7S or 7R)-7- [(2R,4S)-4-[(4- methanesulfonyl- phenoxy)methyl]-2- methylpyrrolidin-1- yl]-5,6,7,8- tetrahydronaphthalene- 2-carbonitrile | Calc'd 425.2 Found 425.1 | prep- HPLC Waters Xbridge BEH C18 | 1st |
| 137 | 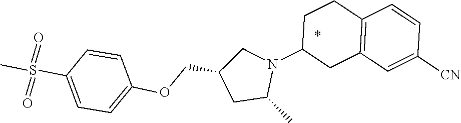 [SMILES: C[C@@H]1C[C@H](COc2ccc(S(C)(=O)=O)cc2)CN1C1CCc2ccc(C#N)cc2C1] | (7R or 7S)-7- [(2R,4S)-4-[(4- methanesulfonyl- phenoxy)methyl]-2- methylpyrrolidin-1- yl]-5,6,7,8- tetrahydronaphthalene- 2-carbonitrile | Calc'd 425.2 Found 425.0 | prep- HPLC Waters Xbridge BEH C18 | 2nd |
| 138 | 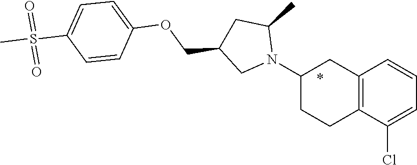 [SMILES: C[C@@H]1C[C@H](COc2ccc(S(C)(=O)=O)cc2)CN1C1CCc2c(Cl)cccc2C1] | (2R,4S)-1-[(2R or 2S)-5-chloro-1,2,3,4- tetrahydronaphthalen- 2-yl]-4-[(4- methanesulfonyl- phenoxy)methyl]-2- methylpyrrolidine | Calc'd 434.1 Found 434.2 | DAICEL CHIRAL- PAK IC | 1st |
| 139 | 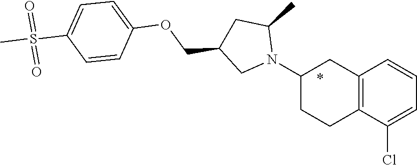 [SMILES: C[C@@H]1C[C@H](COc2ccc(S(C)(=O)=O)cc2)CN1C1CCc2c(Cl)cccc2C1] | (2R,4S)-1-[(2S or 2R)-5-chloro-1,2,3,4- tetrahydronaphthalen- 2-yl]-4-[(4- methanesulfonyl- phenoxy)methyl]-2- methylpyrrolidine | Calc'd 434.1 Found 434.2 | DAICEL CHIRAL- PAK IC | 2nd |

### BIOLOGICAL EXAMPLES

### Example B-1

This example shows that compounds of the present disclosure are able to inhibit calcium transport by APOL1.

A HEK293 clonal cell line was generated to stably express GCaMP6f, a genetically encoded calcium indicator, and inducibly express APOL1 G2 (HEK T-REx/GCaMP6f/APOL1 G2 K6.3). Cells were maintained in the following standard complete medium: DMEM with 4.5 g/L glucose and sodium pyruvate (BioWhittaker, Lonza, BE12-614F), supplemented with 10% FBS Performance Plus (Gibco, 16000044), 1% penicillin-streptomycin (BioWhittaker, DE17-602E), 2 mM ultraglutamine-1 (BioWhittaker cat. BE 17-605/U1), 50 μg/mL Zeocin (InvivoGen, ant-zn), 2.5 μg/mL Blasticidin (InvivoGen, ant-bl-5), and 25 μg/mL Hygromycin (InvivoGen, ant-hg). Standard propagation conditions consisted of plating 9×10⁶, 4×10⁶, 2×10⁶ cells in a T225 flasks to be processed after 2, 3, or 4 days, respectively.

A source plate was generated containing 20 serially diluted compounds in DMSO (duplicate 8-point dose response). Next, 0.8 μL of compounds were transferred from the source plate to a destination plate prefilled with 79.2 μL of Ca²⁺ free Tyrode's buffer (130 mM NaCl, 5 mM KCl, 1 mM MgCl₂, 5 mM NaHCO₃, 20 mM HEPES at pH 7.4). The destination plate was placed on a plate shaker (5 seconds at 2000 rpm) to mix. This process resulted in a destination plate with 2× concentrated compound solutions. All transfer and mixing steps were conducted with an CyBi®-Well dispenser.

Cells were split by gently washing with DPBS (Euroclone, ECB4004L), followed by a 5-minute incubation (humidified, 37° C. with 5% CO₂) with trypsin-EDTA solution (Euroclone, ECB3052D). Detached cells were diluted with standard complete medium without selective agents, counted, and plated in a 384 MTP microplate (GR4332CPL, Twin Helix) (10,000 cells/well in 25 μl/well) using a MATRIX WellMate dispenser. Plates were placed into a humidified incubator (37° C. with 5% CO₂) overnight. The following day, 20 μL of doxycycline (Sigma, D9891) at 20 ng/mL in standard complete medium was added to cells with a CyBi®Drop dispenser to induce APOL1 G2 expression. After a 6-hour incubation (humidified, 37° C. with 5% CO₂), cells were washed 3 times with Ca²⁺ free Tyrode's Buffer (130 mM NaCl, 5 mM KCl, 1 mM MgCl₂, 5 mM NaHCO₃, 20 mM HEPES at pH 7.4) using a BIOTEK Microplate washer, such that 10 μL of buffer remained in each well after the final wash. Assay plates were then stored at room temperature for 10 minutes. Next, 10 μL of diluted compounds were transferred to the assay plate from the 2× compound plate using a CyBi®-Well dispenser. Compound incubation was then carried out at room temperature for 10 minutes. The assay plate was transferred to the FLIProom temperature^{ETRA} and 20 μL of 10 mM Ca²⁺ (final concentration=5 mM) Tyrode's buffer was injected.

Table B1 below summarizes the data from this experiment. Unless otherwise specified, AC₅₀ and values are reported as the geometric mean of at least 2 assay runs on separate days. Each run represents the average of a technical replicate, where each compound was assayed twice in the same plate. A superscript t symbol indicates a value from the average of a technical replicate from a single assay run, where each compound was assayed twice in the same plate.

The AC₅₀ values in Table B1 below reflect the compound's ability to prevent calcium influx by inhibiting APOL1. As shown in the table, compounds of the present disclosure are able to potently inhibit APOL1-mediated calcium transport at sub micromolar concentrations. Compounds in Table B1 are referred to by the corresponding Compound Number in Table 1, which is also referred to in the synthetic examples. When one or more of the numbered compounds are identified by stereochemistry (for example, (R)- or (S)-), the specific stereoisomer for which data is provided in Table B1 may be identified by the elution order of such compound as described in the synthetic examples. To illustrate, Compound 2 is the first-eluting enantiomer of step 4 of Example 1 and Compound 3 is the second-eluting enantiomer of step 4 of Example 1. Further, by way of illustration, Compound 27 is the first-eluting enantiomeric mixture in step 4 of Example 7 and Compound 28 is the second-eluting enantiomeric mixture in step 4 of Example 7. Then, Compound 27 is separated into Compound 29 (the first-eluting enantiomer) and Compound 30 (the second-eluting enantiomer) in Example 8, and Compound 28 is separated into Compound 31 (the first-eluting enantiomer) and Compound 32 (the second-eluting enantiomer) in Example 8. Absolute stereochemistry of such compounds may be identified by methods known in the art.

TABLE B1

| | APOL1 G2 |
|---|---|
| Compound | FLIPR |
| No. | AC₅₀ (μM) |
| 1 | 0.0397 |
| 2 | 1.03 |
| 3 | 0.721 |
| 4 | 1.14 |
| 5 | 0.488 |
| 6 | 0.839 |
| 7 | 1.54 |
| 8 | 1.07 |
| 9 | 1.28 |
| 10 | 1.02 |
| 11 | 1.13 |
| 12 | 0.279 |
| 13 | 0.29 |
| 14 | 0.767 |
| 15 | 1.75 |
| 16 | 1.08 |
| 17 | 0.38 |
| 18 | 0.412 |
| 19 | 0.444 |
| 20 | 0.776 |
| 21 | 0.138 |
| 22 | 1.56 |
| 23 | 0.392 |
| 24 | 0.31 |
| 25 | 0.343 |
| 26 | 0.21 |
| 27 | n/a |
| 28 | n/a |
| 29 | n/a |
| 30 | 0.185 |
| 31 | 0.773 |
| 32 | 0.769 |
| 33 | 0.662 |
| 34 | 0.951 |
| 35 | 0.948 |
| 36 | 0.714 |
| 37 | n/a |
| 38 | 0.59 |
| 39 | 0.554 |
| 40 | 0.534 |
| 41 | 1.18 |
| 42 | 1.03 |
| 43 | 1.36 |
| 44 | 1.38 |
| 45 | 0.998 |
| 46 | 1.61 |
| 47 | 0.926 |
| 48 | 1.13 |
| 49 | 0.558 |
| 50 | 1.07 |
| 51 | 0.439 |
| 52 | 1.02 |
| 53 | 0.999 |
| 54 | 0.857 |
| 55 | 0.876 |
| 56 | 0.714 |
| 57 | 0.802 |
| 58 | 0.581 |
| 59 | 0.794 |
| 60 | 0.477 |
| 61 | 0.698 |
| 62 | 0.462 |
| 63 | 0.188 |
| 64 | 0.287 |
| 65 | 0.346 |
| 66 | 0.621 |
| 67 | 0.337 |
| 68 | 0.708 |
| 69 | 0.346 |
| 70 | 0.236 |
| 71 | 0.492 |
| 72 | 0.183 |
| 73 | 0.351 |
| 74 | 0.479 |
| 75 | 0.419 |
| 76 | 0.25 |
| 77 | 0.189 |
| 78 | 0.114 |
| 79 | n/a |
| 80 | 0.404 |
| 81 | n/a |
| 82 | 0.37 |
| 83 | 0.0946 |
| 84 | 0.182 |
| 85 | 0.221 |
| 86 | 0.117 |
| 87 | n/a |
| 88 | 0.843 |
| 89 | 0.132 |
| 90 | 0.522 |
| 93 | 1.25 |
| 94 | 0.614 |
| 95 | 1.04 |
| 96 | 0.188 |
| 97 | 0.813 |
| 98 | 2.84 |
| 99 | 1.69 |
| 100 | 0.575 |
| 101 | 0.765 |
| 102 | 0.744 |
| 103 | 0.905 |
| 104 | 0.7 |
| 105 | >10.0 |
| 106 | >10.0 |
| 107 | <0.0866 |
| 108 | 0.337 |
| 109 | 0.746 |
| 110 | 0.14 |
| 111 | <0.116 |
| 112 | 0.409 |
| 113 | 0.634 |
| 114 | 0.868 |
| 115 | 0.47 |
| 116 | 1.7 |
| 117 | 0.483 |
| 118 | 1.25 |
| 119 | 2.33 |
| 120 | 0.48 |
| 121 | 1.11 |
| 122 | 1.11 |
| 123 | 0.201 |
| 124 | 4.92 |
| 125 | 2.21 |
| 126 | 1.74 |
| 127 | 2.22 |
| 128 | 1.51 |
| 129 | 4.25 |
| 130 | 1.94 |
| 131 | <0.229 |
| 132 | 0.446 |
| 133 | 1.15 |
| 134 | 0.601 |
| 135 | 0.576 |
| 136 | 0.707 |
| 137 | 0.222 |
| 138 | 1.08 |
| 139 | 1.26 |
| 140 | 0.379 |
| 141 | 0.160 |
| 142 | 0.703 |
| 143 | 0.936 |
| 144 | 0.265 |
| 145 | 1.13 |

### Example B-2

This example shows that the compounds of the present disclosure are able to reduce cell death caused by overexpression of APOL1.

A HEK293 clonal cell line overexpressing APOL1 G2 (HEK293/T-REx APOL1 G2/clone #2) was maintained in 1×DMEM-GlutaMax (Gibco, 10569-010) media with 10% tetracycline-free FBS (Takara Bio USA, 631101), 5 μg/mL Blasticidin (Gibco, A1113903), and 100 μg/mL Zeocin (Invitrogen, R25001) in T75 flasks. In preparation for the assay, this media was aspirated and 2 mL of prewarmed TrypLE Express (Gibco, 12605-010) was added to a flask to detach cells. The flask was then incubated (humidified, 37° C. with 5% CO2) for 3-5 minutes. Afterwards, 8 mL of prewarmed cell assay media (1×DMEM-GlutaMax media with 10% tetracycline-free FBS) was added to the trypsinized cells. The suspension was gently mixed, and cells were counted using a Countess Cell Counting Chamber (Invitrogen). The suspension was diluted using cell assay media to generate a working stock solution (166,667 cells/mL). Using a MultiDrop Combi (Thermo Electron Corp), 30 μL (final cell density=5,000 cells/well) of the working stock solution was dispensed into each well of white 384-well assay ready plates (Nunc™, 164610) containing 6 ng/mL doxycycline, to induce APOL1 expression, and compound. All compounds were plated in a duplicate 8-point dilution series that consisted of 3-fold stepwise dilutions (0.5% DMSO final). Assay plates were incubated (humidified, 37° C. with 5% CO2) for 17 hours. After the incubation, the plates were equilibrated at room temperature for 1 hour. Next, 15 μl of CellTiter-Glo® reagent (Promega, G7570) was added to each well using a MultiDrop Combi. Plates were placed on an orbital shaker (500 rpm) for 5 minutes to induce cell lysis and then incubated at room temperature for 10 minutes. Luminescence was measured on an Envision plate reader. Collaborative Drug Discovery software was utilized for graphing data. Plots were generated using a four parameter logistic curve fit.

Table B2 below provides the results from this experiment. Unless otherwise specified, EC₅₀ values are reported as the geometric mean of at least 2 assay runs on separate days. Each run represents the average of a technical replicate, where each compound was assayed twice in the same plate. A superscript t symbol indicates a value from the average of a technical replicate from a single assay run, where each compound was assayed twice in the same plate. Compounds in Table B2 are referred to by the corresponding Compound Number in Table 1, which is also referred to in the synthetic examples. When one or more of the numbered compounds are identified by stereochemistry (for example, (R)- or (S)-), the specific stereoisomer for which data is provided in Table B2 may be identified by the elution order of such compound as described in the synthetic examples. To illustrate, Compound 2 is the first-eluting enantiomer of step 4 of Example 1 and Compound 3 is the second-eluting enantiomer of step 4 of Example 1. Further, by way of illustration, Compound 27 is the first-eluting enantiomeric mixture in step 4 of Example 7 and Compound 28 is the second-eluting enantiomeric mixture in step 4 of Example 7. Then, Compound 27 is separated into Compound 29 (the first-eluting enantiomer) and Compound 30 (the second-eluting enantiomer) in Example 8, and Compound 28 is separated into Compound 31 (the first-eluting enantiomer) and Compound 32 (the second-eluting enantiomer) in Example 8. Absolute stereochemistry of such compounds may be identified by methods known in the art.

Rescue EC₅₀ values reported in Table B2 below represent the half-maximal effective concentration for reversal of cell death caused by overexpression of APOL1. This example demonstrates that compounds of the present disclosure are able to reduce cell death caused by overexpression of APOL1 at sub micromolar concentration.

TABLE B2

| | APOL1 G2 |
|---|---|
| | HEK293 |
| Compound | Rescue |
| No. | EC₅₀ (μM) |
| 1 | 0.0537 |
| 2 | 0.693 |
| 3 | 0.398 |
| 4 | 2.35 |
| 5 | 0.0476 |
| 6 | 0.661 |
| 7 | 0.195 |
| 8 | 0.15 |
| 9 | 0.516 |
| 10 | 0.744 |
| 11 | 0.137 |
| 12 | 0.0992 |
| 13 | 0.0414 |
| 14 | 0.544 |
| 15 | 1.83 |
| 16 | 0.712 |
| 17 | 0.527 |
| 18 | 0.262 |
| 19 | 0.64 |
| 20 | 1.12 |
| 21 | 0.28 |
| 22 | >18.8 |
| 23 | 0.309 |
| 24 | 0.41 |
| 25 | 1.01 |
| 26 | 0.276 |
| 27 | 0.145 |
| 28 | 0.401 |
| 29 | 0.0584 |
| 30 | 0.0384 |
| 31 | 0.234 |
| 32 | 0.353 |
| 33 | 0.133 |
| 34 | 0.0869 |
| 35 | 0.256 |
| 36 | 0.0363 |
| 37 | 0.769 |
| 38 | 0.065 |
| 39 | 0.121 |
| 40 | 0.0746 |
| 41 | 0.0628 |
| 42 | 0.227 |
| 43 | 0.35 |
| 44 | 0.312 |
| 45 | 0.3 |
| 46 | 0.418 |
| 47 | >3.34 |
| 48 | 0.398 |
| 49 | 0.193 |
| 50 | 0.346 |
| 51 | 0.128 |
| 52 | 0.0869 |
| 53 | 0.279 |
| 54 | 0.0548 |
| 55 | 0.24 |
| 56 | 0.185 |
| 57 | 0.266 |
| 58 | 0.0435 |
| 59 | 0.38 |
| 60 | 0.037 |
| 61 | 0.0633 |
| 62 | 0.201 |
| 63 | 0.0248 |
| 64 | 0.0509 |
| 65 | 0.0498 |
| 66 | 0.0768 |
| 67 | 0.0503 |
| 68 | 0.366 |
| 69 | 0.377 |
| 70 | 0.342 |
| 71 | 0.287 |
| 72 | 0.217 |
| 73 | 0.375 |
| 74 | 0.665 |
| 75 | 0.0706 |
| 76 | 0.222 |
| 77 | 0.176 |
| 78 | 0.0258 |
| 79 | 0.334 |
| 80 | 0.202 |
| 81 | 0.144 |
| 82 | 0.165 |
| 83 | 0.0943 |
| 84 | 0.0286 |
| 85 | 0.186 |
| 86 | 0.0204 |
| 87 | 0.185 |
| 88 | 0.215 |
| 89 | 0.0822 |
| 90 | 0.075 |
| 91 | 1.61 |
| 92 | 2.07 |
| 93 | 1.67 |
| 94 | 0.103 |
| 95 | 1.14 |
| 96 | 0.0277 |
| 97 | 2.1 |
| 98 | >10.0 |
| 99 | 0.252 |
| 100 | 0.948 |
| 101 | >10.0 |
| 102 | >10.0 |
| 103 | 0.655 |
| 104 | 1.01 |
| 105 | >10.0 |
| 106 | >10.0 |
| 107 | 0.245 |
| 108 | 0.664 |
| 109 | 0.224 |
| 110 | 0.0932 |
| 111 | 0.137 |
| 112 | 0.296 |
| 113 | 0.726 |
| 114 | 0.295 |
| 115 | 1.83 |
| 116 | >10.0 |
| 117 | 0.755 |
| 118 | >10.0 |
| 119 | 0.633 |
| 120 | 1.04 |
| 121 | >10.0 |
| 122 | 0.789 |
| 123 | 0.393 |
| 124 | 5.31 |
| 125 | 0.449 |
| 126 | 2.76 |
| 127 | >10.0 |
| 128 | 0.711 |
| 129 | 2.14 |
| 130 | 0.303 |
| 131 | 0.994 |
| 132 | 0.924 |
| 133 | 1.91 |
| 134 | 0.292 |
| 135 | 0.0842 |
| 136 | 0.704 |
| 137 | 0.0358 |
| 138 | 0.149 |
| 139 | 0.26 |
| 140 | 0.756 |
| 141 | 0.234 |
| 142 | 0.231 |
| 143 | 1.20 |
| 144 | 0.289 |
| 145 | 1.77 |

All publications, patent applications, patents, and other references mentioned herein are expressly incorporated by reference in their entireties, to the same extent as if each were incorporated by reference individually.

It is to be understood that, while the disclosure has been described in conjunction with the above embodiments, the foregoing description and examples are intended to illustrate and not limit the scope of the disclosure. Other aspects, advantages and modifications within the scope of the disclosure will be apparent to those skilled in the art to which the disclosure pertains.

What is claimed is:

1. A compound of formula (A): 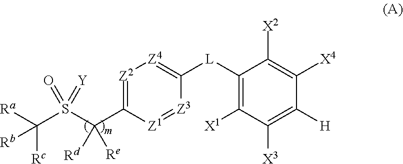
or a stereoisomer or a tautomer thereof, or a pharmaceutically acceptable salt of any of the foregoing, wherein:
m is 0 or 1;
Y is O or —N(C₁₋₆alkyl), wherein the C₁₋₆alkyl of the —N(C₁₋₆alkyl) is optionally substituted with one or more R^{g} substituents;
Z¹, Z², Z³, and Z⁴ are, independently of each other, —N—, —CH— or —C(R^{f})—;
R^{a}, R^{b}, and R^{c} are each independently H, C₁₋₆alkyl, C₃₋₆cycloalkyl, or 3-8 membered heterocycle wherein,
the C₁₋₆alkyl of R^{a}, R^{b}, or R^{c} is optionally substituted with one or more R^{h} substituents, and
the 3-8 membered heterocycle of R^{a}, R^{b}, or R^{c} is optionally substituted with one or more Rⁱ substituents,
or any two of R^{a}, R^{b}, and R^{c} are taken together with the atoms to which they are attached to form a C₃₋₆cycloalkyl or a 3-8 membered heterocycle, wherein the 3-8 membered heterocycle is optionally substituted with one or more Rⁱ substituents, and the other of R^{a}, R^{b}, and R^{c} is H or C₁₋₆alkyl, C₃₋₆cycloalkyl, or 3-8 membered heterocycle wherein,
the C₁₋₆alkyl of R^{a}, R^{b}, or R^{c} is optionally substituted with one or more R^{h} substituents, and
the 3-8 membered heterocycle of R^{a}, R^{b}, or R^{c} is optionally substituted with one or more R¹ substituents;
R^{d} and Rare each independently H or C₁₋₆alkyl,
or R^{d} and R^{e} are taken together with the atoms to which they are attached to form a C₃₋₆cycloalkyl or a 3-6 membered heterocycle;
R^{f} is, independently at each occurrence, —CN, halo, C₁₋₆alkyl, C₁₋₆alkoxy, or —N(R^{j})₂, wherein the C₁₋₆alkyl of R^{f} is optionally substituted with one or more halo;
R^{g} is, independently at each occurrence, —S(O)₂C₁₋₆alkyl;
R^{h} is, independently at each occurrence, —OH, C₁₋₆alkoxy, —N(R^{j})₂, C(O)R^{k}, or —S(O)₂C₁₋₆alkyl;
Rⁱ is, independently at each occurrence, oxo, C₁₋₆alkyl, or C(O)R^{k};
R^{j} is independently at each occurrence H, C₁₋₆alkyl or C(O)C₁₋₆alkyl;
R^{k} is, independently at each occurrence C₁₋₆alkyl or C₁₋₆alkoxy;
L is selected from the group consisting of: 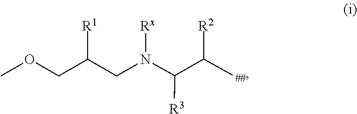
wherein
R^{x} and R¹ are taken together with the atoms to which they are attached to form a 3-8 membered heterocycle, wherein the 3-8 membered heterocycle is substituted with n independently selected R^{m} substituents, wherein n is an integer from 0-6, and R^{m} is C₁₋₆alkyl, or C(O)C₁₋₆alkyl, wherein the C₁₋₆alkyl is optionally substituted with one or more Rⁿ substituents, and Rⁿ is, independently at each occurrence, —OH;
R² is H, —OH, or C₁₋₆alkyl, wherein the C₁₋₆alkyl of R² is optionally substituted with one or more OH; and
R³ is H or C₁₋₆alkyl, wherein the C₁₋₆alkyl of R³ is optionally substituted with one or more OH;
provided that when L is (i), either:
(1) m is 1,
(2) at least one of Z¹, Z², Z³, and Z⁴ is —N— or —C(R^{f})—,
(3) R³ is other than H,
(4) at least one of R^{a}, R^{b}, and R^{c} is heterocycle, or
(5) at least one of R^{g}, Rⁱ, R^{j}, R^{k}, and Rⁿ is present; 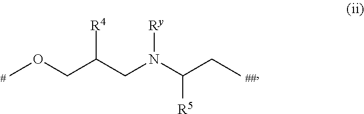
wherein
R^{y}, R⁴, and R⁵ are taken together with the atoms to which they are attached to form a 8-20 membered bicyclic heterocycle, wherein the 8-20 membered bicyclic heterocycle is substituted with n independently selected R^{m} substituents, wherein n is an integer from 0-6, and R^{m} is C₁₋₆alkyl, or C(O)C₁₋₆alkyl, wherein the C₁₋₆alkyl is optionally substituted with one or more Rⁿ substituents, and Rⁿ is, independently at each occurrence, —OH; and 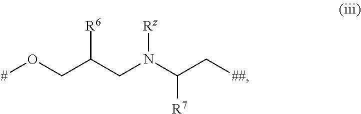
wherein
R^{z} and R⁶ are taken together with the atoms to which they are attached to form a 3-8 membered heterocycleheterocycle substituted with n independently selected R^{m} substituents, wherein n is an integer from 0-6, and R^{m} is C₁₋₆alkyl or C(O)C₁₋₆alkyl, wherein the C₁₋₆ alkyl is optionally substituted with one or more Rⁿ substituents, and Rⁿ is, independently at each occurrence, —OH; and
R⁷ is taken, together with one of X¹ and X² and the atoms to which they are attached, to form a C₄₋₈cycloalkyl;
wherein, for each of (i)-(iii), # denotes the point of attachment to the ring bearing moieties moieties Z¹-Z⁴ and ## denotes the point of attachment to the phenyl ring bearing moieties X¹-X⁴;
X¹, and X² are, independently of each other, H, halo, —CN, C₁₋₆alkyl, C₁₋₆alkoxy, or SF₅, wherein the C₁₋₆alkyl or C₁₋₆alkoxy is optionally substituted with one or more halo,
or one of X¹ and X² is taken together with R⁷ and the atoms to which it is attached to form a C₄₋₈cycloalkyl, and the other of X¹ or X² is H, halo, —CN, C₁₋₆alkyl, C₁₋₆alkoxy, or SF₅, wherein the C₁₋₆alkyl or C₁₋₆alkoxy is optionally substituted with one or more halo; and
X³, and X⁴ are, independently of each other, H, halo, —CN, C₁₋₆alkyl, C₁₋₆alkoxy, or SF₅, wherein the C₁₋₆alkyl or C₁₋₆alkoxy is optionally substituted with one or more halo.

2. The compound of claim 1, wherein the compound is a compound of formula (I): 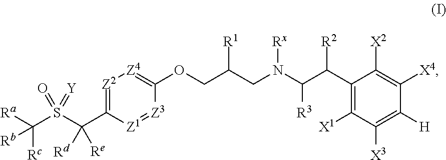
or a stereoisomer or a tautomer thereof, or a pharmaceutically acceptable salt of any of the foregoing.

3. The compound of claim 1 or claim 2, wherein the compound is a compound of formula (I-A): 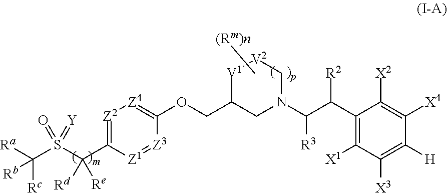
or a stereoisomer or a tautomer thereof, or a pharmaceutically acceptable salt of any of the foregoing, wherein
p is 0, 1, or 2; and
V¹ and V² are each independently —CH₂—, —NH—, or —O—.

4. The compound of any one of claims 1 to 3, or a stereoisomer or tautomer thereof, or a pharmaceutically acceptable salt of any of the foregoing, wherein the compound is a compound of formula (I-A1): 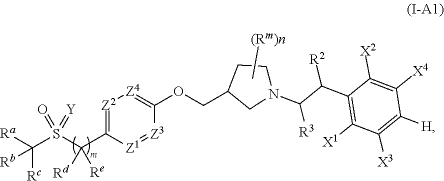
or a stereoisomer or a tautomer thereof, or a pharmaceutically acceptable salt of any of the foregoing.

5. The compound of claim 4, or a stereoisomer or tautomer thereof, or a pharmaceutically acceptable salt of any of the foregoing, wherein n is 0.

6. The compound of claim 4, or a stereoisomer or tautomer thereof, or a pharmaceutically acceptable salt of any of the foregoing, wherein n is 1.

7. The compound of claim 6, or a stereoisomer or tautomer thereof, or a pharmaceutically acceptable salt of any of the foregoing, wherein R^{m} is, independently at each occurrence, C₁₋₆alkyl, wherein the C₁₋₆ alkyl is optionally substituted with one or more —OH.

8. The compound of claim 6 or claim 7, or a stereoisomer or tautomer thereof, or a pharmaceutically acceptable salt of any of the foregoing, wherein R^{m} is, independently at each occurrence, CH₃, or CH₂OH.

9. The compound of any one of claims 1 to 3, wherein the compound is a compound of formula (I-A2): 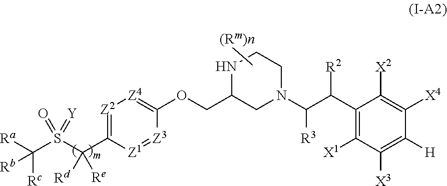
or a stereoisomer or a tautomer thereof, or a pharmaceutically acceptable salt of any of the foregoing.

10. The compound of claim 9, or a stereoisomer or tautomer thereof, or a pharmaceutically acceptable salt of any of the foregoing, wherein n is 0.

11. The compound of claim 1, wherein the compound is a compound of formula (II): 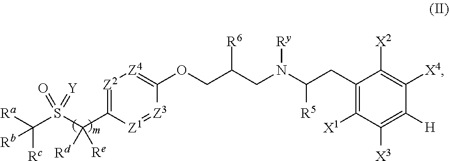
or a stereoisomer or a tautomer thereof, or a pharmaceutically acceptable salt of any of the foregoing.

12. The compound of claim 1 or claim 11, wherein the compound is a compound of formula (II-A): 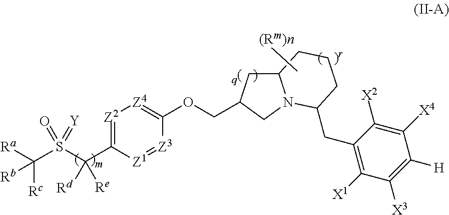
or a stereoisomer or a tautomer thereof, or a pharmaceutically acceptable salt of any of the foregoing, wherein
q is 1, or 2 and
r is 0 or 1.

13. The compound of claim 12, or a stereoisomer or tautomer thereof, or a pharmaceutically acceptable salt of any of the foregoing, wherein each of q and r is 1.

14. The compound of claim 12, or claim 13, or a stereoisomer or tautomer thereof, or a pharmaceutically acceptable salt of any of the foregoing, wherein n is 0.

15. The compound of claim 1, wherein the compound is a compound of formula (III): 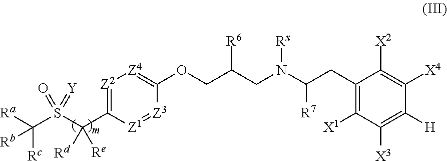
or a stereoisomer or a tautomer thereof, or a pharmaceutically acceptable salt of any of the foregoing.

16. The compound of claim 1 or claim 15, or a stereoisomer or tautomer thereof, wherein the compound is a compound of formula (III-A): 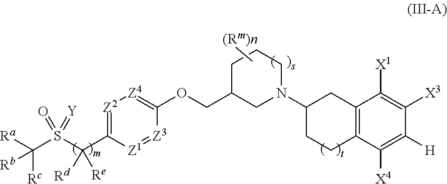
or a stereoisomer or a tautomer thereof, or a pharmaceutically acceptable salt of any of the foregoing, wherein
s is 0, 1, or 2 and
t is 0 or 1.

17. The compound of any one of claims 1, 15, or 16, wherein the compound is a compound of formula (III-A2): 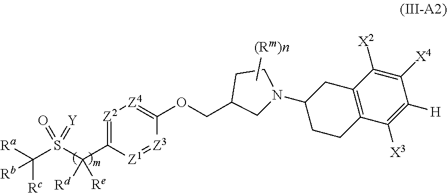
or a stereoisomer or a tautomer thereof, or a pharmaceutically acceptable salt of any of the foregoing.

18. The compound of claim 17, or a stereoisomer or tautomer thereof, or a pharmaceutically acceptable salt of any of the foregoing, wherein n is 1.

19. The compound of claim 17 or claim 18, or a stereoisomer or tautomer thereof, or a pharmaceutically acceptable salt of any of the foregoing, wherein each R^{m} is, independently at each occurrence, C₁₋₆alkyl.

20. The compound of any one of claims 17 to 19, or a stereoisomer or tautomer thereof, or a pharmaceutically acceptable salt of any of the foregoing, wherein R^{m} is, independently at each occurrence, —CH₃.

21. The compound of any one of claims 1, 15 or 16, wherein the compound is a compound of formula (III-A3): 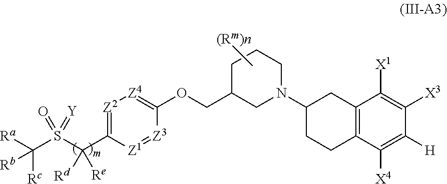
or a stereoisomer or a tautomer thereof, or a pharmaceutically acceptable salt of any of the foregoing.

22. The compound of claim 21, or a stereoisomer or tautomer thereof, or a pharmaceutically acceptable salt of any of the foregoing, wherein n is 0.

23. The compound of any one of claims 1 to 22, or a stereoisomer or tautomer thereof, wherein the compound is a compound of formula (B): 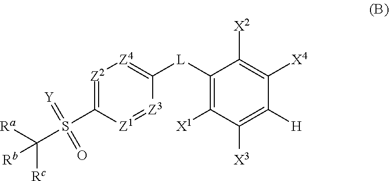
or a stereoisomer or a tautomer thereof, or a pharmaceutically acceptable salt of any of the foregoing.

24. The compound of any one of claims 1 to 23, wherein the compound is a compound of formula (B-2): 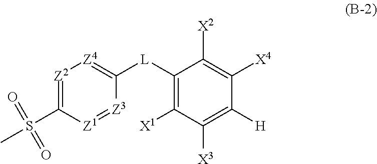
or a stereoisomer or a tautomer thereof, or a pharmaceutically acceptable salt of any of the foregoing.

25. The compound of any one of claims 1 to 23, wherein the compound is a compound of formula (B4): 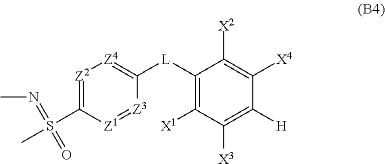
or a stereoisomer or a tautomer thereof, or a pharmaceutically acceptable salt of any of the foregoing.

26. The compound of any one of claims 1 to 22, wherein the compound is a compound of formula (C): 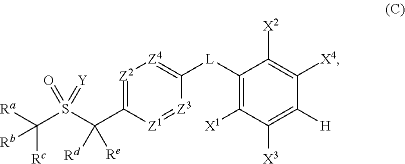
or a stereoisomer or a tautomer thereof, or a pharmaceutically acceptable salt of any of the foregoing.

27. The compound of any one of claims 1 to 26, wherein the ring bearing Z¹, Z², Z³, and Z⁴ is selected from the group consisting of

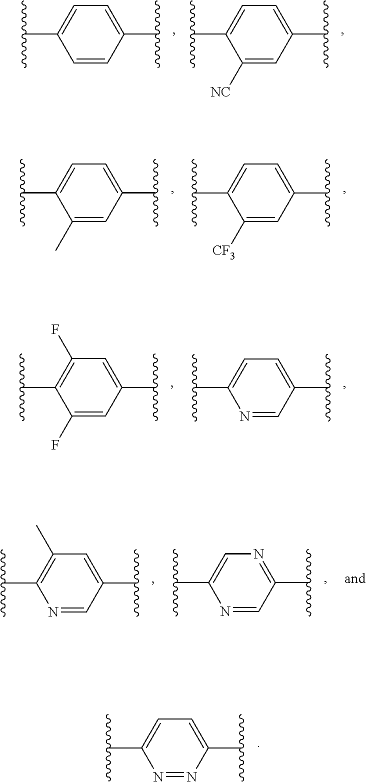

28. The compound of any one of claims 1 to 26, wherein the compound is selected from the group consisting of co compound of claim 1, wherein the compound is a compound of formula (E-Ia), (E-IIa), (E-IIIa), (E-IVa), (E-Va), (E-VIa), or (E-VIIa): 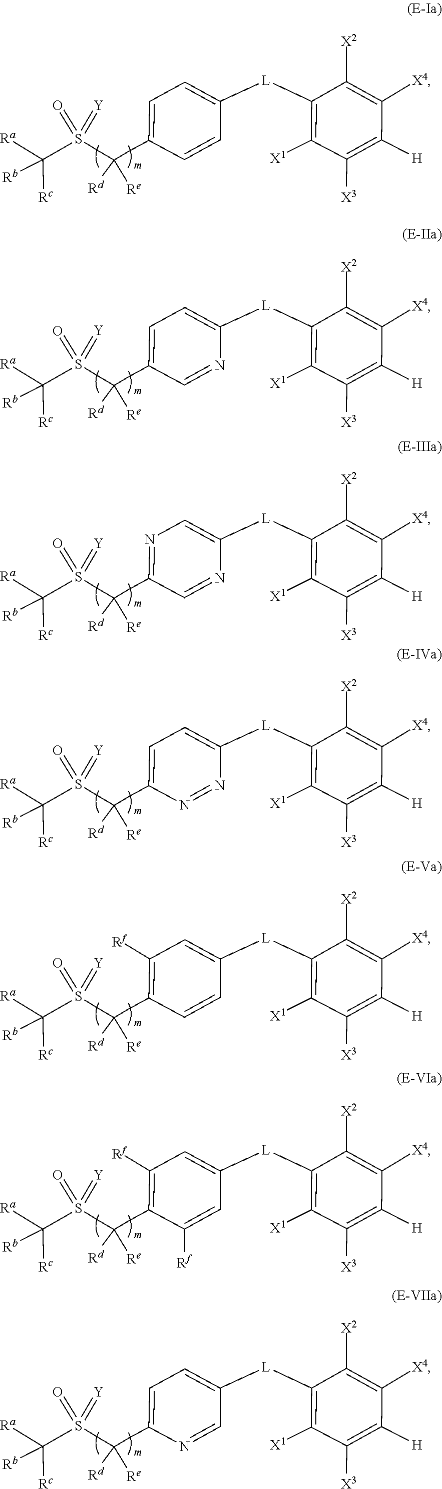
or a stereoisomer or a tautomer thereof, or a pharmaceutically acceptable salt of any of the foregoing.

29. The compound of any one of claims 1 to 28, wherein the compound is selected from the group consisting of compounds 1-12, 14-90, and 93-142 of Table 1, or a stereoisomer or tautomer thereof, or a pharmaceutically acceptable salt of any of the foregoing.

30. A method for preparing a compound of formula (A) as recited in claim 1, or a stereoisomer or tautomer thereof, or a pharmaceutically acceptable salt of any of the foregoing, wherein the method comprises a step of reacting a compound of formula (A-I1): 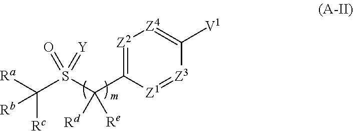
wherein, R^{a}, R^{b}, R^{c}, R^{d}, R^{e}, Z¹, Z², Z³, Z⁴, Y, and m, are as defined for a compound of formula (A); and
V¹ is selected from the group consisting of: 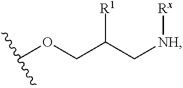
wherein
R^{x} and R¹ are taken together with the atoms to which they are attached to form a 3-8 membered heterocycle, wherein the 3-8 membered heterocycle is substituted with n independently selected R^{m} substituents, wherein n is an integer from 0-6, and R^{m} is C₁₋₆alkyl, or C(O)C₁₋₆alkyl, wherein the C₁₋₆alkyl is optionally substituted with one or more Rⁿ substituents, and Rⁿ is, independently at each occurrence, —OH; and 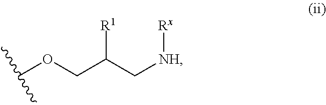
wherein
R^{z} and R⁶ are taken together with the atoms to which they are attached to form a 3-8 membered heterocycleheterocycle substituted with n independently selected R^{m} substituents, wherein n is an integer from 0-6, and R^{m} is C₁₋₆alkyl or C(O)C₁₋₆alkyl, wherein the C₁₋₆ alkyl is optionally substituted with one or more Rⁿ substituents, and Rⁿ is, independently at each occurrence, —OH;
with:
a compound of formula (A-I2): 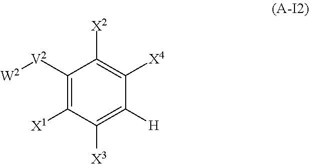
wherein X¹, X², X³, and X⁴ are as defined for a compound of formula (A);
the dashed line represents a single or double bond;
W¹ is oxo, halo or sulfonate ester; and
V² is selected from the group consisting of: 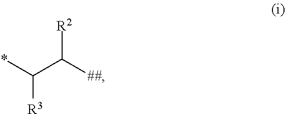
wherein
R² is H, —OH, or C₁₋₆alkyl, wherein the C₁₋₆alkyl of R² is optionally substituted with one or more OH; and
R³ is H or C₁₋₆alkyl, wherein the C₁₋₆alkyl of R³ is optionally substituted with one or more OH;
provided that when V² is (i), either:
(1) m is 1,
(2) at least one of Z¹, Z², Z³, and Z⁴ is —N— or —C(R^{f})—,
(3) R³ is other than H,
(4) at least one of R^{a}, R^{b}, and R^{c} is heterocycle, or
(5) at least one of R^{g}, Rⁱ, R^{j}, R^{k}, and Rⁿ is present; and 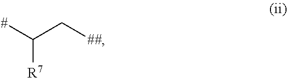
wherein
R⁷ is taken, together with one of X¹ and X² and the atoms to which they are attached, to form a C₄₋₈cycloalkyl;
wherein # denotes the point of attachment to W¹ and ## denotes the point of attachment to the remainder of the molecule.

31. A method for preparing a compound of formula (A) as recited in claim 1, or a stereoisomer or tautomer thereof, or a pharmaceutically acceptable salt of any of the foregoing, wherein the method comprises a step of reacting a compound of formula (A-I3): 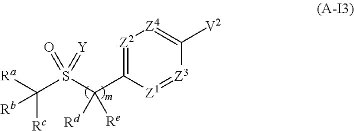
wherein, R^{a}, R^{b}, R^{c}, R^{d}, R^{e}, Z¹, Z², Z³, Z⁴, Y, and m, are as defined for a compound of formula (A); and
V² is halo or OH,
with:
a compound of formula (A-I4): 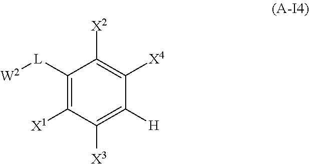
wherein, X¹, X², X³, X⁴, and L are as defined for a compound of formula (A); and
W² is H, or sulfamate;
to give a compound of formula (A) or a stereoisomer or tautomer thereof, or a pharmaceutically acceptable salt of any of the foregoing.

32. A method for preparing a compound of formula (A) as recited in claim 1, or a stereoisomer or tautomer thereof, or a pharmaceutically acceptable salt of any of the foregoing, wherein the method comprises a step of reacting a compound of formula (A-I5): 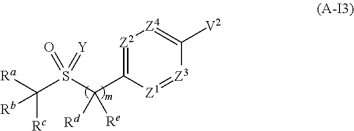
wherein, R^{a}, R^{b}, R^{c}, R^{d}, R^{e}, Z¹, Z², Z³, Z⁴, Y, and m, are as defined for a compound of formula (A); and
V³ is 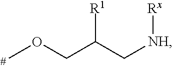
wherein
R^{x} and R¹ are taken together with the atoms to which they are attached to form a 3-8 membered heterocycle, wherein the 3-8 membered heterocycle is substituted with n independently selected R^{m} substituents, wherein n is an integer from 0-6, and R^{m} is C₁₋₆alkyl, or C(O)C₁₋₆alkyl, wherein the C₁₋₆alkyl is optionally substituted with one or more Rⁿ substituents, and Rⁿ is, independently at each occurrence, —OH;
with:
a compound of formula (A-I6): 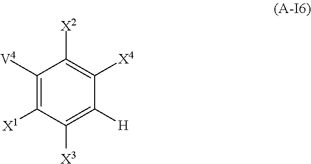
wherein X¹, X², X³, and X⁴ are as defined for a compound of formula (A); and
V⁴ is 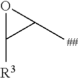
provided that when L is (i), either:
(1) m is 1,
(2) at least one of Z¹, Z², Z³, and Z⁴ is —N— or —C(R^{f})—,
(3) R³ is other than H,
(4) at least one of R^{a}, R^{b}, and R^{c} is heterocycle, or
(5) at least one of R^{g}, Rⁱ, R^{j}, R^{k}, and Rⁿ is present;
to give a compound of formula (A) or a stereoisomer or tautomer thereof, or a pharmaceutically acceptable salt of any of the foregoing.

33. A pharmaceutical composition, comprising (i) a compound of any one of claims 1 to 29, or a stereoisomer or tautomer thereof, or a pharmaceutically acceptable salt of any of the foregoing, and (ii) one or more pharmaceutically acceptable excipients.

34. A method of modulating APOL1 in a cell, comprising exposing the cell to a composition comprising an effective amount of a compound of any one or claims 1 to 29, or a stereoisomer or tautomer thereof, or a pharmaceutically acceptable salt of any of the foregoing, or the pharmaceutical composition of claim 33.

35. A method of inhibiting APOL1 in a cell, comprising exposing the cell to a composition comprising an effective amount of a compound of any one or claims 1 to 29, or a stereoisomer or tautomer thereof, or a pharmaceutically acceptable salt of any of the foregoing, or the pharmaceutical composition of claim 33.

36. A method of treating an APOL1-mediated disease, disorder, or condition in an individual in need thereof, comprising administering to the individual a compound of any one of claims 1 to 29, or a stereoisomer or tautomer thereof, or a pharmaceutically acceptable salt of any of the foregoing, or the pharmaceutical composition of claim 33.

37. The method of claim 36, wherein a therapeutically effective amount of the compound or the pharmaceutical composition is administered.

38. The method of claim 36, or claim 37, wherein the disease, disorder, or condition is a kidney disease.

39. The method of any one of claims 36 to 38, wherein the disease, disorder, or condition is a chronic kidney disease (CKD).

40. The method of claim 36 or claim 37, wherein the disease, disorder, or condition is selected from the group consisting of chronic kidney disease, focal segmental glomerulosclerosis (FSGS), hypertension-attributed kidney disease, human immunodeficiency virus-associated nephropathy (HIVAN), sickle-cell nephropathy, lupus nephritis, diabetic kidney disease, APOL1-associated nephropathy, viral nephropathy, COVID-19 associated nephropathy, preeclampsia, and sepsis.

41. A method of delaying the development of an APOL1-mediated disease, disorder, or condition, comprising administering a compound of any one of claims 1 to 29, or a stereoisomer or tautomer thereof, or a pharmaceutically acceptable salt of any of the foregoing, or the pharmaceutical composition of claim 33, to an individual who is at risk of developing an APOL1-mediated disease, disorder, or condition.

42. The method of claim 41, wherein a therapeutically effective amount of the compound or the pharmaceutical composition is administered.

43. The method of claim 41, or claim 42, wherein the APOL1-mediated disease, disorder, or condition is a kidney disease.

44. The method of any one of claims 41 to 43, wherein the APOL1-mediated disease, disorder, or condition is a chronic kidney disease.

45. The method of claim 41, or claim 42, wherein the APOL1-mediated disease, disorder, or condition is selected from the group consisting of chronic kidney disease, focal segmental glomerulosclerosis (FSGS), hypertension-attributed kidney disease, human immunodeficiency virus-associated nephropathy (HIVAN), sickle-cell nephropathy, lupus nephritis, diabetic kidney disease, APOL1-associated nephropathy, viral nephropathy, COVID-19 associated nephropathy, preeclampsia, and sepsis.

46. The method of any one of claims 36 to 45, wherein the individual has an APOL1 mutation.

47. The method of claim 46, wherein the APOL1 mutation comprises a gain-of-function mutation.

48. A kit, comprising (i) a compound of any one of claims 1 to 29, or a stereoisomer or tautomer thereof, or a pharmaceutically acceptable salt of any of the foregoing, or the pharmaceutical composition of claim 33, and (ii) instructions for use in treating an APOL1-mediated disease, disorder, or condition in an individual in need thereof.

49. The kit of claim 48, wherein the disease, disorder, or condition is a kidney disease.

50. The kit of claim 48 or claim 49, wherein the disease, disorder, or condition is a chronic kidney disease (CKD).

51. The kit of any one of claims 48 to 50, wherein the disease, disorder, or condition is selected from the group consisting of chronic kidney disease, focal segmental glomerulosclerosis (FSGS), hypertension-attributed kidney disease, human immunodeficiency virus-associated nephropathy (HIVAN), sickle-cell nephropathy, lupus nephritis, diabetic kidney disease, APOL1-associated nephropathy, viral nephropathy, COVID-19 associated nephropathy, preeclampsia, and sepsis.

52. The kit of any one of claims 48 to 51, wherein the individual has an APOL1 mutation.

53. The kit of claim 52, wherein the APOL1 mutation comprises a gain-of-function mutation.
